(19) United States
(12) Patent Application Publication
(10) Pub. No.: US 2026/0284202 A1
(43) Pub. Date: Sep. 24, 2026

(54) CONJUGATES OF SAPONIN AND ANTISENSE OLIGONUCLEOTIDES FOR USE IN THE TREATMENT OF NEURODEGENERATIVE DISEASES

(71) Applicant: Sapreme Technologies B.V., Utrecht (NL)

(72) Inventors: Miriam Verena Bujny, Utrecht (NL); Ruben Postel, Utrecht (NL); Guy Hermans, Merelbeke (BE); Ingrid Jolanda de Vries-van Leeuwen, Utrecht (NL)

(73) Assignee: Sapreme Technologies B.V., Utrecht (NL)

(21) Appl. No.: 19/471,747

(22) PCT Filed: Jun. 19, 2024
(86) PCT No.: PCT/EP2024/067069

§ 371 Date: Oct. 2, 2025

(30) Foreign Application Priority Data
Jun. 21, 2023 (NL) 2035136

(51) Int. Cl. A61K 47/54; A61P 25/28; A61P 27/02

(52) U.S. Cl. CPC A61K 47/549; A61P 25/28; A61P 27/02

(57) ABSTRACT

The present invention relates to the fields of therapy and drug delivery. More specifically, disclosed herein are therapeutic methods and pharmaceutical compositions for treating disorders of blood-tissue barrier-shielded organs that harbour substantive populations of post-mitotic neurons, such as the organs derived from neural tube that include the central nervous system and the eye. The disclosed methods and compositions involve localised administration into such organs of an effector component that targets an intracellular biological target, in combination with a saponin component that enhances effective uptake of the effector component into the cells and/or that enhances effective routing of the effector component inside the cells where the biological target is present. For example, the effector component can be an oligonucleotide therapeutic that is targeting a gene product associated with the disorder of the CNS and/or of the eye. Due to cellular uptake-stimulating and/or endosomal escape enhancing effects of the saponin component, the presented herein neuropharmaceutical and ophthalmic compositions for the localised administration into the CNS and/or into the eye, respectively, can be formulated with lower concentrations of the effector component and/or at lower volumes, which confers safety-benefits to the neurons and to the comfort of the patient.

### TECHNICAL FIELD

The present invention relates to the fields of therapy and drug delivery. More specifically, disclosed herein are therapeutic methods and pharmaceutical compositions for treating disorders of blood-tissue barrier-shielded organs that harbour substantive populations of post-mitotic neurons, such as the organs derived from neural tube that include the central nervous system and the eye. The disclosed methods and compositions involve localised administration into such organs of an effector component that targets an intracellular biological target, in combination with a saponin component that enhances effective uptake of the effector component into the cells and/or that enhances effective routing of the effector component inside the cells where the biological target is present. For example, the effector component can be an oligonucleotide therapeutic that is targeting a gene product associated with the disorder of the CNS and/or of the eye. Due to cellular uptake-stimulating and/or endosomal escape enhancing effects of the saponin component, the presented herein neuropharmaceutical and ophthalmic compositions for the localised administration into the CNS and/or into the eye, respectively, can be formulated with lower concentrations of the effector component and/or at lower volumes, which confers safety-benefits to the neurons and to the comfort of the patient.

### BACKGROUND

Patients with serious neural tissue injuries, including neurodegeneration, paralysis, blindness etc. often require lifelong assistance, which not only puts a tremendous burden on them, but also on their families and the society. Consequently, strategies are required to advance treatments of disorders leading to neuronal damage and affecting organs made of neural tissues.

Neurons are specialised electrically excitable cells whose function is to send, receive, and transmit electrochemical signals across different zones of the body. They are highly vulnerable cells with a very limited regenerative capacity and extreme susceptibility to various kinds of stresses, toxic substances, and injuries (Di Virgilio, 2006). Their survival practically depends on other cell types termed glial cells that surround neurons as part of the neural tissue. Due to this vulnerability, in addition to being hidden behind the bony structures of the axial skeleton, most notably our 7 mm thick skull, the body organs harbouring the neural tissue are equipped with a number of adaptational mechanisms and anatomically protective structures, which are believed to exist to protect the post mitotic neurons from stress and damage. This in particular holds true for interrelated organs that are derived from an embryonic precursor structure known as the neural tube, which are the brain, the spinal cord, and the eye (Marchesi et al., 2021).

The substantial majority of the neurons are concentrated within the central nervous system (the CNS), with a large population being also present in the retina of the eye, which is formed from the neural tissue and connects directly to the brain via the optic nerve (Purves et al., 2001). The eye's status of an anatomical extension of the brain is not only reflected by the presence of the neurons and the glia in both of these organs, but also by multiple evident parallels between their vasculature properties and immune response (Nguyen et al., 2021), namely the presence of the blood-tissue barrier and the so-called immune privilege.

The eye and the other CNS organs, i.e. the brain (with its stem) and the spinal cord, are considered to be immune-privileged organs in which the adaptive immunity and inflammation are highly controlled. This feature is believed to exist to protect the sensitive neural cells from potential immune response-mediated injury and death (Hong and Kaer, 1999). Then, the blood-tissue barrier enables this protection by providing an anatomical interface between the capillaries and the cells of these organs and their other components (e.g. cerebrospinal fluid [CSF] of the CNS, vitreous body of the eye, etc.), which interface not only keeps away the infectious agents but also limits the exchange of substances over the capillary walls to defend the neurons from dangerous body metabolites and toxins that may be present in the blood. The barriers of the CNS and of the eye are termed blood-brain barrier (BBB), blood-spinal cord barrier, and blood-ocular barrier including blood-retinal barrier, respectively. Their presence and integrity are vital to neural protection, but in case a pathological process has already begun in the CNS or the eye, it also enormously limits pharmacological intervention options, because most drug types cannot pass the blood-tissue barriers, or to do so require elaborate modifications (Mitusova et al., 2022).

Perhaps, the currently most promising drug types for treating CNS disorders and a number of pathologies of the eye are the nucleic acid therapeutics. They are based on chained polymers of DNA or RNA with frequent synthetic modifications (an overview can be found in Roberts et al., 2020). Their chains can include entire transcripts or mutation repair sequences for gene therapy (Ghoraba et al., 2022) but, more frequently, shorter polymers are used (oligomers, for simplicity termed oligonucleotides) to modulate gene expression when delivered into a diseased cell. Typical examples include antisense oligonucleotides (ASOs, AONs) and RNA interference oligotherapeutics like siRNAs and microRNAs (Roberts et al., 2020). Because of their enormous potential, many are now under development or in clinical trials (Moumné et al., 2022), and several are FDA-approved for the CNS and ophthalmic disorders. To mention a few, these include a splice-modulating 2′-O-MOE ASO nusinersen (Spinraza®) for the treatment of spinal muscular atrophy, a PS-DNA ASO fomivirsen (Vitravene®) against intraocular cytomegalovirus retinitis infection, and a synthetic DNA aptamer pegatinib (Macugen®) against neovascular age-related macular degeneration (AMD).

However, even the smallest oligonucleotide therapeutics are not able to pass the blood-tissue barriers and require local administration into the CNS or the eye directly to avoid the barriers. For example, Spinraza® is administered intrathecally into the CSF, while both Vitravene® and Macugen® require repeated intravitreal injections into the eye. Such interventions are naturally uncomfortable for the patient, partially due to the pressure build up in and around the organ due to piercing and introduction of the therapeutic volume. This can cause side-effects like nausea and tissue layers detachments. Even more importantly, these interventions come at potentially serious risks of neural toxicities from the formulation components and, considering the limited immune defences of these organs due to their immune privilege, also of highly detrimental infections, which increase with repeated administrations.

It does not get better in the context of intraocular injections. Firstly, they are usually painful and have the major drawback of being able to deliver only a small quantity of the therapeutic agent due to limited volume that the eye or its surrounding tissues can accept. Furthermore, the larger the injected volume, the greater the pressure build-up around or inside the eye, which causes further discomfort and imposes further constrains on the volume and therapeutic dose that can be injected. This necessitates repeating of the treatment, which is complicated as not only these injections are painful, they also have to be administered in the clinic by trained personnel, sometimes in the operating theatre, and are also associated with increased risk of eye infection and damage.

For example, repeated intravitreal injections are known to cause retinal detachments, subconjunctival haemorrhage, retinal toxicity, corneal abrasion, endophthalmitis, and potentially devastating intraocular infections (Falavarjani et al., 2013). Consequently, to avoid frequent re-injecting, it is a common practice to maximise the dose per treatment, which, in turn, not only increases intraocular pressure (IOP)-related discomfort, but also potential dose-related toxicity effects (deVries et al., 2020). The occurrence of such or similar adverse events had caused several nucleic acid-based therapeutic agents to fail in clinical trials, including e.g. VEGF-A targeting siRNA bevasiranib.

Given not many treatment alternatives for the most prevalent eye diseases, patients are expected to receive the ocular injections all their lives. Consequently, there exists a need to reduce the frequency of the injections as much as possible, possibly by improving the bioavailability. At the same time, there exists a need to make the injections more bearable and safer for the patients. Consequently, in addition to the need of increased bioavailability, which could be achieved by increasing the dose, there also exist a contradictory need to keep the volume of the injectable formulation as low as possible.

Consequently, there exist a need to reduce the frequency and distress associated with the invasive local treatments, possibly by increasing of the efficacy, bioavailability, and long term-effects of nucleic acid therapeutics, while lowering the injectable volumes. This is not straightforward, especially that even when administered locally and delivered or targeted to the cells or site of the desired therapeutic action, oligonucleotide therapeutics are known to suffer from an extremely inefficient cellular uptake, which prevents them from effectively reaching the cytoplasmic and/or the nuclear intracellular compartments where they are supposed to act upon their generic targets. This perhaps is best reflected by the quantitative estimation that less than 2% from a therapeutic dose of an oligonucleotide drug becomes correctly internalised, possibly due to an estimated 98% thereof being retained within the endosomal compartment and eventually degraded in the lysosomes (Gilleron et al., 2013).

This inefficient cellular uptake results in administration of higher doses, frequently at higher volumes, both of which increase the risks of off-target effects, potentially of cytotoxic nature. Furthermore, higher doses of nucleic acids increase the risk of stimulating the immune system, even regardless of the organs' immune privilege status, which was e.g. observed in response to ASOs injections into a mouse brain (Toonen et al., 2018).

In conclusion, improved compositions of nucleic acid therapeutics are needed for the treatment of neuron-rich tissues in a safe and sustainable manner. Strategies are hence required for delivering nucleic acid therapeutics in a more efficient way, allowing to lower their doses while at the same time increasing the interval between the invasive administrations. Importantly, these strategies cannot cause neurotoxicity and have to result in an increased cellular uptake of the therapeutic without inducing neurotoxic stress or immunostimulatory effects.

It is an objective of the present disclosure to provide such compositions and strategies as explained below.

### SUMMARY

The present disclosure relates to the finding that penta-cyclic triterpene saponins comprising an aglycone core of 12,13-dehydrooleanane type are safe for direct local administration and enhancement of nucleic acid therapeutics in sensitive neuron-rich organs, which are protected by blood-tissue barrier and the apparent immune privilege, notably including the organs derived from the neural tube such as the CNS organs and the eye.

It was interestingly observed and is herewith further demonstrated with in vivo mouse data, that at the tested concentrations, the penta-cyclic triterpene saponins comprising an aglycone core of 12,13-dehydrooleanane type were capable of potentiating the therapeutic effects of the co-administered oligonucleotide therapeutic at doses substantially lower than its usual reference dose, while at the same time not exhibiting any visible neurotoxic effects in the mouse brain.

Based on this finding, provided herein are methods and compositions involving localised administration into neuron-rich organs of an effector component that targets an intracellular biological target, in combination with a saponin component made with this particular saponin type and which enhances effective uptake of the effector component into the cells where the biological target is present. For example, the effector component can be an oligonucleotide therapeutic that is targeting a gene product associated with the disorder of the CNS and/or of the eye. Due to the cellular uptake-stimulating effects of the saponin component, the presented herein neuropharmaceutical and ophthalmic compositions for the localised administration into the CNS and/or into the eye, respectively, can be formulated with lower concentrations of the effector component and/or at lower volumes, which confers safety-benefits to the neurons and to the comfort of the patient.

Saponins of this specific type were characterised and reported in e.g. WO2020126620 as possessing an endosomal-escape enhancing (EEE) activity towards various antibody-drug conjugates (ADCs) in several cancer cells, where they exhibit promising effects in enhancing known cancer treatments. Cancer cells, however, are robust cells, which are targets for cell killing therapeutic strategies. In contrast to the known cancer-targeting treatments, local delivery into sensitive neuron-rich organs requires that the therapeutic composition is safe or at least not cytotoxic to the vulnerable neuronal cells present in these organs, regardless if the given therapeutic composition is targeting the neurons directly or is aimed at targeting other cell types in the same neural tissue compartment.

As disclosed herein, these specific saponins appear safe to neuronal structures of the brain and of the eye following direct localised administration into any one of these organs by injection, while at the same time retaining the ability to enhance nucleic acids delivery. Notably, this enhancement is demonstrated herein in mature and differentiated cells of the brain. Notably, such differentiated cells are very different and more difficult to “transfect” with nucleic acids than cultured immortalised and much more metabolically-active cell lines disclosed in WO2020126620. Consequently, the presented herein enhancement of therapeutic effects of the nucleic acids administered at lower than nominal doses in vivo to the neuron-rich organs in the presence of the saponins, demonstrated the potential of the disclosed herein approaches for developing improved therapeutics compositions for nucleic acid-mediated treatments of neuron-rich organs, such as the brain and the eye.

In line with the above, for the first time disclosed herein is a saponin component for use in a therapeutic method of treating a subject suffering from a disorder of a (n immune-privileged) neuron-rich organ comprising vasculature with blood-tissue barrier properties and preferably being an organ derived from neural tube, the method comprising administration to the subject of:
the saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration is performed directly into the organ or into a body cavity or fluid space that is in (non-blood-tissue barrier obstructed) communication with the cells of the organ

Advantageously, the immune-privileged neuron-rich organs comprising vasculature with blood-tissue barrier properties are selected from the eye and the organs of the CNS, comprising the brain and the spinal cord.

In a related aspect, further disclosed herein are related to the above pharmaceutical compositions for treating disorders of the neuron-rich organs, in particular being neuropharmaceutical compositions for treating disorders within the CNS, for example of the brain; or alternatively being ophthalmic compositions for treating disorders of the eye.

Hence, in detail, disclosed herein further is a pharmaceutical composition, for use in a treatment of a disorder of a (n immune-privileged) neuron-rich organ comprising vasculature with blood-tissue barrier properties, the pharmaceutical composition comprising:
a saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration of the pharmaceutical composition is performed directly into the organ or into a body cavity or fluid space that is in (non-blood-tissue barrier obstructed) communication with the cells of the organ.

In a further and related aspect, provided herein is a method of treatment of a subject suffering from of a disorder of a (n immune-privileged) neuron-rich organ comprising vasculature with blood-tissue barrier properties, the method of treatment comprising:
administration to said subject of
   a saponin component comprising a penta-cyclic triterpene saponin comprising an aglycone core of 12,13-dehydrooleanane type, and
   an effector component comprising a nucleic acid therapeutic intended to be delivered into one or more cells of the organ,
and wherein the administration is performed directly into the organ or into a body cavity or fluid space that is in (non-blood-tissue barrier obstructed) communication with the cells of the organ.

And further, provided herein is use of a saponin component in the manufacture of a medicament for use in a therapeutic method of treatment of a subject suffering from of a disorder of a (n immune-privileged) neuron-rich organ comprising vasculature with blood-tissue barrier properties, wherein the therapeutic method of treatment comprises administration to said subject of
a saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration is performed directly into the organ or into a body cavity or fluid space that is in (non-blood-tissue barrier obstructed) communication with the cells of the organ.

In sum, to address the drawbacks of the prior art, the present disclosure provides saponin components and pharmaceutical compositions for use in the treatment of disorders of neuron-rich organs, the compositions combining a saponin component comprising a penta-cyclic triterpene saponin comprising an aglycone core of 12,13-dehydrooleanane type, and an effector component comprising a nucleic acid therapeutic intended to be delivered into one or more cells of the organ, and wherein the treatment comprises performing the administration directly into the organ or into a body cavity or fluid space that is in (non-blood-tissue barrier obstructed) communication with the cells of the organ.

Further, it should be noted that it is one of the objectives of the disclosed further herein embodiments to provide a solution to the problem of current nucleic acid therapeutics being less efficacious than desired and to not being sufficiently capable to reach and/or enter into to the diseased cells within the neuron-rich organ following the local administration.

Another one of the objectives of the disclosed embodiments is to provide a solution to the problem of low efficiency of nucleic acid delivery and target engagement, which likely is the cause of the effective nucleic acid concentrations being too low at the target site in the neuron-rich organ following the local administration.

A further one of the objectives of the disclosed embodiments is to provide a solution to any one or more of the problems of insufficient delivery of the required quantity of the nucleic acid therapeutic to the target site of action by local delivery into the neuron-rich organ, their abrogated or suboptimal therapeutic efficacy following said delivery route, as well as their off-target activity and/or undesired adverse effects in or around the administration site.

It is yet another one of the several objectives of the disclosed herein embodiments to provide a solution to the problem of insufficient safety characteristics of the currently existing nucleic acid therapeutics, in particular those relating to adverse effects related to toxicity, discomfort, and/or post application complications, when administered locally to the neuron-rich organ of human patients in need thereof, in particular at side-effect inducing excessive doses.

### Definitions

As used herein the term “neuron-rich organ comprising vasculature with blood-tissue barrier properties” is to be construed as one of the organs that contain substantial populations of neural tissue and vasculature which has blood-tissue barrier properties. As used herein, the term is to be construed as in particular referring to the organs of the central nervous system (CNS; the organs being the brain and the spinal cord) and to the eye, which are organs whose substantial and/or functional parts develop from an embryonic structure termed the neural tube (i.e. are organs derived from neural tube) and which share a lot of anatomical and physiological similarities. For example, all of these organs contain neural cells made of neurons and glia, are considered to be immune-privileged, are supplied by blood through vessels (capillaries) that are highly restrictive to passage of many chemical compounds because of the blood-tissue barrier properties (as their name suggests: blood-brain barrier, spinal cord barrier, and blood-ocular barrier including blood-retinal barrier) and are protected by the axial skeleton structure of the skull and the vertebral column.

As used herein the term “eye” shall be understood as referring to a vertebrate eye, preferably human eye, which is a bilateral spherical organ that houses light-sensitive structures necessary for vision. For the sake of simplicity, as used here, the term eye shall be understood as being synonymous to what anatomically is understood as the “eyeball”, i.e. the complex spherical organ covered by a fibrous layer made by sclera and cornea, and placed inside of a bony cavity (orbit) known as the “eye socket” of a vertebrate skull.

The term “saponin” has its regular scientific meaning and refers to a chemical compound from a group of amphipathic glycosides that comprise one or more hydrophilic glycone moieties (usually arranged in chains containing at least one sugar group, but more frequently containing an often branched glycan chain of several sugar groups), which one or more glycone moieties are covalently bound to a lipophilic aglycone core of steroid or terpenoid structure that is termed sapogenin.

In the context of saponins, the terms “aglycone core”, “sapogenin”, and “aglycone core structure”, “aglycone glycoside core (structure)” are used interchangeably and in line with their scientifically accepted meaning in the field. Namely, these terms refer to the lipophilic part of a saponin, which part has a steroid or terpenoid structure and to which one or more glycone moieties are attached (these glycone moieties are sometimes also referred to as “glycone antennae” or “sugar antennae”).

The term “saccharide chain” or “carbohydrate chain” has its regular scientific meaning and here refers to any of a glycan, a carbohydrate antenna, a single saccharide moiety (monosaccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

As it will be apparent from the present description, a specific group of saponins with an aglycone core of terpenoid structure will form part of the presented herein pharmaceutical compositions and therapeutic methods. Because of this aglycone core structure, the saponins are classified as penta-cyclic triterpene saponins which, in particular, comprise an aglycone core of 12,13-dehydrooleanane type. The chemical structure of this aglycone core type is schematically shown in a saponin presented in Scheme of SAPONIN A (in Detailed Description). Examples of aglycone cores of 12,13-dehydrooleanane type include saponin aglycone cores of quillaic acid and of gypsogenin, which in addition in naturally-occurring forms also contain an aldehyde function at position C-23 of the aglycone core. For example, quillaic acid is the aglycone glycoside core structure for SO1861, SO1832, AG1856.

A saponin may be naturally occurring or non-naturally occurring, e.g. modified during isolation process, partial degradation, chemical modification, or can be partially or entirely synthetic.

Consequently, as used herein, the term “saponin” should be construed as referring to any glycoside compound (that is free or conjugated to another compound) as long as this glycoside compound comprises at least one hydrophilic glycone moiety that is covalently bound to a lipophilic aglycone core moiety of a steroid or terpenoid structure, regardless whether this glycoside compound is identical to a naturally-occurring saponin, or appears to largely correspond in structure to a naturally-occurring saponin but possesses at least one chemical group modification on either one of the glycone moiety or aglycone core moiety as compared to its corresponding naturally-occurring saponin, or is a glycoside compound that do not seem to correspond to any naturally-occurring saponin but by the above definition is a saponin, which could have been synthetically obtained through chemical and/or biotechnological synthesis routes and for this reason does not resemble any naturally-occurring saponin but still visibly comprises at least one hydrophilic glycone moiety that is covalently bound to a lipophilic aglycone core moiety of a steroid or terpenoid structure.

As already indicated above, as used herein, the term saponin shall be construed as encompassing:
(i) non-conjugated (“free”) saponins, which are further referred to herein using a term “saponin molecule” in the context of the disclosed herein saponin components of the pharmaceutical compositions and therapeutic methods; and
(ii) saponins that are covalently conjugated to other compound types and hence form part of conjugates comprising at least one saponin as a saponin moiety of the conjugate, which saponin moiety is conjugated to an at least one non-saponin moiety such as a linker for further conjugation, or as an effector molecule like an oligonucleotide, or a targeting ligand recognised by a cell-surface receptor, for example an endocytic receptor etc. Hence, in the context of the disclosed herein saponin components of the pharmaceutical compositions and therapeutic methods, such covalently conjugated saponins will be further referred to herein using a term “saponin moiety”, to discern them from the non-conjugated (“free”) saponins that, as explained above, are referred to herein using the term “saponin molecule”.

As used herein, the term “saponin component” refers to a component of a pharmaceutical composition or of a therapeutic method (to be construed as synonymous to the term “method of treatment”), which component comprises saponin as defined above.

In line with the above explanation, the saponin can be present in said pharmaceutical composition or provided as part of the therapeutic method in an unconjugated form (as used herein, as a “saponin molecule”), or in a form that is covalently bound (conjugated) to at least one other chemical compound that is not a saponin, thus forming a part of a conjugate comprising the saponin (as used herein, as a “saponin moiety” of said conjugate) and the at least other chemical compound that is not a saponin (as used herein, as a “non-saponin moiety” of said conjugate).

For example, as used herein the “saponin molecule” can correspond to a naturally-occurring saponin molecule found in or isolatable from natural sources, such as plant material, or can correspond to a non-naturally-occurring saponin molecule that has a chemical group modification as compared to the naturally-occurring saponin. In case such saponin molecule becomes covalently conjugated to another compound, for example being a linker which can be used for further conjugation steps, the saponin part of such formed conjugate will be referred to as a “saponin moiety”.

For comparison, in case of a saponin component of a pharmaceutical composition, which saponin component comprises a penta-cyclic triterpene saponin comprising an aglycone core of 12,13-dehydrooleanane type and an acid sensitive covalent bond with one or several atoms and which bond can be seen as simply replacing the aldehyde function at position C-23 of the aglycone core for the reason that said one or several atoms cannot be further classified functionally (e.g. said one or several atoms are not a linker with a chemical group for further conjugation reactions; nor a ligand for binding a receptor) or structurally (e.g. said one or several atoms are not an oligonucleotide, a peptide, an oligosaccharide etc.), in such a case, such saponin component will be further referred to using the term “saponin molecule” rather than the term “saponin moiety”.

If however this saponin component comprises a penta-cyclic triterpene saponin comprising an aglycone core of 12,13-dehydrooleanane type and an acid sensitive covalent bond with another functionally or structurally distinct non-saponin moiety (e.g. a linker, a ligand, an oligonucleotide etc.), in such a case, such saponin will be further referred to using the term “saponin moiety” rather than the term “saponin molecule”. The above distinction will be clear to the skilled person and requires not further elaboration.

The term “Saponinum album” has its normal meaning and here refers to a mixture of saponins produced by Merck KGAA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (~63%), QS-21 A-xylo (~32%), QS-21 B-apio (~3.3%), and QS-21 B-xylo (~1.7%).

Similarly, “QS-21A” has its regular scientific meaning and here refers to a mixture of QS-21 A-apio (~65%) and QS-21 A-xylo (~35%).

Similarly, “QS-21B” has its regular scientific meaning and here refers to a mixture of QS-21 B-apio (~65%) and QS-21 B-xylo (~35%).

The term “Quil-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GlcA- at the C-3beta-OH group found in QS-7, QS-17, QS-18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9, 660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi: 10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The terms “SO1861” and “SO1862” refer to the same saponin of Saponaria officinalis, though in deprotonated form or api form, respectively. The molecular mass is 1862 Dalton as this mass is the formal mass including a proton at the glucuronic acid. At neutral pH, the molecule is deprotonated. When measuring the mass using mass spectrometry in negative ion mode, the measured mass is 1861 Dalton.

The term “conjugate” has its regular scientific meaning and here refers to at least a first molecule (further termed “first moiety”) that is covalently bound to at least a second molecule (“second moiety”), therewith forming a covalently coupled assembly comprising or consisting of the first moiety and the second moiety. Typical conjugates are an ADC, an AOC, and SO1861-EMCH (EMCH linked to the aldehyde group of the aglycone glycoside core structure of the saponin, according to formula (I) (see below)). As used herein, the term “conjugate” is thus to be construed as a combination of two or more different moieties (that, as used herein purely to discern between the conjugated and unconjugated state) were referred to before the conjugation as the two or more molecules, which have been and are covalently bound. For example, different moieties forming a conjugate as disclosed herein may include one or more saponins or saponin moieties with one or more ligands that bind to an endocytic receptor present on a surface of a neuron, glial cell, a tumor cell, preferably wherein the ligand is e.g. an antibody or a binding fragment thereof, such as an IgG, a monoclonal antibody (mAb), a single domain antibody such as a VHH domain or another nanobody type, a bivalent nanobody molecule comprising two single domain antibodies, etc. In some aspects, the disclosed herein conjugates may be made by covalently linking different moieties via one or more intermediate moieties such as linkers, such as for example via linking to a central or further linker. In a conjugate, not all of the two or more, such as three, different moieties need to be directly covalently bound to each other. Different moieties in the conjugate may also be covalently bound by being both covalently bound to the same intermediate moiety such as a linker or each by being covalently bound to an intermediate moiety such as a further linker or a central linker wherein these two intermediate moieties such as two (different) linkers, are covalently bound to each other. According to this definition even more intermediate moieties such as linkers, may be present between the two different moieties in the conjugate as long as there is a chain of covalently bound atoms in between.

As used herein, the term “effector component” is to be construed herein as referring to a component of a composition or a treatment, the component comprising or consisting of an effector molecule or moiety. An example of an effector component is a nucleic acid therapeutic or an oligonucleotide therapeutic, in which the nucleic acid or oligonucleotide is the effector molecule or the effector moiety. An effector component comprising an oligonucleotide therapeutic can be referred to as an “oligonucleotide component” in such example.

The term “effector molecule”, or “effector moiety” when referring to the effector molecule as part of e.g. a covalent conjugate such as an effector component comprising a ligand for binding to an endocytic cell-surface receptor and comprising e.g. a nucleic acid, has its regular scientific meaning and here refers to a molecule that can selectively bind to for example any one or more of the target molecules: a protein, a peptide, a carbohydrate, a saccharide such as a glycan, a (phospho) lipid, a nucleic acid such as DNA, RNA, an enzyme, and that regulates the biological activity of such one or more target molecule(s). In the effector molecule as disclosed herein, the effector moiety for example exerts its effect in the cytosol (cytoplasm) and/or in the cell nucleus, and/or is delivered intracellularly in the endosome and/or lysosome and/or is active after exiting or escaping the endosomal-lysosomal pathway (therewith entering the cytoplasm). The effector molecule is for example a molecule selected from any one or more of a small molecule such as a drug molecule, a toxin such as a protein toxin, a nucleic acid or polynucleotide such as a BNA, an ASO, a PMO, an siRNA, an enzyme, a peptide, a protein, or an active fragment or active domain thereof, or any combination thereof. Thus, for example, an effector molecule or an effector moiety is a molecule or moiety selected from any one or more of a small molecule such as a drug molecule, a toxin such as a protein toxin, a nucleic acid or polynucleotide such as a BNA, an ASO, a PMO, an siRNA, an enzyme, a peptide, a protein, or any combination thereof, that can selectively bind to any one or more of the target molecules: a protein, a peptide, a carbohydrate, a saccharide such as a glycan, a (phospho) lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. Typically, an effector molecule can exert a biological effect inside a cell such as a mammalian cell such as a human cell, such as in the cytosol of said cell or in the nucleus of said cell. An effector molecule or moiety as disclosed therein is thus any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Typical effector molecules are thus drug molecules, an enzyme, a nucleic acid such as plasmid DNA or an ASO or an siRNA or a PMO, toxins such as toxins comprised by antibody-drug conjugates (ADCs), polynucleotides such as siRNA, BNA, nucleic acids comprised by an antibody-polynucleotide conjugate (AOC). For example, an effector molecule/moiety is a molecule which can act as a ligand that can increase or decrease (intracellular) enzyme activity, gene expression (e.g. gene silencing), or cell signalling. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway. Within the saponin component as disclosed herein, the saponin is not considered an effector molecule nor an effector moiety in the saponin component as disclosed herein. Thus, in the saponin components comprising a saponin, the saponin is not an effector moiety, and in the effector components comprising an effector moiety, the effector moiety is a different molecule than a conjugated saponin. In the context of the saponin component as disclosed herein, the term saponin refers to those saponins which exert an endosomal/lysosomal escape enhancing activity, when present in the endosome and/or lysosome of a mammalian cell such as a human cell, towards an effector moiety comprised by the effector component as disclosed herein and present in said endosome/lysosome together with the saponin.

As used herein, the terms “nucleic acid” and “polynucleotide” are synonymous to one another and are to be construed as encompassing any polymeric molecule made of units, wherein a unit comprises at least a nucleobase (or simply “base” e.g. being a canonical nucleobase like adenine (A), cytosine (C), guanine (G), thymine (T), or uracil (U), or any known non-canonical, modified, or synthetic nucleobase like 5-methylcytosine, 5-hydroxymethylcytosine, xanthine, hypoxanthine, 7-methylguanine; 5,6-dihydrouracil etc.) or a functional equivalent thereof, which renders said polymeric molecule capable of engaging in hydrogen bond-based nucleobase pairing (such as Watson-Crick base pairing) under appropriate hybridisation conditions with naturally-occurring nucleic acids such as deoxyribonucleic acid (DNA) or ribonucleic acid (RNA), which naturally-occurring nucleic acids are to be understood being polymeric molecules made of units being nucleotides, whereby each nucleotide consists of a pentose sugar, a phosphate group and one of the nucleobases.

Hence, from a chemistry perspective, the term nucleic acid under the present definition can be construed as encompassing polymeric molecules that chemically are DNA or RNA, as well as polymeric molecules that are nucleic acid analogues, also known as xeno nucleic acids (XNA) or artificial nucleic acids, which are polymeric molecules wherein one or more (or all) of the units are modified nucleotides or are functional equivalents of nucleotides. Nucleic acid analogues are well known in the art and due to various properties, such as improved specificity and/or affinity, higher binding strength to their target and/or increased stability in vivo, they are extensively used in research and medicine. Typical examples of nucleic acid analogues include but are not limited to locked nucleic acid (LNA) (that is also known as bridged nucleic acid (BNA)), phosphorodiamidate morpholino oligomer (PMO also known as Morpholino), peptide nucleic acid (PNA), glycol nucleic acid (GNA), threose nucleic acid (TNA), hexitol nucleic acid (HNA), 2′-deoxy-2′-fluoroarabinonucleic acid (FANA or FNA), 2′-deoxy-2′-fluororibonucleic acid (2′-F RNA or FRNA); altritol nucleic acids (ANA), cyclohexene nucleic acids (CeNA) etc.

In line with the above, in some instance, the nucleic acid of the present disclosure may be modified. For example, the nucleic acid may be modified on its backbone. Examples of modifications that can be performed on the backbone of a nucleic acid include, but are not limited to, phosphorothioate (PS), boranophosphate, phosphonoacatate (PACE), morpholine, peptide nucleic acid backbone modification (PNA), and amid-linked bases. The nucleic acid may also be modified on the sugar moiety and/or on the base moiety. Examples of modifications that can be performed on the sugar and/or the base moieties include, but are not limited to, locked nucleic acid (LNA), phosphoramidate (NP), 2′F-RNA, 2′-0 methoxyethyl (2′MOE), 2′O-methyl(2′OMe), 2′-O-fluoro (2′-F) 5-bromouracil, 5-iodouracil, 5-methylcytosine, ethylene bridged nucleic acids (ENA), diaminopurine, 2-thiouracil, 4-thiouracil, pseudouracil, hypoxantine, 2-aminoadenine, 6-methyl or other alkyl derivates of adenine and guanine, 2-propyl and other derivative of adenine and guanine, 6-azo-uracil, 8-halo, 8-amino, 8-thiol, 8-hydroxyk and other 8-substituted adenines and guanines, constrained ethyl sugar moiety (CET), ribofuranosyl, 2′-0,4′-C-methylene and 2′-0,4′-C-ethylene bicyclic nucleotide analogues, acyclic nucleotides (UNA and PNA), and dihydrouridine modification. Other modifications that may be performed on nucleic acids are, but are not limited to, modifications that include deoxyribonucleotide bases incorporated in a ribonucleotide sequence. The incorporations may be limited to the overhang structure in the canonical siRNA architecture or may be distributed in the sequence. Modifications to RNA molecules include, but are not limited to blunt-ended siRNA, 25-27mer siRNA, single strand siRNA, short hairpin siRNA, dumbbell siRNA, asymmetric siRNA, short interspaced siRNA, hybrid between siRNA and antisense oligonucleotides (ASO). Other analogue nucleic acids may be contemplated include those with non-ribose backbones. In addition, mixtures of naturally occurring nucleic acids, analogues, and both may be made. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 5-methylcytidine, pseudouridine etc. Modified 5′ cap structures such as 3′-O-Me-m7G (5′) ppp (5′) G (anti-reverse cap analogue), may also be used for increased translation of mRNA. Nucleic acids include DNA in any form, RNA in any form, including triplex, duplex or single-stranded, antisense, siRNA, ribozymes, deoxyribozymes, polynucleotides, oligonucleotides, chimeras, and derivatives thereof.

In accordance with the cannon, length of a nucleic acid is expressed herein the number of units from which a single strand of a nucleic acid is build. Because each unit corresponds to exactly one nucleobase capable of engaging in one base pairing event, the length is frequently expressed in so called “base pairs” or “bp” regardless of whether the nucleic acid in question is a single stranded (ss) or double stranded (ds) nucleic acid. In naturally-occurring nucleic acids 1 bp corresponds to 1 nucleotide, abbreviated to 1 nt. For example, a single stranded nucleic acid made of 1000 nucleotides (or a double stranded nucleic acid made of two complementary strands each of which is made of 1000 nucleotides) is described as having a length of 1000 base pairs or 1000 bp, which length can also be expressed as 1000 nt or as 1 kilobase that is abbreviated to 1 kb. 2 kilobases or 2 kb are equal to the length of 2000 base pair which equates 2000 nucleotides of a single stranded RNA or DNA. To avoid confusion however, in view of the fact the nucleic acids as defined herein may comprise or consist of units not only chemically being nucleotides but also being functional equivalents thereof, the length of nucleic acids will preferentially be expressed herein in “bp” or “kb” rather than in the equally common in the art denotation “nt”.

In advantageous embodiments, the nucleic acid as disclosed herein are no longer than 1 kb, preferably no longer than 500 bp, most preferably no longer than 250 bp.

In particularly advantageous embodiments, the nucleic acid is an oligonucleotide (or simply an oligo) defined as nucleic acid being no longer than 200 bp, i.e. in accordance with the above provided definition, being any polymeric molecule made of no more than 200 units, wherein each unit comprises a nucleobase or a functional equivalent thereof, which renders said oligonucleotide capable of engaging in hydrogen bond-based nucleobase pairing under appropriate hybridisation conditions with DNA or RNA. Within the ambit of said definition, it will immediately be appreciated that the disclosed herein oligonucleotides can comprise or consist of units not only being nucleotides but also being synthetic equivalents thereof. In other words, from a chemistry perspective, as used herein the term oligonucleotide will be construed as possibly comprising or consisting of RNA, DNA, or a nucleic acid analogue such as but not limited to LNA (BNA), PMO (Morpholino), PNA, GNA, TNA, HNA, FANA, FRNA, ANA, CeNA and/or the like.

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 (typical for a polypeptide, a protein, a protein domain, such as an antibody, a Fab, an scFv, a ligand for a receptor such as EGF) amino acids. Preferably, the amino-acid residues are (typically) linked via (a) peptide bond(s). As disclosed herein, said amino-acid residues are or comprise (modified) (non-) natural amino acid residues.

As used herein, the term “antibody or a binding fragment thereof or a binding domain thereof” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) fragment, a F(ab′) 2 fragment, a Fv fragment or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgGI, IgG2, IgG2A, IgG2B, IgG2C, IgG3, IgG4, IgAQ1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (D), epsilon (e), gamma (g) or mu (m) heavy chain. In some embodiments, the heavy chain of an antibody described herein can comprise a human alpha (a), delta (D), epsilon (e), gamma (g) or mu (m) heavy chain. In a particular embodiment, an antibody described herein comprises a human gamma 1 CHI, CH2, and/or (e.g., and) CH3 domain. In some embodiments, the amino acid sequence of the VH domain comprises the amino acid sequence of a human gamma (g) heavy chain constant region, such as any known in the art. Non-limiting examples of human constant region sequences have been described in the art, e.g., see U.S. Pat. No. 5,693,780 and Kabat E A et al, (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P, et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The term “single domain antibody”, or “sdAb”, in short, or ‘nanobody’, has its regular scientific meaning and here refers to an antibody fragment consisting of a single monomeric variable antibody domain, unless referred to as more than one monomeric variable antibody domain such as for example in the context of a bivalent sdAb, which comprises two of such monomeric variable antibody domains in tandem. A bivalent nanobody is a molecule comprising two single domain antibodies targeting epitopes on molecules present at the extracellular side of a cell, such as epitopes on the extracellular domain of a cell surface molecule that is present on the cell. Preferably the cell-surface molecule is a cell-surface receptor. A bivalent nanobody is also named a bivalent single domain antibody. Preferably the two different single domain antibodies are directly covalently bound or covalently bound through an intermediate molecule that is covalently bound to the two different single domain antibodies. Preferably the intermediate molecule of the bivalent nanobody has a molecular weight of less than 10,000 Dalton, more preferably less than 5000 Dalton, even more preferably less than 2000 Dalton, most preferably less than 1500 Dalton.

The term “GalNAc” has its regular scientific meaning and here refers to N-acetylgalactosamine and to the IUPAC name thereof: 2-(acetylamino)-2-deoxy-D-galactose.

As used herein, the term “covalently linked” refers to a characteristic of two or more molecules being linked together via at least one covalent bond, i.e. directly, or via a chain of covalent bonds, i.e. via a linker comprising at least one or more atoms.

The term “moiety” as used herein will usually refers to a molecule that is bound, linked, conjugated to a further molecule, linker, assembly of molecules, etc., and therewith forming part of a larger molecule, conjugate, assembly of molecules. Typically, a moiety is a first molecule that is covalently bound to a second molecule (second moiety), involving one or more chemical groups initially present on the first and second molecules. For example, when a saponin molecule is covalently linked via at least one linker to one or more GalNAc molecules, both the saponin molecule is a saponin moiety in the formed saponin-GalNAc conjugate and the GalNAc molecule(s) is/are a moiety/moieties in said conjugate. For example, a nucleic acid such as an antisense oligonucleotide, that is conjugated to an endocytic receptor binding ligand such as an antibody or one or more GalNAc molecules, is a nucleic acid moiety in the nucleic acid-GalNAc conjugate.

As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the disclosed herein embodiments can operate in other sequences than described or illustrated herein, unless specified otherwise.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present disclosure, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present disclosure, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The use of terms in brackets in the text, with the exception of chemical and/or mathematical formulae, usually means that the term within brackets specifies a possible option or a possible meaning and should thus not be considered limiting.

The embodiments as described herein can operate in combination and cooperation, unless specified otherwise. Furthermore, the various embodiments, although referred to as “preferred” or “e.g.” or “for example” or “in particular” and the like are to be construed as exemplary manners in which the disclosed herein concepts may be implemented rather than as limiting.

For all Figures, “Figure” and “Fig.” refer to the same.

As used herein, the term “an endocytic receptor” is to be understood as any one of cell surface molecules, likely receptors or transporters that are accessible to their specific ligands from the external side or surface of cell membrane (also known as plasmalemma) and capable of undergoing internalisation via endocytic pathway e.g., upon external stimulation, such as ligand binding to the receptor. In some embodiments, an endocytic receptor can be internalized by clathrin-mediated endocytosis, but can also be internalized by a clathrin-independent pathway, such as, for example, phagocytosis, macropinocytosis, caveolae- and raft-mediated uptake or constitutive clathrin-independent endocytosis. In some embodiments, the endocytic receptor comprises an intracellular domain, a transmembrane domain, and/or (e.g., and) an extracellular domain, which may optionally further comprise a ligand-binding domain. In some embodiments, the endocytic receptor becomes internalized by the cell after ligand binding. In some embodiments, a ligand may be a specific-cell-targeting agent, for example a natural ligand (or a synthetic fragment thereof) or an antibody or a binding fragment thereof.

As used herein, the term “ligand” is to be understood as any molecule that binds to or can be recognised by a receptor. Typical ligand can be an antibody, a binding fragment of an antibody, simply fragment of an antibody. Alternatively, a typical ligand can also be a protein, a peptide, a polysugar, a glycoprotein, or a fragment of any one thereof which fragment is capable of being recognised by an endocytic receptor.

As used herein, the term “covalently linked” refers to a characteristic of two or more molecules being linked together via at least one covalent bond, i.e. directly, or via a chain of covalent bonds, i.e. via a linker comprising at least one or more atoms.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any polynucleotide (oligonucleotide) molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO, AON), a short or small interfering RNA (siRNA; silencing RNA), an anti-sense DNA, anti-sense RNA, etc.

The term “subject” as used herein refers to a human suffering from or at risk of a certain health-related disorder, such as a disease or other pathological condition. The term “subject” and “patient” herein are used interchangeably.

The term ‘treatment’ as used herein has its conventional meaning and refers to a medical intervention or management of a subject with the intention to cure, ameliorate, stabilize, or prevent a health-related disorder, such as an ocular disorder. This term “treatment” includes e.g. active treatment that is a type of an action directed specifically toward the improvement of a health-related disorder, and also includes causal treatment that is a treatment directed towards a removal of the cause of the associated therewith health-related disorder. The term ‘prophylaxis’ as used herein means a medical intervention or management of a subject with the intention to maintain health or the normal bodily functions. As used herein, prophylaxis is to be construed as falling within the scope of a treatment, unless indicated otherwise.

As used herein, the term “ocular disorder” is to interpreted broadly as referring to any health-related disorder of the eye, in particular such that affects a subject's vision or sense of comfort related to at least one of the subject's eyes. Usually, the term “ocular disorder” will related to an eye diseases or a pathological condition related to at least one of the subject's eyes.

Analogously, as used herein, the term “CNS disorder” is to be interpreted broadly as referring to any health-related disorder of the CNS, in particular such involving changes in the brain and/or the spinal cord. Usually, the term “CNS disorder” will related to a neurological diseases or a pathological condition related to at least a part of the subjects' brain or spinal cord.

As used herein the term “administration” is to be construed as referring to the way of providing a substance, such as compound, or a pharmaceutical composition to a subject. Conversely, as used herein the term “delivery” is to be construed as referring to the way a compound reaches its destination site, e.g. specific zone, cell or tissue type like retina of the eye. For example, as used herein, administration can relate to intrathecal, intravenous, topical, intranasal, intraocular, etc. way of providing a compound into the subject's body, with an intended delivery to e.g. cerebellum or the retina as the destination site. Usually, the terms “administering” or “administration” will be construed as remating to the provision of a substance that is physiologically and/or (e.g., and) pharmacologically useful (e.g., to treat a condition in the subject).

The term “carrier” as used herein has its conventional meaning and refers to a pharmaceutically acceptable diluent, adjuvant, excipient or vehicle with which a pharmaceutically active ingredient is administered.

The term ‘excipient’ as used herein has its conventional meaning and refers to a pharmaceutically acceptable ingredient, which is commonly used in the pharmaceutical technology for preparing a granulate, solid or liquid oral dosage formulation.

### BRIEF DESCRIPTION OF THE DRAWINGS

FIG. 1: In vivo efficacy enhancement by local co-administration of saponin components to ASO compounds in the CNS: intraventricular administration of either 10 μg Malat1 ASO, 3 μg Malat1 ASO, or co-administering 3 μg Malat1 ASO with a saponin component Saponin (here, SO1861) intraventricularly to the right lateral ventricle compared to controls (saponin component only and vehicle groups) and the effect in different brain regions close to or peripheral to the injection site. All data are shown as mean±SEM, n=3. Two-way ANOVA, Tukey's post hoc comparison: *p<0.05, **p<0.01, ***p<0.001, ****p<0.0001.

FIGS. 2A, 2B: Specificity of enhancement by co-administration of saponin components to ASO compounds measured by Malat1 RNA knockdown in neuronal cells. (A) Titration of Malat1 ASO, with and without co-administration of a fixed amount of a triterpenoid saponin component Saponin (here SO1861 as an example of a pentacyclic 12,13-dehydrooleanane-type saponin) compared to the steroid(-like) saponins/molecules digitonin, tomatine or digoxin; (B) Titration of triterpenoid saponin component Saponin (SO1861) compared to the steroid(-like) saponins/molecules digitonin, digoxin, glycyrrhizin and tomatine in co-administration with a fixed amount of 200 nM ASO.

FIG. 3: Improved efficacy of a saponin component comprising a payload (ASO-Saponin), said saponin component obtained by covalent conjugation of the saponin molecule SO1861 to the payload Malat1 ASO, providing saponin component ASO-Saponin (Malat1-ASO-SC-SO1861), for delivery in neuronal cells compared to Malat1 ASO alone.

FIG. 4: Improved efficacy of a PMO targeting the CNS-disease relevant gene Sod1 by saponin components Saponin (here 3 μM SO1861-SC-Mal) in neuronal cells: PMO efficacy is measured by increase in aberrant transcript induction (leading to mRNA degradation) only with co-administration of a fixed amount of saponin component.

FIG. 5: In vivo evaluation of tolerability and effect in the eye of saponin components Saponin (SO1861) with or without an LNA payload after local intravitreal co-administration, assessed by corneal thickness in the treated eye (right, OD).

FIG. 6: Enhancement of STAT3 mRNA reduction by co-dosing of saponin components with different PMOs or ASO (with different mechanisms of action) in targeted, conjugated or free form: (A) STAT3 expression modulation by co-administration of a saponin component Saponin (3 μM SO1861-SC-Mal) to STAT3_ST6 PMO (exon skip leading to premature termination codon and thus STAT3 mRNA reduction) in neuronal cells; (B) STAT3 expression modulation by either free STAT3_ST6 PMO (with or without co-administration of a saponin component Saponin (SO1861-SC-Mal) or a saponin component consisting of an antibody-targeted PMO-SO1861-conjugate, based on conjugating SO1861-SC (cetuximab (Cet-SO1861-STAT3_ST6 PMO) in A431 cells; (C) STAT3 expression modulation by co-administration of different (targeted and non-targeted) saponin components (Saponin (SO1861), Saponin (1) (SO1861-AH-Block), Saponin (2) (Conjugated SO1861-AH)) to a RNA degrading STAT3 mRNA-targeting ASO (ribonuclease H mediated RNA degradation); (D) STAT3 expression modulation by a splice switching PMO (STAT3_ST2), resulting in increase of STAT3B isoform: PMO added, either in free form (STAT3-ST2) or in a targeting-ligand conjugate form (Cet-STAT3_ST2 PMO), with or without co-administration of a saponin component Saponin (SO1861-SC-Mal), or in an antibody-targeted PMO-SO1861-conjugate (saponin component is Cet-Saponin-STAT3_ST2 PMO in which the Saponin is SO1861) in A431 cells.

FIG. 7: Efficacy enhancement of siRNAs by co-dosing of saponin components Saponin (1.3 μM SO1861) in cells from human brain tissue (U87, isolated from a malignant glioma): (A) Efficacy of AHA1 siRNA and (B) efficacy of MMP14 siRNA (with chemically modified variants for improved stability against siRNA degradation) in combination with saponin component Saponin (1.3 μM SO1861), measured by RNA levels.

FIG. 8: Synthesis and chemical structure of SO1861-SC-azide.

FIG. 9: Synthesis and chemical structure of GN3-SC-SO1861.

FIG. 10A, 10B, 10C: Efficacy enhancement by co-administration of targeted saponin components and targeted siRNA in vivo: efficacy and durability of effect (here, serum TTR protein reduction) of co-administration of saponin component Saponin (GN3-SC-SO1861) and an oligonucleotide, here GN3-siTTR. GN3-siTTR was always administered on day 0 and saponin component was administered at the timepoints indicated by the arrow; n=6 mice in all groups except vehicle, where n=3; shown is mean TTR serum level±SD. (A) GN3-siTTR administered together with saponin component at day 0, (B) GN3-siTTR administered at day 0, saponin component administered at day 7 (arrow), (C) GN3-siTTR administered at day 0, saponin component administered at day 28 (arrow).

FIG. 11: Structure of trivalent GalNAc-oligonucleotide, for example trivalent GalNAc-siRNA also referred to as GN3-siRNA, or in a specific example GN3-siTTR.

FIG. 12. In vivo efficacy enhancement by local co-administration of saponin components to (targeted)-ASO compounds in the CNS: Malat1 expression analysis upon intraventricular administration to the right lateral ventricle of vehicle (DPBS; Group A), Malat1 ASO with co-administration of saponin component (Group B), saponin component alone (Group C), 1-component Malat1 ASO-Saponin (Group D+Group E), targeted aCD71-Malat1 ASO alone (Group F) or aCD71-Malat1 ASO with co-administration of saponin component (Group G), in brainstem (A), striatum (B), thalamus (C), cerebral cortex—right (D), cerebral cortex—left (E), hippocampus—right (F), hippocampus—left (G), cerebellum (H), and all other brain areas (I); The saponin component Saponin is SO1861-SC-Mal, the saponin component Malat1 ASO-Saponin comprises the saponin SO1861 and the saponin component Saponin (1) is SO1861 in this example. All data are shown as mean±SEM, n=3 (or n=2 for treatment group D). One-way ANOVA, Tukey's post hoc comparison: *p<0.05, **p<0.01, ***p<0.001, ****p<0.0001.

FIG. 13. Summary of Malat1 expression analysis of FIG. 12. Relative Malat1 expression compared to vehicle, in various brain regions after by local co-administration of saponin components to (targeted)-ASO compounds; sorted by efficacy of treatment group B. The saponin component Saponin is SO1861-SC-Mal, the saponin component Malat1 ASO-Saponin comprises the saponin SO1861 and the saponin component Saponin (1) is SO1861 in this example.

FIG. 14. In vivo efficacy enhancement by local co-administration of saponin components to (targeted)-PMO compounds in the CNS: Sod1 expression analysis upon intraventricular administration of vehicle (DPBS; Group A), saponin component alone (Group C), SOD1 PMO alone (Group H) or SOD1 PMO with co-administration of saponin component (Group I), targeted aCD71-SOD1 PMO alone (Group J) or aCD71-SOD1 PMO with co-administration of saponin component (Group K), in brainstem (A), striatum (B), thalamus (C), cerebral cortex-right (D), cerebral cortex-left (E), hippocampus-right (F), hippocampus-left (G), cerebellum (H), and all other brain areas (I); The saponin component Saponin is SO1861-SC-Mal and the saponin component Saponin (1) is SO1861 in this example. All data are shown as mean±SEM, n=3. One-way ANOVA, Tukey's post hoc comparison: *p<0.05, **p<0.01, ***p<0.001, ****p<0.0001.

FIG. 15. Summary of Sod1 expression analysis of FIG. 14. Relative Sod1 expression compared to vehicle, in various brain regions after by local co-administration of saponin components to (targeted)-PMO compounds; sorted by efficacy of treatment group K. The saponin component Saponin is SO1861-SC-Mal and the saponin component Saponin (1) is SO1861 in this example.

FIG. 16. Covalent conjugation of a saponin component to a payload improves payload efficacy; (A) Malat1 expression analysis upon treatment with Malat1 ASO alone, conjugated Malat1 ASO-Saponin (the saponin component comprising SO1861), or Malat1 ASO with co-administration of 400 nM saponin (1) (the saponin component, being SO1861-SC) in Neuro-2a cells; (B) Malat1 expression analysis after treatment with Malat1 ASO alone, conjugated Malat1 ASO-Saponin (the saponin component comprising SO1861), or titration of unconjugated Malat1 ASO+Saponin (1) (the saponin component, being SO1861-SC), in Neuro-2a cells.

FIG. 17. Saponin components enhance mRNA reduction when co-dosed with (targeted) ASO in neuronal cells; (A) MALAT1 expression modulation by MALAT1 ASO or MALAT1 ASO co-dosed with a saponin component, Saponin (1) at 400 nM or 4 UM dose; (C) MALAT1 expression modulation by MALAT1 ASO or MALAT1 ASO co-dosed with a saponin component, Saponin (2); (C) MALAT1 expression modulation by CD71-targeted aCD71-Malat1 ASO or aCD71-Malat1 ASO co-dosed with saponin component Saponin (2); The saponin component Saponin (1) is SO1861-SC and the saponin component Saponin (2) is SO1861-AH (Block) in this example.

FIG. 18. Saponin components enhance mRNA reduction when co-dosed with (targeted) ASO in neuronal cells; (A) SOD1 aberrant transcript induction by SOD1 ASO or SOD1 ASO co-dosed with saponin component; (B) SOD1 expression modulation by SOD1 ASO or SOD1 ASO co-dosed with saponin component; (C) SOD1 aberrant transcript induction by CD71-targeted aCD71-SOD1 ASO or aCD71-SOD1 ASO co-dosed with saponin component; (D) SOD1 expression modulation by CD71-targeted aCD71-SOD1 ASO or aCD71-SOD1 ASO co-dosed with saponin component; (E) SOD1 aberrant transcript induction by aCD71-SOD1 ASO or 1 component conjugates aCD71-(Saponin-SOD1 PMO) high and aCD71-(Saponin-SOD1 PMO) low. (F) SOD1 expression modulation by aCD71-SOD1 ASO or 1 component conjugates aCD71-(Saponin-SOD1 PMO) high and aCD71-(Saponin-SOD1 PMO) low; The saponin component Saponin (1) is SO1861-AH (Block) and the saponin component Saponin is SO1861-SC in this example.

FIG. 19. Saponin components enhance mRNA reduction when co-dosed with various (targeted) ASOs in retinal pigment epithelia cells; (A) MALAT1 expression modulation by MALAT1 ASO or MALAT1 ASO co-dosed with saponin component; (B) SOD1 expression modulation by SOD1 ASO or SOD1 ASO co-dosed with saponin component; (C) SOD1 expression modulation by CD71-targeted aCD71-SOD1 ASO or aCD71-SOD1 ASO co-dosed with saponin component; the saponin component Saponin (1) is SO1861-EMCH in this example.

FIG. 20. Saponin components induce exon skip and enhance mRNA reduction when co-dosed with multiple targeted-PMOs in retinal pigment epithelia cells; (A) SOD1 exon 2 skip modulation by aCD71-PMO (1) or aCD71-PMO (1) co-dosed with saponin component; (B) SOD1 exon 3 or exon 2/3 skip modulation by aCD71-PMO (2) or aCD71-PMO (2) co-dosed with saponin component; (C) Amount of residual full length SOD1 gene product upon treatment with aCD71-PMO (1) or aCD71-PMO (2) in the presence of saponin component; the saponin component Saponin (1) is SO1861-EMCH in this example.

### DETAILED DESCRIPTION

The innovative concepts as presented herein will be described with respect to particular aspects and embodiments of the disclosure, which should be regarded as descriptive and not as limiting beyond of what is described in the claims. The aspects and/or the embodiments as described herein can operate in combination and cooperation, unless specified otherwise. While the disclosed herein innovative concepts are described with reference to these aspects and embodiments, it is contemplated that alternatives, modifications, permutations and equivalents thereof will become apparent to one having ordinary skill in the art upon reading the specification and upon contemplation of the drawings and/or graphs. The disclosed matter is not limited in any way to the illustrated embodiment and changes thereto can be made without departing from the scope which is defined by the appended claims.

Disclosed herein are improved pharmaceutical compositions for the treatment disorders affecting the organs derived from neural tube, i.e. the organs of the CNS and the eye, the compositions comprising a saponin component and a nucleic acid therapeutic capable of treating or ameliorating an disorder of the CNS or of the eye, wherein the compositions are administered locally to said organ, i.e. into said organ, or into the body cavity housing and protecting said organ, or into a fluid space that is in non-blood-tissue barrier obstructed fluid communication with the cells of the organ.

The saponin of the disclosed herein novel compositions for the CNS and the eye local delivery is an endosomal-escape enhancing (EEE) saponin.

Without wishing to be bound by any theory, the disclosed herein pharmaceutical compositions were conceived based on the observation that a specific group of penta-cyclic triterpene saponin comprising an aglycone core of 12,13-dehydrooleanane type appear to exhibit potent endosomal-escape enhancing properties. Saponins of this specific type were characterised and reported in e.g. WO2020126620 as possessing an endosomal-escape enhancing (EEE) activity towards various antibody-drug conjugates (ADCs) in several cancer cells. This group of saponins was further disclosed in WO2020126626, WO2020126627, WO2020126620, WO2020126627, WO2020126064, WO2020126604, WO2020126600, and WO2020126609 as being capable of dramatically improving cancer treatment using oligonucleotide therapeutics, as demonstrated by enhanced by the saponin silencing of the HSP27 gene transcript using HSP27-specific BNA-based oligonucleotide in different tumour models cell lines.

As explained herein and further demonstrated in the accompanying examples, not only was the inclusion of a penta-cyclic triterpene saponin comprising an aglycone core of 12,13-dehydrooleanane type safe in combination with an oligonucleotide therapeutic following local administration to the mouse brain in vivo, but also it visibly increased the oligonucleotide therapeutic's bioavailability.

In line with these findings, in a first general aspect, a saponin component is provided for use in a therapeutic method of treating a subject suffering from a disorder of a (n immune-privileged) neuron-rich organ comprising vasculature with blood-tissue barrier properties, such as organs derived from neural tube, the method comprising administration to the subject of:
the saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration is performed directly into the organ or into a body cavity or fluid space that is in (non-blood-tissue barrier obstructed) communication with the cells of the organ.

In line a further general aspect, a pharmaceutical composition is provided for use in a treatment of a disorder of a (n immune-privileged) neuron-rich organ comprising vasculature with blood-tissue barrier properties, such as organs derived from neural tube, the pharmaceutical composition comprising:
the saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration of the pharmaceutical composition is performed directly into the organ or into a body cavity or fluid space that is in (non-blood-tissue barrier obstructed) communication with the cells of the organ.

In advantageous embodiments, a targeting option is provided, to e.g. target certain cells while keeping others not targeted. Hence, in an advantageous embodiment, compatible with the above aspects, a saponin component or a pharmaceutical composition for the disclosed herein use is provided,
wherein the saponin component further comprises a first ligand recognised by a first endocytic receptor, and/or wherein the effector component further comprises a second ligand recognised by a second endocytic receptor, possibly wherein the second endocytic receptor is the same as the first endocytic receptor, further possibly wherein the second ligand is the same as the first ligand, alternatively wherein the second endocytic receptor differs from the first endocytic receptor with the proviso that the two different endocytic receptors are both present on the same cell; preferably wherein the first ligand and/or the second ligand is a proteinaceous ligand, for example a naturally existing peptide or protein ligand (e.g. a cytokine or a growth factor, like EGF) or a receptor-interacting part thereof, or is an antibody or a binding fragment thereof, such as a F(ab′) 2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}.

As it will be described later in greater detail in the context of ligands, possible embodiments using non-proteinaceous ligands are also feasible. For example, typical such ligands include vitamin A, glutamate, or sugar chains such as of glucose or mannose 6 phosphate units. A well-known and broadly utilised in liver-related applications is a targeting option using non-proteinaceous ligands comprising one or more GalNAc moieties to specifically target asialoglycoprotein receptor (ASGPR). Hence, in a possible embodiment, the first ligand and optionally the second ligand is at least partially a non-proteinaceous ligand that is recognised by endocytic receptors on cell-surface of e.g. neurons or glia cells, for example targeting the glucose transporter, possibly comprising one or more glucose units.

### The Saponin Component

In line with the above, the “saponin component” as disclosed herein comprises a penta-cyclic triterpene saponin comprising an aglycone core of 12,13-dehydrooleanane type at their structure (also referred to as sapogenin or aglycone), usually shown as a penta-cyclic C30 terpene skeleton, and frequently comprising an aldehyde function at position C-23 in their naturally occurring state. Examples of such known saponins are shown in Table 2A below and in the Scheme of SAPONIN A below.

A notable feature of these saponins is the aldehyde function at position C-23 of the saponin's aglycone core structure. Without wishing to be bound by any theory, it was observed that the presence of said aldehyde function (sometimes referred as “aldehyde group”; which in the present context should be construed as synonymous) in the aglycone core can be particularly beneficial for the capacity of the saponin to stimulate and/or potentiate the endosomal escape of the therapeutic nucleic acids.

Hence, in an advantageous embodiment, a saponin component (or a pharmaceutical composition) for the disclosed herein use is provided, wherein the penta-cyclic triterpene saponin further comprises
an aldehyde function at position C-23 of the aglycone core, or
an acid-sensitive covalent bond configured to break under acidic conditions so as to create the aldehyde function at position C-23 of the aglycone core, preferably wherein the acid-sensitive covalent bond is selected from any one or more of: a semicarbazone bond, a hydrazone bond, an imine bond, an acetal bond including a 1,3-dioxolane bond, a ketal bond, an ester bond, and/or an oxime bond, and preferably is selected from a semicarbazone bond and a hydrazone bond.

Most of the naturally-occurring known penta-cyclic triterpene saponin comprising an aglycone core of 12,13-dehydrooleanane type that also comprise the aldehyde function at position C-23 in their native or unconjugated form, are saponins for which the aglycone core is either quillaic acid or gypsogenin. An exemplary chemical structure of such a saponin is schematically depicted the Scheme of SAPONIN A:

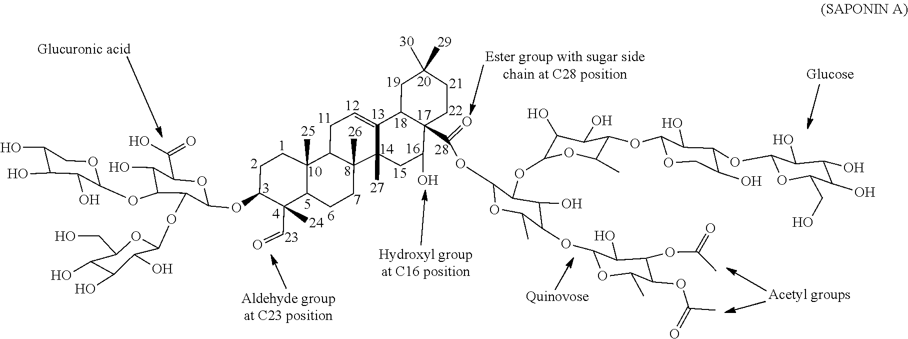

In line with this, it was observed that saponins comprising a quillaic acid aglycone or a gypsogenin aglycone core structure are particularly suitable for the purposes of the present disclosure. Hence, in an next embodiment, compatible with preceding embodiments, a saponin component or a pharmaceutical composition, for the disclosed herein use is provided, wherein the penta-cyclic triterpene saponin comprises the aglycone core selected from quillaic acid, gypsogenin, and an aldehyde-substituted derivative of either one of quillaic acid or gypsogenin defined as a quillaic acid-based or gypsogenin-based aglycone core, respectively, wherein the aldehyde function at position C-23 is substituted by the acid sensitive covalent bond configured to break under acidic conditions so as to create the aldehyde function at position C-23 of the aglycone core,
preferably wherein the quillaic acid or the quillaic acid-based aglycone core is selected from:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo, or the aldehyde-substituted derivative of any one thereof, respectively; or
wherein the gypsogenin or the gypsogenin-based aglycone core is selected from:
   SA1641, gypsoside A, NP-017772, NP-017774, NP-017777, NP-017778, NP-018109, NP-017888, NP-017889, NP-018108, SO1658 and Phytolaccagenin, or the aldehyde-substituted derivative of any one thereof, respectively.
      Saponins can comprise one or more saccharide chains attached to the aglycone core structure. Preferred saponins of the composition for use according to the disclosure comprise a single chain (i.e. are mono-desmosidic) or two chains (i.e. are bis-desmosidic) attached to the aglycone core structure. In line with this, in an next embodiment, compatible with preceding embodiments, a saponin component or a pharmaceutical composition for the disclosed herein use is provided, wherein the penta-cyclic triterpene saponin is mono-desmosidic or bi-desmosidic, preferably comprising a first saccharide chain bound to a position C-3 of the aglycone core, more preferably wherein the first saccharide chain is selected from Group A listed in Table 1A, even more preferably wherein the first saccharide chain comprises a glucuronic acid group, preferably a terminal glucuronic acid group, most preferably wherein the first saccharide chain comprises:

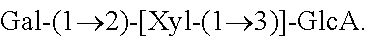

In a particular embodiment, compatible with the preceding ones, a saponin component or a pharmaceutical composition for the disclosed herein use is provided, wherein the penta-cyclic triterpene saponin is isolated from Saponaria officinalis, and is preferably any one or more of Saponarioside B, SO1542, SO1584, SO1658, SO1674, SO1700, SO1730, SO1772, SO1832, SO1861, SO1862 and SO1904, more preferably any one or more of SO1832, SO1861 and SO1862, even more preferably SO1832 or SO1861, most preferably SO1861.

The saponin component as disclosed herein can include one or more unconjugated saponin molecules, and/or saponin molecules that have been conjugated and are further referred to as saponin moieties (purely to discern them from their unconjugated free molecule counterparts).

Hence, in a possible embodiment, compatible with preceding embodiments, a saponin component or a pharmaceutical composition for the disclosed herein use is provided, wherein the saponin component comprises an unconjugated saponin molecule (defined as the penta-cyclic triterpene saponin that is not covalently conjugated to a non-saponin moiety, possibly wherein the saponin component consists of the unconjugated saponin molecule).

In an alternative embodiment, compatible with the previous one and other preceding embodiments, a saponin component or a pharmaceutical composition for the disclosed herein use is provided, wherein the saponin component comprises a saponin moiety that is covalently conjugated with at least one non-saponin moiety; preferably via an acid-sensitive covalent bond that breaks under acidic conditions, more preferably being an acid-sensitive covalent bond at the position C-23 of the aglycone core, even more preferably wherein the acid sensitive covalent bond at the position C-23 of the aglycone core is configured to break under acidic conditions so as to create the aldehyde function at position C-23 of the aglycone core thus resulting in a release of the penta-cyclic triterpene saponin comprising the aldehyde function at the position C-23 of the aglycone core from the non-saponin moiety, even more preferably wherein the acid-sensitive covalent bond is selected from any one or more of: a semicarbazone bond, a hydrazone bond, an imine bond, an acetal bond including a 1,3-dioxolane bond, a ketal bond, an ester bond, and/or an oxime bond, most preferably being selected from a semicarbazone bond and a hydrazone bond, and/or wherein the saponin moiety is covalently conjugated with the at least one non-saponin moiety by an acid-stable bond, preferably via a glucuronic acid group if said group is present.

In a related embodiment, a saponin component or a pharmaceutical composition for the disclosed herein use is provided, wherein the non-saponin moiety comprises any one or more of:
a linker,
the first ligand of claim 2,
the effector component, and/or
a scaffold molecule,
preferably; wherein the saponin moiety is directly covalently conjugated with the linker,
more preferably wherein the linker comprises or is covalently conjugated to the saponin moiety via the acid sensitive covalent bond, more preferably at the position C-23 of the aglycone core, or via the acid-stable bond, preferably at the glucuronic acid group if said group is present;
even more preferably wherein the linker is further covalently conjugated to the first ligand and/or to the effector component, possibly via the scaffold molecule;
for example wherein the scaffold molecule is a multi-functional linker scaffold molecule or a polymeric scaffold molecule possibly comprising a dendron, such as a poly-amidoamine (PAMAM) dendrimer, or a poly-ethylene glycol, such as any of PEG3-PEG30.

As used herein, the term scaffold molecule is to relate to a moiety of a conjugate which can serve as a scaffold for conjugating other moieties to a conjugate. In the present context, such scaffold molecule can be used for effectuating covalent linking between the saponin moiety, an effector moiety, and further possibly the first ligand. The linking to scaffold molecule can be effectuated either directly, or via the first, second, of any further linker.

Typical scaffold molecules as known in the art are based on an oligomeric or polymeric structure, frequently either being a dendron such as a poly-amidoamine (PAMAM) dendrimer, or a poly-ethylene glycol such as any of PEG3-PEG30. In advantageous embodiments of the disclosure, any one of such scaffold molecules can be used. For example, it can advantageously be a polymeric or oligomeric structure being any one of PEG4-PEG12 or any one of a G2 dendron, a G3 dendron, a G4 dendron and a G5 dendron, more preferably being a G2 dendron or a G3 dendron or a PEG3-PEG30. Dendrons appear particularly advantageous for eye applications. This is because eye therapeutic formulations suffer from low retention issues, which leads to frequent injections. Provision of a scaffold molecule that can be retained for a prolonged time in the eye fluid or also in the CNS can provide longer term exposure advantage.

In another example, compatible with the above ones, a multi-functional linker can be used as a scaffold (termed above “multi-functional linker scaffold molecule”). A multi-functional linker scaffold molecule can be made from a trifunctional linker, such as the one shown by Structure A in the example below, here represented in non-conjugated form:

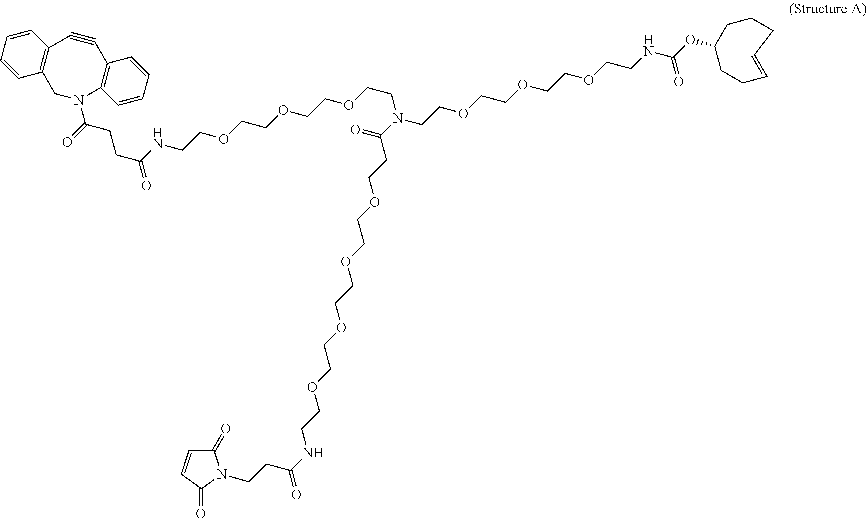

In a possible embodiment, a conjugate can be comprising 1-4 of such the trifunctional linkers for every molecule of the targeting ligand comprised by the conjugate, more preferably being 1-2 trifunctional linkers, most preferably being 1.2-1.8. trifunctional linkers on average.

In a conjugated form, the trifunctional linker in its conjugated form is represented by Structure B:

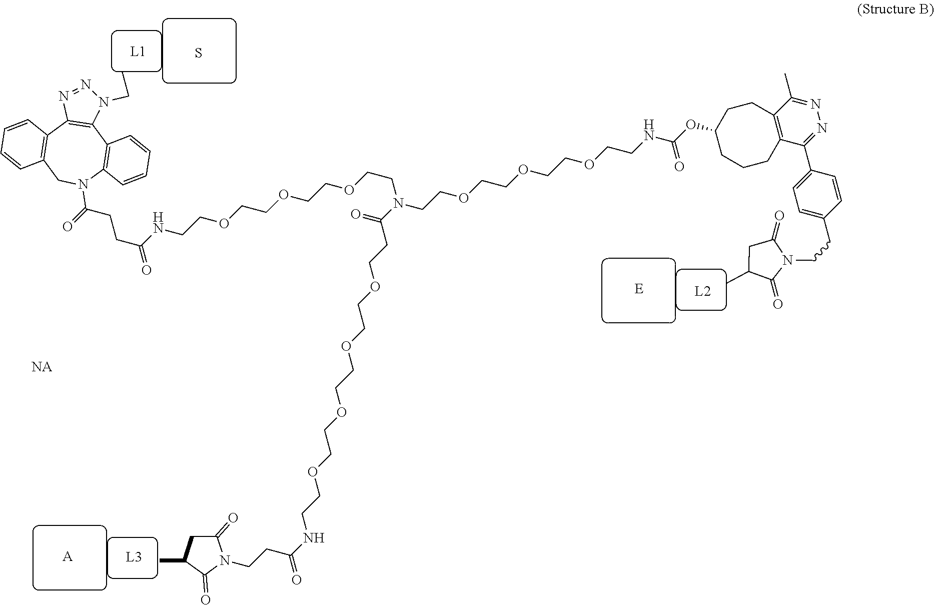

wherein:
S is the at least one saponin moiety,
L1 is a linker bound to the saponin moiety;
NA is the effector component comprising an nucleic acid,
L2 is a linker bound to the effector component;
A is one or more molecules of the first ligand, preferably being an antibody or a binding fragment thereof,
L3 is a linker bound to the first ligand,
wherein L1, L2 and L3 are the same or different.

In a particularly advantageous embodiment, a saponin component or a pharmaceutical composition for the disclosed herein use is provided, wherein the saponin moiety is covalently conjugated with the non-saponin moiety comprising the effector component, which conjugation results in bringing the saponin component and the effector component together in a conjugate further termed a saponin-effector component,
preferably wherein the saponin-effector component further comprises the linker, more preferably wherein the linker is directly covalently conjugated to the saponin moiety
possibly wherein the saponin-effector component further comprises the first ligand (resulting in a conjugate that is further termed targeted saponin-effector component.

In possible embodiments, the targeted saponin-effector component comprises 1-16 saponin moieties and 1-5 molecules of the nucleic acid (also termed effector moieties) per 1 ligand moiety, preferably wherein the targeted saponin-effector component comprises 2-8 saponin moieties per 1 ligand moiety;
preferably 3-6 saponin moieties per 1 ligand moiety; more preferably 4-5 saponin moieties per 1 ligand moiety; most preferably wherein the targeted saponin-effector component comprises on average 4-4.5 saponin moieties per 1 molecule of the ligand.

In an advantageous embodiment, compatible with preceding embodiments, a saponin component or a pharmaceutical composition for the disclosed herein use is provided, wherein the administration comprises provision of the effector component and the saponin component formulated as a single pharmaceutical formulation, or formulated as at least two (physically separated, e.g. provided in different containers or packages) pharmaceutical formulations that can be administered either simultaneously or sequentially, wherein the first pharmaceutical formulation comprises the saponin component and the second pharmaceutical formulation comprises the effector component,

In certain advantageous embodiments, the administration can be followed with a boosting application of the saponin component that is further referred to as a boosting saponin component.

We have observed that such boosting application (booster) of the saponin component (the boosting saponin component) can result in an extension of the duration of effects of the nucleic acid therapeutic and/or in an extension of the dosing interval of the nucleic acid therapeutic and/or in a reduction of the dosing frequency of the nucleic acid therapeutic and/or in a (delayed) potentiation in the effect of the nucleic therapeutic.

In advantageous embodiments, the administration is further followed after an interval of at least 1 day, preferably at least one week, with a boosting application of the saponin component that is further referred to as a boosting saponin component, wherein the boosting saponin component is provided without the effector component and preferably comprises the unconjugated saponin molecule of or the saponin moiety of any one of the, preferably wherein the saponin moiety is covalently conjugated with the non-saponin moiety being at least the linker or at least the first ligand or at least the linker and the first ligand.

In possible embodiments, the interval is at least 1 day after the administration, preferably at least 2 days, at least 3 days, at least 1 week, at least 2 weeks, at least 3 weeks, at least 1 month, at least 2 months, at least 3 months, at least 4 months, at least 5 months, or at least 6 months after the administration.

In certain embodiments, the boosting application can be performed directly into the organ or into a body cavity or fluid space that is in communication with the cells of the organ.

In particular embodiments, the boosting application can be performed at a site of the administration, or if the administration involved application at multiple sites, the boosting application can be made in one of these sites.

For example, in case of multiple sites of the administration or repeated administrations or in case of the administration encompassing multiple partial administrations, e.g. wherein the administration involves provision of two or more pharmaceutical formulations as separate and possibly timed doses, the site of the administration is to be construed as at least one of the sites of the administration.

Alternatively, in certain advantageous embodiments, the boosting application can be performed into the organ or into a body cavity or fluid space that is in communication with the cells of the organ, but by an application route that is less invasive and/or goes less deep into the subject's body as compared with the route of the administration.

For example, if the administration was made intrathecally into the CNS, it can be that the boosting administration is done only epidurally, which is a more standardised and less complicated intervention procedure that is better tolerated by patients and more commonly used by anaesthesiologists.

In another example, if the administration was made intravitreally to the eye, the boosting application of the saponin component could be applied in a less painful periocular route or even topically.

In a next embodiment, compatible with preceding embodiments, a saponin component or a pharmaceutical composition for the disclosed herein use is provided, wherein the administration comprises provision of the single pharmaceutical formulation selected from any one or more of the following:
2-component free-saponin formulation defined as comprising the saponin component consisting of the unconjugated saponin molecule, wherein the penta-cyclic triterpene saponin, and wherein the 2-component free-saponin formulation further comprises the effector component that possibly comprises a second ligand recognised by a second endocytic receptor;
2-component linker-saponin formulation defined as comprising the saponin component comprising the saponin moiety, wherein the saponin moiety is covalently conjugated with the linker; wherein the 2-component linker-saponin formulation further comprises the effector component that possibly comprises a second ligand recognised by a second endocytic receptor;
2-component targeted-saponin formulation defined as comprising the saponin component comprising the saponin moiety, wherein the saponin moiety is covalently conjugated with the first ligand, and preferably wherein the non-saponin moiety comprises the linker; and wherein the 2-component targeted-saponin formulation further comprises the effector component that possibly comprises the second ligand;
1-component formulation defined as comprising the saponin-effector component,
possibly wherein the saponin-effector component is a targeted saponin-effector component further comprising the first ligand.

In a next embodiment, compatible with preceding embodiments, a saponin component or a pharmaceutical composition for the disclosed herein use is provided, wherein the administration comprises provision of the at least two pharmaceutical formulations comprising a combination of the first pharmaceutical formulation with the second pharmaceutical formulation selected from any one or more of the following:
non-targeted combination defined as comprising
the first pharmaceutical formulation, wherein the saponin component does not comprise a ligand and preferably comprises or consists of the unconjugated saponin molecule and/or the saponin moiety, wherein the saponin moiety is covalently conjugated with the linker, and
the second pharmaceutical formulation, wherein the effector component does not comprise a ligand;
targeted-effector combination defined as comprising
the first pharmaceutical formulation, wherein the saponin component does not comprise a ligand and preferably comprises or consists of the unconjugated saponin molecule and/or the saponin moiety, wherein the saponin moiety is covalently conjugated with the linker, and
the second pharmaceutical formulation, wherein the effector component comprises the second ligand;
targeted-saponin combination defined as comprising
the first pharmaceutical formulation, wherein the saponin component comprises the saponin moiety, wherein the saponin moiety is covalently conjugated with the first ligand, and preferably wherein the non-saponin moiety comprises the linker, and
the second pharmaceutical formulation, wherein the effector component possibly comprises the second ligand.

In sum, as disclosed herein, the saponin component is
a penta-cyclic triterpene saponin of the 12,13-dehydrooleanane type; and
preferably comprising an aldehyde function at position C-23 of the aglycone core, or an acid-sensitive covalent bond configured to break under acidic conditions so as to create the aldehyde function at position C-23 of the aglycone core, preferably wherein the acid-sensitive covalent bond is selected from any one or more of: a semicarbazone bond, a hydrazone bond, an imine bond, an acetal bond including a 1,3-dioxolane bond, a ketal bond, an ester bond, and/or an oxime bond, and preferably is selected from a semicarbazone bond and a hydrazone bond.
mono-desmosidic or bi-desmosidic, preferably bi-desmosidic; and/or
comprising a first saccharide chain bound to its aglycone core structure, selected from Group A listed in Table 1A and/or comprising a second saccharide chain bound to its aglycone core structure, selected from Group B listed in Table 1A, and preferably a first saccharide chain and a second saccharide chain are comprised by the saponin molecule or saponin moiety:

TABLE 1A
GLYCANS

Group A
Ara/Xyl-(1→4)-Rha/Fuc-(1→2)-Glc/Gal-(1→2)-Rha/Fuc-(1→2)-GlcA-
Gal-
Gal-(1→2)-[Xyl-(1→3)]-GlcA-
Glc-
Glc-(1→2)-[Glc-(1→4)]-GlcA-
Glc-(1→2)-Ara-(1→3)-[Gal-(1→2)]-GlcA-
GlcA-
Rha-(1→2)-Ara-
Rha-(1→2)-Gal-(1→3)-[Glc-(1→2)]-GlcA-
Xyl-(1→2)-Ara-(1→3)-[Gal-(1→2)]-GlcA-
Group B
[4,6-di-OAc-Glc-(1→3)]-[Xyl-(1→4)]-Rha-(1→2)-[3,4-di-OAc-Qui-(1→4)]-Fuc-
6-OAc-Glc-(1→3)-[Xyl-(1→4)]-Rha-(1→2)-Fuc-
6-OAc-Glc-(1→3)-Xyl-(1→4)-Rha-(1→2)-[3-OAc-Rha-(1→3)]-Fuc-
Api-(1→3)-Xyl-(1→4)-[Glc-(1→3)]-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-[Glc-(1→3)]-Rha-(1→2)-[Rha-(1→3)]-40Ac-Fuc-
Api-(1→3)-Xyl-(1→4)-[Glc-(1→3)]-Rha-(1→2)-[Rha-(1→3)]-4-OAc-Fuc-
Api-(1→3)-Xyl-(1→4)-[Glc-(1→3)]-Rha-(1→2)-Fuc-
Api-(1→3)-Xyl-(1→4)-Rha-(1→2)-[R-(→3)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-Rha-(1→2)-[Rha-(1→3)]-4-OAc-Fuc-
Api/Xyl-(1→3)-Xyl-(1→4)-[Glc-(1→3)]-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api/Xyl-(1→3)-Xyl-(1→4)-[Glc-(1→3)]-Rha-(1→2)-[R-(→4)]-Fuc-
wherein R is 5-O-[5-O-Rha-(1→2)-Ara/Api-3,5-dihydroxy-6-methyl-octanoyl]-3,5-dihydroxy-
6-methyl-octanoic acid
Api/Xyl-(1→3)-Xyl-(1→4)-[Glc-(1→3)]-Rha-(1→2)-[Rha-(1→3)]-40Ac-Fuc-
Ara/Xyl-
Ara/Xyl-(1→3)-Ara/Xyl-(1→4)-Rha/Fuc-(1→2)-[4-OAc-Rha/Fuc-(1→4)]-Rha/Fuc-
Ara/Xyl-(1→4)-Rha/Fuc-(1→4)-[Glc/Gal-(1→2)]-Fuc-
Glc-(1→3)-[Glc-(1→6)]-Gal-
Glc-(1→3)-[Xyl-(1→3)-Xyl-(1→4)]-Rha-(1→2)-Fuc-
Glc-(1→3)-[Xyl-(1→4)]-Rha-(1→2)-[Qui-(1→4)]-Fuc-
Glc-(1→3)-[Xyl-(1→4)]-Rha-(1→2)-Fuc-
Glc-(1→3)-Xyl-(1→4)-[Glc-(1→3)]-Rha-(1→2)-4-OAc-Fuc-
Glc-(1→3)-Xyl-(1→4)-Rha-(1→2)-[3-OAc--Rha-(1→3)]-Fuc-
Glc-(1→3)-Xyl-(1→4)-Rha-(1→2)-[Xyl-(1→3)-4-OAc-Qui-(1→4)]-Fuc-
Glc-(1→3)-Xyl-(1→4)-Rha-(1→2)-4-OAc-Fuc-
Glc/Gal-
Rha-(1→2)-[Ara-(1→3)-Xyl-(1→4)]-Rha-
Rha-(1→2)-[Xyl-(1→4)]-Rha-
Xyl-(1→3)-Xyl-(1→4)-Rha-(1→2)-[3,4-di-OAc-Qui-(1→4)]-Fuc-
Xyl-(1→3)-Xyl-(1→4)-Rha-(1→2)-[Qui-(1→4)]-Fuc-
Xyl-(1→3)-Xyl-(1→4)-Rha-(1→2)-[R-(→3)]-Fuc-
wherein R is 5-O-[5-O-Ara/Api-3,5-dihydroxy-6-methyl-octanoyl]-3,5-dihydroxy-6-methyl-
octanoic acid
Xyl-(1→3)-Xyl-(1→4)-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Xyl-(1→3)-Xyl-(1→4)-Rha-(1→2)-[Xyl-(1→3)-4-OAc-Qui-(1→4)]-Fuc-
Xyl-(1→4)-[Gal-(1→3)]-Rha-(1→2)-4-OAc-Fuc-
Xyl-(1→4)-[Gal-(1→3)]-Rha-(1→2)-Fuc-
Xyl-(1→4)-[Glc-(1→3)]-Rha-(1→2)-[R-(→4)]-3-OAc-Fuc-
wherein R is 4E-Methoxycinnamic acid)
Xyl-(1→4)-[Glc-(1→3)]-Rha-(1→2)-3,4-di-OAc-Fuc-
Xyl-(1→4)-[Glc-(1→3)]-Rha-(1→2)-Fuc-
Xyl-(1→4)-Rha-(1→2)-[R-(→4)]-Fuc-
wherein R is 4E-Methoxycinnamic acid
Xyl-(1→4)-Rha-(1→2)-[R-(→4)]-Fuc-
wherein R is 4Z-Methoxycinnamic acid

and/or
preferably comprising a first saccharide chain bound to position C-3 of its aglycone core structure, selected from Group A listed in Table 1A, wherein preferably said first saccharide chain of the saponin molecule comprises a glucuronic acid group or optionally said first saccharide chain of the saponin moiety comprises a glucuronic acid group; and/or
preferably comprising the first saccharide chain which comprises a terminal glucuronic acid residue and/or comprising the second saccharide chain which comprises at least four sugar residues in a branched configuration; and/or
preferably comprising the first saccharide chain Gal-(1→2)-[Xyl-(1→3)]-GlcA and/or a branched second saccharide chain of at least four sugar residues comprising a terminal fucose residue and/or a terminal rhamnose residue, preferably selected from Table 1A; and/or
preferably comprising a first saccharide chain at position C-3 of the saponin's aglycone core structure and/or a second saccharide chain at position C-28 of the saponin's aglycone core structure, preferably wherein the first saccharide chain is a carbohydrate substituent at the C-3beta-OH group of the saponin's aglycone core structure and/or wherein the second saccharide chain is a carbohydrate substituent at the C-28-OH group of the saponin's aglycone core structure; and/or
optionally comprising at least one acetoxy (Me(CO)O—) group in the first saccharide chain and/or in the second saccharide chain, preferably in the second saccharide chain; and/or
comprising an aglycone core structure selected from:
quillaic acid;
gypsogenin;
2alpha-hydroxy oleanolic acid;
16alpha-hydroxy oleanolic acid;
hederagenin (23-hydroxy oleanolic acid);
16alpha,23-dihydroxy oleanolic acid;
protoaescigenin-21 (2-methylbut-2-enoate)-22-acetate;
23-oxo-barringtogenol C-21,22-bis(2-methylbut-2-enoate);
23-oxo-barringtogenol C-21 (2-methylbut-2-enoate)-16,22-diacetate;
3,16,28-trihydroxyoleanan-12-en;
gypsogenic acid; and
a derivative thereof; and/or
preferably comprising an aglycone core structure selected from quillaic acid, gypsogenin, and a derivative thereof; and/or
preferably comprising the aglycone core structure quillaic acid; and/or
selected from any one or more of the saponins listed in Table 2A:

TABLE 2A
Saponins displaying (late) endosomal/lysosomal escape enhancing activity,
and with a aglycone core of the 12,13-dehydrooleanane type⁴⁾

| | Aglycone core with | Carbohydrate | |
|---|---|---|---|
| | an aldehyde group | substituent at the | Carbohydrate substituent at the C-28-OH |
| Saponin Name | at the C-23 position | C-3beta-OH group | group |
| NP-017777 | Gypsogenin | Gal-(1→2)-[Xyl- | Xyl-(1→4)-Rha-(1→2)-[R-(→4)]-Fuc- (R = 4E- |
| | | (1→3)]-GlcA- | Methoxycinnamic acid) |
| NP-017778 | Gypsogenin | Gal-(1→2)-[Xyl- | Xyl-(1→4)-Rha-(1→2)-[R-(→4)]-Fuc- (R = 4Z- |
| | | (1→3)]-GlcA- | Methoxycinnamic acid) |
| NP-017774 | Gypsogenin | Gal-(1→2)-[Xyl- | Xyl-(1→4)-[Gal-(1→3)]-Rha-(1→2)-4-OAc-Fuc- |
| | | (1→3)]-GlcA- | |
| NP-018110^{c}, NP- | Gypsogenin | Gal-(1→2)-[Xyl- | Xyl-(1→4)-[Glc-(1→3)]-Rha-(1→2)-3,4-di-OAc- |
| 017772^{d} | | (1→3)]-GlcA- | Fuc- |
| NP-018109 | Gypsogenin | Gal-(1→2)-[Xyl- | Xyl-(1→4)-[Glc-(1→3)]-Rha-(1→2)-[R-(→4)]-3- |
| | | (1→3)]-GlcA- | OAc-Fuc- (R = 4E-Methoxycinnamic acid) |
| NP-017888 | Gypsogenin | Gal-(1→2)-[Xyl- | Glc-(1→3)-Xyl-(1→4)-[Glc-(1→3)]-Rha-(1→2)- |
| | | (1→3)]-GlcA- | 4-OAc-Fuc- |
| NP-017889 | Gypsogenin | Gal-(1→2)-[Xyl- | Glc-(1→3)-Xyl-(1→4)-Rha-(1→2)-4-OAc-Fuc- |
| | | (1→3)]-GlcA- | |
| NP-018108 | Gypsogenin | Gal-(1→2)-[Xyl- | Ara/Xyl-(1→3)-Ara/Xyl-(1→4)-Rha/Fuc-(1→2)- |
| | | (1→3)]-GlcA- | [4-OAc-Rha/Fuc-(1→4)]-Rha/Fuc- |
| SA1641^{a}, AE X55^{b} | Gypsogenin | Gal-(1→2)-[Xyl- | Xyl-(1→3)-Xyl-(1→4)-Rha-(1→2)-[Qui-(1→4)]- |
| | | (1→3)]-GlcA- | Fuc- |
| SO1658 | Gypsogenin | Gal-(1→2)-[Xyl- | Glc-(1→3)-[Xyl-(1→3)-Xyl-(1→4)]-Rha-(1→2)- |
| | | (1→3)]-GlcA- | Fuc- |
| gypsoside A⁶⁾ | Gypsogenin | Gal-(1→4)-Glc | Xyl-(1→3)-Fuc-(1→4)-[Xyl-(1→3)-Xyl-(1→3)]- |
| | | (1→4)-[Ara-(1→3)]- | Rha- |
| | | GlcA- | |
| phytolaccagenin | Gypsogenin | Absent | absent |
| Gypsophila saponin | Quillaic acid | Gal-(1→2)-[Xyl- | Glc-(1→3)-[Xyl-(1→4)]-Rha-(1→2)-Fuc- |
| 1 (Gyp1) | | (1→3)]-GlcA- | |
| NP-017674 | Quillaic acid | Gal-(1→2)-[Xyl- | Api-(1→3)-Xyl-(1→4)-[Glc-(1→3)]-Rha-(1→2)- |
| | | (1→3)]-GlcA- | Fuc- |
| NP-017810 | Quillaic acid | Gal-(1→2)-[Xyl- | Xyl-(1→4)-[Gal-(1→3)]-Rha-(1→2)-Fuc- |
| | | (1→3)]-GlcA- | |
| AG1 | Quillaic acid | Gal-(1→2)-[Xyl- | Xyl-(1→4)-[Glc-(1→3)]-Rha-(1→2)-Fuc- |
| | | (1→3)]-GlcA- | |
| NP-003881 | Quillaic acid | Gal-(1→2)-[Xyl- | Ara/Xyl-(1→4)-Rha/Fuc-(1→4)-[Glc/Gal- |
| | | (1→3)]-GlcA- | (1→2)]-Fuc- |
| NP-017676 | Quillaic acid | Gal-(1→2)-[Xyl- | Api-(1→3)-Xyl-(1→4)-[Glc-(1→3)]-Rha-(1→2)- |
| | | (1→3)]-GlcA- | [R-(→4)]-Fuc- |
| | | | (R = 5-O-[5-O-Ara/Api-3,5-dihydroxy-6-methyl- |
| | | | octanoyl]-3,5-dihydroxy-6-methyl-octanoic acid) |
| NP-017677 | Quillaic acid | Gal-(1→2)-[Xyl- | Api-(1→3)-Xyl-(1→4)-Rha-(1→2)-[R-(→4)]- |
| | | (1→3)]-GlcA- | Fuc- |
| | | | (R = 5-O-[5-O-Ara/Api-3,5-dihydroxy-6-methyl- |
| | | | octanoyl]-3,5-dihydroxy-6-methyl-octanoic acid) |
| NP-017706 | Quillaic acid | Gal-(1→2)-[Xyl- | Api-(1→3)-Xyl-(1→4)-Rha-(1→2)-[Rha-(1→3)]- |
| | | (1→3)]-GlcA- | 4-OAc-Fuc- |
| NP-017705 | Quillaic acid | Gal-(1→2)-[Xyl- | Api-(1→3)-Xyl-(1→4)-[Glc-(1→3)]-Rha-(1→2)- |
| | | (1→3)]-GlcA- | [Rha-(1→3)]-4-OAc-Fuc- |
| NP-017773 | Quillaic acid | Gal-(1→2)-[Xyl- | 6-OAc-Glc-(1→3)-Xyl-(1→4)-Rha-(1→2)-[3- |
| | | (1→3)]-GlcA- | OAc-Rha-(1→3)]-Fuc- |
| NP-017775 | Quillaic acid | Gal-(1→2)-[Xyl- | Glc-(1→3)-Xyl-(1→4)-Rha-(1→2)-[3-OAc--Rha- |
| | | (1→3)]-GlcA- | (1→3)]-Fuc- |
| SA1657 | Quillaic acid | Gal-(1→2)-[Xyl- | Xyl-(1→3)-Xyl-(1→4)-Rha-(1→2)-[Qui-(1→4)]- |
| | | (1→3)]-GlcA- | Fuc- |
| AG2 | Quillaic acid | Gal-(1→2)-[Xyl- | Glc-(1→3)-[Xyl-(1→4)]-Rha-(1→2)-[Qui- |
| | | (1→3)]-GlcA- | (1→4)]-Fuc- |
| GE1741 | Quillaic acid | Gal-(1→2)-[Xyl- | Xyl-(1→3)-Xyl-(1→4)-Rha-(1→2)-[3,4-di-OAc- |
| | | (1→3)]-GlcA- | Qui-(1→4)]-Fuc- |
| SO1542 | Quillaic acid | Gal-(1→2)-[Xyl- | Glc-(1→3)-[Xyl-(1→4)]-Rha-(1→2)-Fuc- |
| | | (1→3)]-GlcA- | |
| SO1584 | Quillaic acid | Gal-(1→2)-[Xyl- | 6-OAc-Glc-(1→3)-[Xyl-(1→4)]-Rha-(1→2)-Fuc- |
| | | (1→3)]-GlcA- | |
| SO1674 | Quillaic acid | Gal-(1→2)-[Xyl- | Glc-(1→3)-[Xyl-(1→3)-Xyl-(1→4)]-Rha-(1→2)- |
| | | (1→3)]-GlcA- | Fuc- |
| SO1700³⁾ | Quillaic acid | Gal-(1→2)-[Xyl- | Xyl-(1→4)-Rha-(1→2)-[Xyl-(1→3)-4-OAc-Qui- |
| | | (1→3)]-GlcA- | (1→4)]-Fuc- |
| Saponarioside B¹⁾ | Quillaic acid | Gal-(1→2)-[Xyl- | Xyl-(1→3)-Xyl-(1→4)-Rha-(1→2)-[4-OAc-Qui- |
| | | (1→3)]-GlcA- | (1→4)]-Fuc- |
| SO1730³⁾ | Quillaic acid | Gal-(1→2)-[Xyl- | Glc-(1→3)-Xyl-(1→4)-Rha-(1→2)-[-4-OAc-Qui- |
| | | (1→3)]-GlcA- | (1→4)]-Fuc- |
| SO1772³⁾ | Quillaic acid | Gal-(1→2)-[Xyl- | 6-OAc-Glc-(1→3)-[Xyl-(1→4)]-Rha-(1→2)-[4- |
| | | (1→3)]-GlcA- | OAc-Qui-(1→4)]-Fuc-- |
| SO1832¹⁾ | Quillaic acid | Gal-(1→2)-[Xyl- | Xyl-(1→3)-Xyl-(1→4)-Rha-(1→2)-[Xyl-(1→3)-4- |
| (protonated | | (1→3)]-GlcA- | OAc-Qui-(1→4)]-Fuc- |
| SO1831) = | | | |
| Saponarioside A | | | |
| SO1861 | Quillaic acid | Gal-(1→2)-[Xyl- | Glc-(1→3)-Xyl-(1→4)-Rha-(1→2)-[Xyl-(1→3)-4- |
| (deprotonated | | (1→3)]-GlcA- | OAc-Qui-(1→4)]-Fuc- |
| SO1862) | | | |
| SO1862 | Quillaic acid | Gal-(1→2)-[Xyl- | Glc-(1→3)-Xyl-(1→4)-Rha-(1→2)-[Xyl-(1→3)-4- |
| (protonated | | (1→3)]-GlcA- | OAc-Qui-(1→4)]-Fuc- |
| SO1861), also | | | |
| referred to as | | | |
| Sapofectosid⁵⁾ | | | |
| SO1904³⁾ | Quillaic acid | Gal-(1→2)-[Xyl- | 6-OAc-Glc-(1→3)-[Xyl-(1→4)]-Rha-(1→2)-[Xyl- |
| | | (1→3)]-GlcA- | (1→3)-4-OAc-Qui-(1→4)]-Fuc- |
| QS-7 (also referred | Quillaic acid | Gal-(1→2)-[Xyl- | Api/Xyl-(1→3)-Xyl-(1→4)-[Glc-(1→3)]-Rha- |
| to as QS1861) | | (1→3)]-GlcA- | (1→2)-[Rha-(1→3)]-40Ac-Fuc- |
| QS-7 api (also | Quillaic acid | Gal-(1→2)-[Xyl- | Api-(1→3)-Xyl-(1→4)-[Glc-(1→3)]-Rha-(1→2)- |
| referred to as | | (1→3)]-GlcA- | [Rha-(1→3)]-40Ac-Fuc- |
| QS1862) | | | |
| QS-17 | Quillaic acid | Gal-(1→2)-[Xyl- | Api/Xyl-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Rha-(1→2)-Ara/Api-3,5- |
| | | | dihydroxy-6-methyl-octanoyl]-3,5-dihydroxy-6- |
| | | | methyl-octanoic acid) |
| QS-18 | Quillaic acid | Gal-(1-+2)-[Xyl- | Api/Xyl-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Ara/Api-3,5-dihydroxy-6-methyl- |
| | | | octanoyl]-3,5-dihydroxy-6-methyl-octanoic acid) |
| QS-21 A-apio | Quillaic acid | Gal-(1→2)-[Xyl- | Api-(1→3)-Xyl-(1→4)-Rha-(1→2)-[R-(→4)]- |
| | | (1→3)]-GlcA- | Fuc- |
| | | | (R = 5-O-[5-O-Ara/Api-3,5-dihydroxy-6-methyl- |
| | | | octanoyl]-3,5-dihydroxy-6-methyl-octanoic acid) |
| QS-21 A-xylo | Quillaic acid | Gal-(1→2)-[Xyl- | Xyl-(1→3)-Xyl-(1→4)-Rha-(1→2)-[R-(→4)]-Fuc- |
| | | (1→3)]-GlcA- | (R = 5-O-[5-O-Ara/Api-3,5-dihydroxy-6-methyl- |
| | | | octanoyl]-3,5-dihydroxy-6-methyl-octanoic acid) |
| QS-21 B-apio | Quillaic acid | Gal-(1→2)-[Xyl- | Api-(1→3)-Xyl-(1→4)-Rha-(1→2)-[R-(→3)]- |
| | | (1→3)]-GlcA- | Fuc- |
| | | | (R = 5-O-[5-O-Ara/Api-3,5-dihydroxy-6-methyl- |
| | | | octanoyl]-3,5-dihydroxy-6-methyl-octanoic acid) |
| QS-21 B-xylo | Quillaic acid | Gal-(1→2)-[Xyl- | Xyl-(1→3)-Xyl-(1→4)-Rha-(1→2)-[R-(→3)]-Fuc- |
| | | (1→3)]-GlcA- | (R = 5-O-[5-O-Ara/Api-3,5-dihydroxy-6-methyl- |
| | | | octanoyl]-3,5-dihydroxy-6-methyl-octanoic acid) |
| QS-21 | Quillaic acid | Gal-(1→2)-[Xyl- | Combination of the carbohydrate chains |
| | | (1→3)]-GlcA- | depicted for QS-21 A-apio, A-xylo, B-apio, B- |
| | | | xylo, for this position at the aglycone (see also |
| | | | the structure depicted as (Scheme Q)) |
| Agrostemmoside E | Quillaic acid | Gal-(1→2)-[Xyl- | [4,6-di-OAc-Glc-(1→3)]-[Xyl-(1→4)]-Rha- |
| (AG1856, AG2.8)²⁾ | | (1→3)]-GlcA- | (1→2)-[3,4-di-OAc-Qui-(1→4)]-Fuc- |

| | Aglycone core | | |
|---|---|---|---|
| | without an aldehyde | Carbohydrate | |
| | group at the C-23 | substituent at the | Carbohydrate substituent at the C-28-OH |
| Saponin Name | position | C-3beta-OH group | group |
| NP-005236 | 2alpha- | GlcA- | Glc/Gal- |
| | Hydroxyoleanolic acid | | |
| AMA-1 | 16alpha- | Glc- | Rha-(1→2)-[Xyl-(1→4)]-Rha- |
| | Hydroxyoleanolic acid | | |
| AMR | 16alpha- | Glc- | Rha-(1→2)-[Ara-(1→3)-Xyl-(1→4)]-Rha- |
| | Hydroxyoleanolic acid | | |
| alpha-Hederin | Hederagenin (23- | Rha-(1→2)-Ara- | Not present |
| | Hydroxyoleanolic | | |
| | acid) | | |
| NP-012672 | 16alpha,23- | Ara/Xyl-(1→4)- | Ara/Xyl- |
| | Dihydroxyoleanolic | Rha/Fuc-(1→2)- | |
| | acid | Glc/Gal-(1→2)- | |
| | | Rha/Fuc-(1→2)- | |
| | | GlcA- | |
| beta-Aescin | Protoaescigenin-21(2- | Glc-(1→2)-[Glc- | Not present |
| (described: Aescin | methylbut-2-enoate)- | (1→4)]-GlcA- | |
| Ia) | 22-acetat | | |
| Aescinate⁷⁾ | Aglycone core without | present | Not present |
| | an aldehyde group at | | |
| | the C-23 position | | |
| Aescin (Escin) ⁸⁾ | Aglycone core without | Glc-(1→4)-[Xyl- | Not present |
| | an aldehyde group at | (1→2)]-GlcA- | |
| | the C-23 position | | |
| dipsacoside B⁹⁾ | Aglycone core without | present | present |
| | an aldehyde group at | | |
| | the C-23 position | | |
| esculentoside A¹⁰⁾ | Aglycone core without | present | Not present |
| | an aldehyde group at | | |
| | the C-23 position | | |
| Teaseed saponin I | 23-Oxo- | Glc-(1→2)-Ara- | Not present |
| | barringtogenol C - | (1→3)-[Gal-(1→2)]- | |
| | 21,22-bis(2- | GlcA- | |
| | methylbut-2-enoate) | | |
| Teaseedsaponin J | 23-Oxo- | Xyl-(1→2)-Ara- | Not present |
| | barringtogenol C - | (1→3)-[Gal-(1→2)]- | |
| | 21,22-bis(2- | GlcA- | |
| | methylbut-2-enoate) | | |
| Assamsaponin F | 23-Oxo- | Glc-(1→2)-Ara- | Not present |
| | barringtogenol C - | (1→3)-[Gal-(1→2)]- | |
| | 21(2-methylbut-2- | GlcA- | |
| | enoate)-16,22- | | |
| | diacetat | | |
| Primula acid 1 | 3,16,28- | Rha-(1→2)-Gal- | Not present |
| | Trihydroxyoleanan- | (1→3)-[Glc-(1→2)]- | |
| | 12-en | GlcA- | |
| AS64R | Gypsogenic acid | Absent | Glc-(1→3)-[Glc-(1→6)]-Gal- |
| Macranthoidin A¹¹⁾ | Aglycone core without | present | present |
| | an aldehyde group at | | |
| | the C-23 position | | |
| saikosaponin A¹²⁾ | Aglycone core without | present | absent |
| | an aldehyde group at | | |
| | the C-23 position | | |
| saikosaponin D¹³⁾ | Aglycone core without | present | absent |
| | an aldehyde group at | | |
| | the C-23 position | | |

| | | Carbohydrate | |
|---|---|---|---|
| | | substituent at the | |
| | | C-23-OH group | |
| AS6.2 | Gypsogenic acid | Gal- | Glc-(1→3)-[Glc-(1→6)]-Gal- |

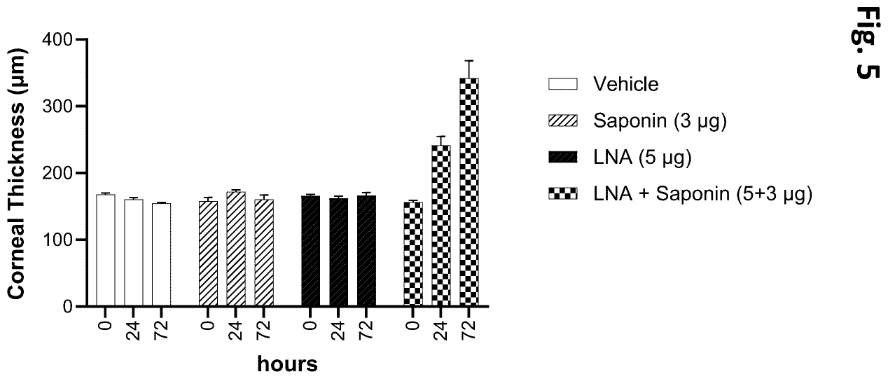

^{a, b}Different names refer to different isolates of the same structure
^{c, d}Different names refer to different isolates of the same structure
¹⁾Jia et al., Major Triterpenoid Saponins from Saponaria officinalis, J. Nat. Prod. 1998, 61, 11, 1368-1373, Publication Date: Sep. 19, 1998, https://doi.org/10.1021/np980167u
²⁾The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.
³⁾Structures of SO1700, SO1730, SO1772, SO1904 are given in Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁴⁾See for example:
- thesis by Dr Stefan Böttger (2013): Untersuchungen zur synergistischen Zytotoxizität zwischen Saponinen und Ribosomen inaktivierenden Proteinen Typ I; and
- Sama et al., Structure-Activity Relationship of Transfection-Modulating Saponins - A Pursuit for the Optimal Gene Trafficker, Planta Med. Volume 85, pp. 513-518, 2019 (doi:10.1055/a-0863-4795); and
- Fuchs et al., Glycosylated Triterpenoids as Endosomal Escape Enhancers in Targeted Tumor Therapies, Biomedicine, Volume 5, issue 14, 2017 (doi:10.3390/biomedicines5020014).
⁵⁾Sama et al., Sapofectosid - Ensuring non-toxic effective DNA and RNA delivery, International Journal of Pharmaceutics, Volume 534, Issues 1-2, 20 Dec. 2017, Pages 195-205 (dx.doi.org/10.1016/j.ijpharm.2017.10.016) & Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁶⁾See for example: doi:10.1016/s0040-4039(01)90658-6, Tetrahedron Letters No. 8, pp. 477-482, 1963 and and “Gipsoside.” National Center for Biotechnology Information. PubChem Compound Database, U.S. National Library of Medicine, 8 Aug. 2005, pubchem.ncbi.nlm.nih.gov/compound/Gipsoside.
⁷⁾The structure of Sodium Aescinate is for example given in the National Library of Medicine PubChem Compound Database (“Sodium Aescinate.” National Center for Biotechnology Information. PubChem Compound Database, U.S. National Library of Medicine, 26 Mar. 2005, pubchem.ncbi.nlm.nih.gov/compound/Sodium-aescinate,)
⁸⁾ The structure of Aescin (also referred to as Escin) is for example given in the National Library of Medicine PubChem Compound Database (“Escin.” National Center for Biotechnology Information. PubChem Compound Database, U.S. National Library of Medicine, 12 Jul. 2007, pubchem.ncbi.nlm.nih.gov/compound/16211024#section=Other-Identifiers.)
⁹⁾The structure of Dipsacoside B is for example given in the National Library of Medicine PubChem Compound Database (“Dipsacoside B.” National Center for Biotechnology Information. PubChem Compound Database, U.S. National Library of Medicine, 5 Dec. 2007, pubchem.ncbi.nlm.nih.gov/compound/21627940.)
¹⁰⁾The structure of Esculentoside A is for example given in the National Library of Medicine PubChem Compound Database (“Esculentoside a.” National Center for Biotechnology Information. PubChem Compound Database, U.S. National Library of Medicine, 26 Oct. 2006, pubchem.ncbi.nlm.nih.gov/compound/11657924.)
¹¹⁾The structure of Macranthoidin A is for example given in the National Library of Medicine PubChem Compound Database (“Macranthoidin a.” National Center for Biotechnology Information. PubChem Compound Database, U.S. National Library of Medicine, 9 Feb. 2007, pubchem.ncbi.nlm.nih.gov/compound/14564503.)
¹²⁾The structure of Saikosaponin A is for example given in the National Library of Medicine PubChem Compound Database (“Saikosaponin a.” National Center for Biotechnology Information. PubChem Compound Database, U.S. National Library of Medicine, 26 Jun. 2005, pubchem.ncbi.nlm.nih.gov/compound/167928.)
¹³⁾The structure of Saikosaponin D is for example given in the National Library of Medicine PubChem Compound Database (“Saikosaponin d.” National Center for Biotechnology Information. PubChem Compound Database, U.S. National Library of Medicine, 1 Aug. 2005, pubchem.ncbi.nlm.nih.gov/compound/107793.)

and/or
a) selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) comprising a gypsogenin aglycone core structure and is selected from list B:
   SA1641, gypsoside A, NP-017772, NP-017774, NP-017777, NP-017778, NP-018109, NP-017888, NP-017889, NP-018108, SO1658 and Phytolaccagenin; or
c) comprising a quillaic acid aglycone core structure and is selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo; or
d) comprising a 12,13-dehydrooleanane type aglycone core structure without an aldehyde group at the C-23 position of the aglycone and is selected from list D:
   Aescin Ia, aescinate, alpha-Hederin, AMA-1, AMR, AS6.2, AS64R, Assamsaponin F, dipsacoside B, esculentoside A, macranthoidin A, NP-005236, NP-012672, Primula acid 1, saikosaponin A, saikosaponin D, Teaseed saponin I and Teaseedsaponin J,
preferably, any one or more selected from list A, B or C, more preferably, selected from list B or C, even more preferably selected from list C; and/or
   any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832 having a formula according to formula ‘SO1832’

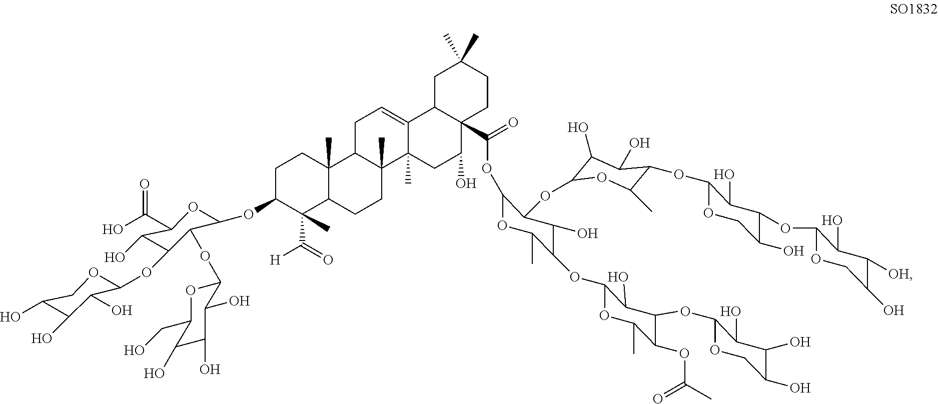

SO1861 having a formula according to formula ‘SO1861’

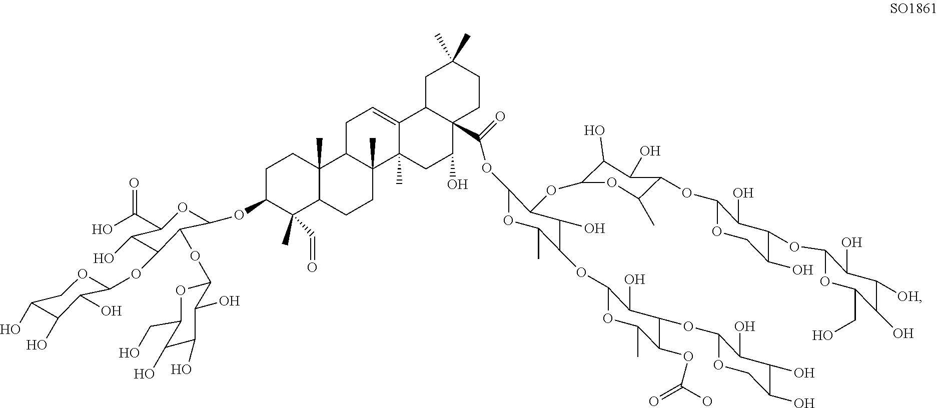

SO1862 and SO1904, preferably any one or more of QS-21, SO1832, SO1861, SA1641, AG1856 and GE1741, more preferably AG1856, SO1832 or SO1861, most preferably SO1861 or SO1832; and/or
a saponin isolated from Saponaria officinalis, preferably any one or more of Saponarioside B, SO1542, SO1584, SO1658, SO1674, SO1700, SO1730, SO1772, SO1832, SO1861, SO1862 and SO1904, more preferably any one or more of SO1832, SO1861 and SO1862, even more preferably SO1832 or SO1861, most preferably SO1861; and/or
a saponin molecule, wherein the carboxyl group of the glucuronic acid unit in the first saccharide chain bound to C-3 of the aglycone core structure of the saponin molecule is transformed into an amide bond through reaction with 2-amino-2-methyl-1,3-propanediol (AMPD) as shown for SO1861 in formula (3):

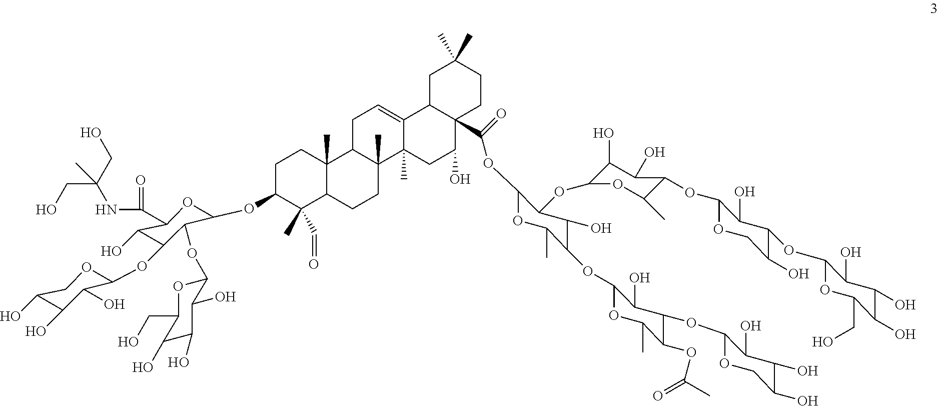

or a saponin molecule having a formula according to one of the following formulas (9)-(12):

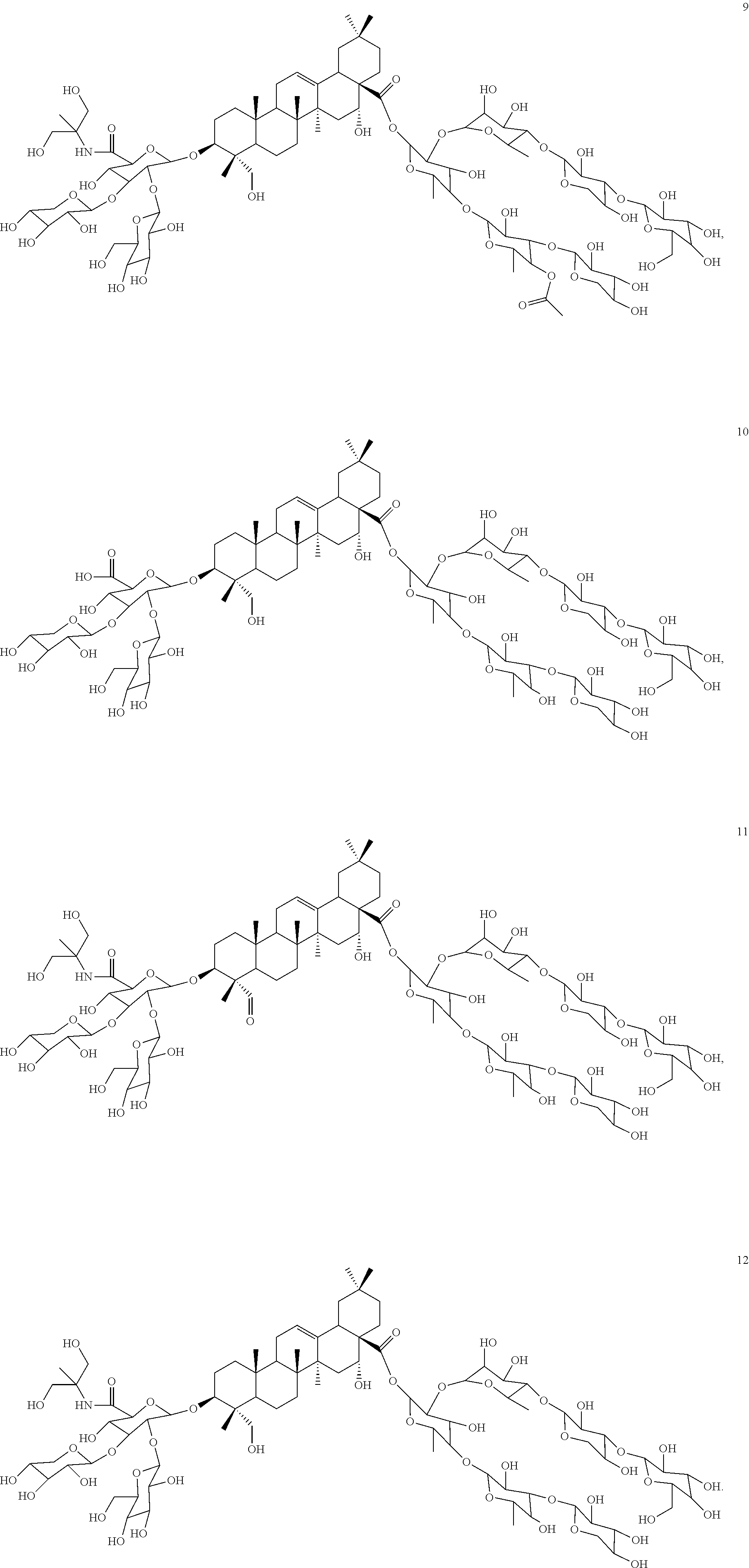

In certain preferred embodiments, the saponin comprises a glucuronic acid group in the carbohydrate substituent at the C-3beta-OH group, and preferably the saponin is selected from the group consisting of (refer to Table 2A for the structural details): NP-017777, NP-017778, NP-017774, NP-018110, NP-017772, NP-018109, NP-017888, NP-017889, NP-018108, SA1641, AE X55, SO1658, gypsoside A, Gypsophila saponin 1 (Gyp1), NP-017674, NP-017810, AG1, NP-003881, NP-017676, NP-017677, NP-017706, NP-017705, NP-017773, NP-017775, SA1657, AG2, GE1741, SO1542, SO1584, SO1674, SO1700, Saponarioside B, SO1730, SO1772, SO1832 (protonated SO1831; also referred to as Saponarioside A), SO1861 (deprotonated SO1862), SO1862 (protonated SO1861; also referred to as Sapofectosid), SO1904, QS-7 (also referred to as QS1861), QS-7 api (also referred to as QS1862), QS-17, QS-18, QS-21 A-apio, QS-21 A-xylo, QS-21 B-apio, QS-21 B-xylo, QS-21, Agrostemmoside E (also referred to as AG1856 or AG2.8), NP-005236, NP-012672, beta-Aescin (described: Aescin Ia), Aescinate, Teaseed saponin I, Teaseedsaponin J, Assamsaponin F, Primula acid 1.

In certain preferred embodiments, the saponin does not comprise an aldehyde function linked to the C-4 atom of the aglycon core structure, and preferably the saponin is selected from the group consisting of (refer to Table 2A for the structural details): NP-005236, AMA-1, AMR, alpha-Hederin, NP-012672, beta-Aescin (described: Aescin Ia), Aescinate, dipsacoside B, esculentoside A, Teaseed saponin I, Teaseedsaponin J, Assamsaponin F, Primula acid 1, AS64R, Macranthoidin A, saikosaponin A, saikosaponin D, AS6.2.

In certain preferred embodiments, the saponin comprises a glucuronic acid group in the carbohydrate substituent at the C-3beta-OH group and the saponin does not comprise an aldehyde function linked to the C-4 atom of the aglycon core structure, and preferably the saponin is selected from the group consisting of (refer to Table 2A for the structural details): NP-005236, NP-012672, beta-Aescin (described: Aescin la, Aescinate, dipsacoside B, esculentoside A, Teaseed saponin I, Teaseedsaponin J, Assamsaponin F, Primula acid 1, Macranthoidin A, saikosaponin A, saikosaponin D.

In some particular embodiments, possibly compatible with preceding ones, saponin components or compositions for the disclosed herein use can be provided, wherein one, two or three, preferably one or two, more preferably one, of:
an aldehyde group in the aglycone core structure of the at least one saponin has been derivatised when present,
a carboxyl group of a glucuronic acid moiety in a first saccharide chain of the at least one saponin has been derivatised when present in the at least one saponin, and
at least one acetoxy (Me(CO)O—) group in a second saccharide chain of the at least one saponin has been derivatised if present.

In more particular embodiments, saponin components or compositions for the disclosed herein use can be provided wherein the at least one saponin comprises:
i. an aglycone core structure comprising an aldehyde group which has been derivatised by:
   reduction to an alcohol;
   transformation into a hydrazone bond through reaction with N-ε-maleimidocaproic acid hydrazide (EMCH) wherein the maleimide group of the EMCH is optionally derivatised by formation of a thioether bond with mercaptoethanol;
   transformation into a hydrazone bond through reaction with N-[β-maleimidopropionic acid] hydrazide (BMPH) wherein the maleimide group of the BMPH is optionally derivatised by formation of a thioether bond with mercaptoethanol; or
   transformation into a hydrazone bond through reaction with N-[κ-maleimidoundecanoic acid] hydrazide (KMUH) wherein the maleimide group of the KMUH is optionally derivatised by formation of a thioether bond with mercaptoethanol; or
ii. a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl) maleimide (AEM); or
iii. a second saccharide chain comprising an acetoxy group (Me(CO)O—) which has been derivatised by transformation into a hydroxyl group (HO—) by deacetylation; or
iv. any combination of two or three derivatisations i., ii. and/or iii., preferably any combination of two derivatisations of i., ii. and iii.

In a specific embodiment, a saponin component or a composition for the disclosed use is provided wherein the aldehyde function in position C-23 of the aglycone core structure of the at least one saponin is covalently bound to linker EMCH, which EMCH is covalently bound via a thio-ether bond to a sulfhydryl group in the oligomeric molecule or in the polymeric molecule of the covalent saponin conjugate, such as a sulfhydryl group of a cysteine. Binding of the EMCH linker to the aldehyde group of the aglycone of the saponin results in formation of a hydrazone bond. Such a hydrazone bond is a typical example of a cleavable bond under the acidic conditions inside endosomes and lysosomes.

When the saponin component comprises the saponin moiety, the saponin moiety is any one of the here-above defined saponin molecules with covalently bound thereto:
a linker, such as a linker suitable for covalently binding the saponin molecule to a further molecule, wherein the linker comprises or is for example:
a. a polyethylene glycol (PEG) with a length of any number between 2 and 60 (PEG2, PEG3, PEG4, PEG5, PEG6, PEG7-PEG10, PEG11-PEG25, PEG25-PEG50, etc.);
b. a peptide;
c. a linear or branched or cyclic alkyl, a linear or branched or cyclic alkenyl, a linear or branched or cyclic alkynyl;
d. a polymeric structure or an oligomeric structure, for example:
wherein the polymeric or oligomeric structure is selected from:
   i. poly- or oligo (amines), such as polyethylenimine and poly(amidoamine),
   ii. polyethylene glycols,
   iii. poly- or oligo (esters), such as poly(lactids),
   iv. poly(lactams),
   v. polylactide-co-glycolide copolymers,
   vi. poly- or oligosaccharides, such as cyclodextrin and polydextrose,
   vii. poly- or oligo (amino acids), such as proteins, peptides and polylysine, and viii. DNA oligomers or polymers, RNA polymers, stabilized RNA polymers and PNA (peptide nucleic acid) polymers, and/or
   ix. dendron of type G2, G3, G4 or G5;
a linker, such as a linker as hereabove defined, with a further molecule covalently bound to the linker
wherein said further molecule is any one or more of:
a. a further linker, such as a linker as hereabove defined; and/or
b. an effector moiety, wherein the effector moiety is an oligonucleotide therapeutic and/or
c. a ligand for binding to an endocytic cell-receptor,
wherein the ligand is a proteinaceous ligand or a non-proteinaceous ligand or a combination thereof, preferably wherein the ligand is a proteinaceous ligand, and for examples is:
a. a protein ligand capable of binding to a (n) endocytic cell-surface receptor, which binding results in internalization of the protein ligand, for example a cytokine or EGF;
b. an antibody, wherein the antibody is defined as an immunoglobulin (lg) or a functional binding fragment or binding domain thereof.

The saponin component is suitable for passive or active transfer from outside a cell to inside said cell. Moreover, the saponin is suitable for transfer from outside a cell into said cell, being the transfer in the endosomes of said cell. The saponin component is suitable for entry into a cell upon binding of a ligand for binding to an endocytic cell-receptor, bound to the saponin moiety comprised by the saponin component, to said endocytic cell receptor, via endocytosis. Upon binding of the ligand, endocytosis occurs and the saponin component is delivered in the endosomes of the cell bearing the cell receptor.

Notable examples of such cell-surface receptors are: CD71 and CD63.

Ligands for binding to such endocytic cell-surface receptors are for example comprised by the saponin component and/or by the effector component (such as the nucleic acid component) when the effector molecule or effector moiety comprised by the effector component should exert its therapeutic or prophylactic activity in a tumor cell.

Examples of endocytic receptors that can be selected for targeting by a ligand comprised by the saponin component (and/or comprised by the effector component such as the nucleic acid component) are: transferrin receptor (CD71), insulin-like growth factor 1 (IGF-I) receptor (IGF1R), tetraspanin CD63; muscle-specific kinase (MuSK), glucose transporter GLUT4, cation independent mannose 6 phosphate receptor (CI-MPR), and LDL receptor. Ligands for binding to such endocytic cell-surface receptors are for example comprised by the saponin component and/or by the effector component (such as the nucleic acid component) when the effector molecule or effector moiety comprised by the effector component should exert its therapeutic or prophylactic activity in a muscle cell.

When the proteinaceous ligand comprised by the saponin component (and suitable for binding to an endocytic cell-surface receptor) is an antibody, the antibody is for example selected from IgG, IgM, IgE, IgA, or IgD, or any antigen-binding fragment thereof, preferably is selected from a monoclonal antibody, polyclonal antibody, human antibody, humanized antibody, chimeric antibody, resurfaced antibody, anti-idiotypic antibody, mouse antibody, rat antibody, rat/mouse hybrid antibody, llama antibody, llama heavy-chain only antibody, heavy-chain only antibody, a molecule comprising or consisting of a Vhh domain, a Vh domain, a Fab, an scFv, an Fv, a single domain antibody (sdAb), an F (ab) 2, Fcab fragment. A monoclonal antibody and a Fab and a single sdAb or a string of covalently linked sdAb's is preferred.

The linker covalently bound to the saponin molecule, forming the saponin component comprising the saponin moiety and the linker (and in some embodiments a ligand covalently bound to the linker), is in preferred embodiments covalently bound to the saponin via a bond that is cleavable under conditions present in the endosome of mammalian cells, for example human cells. Such cleavable bond is for example subject to cleavage under acidic, reductive, enzymatic and/or light-induced conditions; preferably wherein the cleavable bond is selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond
   preferably being an acid-sensitive bond subject to cleavage in vivo under acidic conditions present in endosomes and/or lysosomes of human cells, preferably at pH 4.0-6.5, and more preferably at pH≤5.5; more preferably being an acid-sensitive bond selected from any one or more of: a semicarbazone bond, a hydrazone bond, an imine bond, an acetal bond including a 1,3-dioxolane bond, a ketal bond, an ester bond, and/or an oxime bond, even more preferably selected from a semicarbazone bond and a hydrazone bond; most preferably being a hydrazone bond.

In an embodiment of the invention, the saponin molecule comprises a glucuronic acid function with a carboxylic acid functional group in a carbohydrate substituent at the C-3beta-OH group of the saponin, wherein the carboxylic acid functional group is transformed into an active ester.

In an embodiment of the invention, the saponin moiety comprises a glucuronic acid function with a carboxylic acid functional group in a carbohydrate substituent at the C-3beta-OH group of the saponin, wherein the carboxylic acid functional group is transformed into an active ester upon binding of a linker to said carboxylic acid functional group. In an embodiment, a ligand as hereabove defined is covalently bound to said linker which linker is bound to the saponin moiety. An example of such a saponin moiety comprising an active ester is the moiety resulting from activation of the carboxylic group of the saponin molecule selected for providing the saponin moiety, via 1-[Bis(dimethylamino)methylene]-1H-1,2,3-triazolo[4,5-b]pyridinium 3-oxide hexafluorophosphate (HATU).

In embodiments, the linker that is bound to the saponin molecule in the saponin component further comprises an oligomeric or polymeric structure either being a dendron such as a poly-amidoamine (PAMAM) dendrimer, or a poly-ethylene glycol such as any of PEG3-PEG30; preferably the polymeric or oligomeric structure being any one of PEG4-PEG12 or any one of a G2 dendron, a G3 dendron, a G4 dendron and a G5 dendron, more preferably being a G2 dendron or a G3 dendron or a PEG3-PEG30.

For example, the saponin component comprises a saponin moiety comprising a covalently bound linker and is a molecule according to any one of formula (I)-(V):

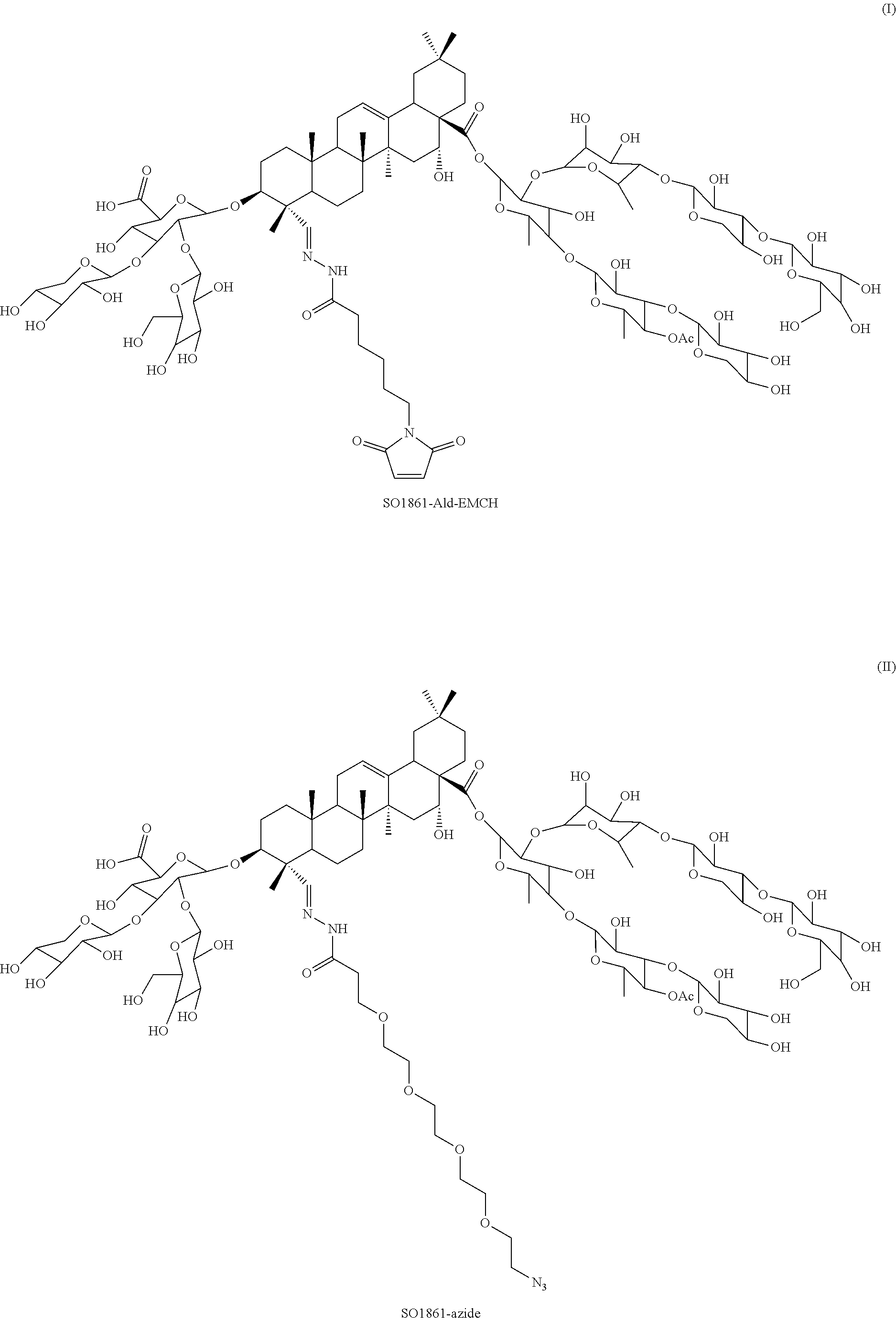

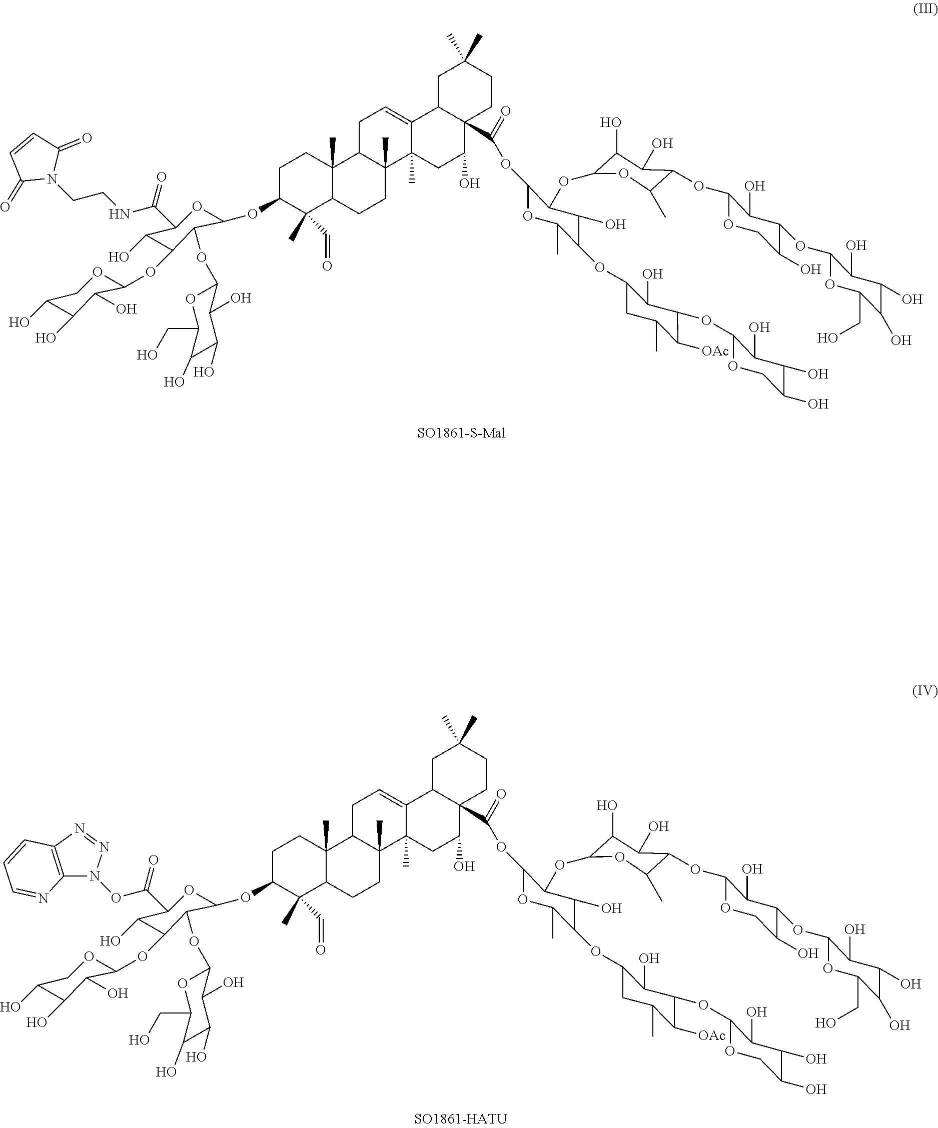

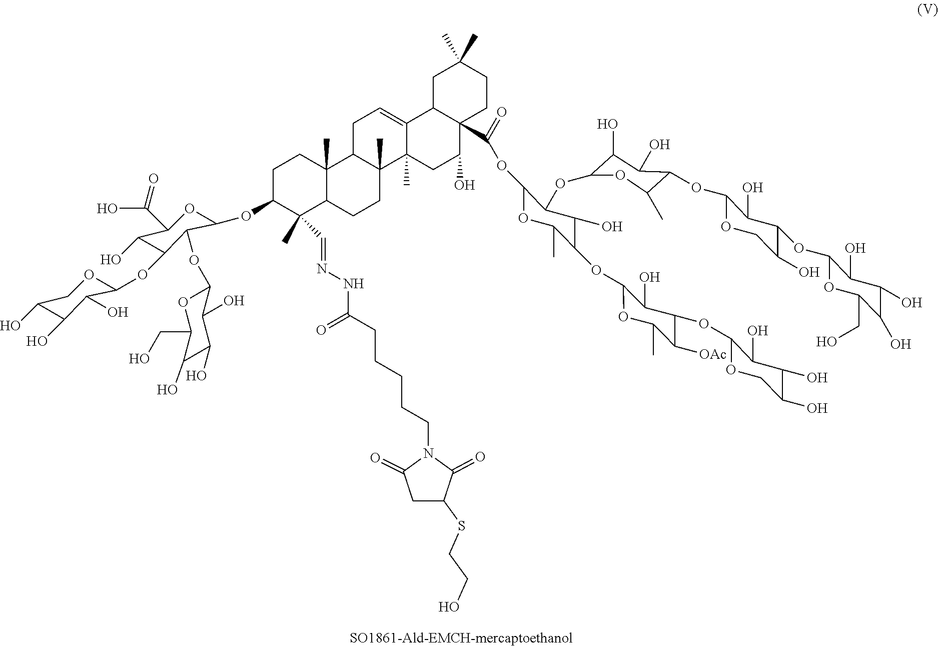

and/or for example the saponin component comprises
a saponin, wherein the carboxyl group of the glucuronic acid unit in the first saccharide chain bound to C-3 of the aglycone core structure of the saponin is transformed into an amide bond through reaction with N-(2-aminoethyl) maleimide (AEM) as shown for SO1861 in formula (18):

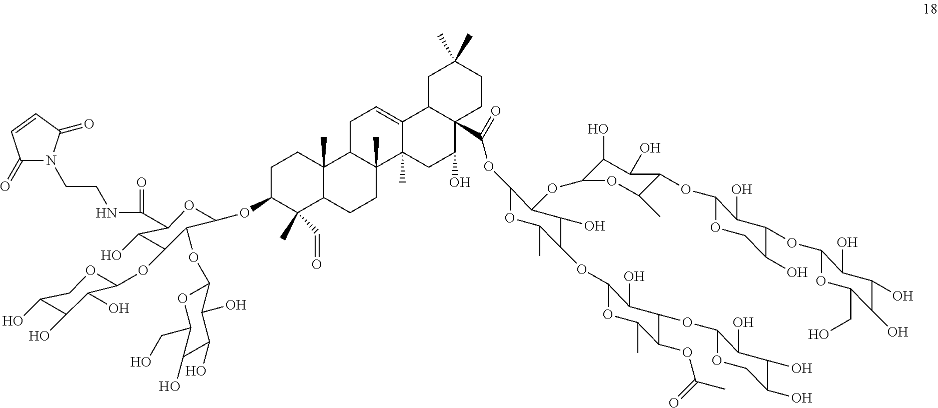

or a saponin having a formula according to one of the following formulas (14)-(16) and (19)-(21):

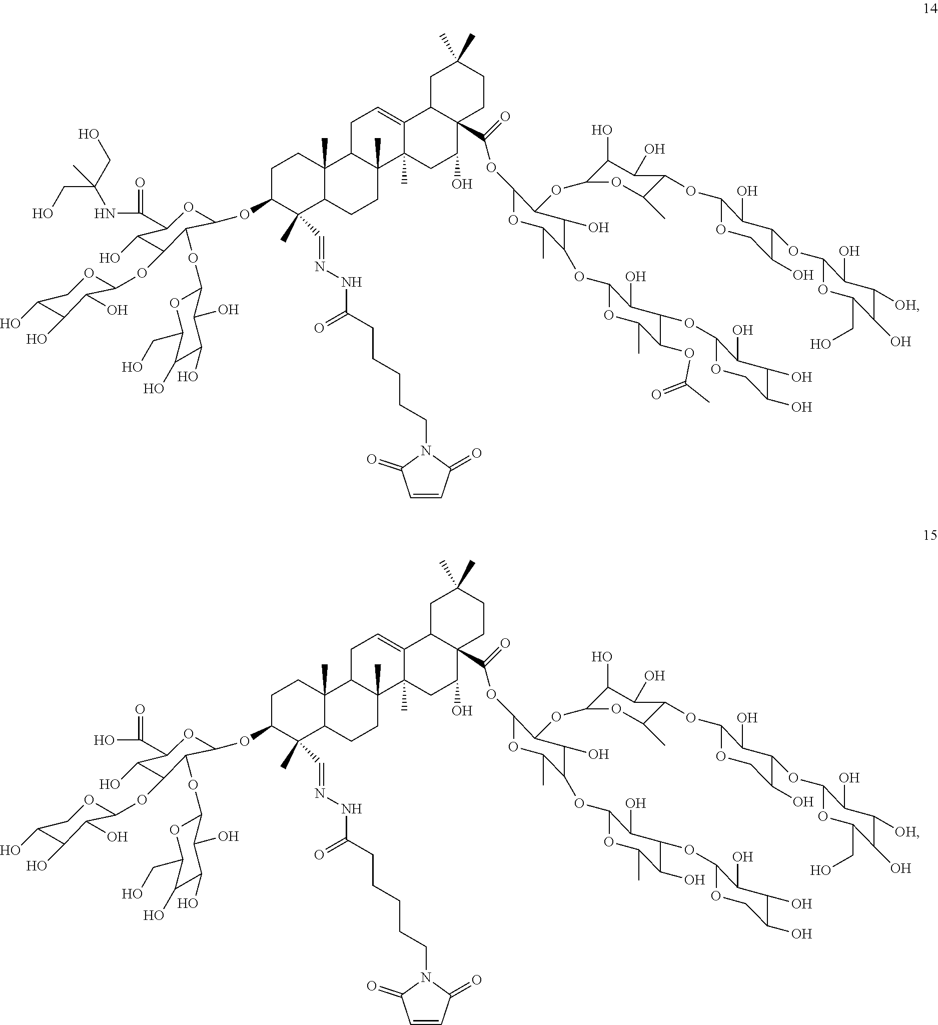

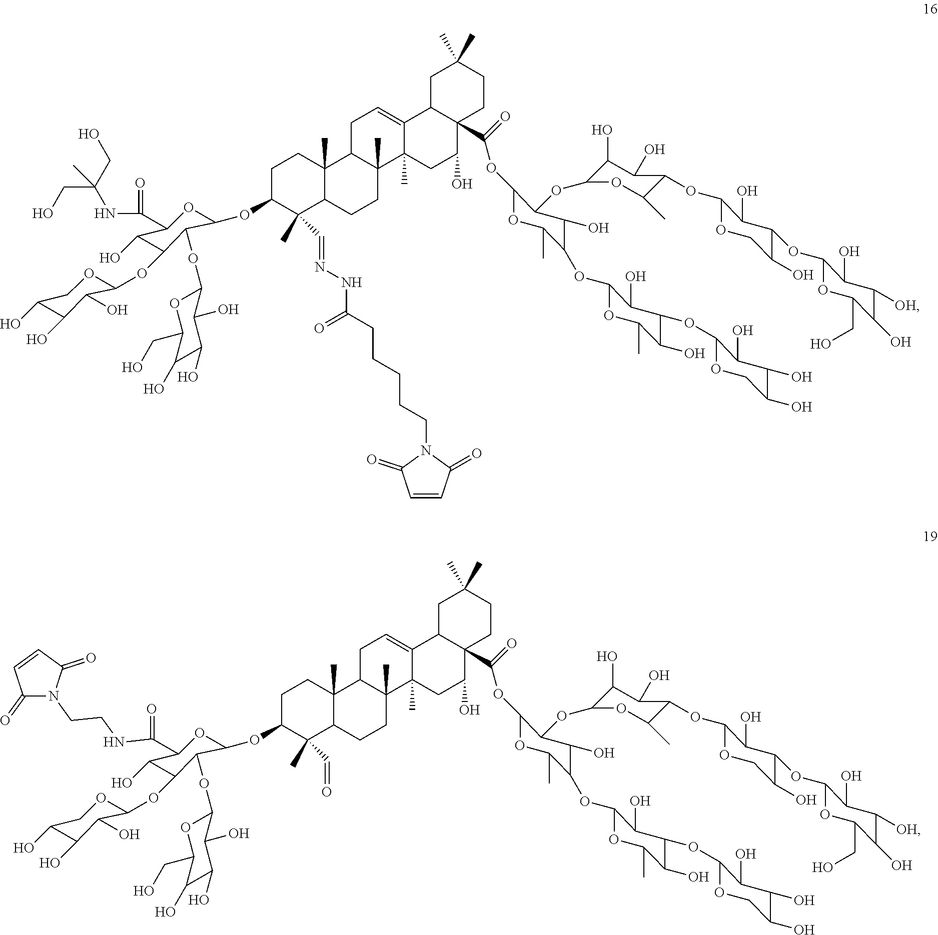

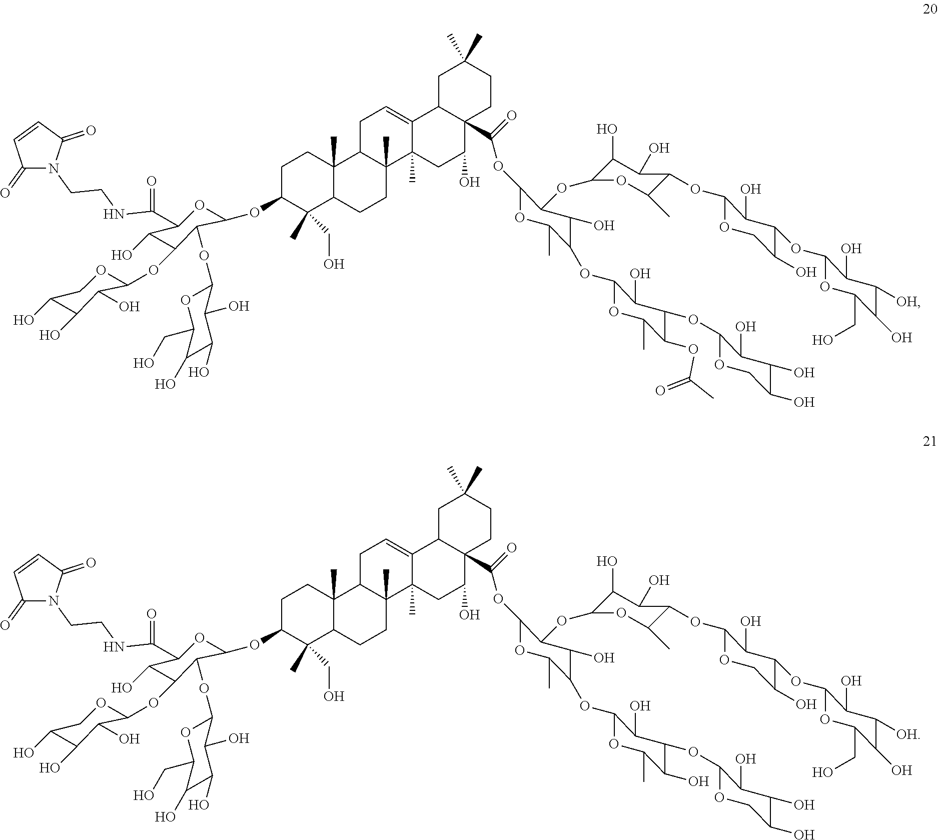

In a preferred embodiment, the saponin component is the molecule according to formula (I) here above or is SO1861 or is a conjugate of SO1861 and the first ligand.

### The Effector Component

The development of the presented herein advantageous compositions was based on the surprising realisation that thanks to the inclusion of the endosomal-escape-enhancing saponin in the presented herein conjugates, any nucleic acid can be delivered with an improved efficiently into cells within the CNS organ and/or the eye by local administration to aid the treatment of an underlying disorder.

As explained herein before, the term “effector component”, refers to a component comprising an effector moiety or consisting of an effector molecule, wherein the effector moiety or the effector molecule is a nucleic acid therapeutic, preferably an oligonucleotide therapeutic.

In a next embodiment, compatible with preceding embodiments, a saponin component or a pharmaceutical composition for the disclosed herein use is provided, wherein the nucleic acid therapeutic is selected from:
a gene therapy therapeutic that is capable of treating or ameliorating the disorder by replacing or restoring the function of an abnormal or non-functional gene implicated in the disorder with a functioning variant or by introduction of a reparation within said gene;
or
an oligonucleotide therapeutic defined as a nucleic acid therapeutic that is not longer than 200 nt, preferably has a size of 5-150 nt, more preferably 8-100 nt, most preferably 10-50 nt, preferably wherein the oligonucleotide therapeutic that is capable of treating or ameliorating the disorder by modulating the expression of a gene implicated in the disorder.

In a next embodiment, compatible with preceding embodiments, a saponin component or a pharmaceutical composition for the disclosed herein use is provided, wherein the nucleic acid therapeutic comprises DNA and/or RNA and/or a synthetic nucleic acid (aka. xeno-nucleic acid, XNA) defined as modified equivalent of DNA and/or of RNA and comprising one or more nucleotide analogues and/or backbone modifications, preferably wherein the nucleic acid therapeutic is selected from:
DNA therapeutic, for example comprising double-stranded DNA (dsDNA), possibly circular such as of plasmid or mini-circle DNA; and/or for example comprising single stranded DNA (ssDNA), preferably wherein the DNA therapeutic is selected from plasmid, mini-circle DNA, CRISPR-gene editing related constructs, DNA aptamer, and/or DNA antisense oligonucleotide (ASO, AON) for example DNA anti-microRNA ASO (anti-miRNA ASO, anti-miR ASO), most preferably is a DNA ASO;
RNA therapeutic, for example comprising double-stranded RNA (dsRNA), such as of short interfering RNA (siRNA) or small activating RNA (saRNA), and/or for example comprising single stranded RNA (ssRNA) such as of mRNA or microRNA (miRNA), possibly wherein the RNA therapeutic comprises non-coding RNA (ncRNA) such as transfer RNA (tRNA), ribosomal RNA (rRNAs), circular RNA (circRNA) such as ecircRNA or ciRNA, small non-coding RNA such as miRNA, siRNA, piRNA, snoRNA, snRNA, exRNA, scaRNA, or long non-coding RNA (lncRNA) such as long intervening/intergenic noncoding RNAs (lincRNAs); preferably wherein the RNA-based therapeutic is selected from RNA ASO, siRNA, miRNA, RNA miRNA inhibitor (anti-microRNA, anti-miRNA, anti-miR) and/or RNA miRNA inhibitor ASO, RNA aptamer, ribozyme, RNA decoy, short hairpin RNA (shRNA), anti-hairpin-shaped microRNA; most preferably wherein the RNA therapeutic is selected from RNA ASO, siRNA, miRNA, and/or RNA aptamer;
mixed DNA/RNA and/or synthetic nucleic acid therapeutic, preferably comprising or consisting of any one of the following modifications: phosphoramidate morpholino oligomer (PMO, Morpholino), peptide nucleic acid (PNA), phosphorothioate-modified antisense oligonucleotide (PS-ASO), 2′-O-methyl(2′-OMe) phosphorothioate RNA, 2′-O-methoxyethyl(2′-O-MOE) RNA (2′-O-methoxyethyl-RNA (2′-MOE, MOE)), locked nucleic acid (LNA, bridged nucleic acid, BNA; for example 2′-O,4′-aminoethylene bridged nucleic acid (BNA-NC), BNA-based siRNA, BNA-based antisense oligonucleotide (BNA-ASO), BNA-based anti-microRNA etc.), 2′-deoxy-2′-fluoroarabino nucleic acid (FANA), 3′-fluoro hexitol nucleic acid (FHNA), glycol nucleic acid (GNA), threose nucleic acid (TNA), more preferably wherein the mixed DNA/RNA and/or synthetic nucleic acid therapeutic comprises or consists of a gapmer (mixmer), synthetic gapmer, synthetic CpG oligonucleotide, synthetic RNA decoy, synthetic ASO and/or synthetic anti-microRNA, for example anti-microRNA ASO such miRNA-masking ASO (miR-Mask, BlockmiR, usually being a single-stranded 2′-O-methyl-modified oligoribonucleotide) or antagomir (miRNA antagonist) or other LNA-based or 2-O-methyl RNA-based anti-microRNA.
more preferably wherein the nucleic acid therapeutic is a mixed DNA/RNA and/or synthetic nucleic acid therapeutic selected from: synthetic ASO, substantially DNA-based synthetic ASO, substantially RNA-based synthetic ASO preferably comprising 2′-MOE modification, substantially DNA-based synthetic aptamer, substantially RNA-based synthetic aptamer, synthetic gapmer, synthetic siRNA, synthetic miRNA, synthetic anti-miRNA and/or synthetic anti-miRNA ASO.
   For example, targeting the CNS with 2′-MOE-containing ASOs is considered advantageous because of their high stability in the cerebrospinal fluid (CSF) after intrathecal injection, which makes them particularly suitable for CNS targeting (Khorkova et al., 2017).

In a next embodiment, compatible with preceding embodiments, a saponin component or a pharmaceutical composition for the disclosed herein use is provided,
wherein the nucleic acid therapeutic is an oligonucleotide therapeutic, preferably an siRNA therapeutic or an antisense oligonucleotide (ASO) therapeutic, preferably comprising one or more nucleotide analogues and/or backbone modifications, more preferably being a mutation specific therapeutic, for example being a mutation specific ASO comprising one or more nucleotide analogues and/or backbone modifications, possibly designed to silence a gene implicated in the disorder and/or to induce exon skipping.

In an next embodiment, compatible with preceding embodiments, a saponin component or a pharmaceutical composition for the disclosed herein use is provided, wherein the nucleic acid therapeutic targets a gene selected from: HTT, LRRK2, SNCA, Parkin gene, PINK1, DJ-1, DRP-1, SCN1A, SOD1, TDP-43, FUS, C9orf72, NEK1, UBQLN2, ATXN2, SMN2, SMN1, MAPT (tau gene), APP (amyloid precursor protein gene), BACE1, IL-4, IL-6, IL-7, IL-12RB2, IL-1R1, MBP, MIR29B, AR, FAS, C2orf72 UBE3A, UBE2A, GFAP, DMD, DYN2, DGAT2, MFSD8 (CLN7), TTR, VEGF e.g. VEGF-A, VEGFR1, VEGFR2, RHO, NF2, CMV virus IE2, CEP290, USH2A, CASP2, TRPV1, RPGR, ITGA4, PCED, USH2A, GJA1, C5, OPA1, TGFB2, RTP801, ADRB2, COCH, VEGF-165, P2RX7, JUN, BAX, APAF1, IKBKB, RDS, GUCY1A1, GUCY1A2, CNG (e.g. CNGA1, CNGA2, CNGA3, CNGB1, CNGB3), DDIT4, HIF1A, FN1, CTGF, TXNIP, CYP4B1, CNR1 and CNR2, STAT3, KRAS, TGFB2, MIR21, BCL2, TP53, FOXP3, GRB2, ADRB2, PTGS2/TGFB1, CEBPA, Malat1, AHA1, and MMP14, preferably wherein the gene is any one of the following genes: HTT, SOD1, MFSD8 (CLN7), SMN1, SMN2, TTR, Malat1, AHA1, or MMP14.

In a next embodiment, compatible with preceding embodiments, a saponin component or a pharmaceutical composition for the disclosed herein use is provided, wherein the nucleic acid therapeutic is an oligonucleotide therapeutic, that is preferably capable of silencing a gene or disabling a gene product (e.g. inhibiting mRNA or miRNA, or can be an aptamer like pegatinib), more preferably wherein the oligonucleotide therapeutic is selected from the group consisting of:
nusinersen (ASO for SMN2 splicing in SMA); inotersen (ASO against TTR in hATTR), eplontersen (ASO against TTR in hATTR), vutrisiran (siRNA against TTR in hATTR), patisiran (siRNA against TTR in hATTR), tofersen (ASO against SOD1 in ALS), QRX-704 (ASO against HTT), jacifusen (ION-363; ASO against FUS); tominersen (IONIS-HTTRx or RG6042; ASO against HTT), WVE-003, (ASO against HTT); zilganersen (ASO against GFAP in Alexander disease); atesidorsen, cimdelirsen (ASOs against GHR in acromegaly), ATL-1102 (ASO against CD49d in relapsing forms of MS); BIIB-080 (ASO against TAU/MAPT in Alzheimer's disease, frontotemporal degeneration, AD dementia); GTX-102 (ASO against UBE2A); ION-464 (ASO against SNCA), ION-541 (ASO against ATXN2); ION-859 (ASO against LRRK2), IONIS-PKKRx (ASO against KLKB1), STK-001 (ASO for splicing SCN1A), WVE-004 (ASO against C9orf72), trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma); lademirsen (anti-miR-21), fomivirsen (ASO against CMV virus IE2), pegatinib (aptamer that binds and blocks VEGF), bevasiranib (siRNA against VEGF-A), SIRNA-027 (siRNA against VEGFR-1), aganirsen (ASO against IRS1), sepofarsen (ASO for CEP290 splicing), lufepirsen (CODA-001; ASO against, connexin 43 (GJA1)), IONIS-FB-LRx (ASO against CFB), QR-1123 (ASO against RHO), ultevursen (QR-421a; ASO for USH2A), QPI-1007 (siRNA against in NAION), tivanisiran (siRNA against TRPV1); and bamosiran (siRNA against ADRB2).

An overview of oligonucleotide therapeutics and their indications can be found in the TABLE 2B

TABLE 2B
preferred oligonucleotide therapeutics (prepared based on Moumné et al. 2022)

| | | | Mode | | |
|---|---|---|---|---|---|
| | | | of | | |
| Name | type | Target Gene | Action | Diseases and indications | Stage |
| Nusinersen | ASO | SMN2 | SM | spinal muscular atrophy (SMA), and/or | Marketed |
| | | | | (other) SMN2-related diseases | |
| Inotersen | ASO | TTR | EI | Hereditary transthyretin amyloidosis | Marketed |
| | | | | (hATTR), and/or (other) TTR-related | |
| | | | | diseases | |
| Eplontersen | ASO | TTR | EI | chronic heart failure and high blood | Phase III |
| | | | | pressure, particularly for patients with | |
| | | | | resistant hypertension due to elevated | |
| | | | | aldosterone, hereditary transthyretin- | |
| | | | | mediated amyloidosis (hATTR) and/or | |
| | | | | (other) TTR-related diseases | |
| Vutrisiran | siRNA | TTR | EI | polyneuropathy of hereditary | Pre- |
| | | | | transthyretin-mediated amyloidosis | registration |
| | | | | (hATTR amyloidosis), and/or (other) | |
| | | | | TTR-related diseases | |
| Patisiran | siRNA | TTR | EI | polyneuropathy associated with | Marketed |
| | | | | hereditary transthyretin-mediated | |
| | | | | amyloidosis (hATTR), and/or (other) | |
| | | | | TTR-related diseases | |
| Tofersen | ASO | SOD1 | EI | amyotrophic lateral sclerosis (ALS), | Phase III |
| | | | | and/or (other) SOD1-related diseases | |
| Jacifusen | ASO | FUS | EI | fused in sarcoma (FUS)-protein | Phase III |
| | | | | associated myotrophic lateral sclerosis | |
| | | | | (ALS), and/or (other) FUS-related | |
| | | | | diseases | |
| Tominersen | ASO | HTT | EI | Huntington's disease (HD), and/or | Phase III |
| | | | | (other) HTT-related diseases | |
| WVE-003 | ASO | HTT | EI | cardiovascular diseases, and/or (other) | Phase II |
| | | | | HTT-related diseases | |
| Zilganersen | ASO | GFAP | EI | Alexander Disease (AxD), and/or (other) | Phase III |
| | | | | GFAP-related diseases | |
| Atesidorsen | ASO | GHR | EI | excessive growth hormone (GH)- | Phase II |
| | | | | associated diseases, including | |
| | | | | acromegaly, and/or (other) GHR-related | |
| | | | | diseases | |
| Cimderlirsen | ASO | GHR | EI | Acromegaly, and/or (other) GHR-related | Phase II |
| | | | | diseases | |
| ATL-1102 | ASO | ITGA4 | EI | Duchenne muscular dystrophy (DMD) | Phase II |
| | | | | and relapsing forms of multiple sclerosis | |
| | | | | (MS), and/or (other) ITGA4-related | |
| | | | | diseases | |
| BIIB-080 | ASC | MAPT | EI | Alzheimer's Disease, frontotemporal | Phase II |
| | | | | degeneration, mild cognitive impairment | |
| | | | | due to Alzheimer's disease, mild | |
| | | | | Alzheimer's disease dementia and/or | |
| | | | | (other) MAPT-related diseases | |
| GTX-102 | ASO | UBE2A | EI | angelman syndrome (AS), and/or | Phase II |
| | | | | (other) UBE2A-related diseases | |
| ION-464 | ASO | SNCA | EI | Parkinson's disease (PD), multiple | Phase II |
| | | | | system atrophy (MSA) and related | |
| | | | | synucleinopathies, and/or (other) | |
| | | | | SNCA-related diseases | |
| ION-541 | ASO | ATXN2 | EI | amyotrophic lateral sclerosis (ALS), | Phase II |
| | | | | and/or (other) ATXN2-related diseases | |
| ION-859 | ASO | LRRK2 | EI | Parkinson's disease (PD), and/or (other) | Phase II |
| | | | | LRRK2-related diseases | |
| IONIS-PKKRx | ASO | KLKB1 | EI | hereditary angioedema (HAE), and/or | Phase II |
| | | | | (other) KLKB1-related diseases | |
| STK-001 | ASO | SCN1A | SM | dravet syndrome, and/or (other) | Phase II |
| | | | | SCN1A-related diseases | |
| WVE-004 | ASO | C9orf72 | EI | amyotrophic lateral sclerosis (ALS) and | Phase II |
| | | | | frontotemporal disorders (FTD) , and/or | |
| | | | | (other) C9orf72-related diseases | |
| Trabedersen | ASO | TGFB2 | EI | Cancers associated with TGFB2- | Phase III |
| | | | | overexpression, including brain cancer, | |
| | | | | colorectal cancer, melanoma and | |
| | | | | pancreatic cancer, and/or (other) | |
| | | | | TGFB2-related diseases | |
| ISTH-0036 | ASO | TGFB2 | EI | primary open-angle glaucoma (POAG), | Phase II |
| | | | | and/or (other) TGFB2-related diseases | |
| STP-705 | siRNA | PTGS2/TGFB1 | El | basal cell cancer, Bowen's disease, | Phase II |
| | | | | hypertrophic scars, keloids, | |
| | | | | cholangiocarcinoma, liver cancer, | |
| | | | | obesity, bladder cancer, and/or (other) | |
| | | | | PTGS2- or TGFB1-related diseases | |
| Danvatirsen | ASO | STAT3 | EI | Cancers involving STAT, including | Phase II |
| | | | | lymphoma, lung cancer and head and | |
| | | | | neck squamous cell carcinoma | |
| | | | | (HNSCC), and/or (other) STAT3-related | |
| | | | | diseases | |
| AZD-8701 | ASO | FOXP3 | EI | cancer associated with advanced solid | Phase II |
| | | | | tumours, including clear cell renal cell | |
| | | | | cancer (ccRCC), non-small-cell lung | |
| | | | | cancer (NSCLC), triple negative breast | |
| | | | | neoplasms (TNBN), squamous cell | |
| | | | | cancer of head and neck (HNSCC), | |
| | | | | small cell lung cancer (SCLC), | |
| | | | | gastroesophageal cancer, melanoma, | |
| | | | | cervical cancer, and/or (other) FOXP3- | |
| | | | | related diseases | |
| siG-12D- | siRNA | KRAS | EI | pancreatic cancer, and/or (other) KRAS- | Phase II |
| LODER | | | | related diseases | |
| IONISAR- | ASO | AR | EI | prostate cancer, and/or (other) AR- | Phase II |
| 2.5Rx | | | | related diseases | |
| SR-063 | siRNA | AR | EI | AR-V7 positive prostate cancer, and/or | Phase II |
| | | | | (other) AR-related diseases | |
| Prexigebersen | ASO | GRB2 | EI | RAS-activated cancers, including | Phase II |
| | | | | chronic myeloid leukaemia (CML), acute | |
| | | | | myeloid leukemia (AML), acute | |
| | | | | lymphocytic leukemia (ALL) and | |
| | | | | myelodysplastic syndromes (MDS), | |
| | | | | and/or (other) GRB2-related diseases | |
| MTL-CEBPA | saRNA | CEBPA | EA | liver cancer, and/or (other) CEBPA- | Phase II |
| | | | | related diseases | |
| oblimersen | ASO | BCL-2 | EI | melanoma, chronic lymphocytic | Phase II |
| | | | | leukemia | |
| Lademirsen | anti-miR | miR-21 | EI | Alport Syndrome, and/or (other) MIR21- | Phase II |
| | | | | related diseases | |
| Fomivirsen | ASO | CMV virus IE1 | EI | cytomegalovirus retinitis (CMV), and/or | Marketed |
| | | and IE2 | | (other) CMV-virus IE2-related diseases | and |
| | | | | | withdrawn |
| Pegaptanib | Aptamer | VEGF-165 | AI | Neovascular acute myeloid | marketed |
| | | | | degeneration (AMD), and/or (other) | |
| | | | | VEGF-165-related diseases | |
| Bevasiranib | siRNA | VEGF-A | EI | Acute myeloid degeneration (AMD), | Phase III |
| | | | | and/or (other) VEGF-A-related diseases | and |
| | | | | | withdrawn |
| siRNA-027 | siRNA | VEGFR-1 | EI | Acute myeloid degeneration (AMD), | Phase III |
| | | | | and/or (other) VEGFR-1-related | |
| | | | | diseases | |
| Aganirsen | ASO | IRS1 | EI | ocular neovascularization in patients | Phase III |
| | | | | with front of the eye (cornea) or back of | |
| | | | | the eye (retinal) diseases, including | |
| | | | | progressive corneal neovascularization, | |
| | | | | such as in patients with infectious | |
| | | | | keratitis and wet age related macular | |
| | | | | degeneration (AMD), and/or (other) | |
| | | | | IRS1-related diseases | |
| Sepofarsen | ASO | CEP290 | SM | CEP290-mediated Leber congenital | Phase III |
| | | | | amaurosis 10 (LCA10), and/or (other) | |
| | | | | CEP290-related diseases | |
| Lufepirsen | ASO | GJA1 | EI | persistent Corneal Epithelial Defects | Phase II |
| | | | | (PCED), near sightedness after PRK | |
| | | | | laser eye surgery, and/or (other) GJA1- | |
| | | | | related diseases | |
| IONIS-FB-LRx | ASO | CFB | EI | complement-mediated diseases, | Phase II |
| | | | | including IgA nephropathy and Age- | |
| | | | | related macular degeneration (AMD) | |
| QR-1123 | ASO | RHO | EI | retinitis pigmentosa (RP), and/or (other) | Phase II |
| | | | | RHO-related diseases | |
| Ultevursen | ASO | USH2A | SM | retinitis pigmentosa (RP), and/or (other) | Phase II |
| | | | | USH2A-related diseases | |
| QPI-1007 | siRNA | CASP2 | EI | optic neuropathies including glaucoma | Phase III |
| | | | | and Acute Nonarteritic Anterior Ischemic | |
| | | | | Optic Neuropathy (NAION), and/or | |
| | | | | (other) CASP2-related diseases | |
| Tivanisiran | siRNA | TRPV1 | EI | dry eye disease, pathologies that cause | Phase III |
| | | | | ocular pain, such as corneal lesions and | |
| | | | | refractive surgery, and/or (other) | |
| | | | | TRPV1-related diseases | |
| Bamosiran | siRNA | ADRB2 | EI | glaucoma and ocular hypertension, | Phase II |
| | | | | and/or (other) ADRB2-related diseases | |

SM—Splicing modulation; EI—Expression inhibition; EA—Expression activation; AI—Activity inhibition

In an next embodiment, compatible with preceding embodiments, a saponin component or a pharmaceutical composition for the disclosed herein use is provided, wherein the first endocytic receptor and/or the second endocytic receptor is selected from
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR,
Tropomyosin receptor kinase A (TrkA) receptor (NGF receptor)
IL13-R (interleukin-13 receptor)
AMPAR/NMDAR (AMPA- and NMDA-type glutamate receptors)
vascular endothelial growth factor receptor 1 or 2 (VEGFR1 or VEGFR2)
STRA6 (Retinol-binding protein (RBP) receptor).

Particularly suitable receptors for the disclosed herein applications are the following: transferrin receptor (CD71), tetraspanin (CD63), insulin-like growth factor 1 (IGF-I) receptor (IGF1R), InsR (insulin receptor), glucose transporter GLUT4, cation independent mannose 6 phosphate receptor (CI-MPR), LDL receptor, TrkA receptor, IL13-R, AMPAR/NMDAR, TGFb receptor, vascular endothelial growth factor receptor 1 and 2 (VEGFR1 and VEGFR2), and STRA6 (Retinol-binding protein (RBP) receptor). STRA6 is e.g. interesting for retinal cell delivery by virtue of being expressed on retinal pigment epithelia (RPE) cells.

Further examples of known cell-surface receptors are: CD71, CD63, CA125, EpCAM (17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR). Preferred endocytic cell-surface receptors for e.g. tumor targeting are: HER2, c-Met, VEGFR2, CXCR7, CD71, EGFR and EGFR1.

In a next embodiment, compatible with preceding embodiments, a saponin component or a pharmaceutical composition for the disclosed herein use is provided, wherein the first ligand and/or the second ligand is selected from:
antibody or a binding fragment thereof binding to any one of the receptors listed in the preceding embodiment;
natural ligand or a fragment thereof recognised by any one of the receptors listed in preceding embodiment;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFβ or a fragment thereof;
EGF or a fragment thereof;
neurotrophin (Nerve Growth Factor, NGF) or fragment thereof;
Interleukin 13 (IL-13) or a fragment thereof;
glutamate or multiple units thereof;
vascular endothelial growth factor A (VEGF-A) or a fragment thereof;
retinol (vitamin A) or other forms of vitamin A;
retinol-binding protein (RBP) or a fragment thereof;
antibody or a binding fragment thereof binding to an endocytic receptor selected from: CD71, CD63, IGF1R, GLUT4, CI-MPR, LDL receptor;
more preferably wherein the first ligand and/or the second ligand is an antibody or a binding fragment thereof binding to CD71, even preferably being a monoclonal or a single domain antibody binding to CD71, most preferably being a monoclonal antibody binding to CD71.

In embodiments of the invention, the effector moiety is any one of the here-above defined effector molecules, with covalently bound thereto:
a linker selected from the any one or more linkers hereabove defined for the saponin moiety;
a linker, such as a linker as hereabove defined, with a further molecule covalently bound to the linker
wherein said further molecule is defined as hereabove defined for the saponin moiety, and is any one or more of:
d. a further linker, such as a linker as hereabove defined;
e. a ligand for binding to an endocytic cell-receptor,
   wherein the ligand is a proteinaceous ligand or a non-proteinaceous ligand or a combination thereof,
   wherein the proteinaceous ligand is for example:
   a. a protein ligand capable of binding to a cell-surface receptor, which binding results in internalization of the protein ligand, for example a cytokine or EGF;
   b. an antibody, as defined hereabove for the saponin moiety.

In embodiments of the invention wherein the effector component comprises an effector moiety conjugated with a ligand for binding to an endocytic cell-surface receptor, the effector component either comprises the same ligand as the saponin component, or the effector component comprises a ligand that differs from the ligand comprised by the saponin component. When the ligands comprised by the effector component and the saponin component differ, those different ligands typically both bind to an endocytic cell-surface receptor present on the same cell. Such endocytic receptor can be the same endocytic receptor or can be two different endocytic receptors.

For example, the ligand comprised by the effector component can be an antibody capable of binding to a first tumor-cell specific receptor present on a tumor cell, and the ligand comprised by the saponin component can be an antibody or a ligand such as EGF capable of binding to a second tumor-cell specific receptor present on said same tumor cell.

In a preferred embodiment the saponin component comprises an oligonucleotide therapeutic.

In a preferred embodiment, the saponin component comprises a ligand capable of binding to an endocytic cell-surface receptor.

In a preferred embodiment, the oligonucleotide component comprises a ligand capable of binding to an endocytic cell-surface receptor.

In a preferred embodiment, the saponin component comprises both a ligand capable of binding to an endocytic cell-surface receptor as here above defined and an oligonucleotide as here above defined.

A preferred embodiment is the therapeutic combination of, or therapeutic composition comprising any one of the saponin component as here above defined and any one of the oligonucleotide component as here above defined.

A preferred embodiment is the therapeutic combination of, or therapeutic composition comprising any one of the saponin component as here above defined and any one of the oligonucleotide component as here above defined.

A preferred embodiment is the therapeutic combination of, or therapeutic composition comprising any one of the saponin component as here above defined wherein the saponin component comprises a ligand as here above defined and any one of the oligonucleotide component as here above defined wherein the oligonucleotide component comprises a ligand as here above defined, for targeting an endocytic cell-surface molecule present on the same cell as the endocytic cell-surface molecule targeted by the ligand comprised by the saponin component.

A preferred embodiment is the saponin component comprising a saponin moiety and an oligonucleotide.

A preferred embodiment is the saponin component consisting of a saponin molecule.

A preferred embodiment is the oligonucleotide component consisting of an oligonucleotide molecule.

A preferred embodiment is a therapeutic combination of, or therapeutic composition comprising a saponin molecule and an oligonucleotide molecule.

A preferred embodiment is a therapeutic combination of, or therapeutic composition comprising any of the saponin component a as here above defined and any one of the oligonucleotide component as here above defined.

A preferred embodiment is a therapeutic composition comprising or consisting of a saponin component comprising an oligonucleotide and comprising a ligand as here above defined.

Any of the therapeutic composition or any of the therapeutic composition preferably comprises a therapeutically acceptable excipient and/or a therapeutically acceptable diluent.

Last but not least, provided herein is an embodiment of the disclosed herein pharmaceutical compositions further comprising any one or more component selected from the following: a pharmaceutically acceptable excipient and/or pharmaceutically acceptable diluent and/or analgesic agent and/or immunosuppressants and/or anti-inflammatory agent and/or antibiotic.

Anti-inflammatory agents include but are not limited to non-steroidal anti-inflammatory agents such as bromfenac, nepafenac, ketorolac, diclofenac, flurbiprofen; corticosteroids such as dexamethasone, difluprednate, loteprednol, fluocinolone, fluorometholone, triamcinolone, rimexolone, prednisone, prednisolone; and integrin antagonists such as lifitegrast. Immunosuppressants include but are not limited to antimetabolites such as azathioprine, methotrexate and mycophenolate mofetil; calcineurin inhibitors such as cyclosporine, tacrolimus and voculosporin; alkylating agents such as cyclophosphamide and chlorambucil; TNF inhibitors such as etanercept, infliximab, adalimumab; lymphocyte inhibitors such as rituximab and abatacept; interferons such as interferon alpha and interleukin antagonists such as IL-1 antagonist anakinra and IL-2 antagonist daclizumab. Antibiotics include but are not limited to ofloxacin, moxifloxacin, levofloxacin, ciprofloxacine, gatifloxacin, azithromycin, besifloxacin, tobramycin, polymyxin b, trimethoprim, trifluridine. Vidarabine, gentamicin, chloramphenicol. Neomycin, erythromycin and bactiricin, Analgesics include but are not limited to the non-steroidal anti-inflammatory agents and corticosteroids as mentioned above, and local anesthetics such as tetracaine, proparacaine and lidocaine.

### The CNS and CNS-Specific Applications

Central Nervous System disorders are a major burden for patients, their family members and society and are associated with high costs. Majority of these disorders are associates with the brain.

In line with the above, in particularly advantageous embodiments, a saponin component or a (neuro) pharmaceutical composition is provided, wherein the organ is part of the central nervous system (CNS), preferably is the brain.

The complexity of the brain makes it difficult to pinpoint one cause and often both genetic and environmental factors play a role in their pathophysiology.

The importance and recognition of the genetic component may vary amongst neurodegenerative disorders of which Huntington's disease, for example, is clearly linked to the Huntingtin (HTT) gene, whilst other diseases involve lesions in many different genes, e.g. Alzheimer's disease (AD) and amyotrophic lateral sclerosis (ALS), or have a larger environmental component such as Parkinson's disease (PD). For oncology, the location and the size of the tumour in the CNS are factors that influence the severity of the disorder and the chances of survival. Glioblastoma (GBM) is one of the best-known cancers in the CNS and also one of the most lethal ones.

For both oncological disorders in the CNS, as well other CNS disorders such as neurodegenerative disorders, a challenge lie in reaching the target site, since the brain is protected by the BBB limiting access to pharmacological interventions. In particular, BBB makes the standard systemic administration routes like intravenous or subcutaneous administration, practically impossible for oligonucleotide therapeutics. Consequently, other administration routes must be considered including epidural, intrathecal, intracerebroventricular or intranasal delivery, to name a few. For oncological disorders, also postoperative injection to the intratumoural cavity can be considered.

Anatomically, the brain and spinal cord are enveloped by four membranes known as meninges, whose function is to protect the central nervous system. Starting from the most distant from the neural tissue of the brain and the spinal cord, these are: the dura mater (being the closest meninx to the bones of the skull and the vertebral column), the arachnoid mater, the subarachnoidal lymphatic-like membrane (SLYM), and the pia mater. The arachnoid mater and pia mater are sometimes called together the leptomeninges.

Usually, three distinct spaces are defined with respect to the dura matter and the leptomeninges. The first and the outermost is the epidural space between the skull or bones of the vertebral column and the dura mater of the brain and the spinal cord. The spinal cord ends between the first and second lumbar vertebra, at which point, only cerebrospinal fluid is present. This is a relatively safe site for preforming epidural injections and the site of the lumbar puncture (“spinal tap”), frequently used for analgesics and anesthesia. Below the epidural space, there is the subdural space between the dura mater and the arachnoid mater, which under normal conditions, is not a space but can be opened in case of traumas such as a brain bleed or other medical condition. Last one is the subarachnoid space between the arachnoid mater and pia mater, which is filled with the cerebrospinal fluid (CSF) that cushions and protects your brain and spinal cord and is in direct contact with their tissues and cells.

With respect to the above-described anatomical sites, one can define different administration sites, which will be known to the persons of medical profession.

In an next embodiment, compatible with preceding embodiments, a saponin component or a (neuro) pharmaceutical composition for the disclosed herein use is provided, wherein the administration is selected from epidural, intrathecal, intracerebroventricular, intracisternal, intraparenchymal, intranasal and/or comprises a postoperative injection to the intratumoural cavity formed after surgery within the CNS; preferably wherein the administration is selected from intrathecal, intracerebroventricular, intracisternal, and/or intranasal; more preferably wherein the administration is intrathecal.

Because of the direct accessibility to neural tissue, the preferred route is intrathecal, which means that the administration is made into the subarachnoid space (which has the advantages that the neuropharmaceutical composition comprising the saponin component and the effector component reaches the CSF).

A further advantageous route is intranasal, which uses olfactory neural cells to reach the brain. The olfactory neural cells are bipolar neurons that extend their dendritic processes into the mucus layer, terminating as olfactory receptors, and project into the olfactory bulb. This provides a direct portal between the nose and the central nervous system. Furthermore, their unmyelinated axons are covered by olfactory ensheathing cells (OECs) and olfactory nerve fibroblasts that are in continuity with meninges and, consequently, with the subarachnoid space (Cassano et al., 2021)

In a next embodiment, compatible with preceding embodiments, a saponin component or a (neuro) pharmaceutical composition for the disclosed herein use is provided, wherein the administration is made into the dura mater, or into the arachnoid mater, or into the subarachnoid space, or into the pia mater, and/or into the brain tissue; preferably wherein the administration is made into the arachnoid mater and/or into the subarachnoid space; more preferably wherein the administration is made into the subarachnoid space, so that the neuropharmaceutical composition comprising the saponin component and the effector component reaches the CSF.

In a further embodiment, compatible with preceding embodiments, a saponin component or a (neuro) pharmaceutical composition) for the disclosed herein use is provided, wherein the CNS disorder is selected from:
a neurodegenerative disorder, preferably selected from any one or more of Huntington's disease (HD), Alzheimer's disease (AD), Parkinson's disease (PD), amyotrophic lateral sclerosis (ALS), spinal muscular atrophy (SMA), multiple system atrophy (MSA), multiple sclerosis (MS), and/or dementia with Lewy body (DLB);
neurological disorder, preferably selected from stroke, epilepsy such as Dravet syndrome (DS), and/or a spinal cord disease;
an oncological disorder, preferably selected from any one or more of glioblastoma, meningioma, (oligodendro) glioma, astrocytoma, ependymoma, medulloblastoma, CNS lymphoma, metastasis to the CNS; more preferably selected from glioblastoma, meningioma, (oligodendro) glioma, and/or metastasis to the CNS;
immune disorder, preferably selected from an autoimmune disease of the CNS, an immunity-related disease caused by a gene defect, a disease caused by an infection or inflammation, more preferably selected from meningitis, encephalitis, prion disease, and/or coronavirus disease 2019 (COVID-19);
a psychiatric disorder, preferably selected from any one or more of Tourette syndrome (TS), mood disorder, personality disorder, anxiety disorder, substance use or addictive disorder, obsessive-compulsive disorder, neurodevelopmental disorder, eating disorder; more preferably is selected from an anxiety disorder, obsessive-compulsive disorder, eating disorder, and/or a mood disorder preferably being a treatment-refractory mood disorder.

In an embodiment, compatible with preceding embodiments, a saponin component or a (neuro) pharmaceutical composition for the disclosed herein use is provided, wherein the CNS disorder is selected from: spinal muscular atrophy (for which the therapeutic nusinersen has been approved), hereditary transthyretin amyloidosis (hATTR), amyotrophic lateral sclerosis (ALS) preferably being SOD1-associated amyotrophic lateral sclerosis (for which the therapeutic tofersen has been developed), Huntington's disease (for which the therapeutic tominersen has been developed), Alzheimer's disease, Parkinson's disease, Batten disease (for which a proof of concept personalised therapeutic Milasen was made), frontotemporal dementia, pinocerebellar ataxia type 3, multiple system atrophy; Rett syndrome, Alexander disease; Angelman syndrome; Lafora disease; GFAP astrocytopathy, a prion disease, and a neurological disorders related to acromegaly.

In a further embodiment, compatible with preceding embodiments, a saponin component or a (neuro) pharmaceutical composition) for the disclosed herein use is provided, wherein the nucleic acid therapeutic targets a gene selected from: HTT, LRRK2, SNCA, Parkin gene, PINK1, DJ-1, DRP-1, SCN1A, SOD1, TDP-43, FUS, C9orf72, NEK1, UBQLN2, ATXN2, SMN2, SMN1, MAPT (tau gene), APP (amyloid precursor protein gene), BACE1, IL-4, IL-6, IL-7, IL-12RB2, IL-1R1, MBP, MIR29B, AR, FAS, C2orf72 UBE3A, UBE2A, GFAP, DMD, DYN2, DGAT2, MFSD8 (CLN7), TTR, VEGF e.g. VEGF-A, VEGFR1, VEGFR2, RHO, NF2, CMV virus IE2, CEP290, USH2A, CASP2, TRPV1, RPGR, ITGA4, PCED, USH2A, GJA1, C5, OPA1, TGFB2, RTP801, ADRB2, COCH, VEGF-165, P2RX7, JUN, BAX, APAF1, IKBKB, RDS, GUCY1A1, GUCY1A2, CNG (e.g. CNGA1, CNGA2, CNGA3, CNGB1, CNGB3), DDIT4, HIF1A, FN1, CTGF, TXNIP, CYP4B1, CNR1 and CNR2, STAT3, KRAS, TGFB2, MIR21, BCL2, TP53, FOXP3, GRB2, ADRB2, PTGS2/TGFB1, CEBPA, Malat1, AHA1, and MMP14, preferably wherein the gene is any one of the following genes: HTT, SOD1, MFSD8 (CLN7), SMN1, SMN2, TTR, Malat1, AHA1, or MMP14.

In a further embodiment, compatible with preceding embodiments, a saponin component or a (neuro) pharmaceutical composition for the disclosed herein use is provided, wherein the nucleic acid therapeutic is an oligonucleotide therapeutic selected from the group consisting of: nusinersen (ASO for SMN2 splicing in SMA); inotersen (ASO against TTR in hATTR), eplontersen (ASO against TTR in hATTR), vutrisiran (siRNA against TTR in hATTR), patisiran (siRNA against TTR in hATTR), tofersen (ASO against SOD1 in ALS), QRX-704 (ASO against HTT), jacifusen (ION-363; ASO against FUS); tominersen (IONIS-HTTRx or RG6042; ASO against HTT), WVE-003, (ASO against HTT); zilganersen (ASO against GFAP in Alexander disease); atesidorsen, cimdelirsen (ASOs against GHR in acromegaly), ATL-1102 (ASO against CD49d in relapsing forms of MS); BIIB-080 (ASO against TAU/MAPT in Alzheimer's disease, frontotemporal degeneration, AD dementia); GTX-102 (ASO against UBE2A); ION-464 (ASO against SNCA), ION-541 (ASO against ATXN2); ION-859 (ASO against LRRK2), IONIS-PKKRx (ASO against KLKB1), STK-001 (ASO for splicing SCN1A), WVE-004 (ASO against C9orf72), trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma); lademirsen (anti-miR-21),

In an embodiment, compatible with preceding embodiments, a saponin component or a (neuro) pharmaceutical composition for the disclosed herein use is provided, comprising the first ligand and/or the second ligand, wherein the first endocytic receptor and/or the second endocytic is present on the cells and/or tissue within the CNS, preferably wherein the cells are selected from any one of more of neurons, astrocytes, oligodendrocytes, microglia, endothelial cells, blood cells, and/or tumour cells, more preferably wherein the cells are selected from any one of more of neurons, astrocytes, oligodendrocytes, microglia, endothelial cells, and/or tumour cells;

most preferably wherein the first endocytic receptor and/or the second endocytic receptor is selected from:
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR
Tropomyosin receptor kinase A (TrkA) receptor (NGF receptor)
IL13-R (interleukin-13 receptor)
AMPAR/NMDAR (AMPA- and NMDA-type glutamate receptors).

In an embodiment, compatible with preceding embodiments, a saponin component or a (neuro) pharmaceutical composition for the disclosed herein use is provided, comprising the first ligand and/or the second ligand, wherein the first ligand and/or the second ligand is selected from:
antibody or a binding fragment thereof binding to any one of the receptors listed in the preceding embodiment;
natural ligand or a fragment thereof recognised by any one of the receptors listed in preceding embodiment;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFβ or a fragment thereof;
EGF or a fragment thereof;
neurotrophin (nerve growth factor, NGF) or fragment thereof;
Interleukin 13 (IL-13) or a fragment thereof;
glutamate or multiple units thereof;
antibody or a binding fragment thereof binding to an endocytic receptor selected from: CD71, CD63, IGF1R, GLUT4, CI-MPR, LDL receptor;
more preferably wherein the first ligand and/or the second ligand is an antibody or a binding fragment thereof binding to CD71, even preferably being a monoclonal or a single domain antibody binding to CD71, most preferably being a monoclonal antibody binding to CD71.

In a further embodiment, compatible with preceding embodiments, a saponin component or a (neuro) pharmaceutical composition for the disclosed herein use is provided, wherein the effector component comprises an oligonucleotide therapeutic targeting any one of STAT3, SOD1, Malat1, AHA1, MMP14, TTR, and HTT, or is an oligonucleotide therapeutic selected from nusinersen, tominersen, tofersen, inotersen, eplontersen, vutrisiran, patisiran, and trabedersen; and wherein the saponin component preferably comprises SO1861 or SO1861 wherein the aldehyde function at position C-23 is substituted by the acid sensitive covalent bond configured to break under acidic conditions so as to create the aldehyde function at position C-23 of the aglycone core,
preferably wherein the administration is intrathecal, and preferably comprises the 2-component free saponin formulation or the 2-component linker-saponin formulation or 1-component formulation as defined above.

### The Eye and Ocular Applications

It is further one of the objectives of the present disclosure to improve bioavailability of ocular nucleic acid therapeutics. The presented herein solutions are believed to possess advantages that will allow to reduce the therapeutic concentrations, doses, and possibly also injectable volumes of the existing nucleic acid therapeutics, hopefully also leading to less failures in clinical trial of extremely promising but not yet approved new drugs for ocular delivery.

Hence, in a particular embodiment, compatible with preceding embodiments, a saponin component or a pharmaceutical (ophthalmic) composition for the disclosed herein use is provided, wherein the organ is the eye and the disorder is further referred to as the ocular disorder.

Despite its peripheral location, the neural portion of the eye (the retina), is classified as a part of the CNS (Purves D et al., 2001). The eye, the brain, and the spinal cord, are related organs that are developmentally derived from the same embryonic CNS precursor in all vertebrates that is known as the neural tube (Marchesi et al., 2021). The eye not only possesses neural similarities to the brain but also vascular ones; the blood-retinal barrier (BRB) is very similar to the BBB, both of which are composed of non-fenestrated endothelial cells connected by tight junctions (Jindal, 2015). Neurodegenerative pathologies of the CSN, notably including the Alzheimer's disease (AD) and the Parkinson's disease (PD), show peculiar alterations at the ocular level (Guidoboni, 2020) and several studies have shown that a substantial number of ocular disorders present characteristics of neurodegenerative diseases at a histopathological and/or gene expression level and a correlation is observed between retinal biomarkers and neurological conditions including AD, PD, ALS, multiple sclerosis (MS), and prion diseases (Yap et al., 2019).

In brief, for example glaucoma is a progressive optic nerve degeneration and can be considered a neurodegenerative disorder of both the eye and the brain (Chan, 2021; Stein, 2021). AMD, in addition to causing visual recognition difficulties, is associated with higher rates of cognitive decline and higher risk of dementia (Zhuang, 2021; Ashok, 2020). There is also evidence that e.g. retinitis pigmentosa (RP) is correlated with significant reductions in grey matter volume, mainly in the occipital cortex of RP patients (Rita Machado, 2017). ALS is a fatal progressive loss of the upper motor neurons located in the brain and the lower motor neurons located in the brain stem and the spinal cord but ALS patients' autopsies frequently show asymmetric contralateral damage and axonal degeneration of retinal ganglion cells, together with significant thinning of the retinal nerve fibre layer (RNFL) and reduced total macular thickness (Soldatov, 2021).

The above similarities are further supported by the fact that many genes that are associated with neurodegeneration are also associated with retinal diseases, which is not considered unexpected in the field given that the retina shares an ontogenetic relationship with the brain (Soldatov, 2021). Examples of CNS/ocular disease relevant gene products include e.g. the antioxidant enzyme super oxide dismutase 1 (SOD1) that is often found (among other diverse components) in the proteinaceous inclusions observed in the spinal cord of ALS patients. SOD1 was reported to protect retinal cells from oxidative damage (Dong et al., 2006) and Sod1-deficient mice have features typical of age-related macular degeneration (AMD) in humans (Imamura et al., 2006). There exist genetic associations between glaucoma and ALS, including polymorphic variants of OPTN, TBK1, PFN1, and ATXN2 genes, and several other genes or their products linked to the formation of aggregates in motor neurons in ALS were also inked to vison impairment and/or retinal damage, including TDP-43, OPTN, C9ORF72 (Soldatov, 2021). Other examples include non-coding RNAs like lncRNA Malat1 that is overexpressed in glaucoma and implicated in diabetic retinopathy and other retinal pathologies (Song & Kim, 2021; Carella et al., 2021, Nasrolahi et al., 2023) or miR-124 that is known to be involved in neurogenesis in both the optic vesicle and the forebrain (Liu et al., 2011).

Despite the above evident similarities between the eye and the (other) organs of the CNS and bacuse ocular applications are very specific to the anatomy of the eye and its surrounding structures, for better understanding of the eye-related embodiments of the disclosure, the following definitions are provided:

### Ocular-Application Related Definitions

As used herein, the terms “local” or “locally to the eye”, in the context of administration are to be construed as involving any one of the following routes of administration: directly onto the eyeball (i.e. “topically), or into the eyeball (i.e. “intraocularly”), or into vicinity of the eyeball (i.e. “periocularly”). Usually, the pharmaceutical composition will be introduced from any container or other device in which it was retained in prior to the action of administering (examples of the containers or other devices include an applicator, syringe, capsule, strip etc.) to contact the surface of the eyeball or will be introduced into the interior of the eyeball or the periocular space (i.e. the space around the eyeball, for example within the orbit). Examples of such local administration include topical administration onto the eyeball's surface (for example by eye drop or ointment formulations), administrations into the vicinity of the eyeball by means of a subconjunctival injection or an implant placement in any periocular zone, and, most preferably by intraocular injection and/or implant placement e.g. intravitreally, suprachoroidally, etc.

As used herein the term “intraocular” in the context of administration, shall be understood as involving administration into any of the structures (including, layers, tissues, segments, or chambers etc.) present within the eyeball, which will usually involve passing through the fibrous layer of the eyeball, i.e. through any one of the sclera and cornea. In line with this definition, the following routes shall be understood as non-limiting examples of intraocular administration: intracameral, scleral or intrascleral, suprachoroidal, subretinal, retinovitreal, intravitreal etc.

As used herein, the term “intracameral” in the context of a route of administration, for example via injection, is to be construed as referring to a local intraocular administration into any of the spaces containing the aqueous humour, normally, into the anterior chamber of the eyeball. Intracameral injection can be referred to as ICM injection.

For better understanding the local delivery into the eye, it is worth mentioning that the eye is frequently seen as containing two segments: the anterior and the posterior one.

As used herein the terms “anterior segment” are “anterior cavity” are used synonymously and are to be construed as referring to (roughly) the front third of the eye, which includes the following eye structures located in front of the vitreous humour: the cornea, iris, ciliary body, and lens. The anterior segment contains two spaces filled with aqueous fluid, which, as used herein, is also referred to “aqueous humour”. These spaces are: (i) the anterior chamber that lies between the posterior surface of the cornea and the iris; and (ii) the posterior chamber that lies between the iris and the front face of the vitreous humour. In line with the above, as used herein the term “posterior chamber” shall be used to mean the posterior chamber of the anterior segment of the eye. Analogously, the term “anterior chamber” shall be construed as referring to the anterior chamber of the anterior segment of the eye.

The aqueous humour filling both of the spaces (i) and (ii) of the anterior segment is a transparent water-like fluid similar to plasma but with lesser protein concentrations, although containing immunoglobins. The aqueous humour provides nutrients (e.g. amino acids and glucose) to the avascular surrounding structures of the eye, maintains the intraocular pressure, inflates the globe of the eye, and prevents eye dryness. The aqueous humour is continually produced by the ciliary body and its production is balanced by an equal rate of drainage, primarily by trabecular meshwork. The aqueous humour is normally at 15 mmHg above atmospheric pressure, so when a syringe is injected into the anterior chamber, it flows easily.

As used herein, the term “intracameral” in the context of a route of administration, for example via injection, is to be construed as referring to a local intraocular administration into any of the spaces containing the aqueous humour, normally, into the anterior chamber of the eyeball. Intracameral injection can be referred to as ICM injection.

The aqueous humour is not to be confused with the “vitreous humour”, which is located in the space between the lens and the retina, which space is also known as “vitreous chamber” and is located inside of the “posterior segment” or the “posterior cavity” of the eyeball (eye).

As used herein, the terms “posterior segment” and “posterior cavity” are to be construed as synonyms referring to the back two-thirds of the eye, which includes the vitreous chamber with the vitreous humour, the layer of collagen that covers the vitreous humour and is further termed “hyaloid membrane” (that is synonymous to the terms “vitreous membrane” or “vitreous cortex”), and all of the eye structures behind the anterior part of the hyaloid membrane including: the retina, choroid, and optic nerve.

The term “vitreous humour” as used herein is synonymous to the term “vitreous body”, or simply “vitreous”, and refers to a thick, clear gel-like substance that is enclosed within the hyaloid membrane and maintains the shape of the eye. Vitreous body contains 99% water and no cells, for allowing effective light passage without deflection towards the retina. As used herein the term “anterior hyaloid membrane” refers to the part of the hyaloid membrane that separates the front of the vitreous body from the lens, while the term “posterior hyaloid membrane” refers to the part of the hyaloid membrane that separates the vitreous body from the retina.

In line with the above, it will be considered that the eye (referring to the human or other vertebrate eye) comprises three chambers being: anterior, posterior, and vitreous, and that it can also be considered that the eye has two segments (cavities) on both sides of the lens: anterior and posterior. Both the anterior and posterior chambers are located within the anterior cavity, while the vitreous chamber is located in the posterior cavity. The vitreous chamber is the largest of the three chambers and is located behind the lens and in front of the optic nerve and the retina.

As used herein, the term “intravitreal” in the context of administration is to be construed as referring to a local mode of intraocular administration, e.g. via injection, into the vitreous humour (i.e. vitreous body, or, simply, the vitreous). Intravitreal injection can be referred to as IVI injection.

As used herein the term “periocular” in the context of administration, shall be understood as involving administration into vicinity of the eyeball within the space defined by the orbit, or where the orbit ends, the inner side of the eyelids, usually being the space between the sclera and the orbit wall. In line with this definition, the following routes shall be understood as non-limiting examples of periocular administration: subconjunctival, subtenon (sub-tenon or sub-Tenon's) e.g. anterior subtenon or posterior subtenon, juxtascleral e.g. posterior juxtascleral, peribulbar, retrobulbar, etc.

As used herein, in the context of administration, the term “topical” shall be understood as referring to superficial, usually non-invasive, administration onto the eyeball, i.e. the cornea and/or the sclera.

### Ocular-Application Related Embodiments and Context

Ocular disorders represent a major disease burden worldwide. They are frequently associated with aging and, consequently, their incidence and complexity is constantly increasing together with the increasing life-expectancy in human population. Age-related macular degeneration (AMD), glaucoma, dry eyes, cataracts, and temporal arteritis are a few typical conditions that affect an aging eye. Irrespective of aging, they can also result from another underlying condition like diabetes, an example of which is diabetic retinopathy (DR), or they can be caused by an underlying genetic condition, such as in case of retinitis pigmentosa (RP), Leber congenital amaurosis (LCA), and USH2A retinopathy (Singh et al., 2018). In terms of pathophysiological origin, ocular disorders can further be infectious, inflammatory, and/or autoimmune in nature.

Degeneration of ocular neurons is the leading cause of irreversible blindness in e.g. glaucoma, AMD, DR, and RP, but different degrees thereof are eventually observed in most of the ocular disorders including Stargardt disease, cone-rod dystrophy, achromatopsia etc. (Jindal, 2015; Ahmad et al., 2020). Furthermore, sufficient evidence exists that, in line with the established histological and physiological link between the eye and the rest of the CNS, most neurodegenerative process in the latter also tend to involve the retinal neurons in the pathogenetic process (Yap et al., 2019).

Eye is a complex organ with internal structures arranged concentrically in three tissue layers; from the exterior being the sclera and cornea, in the middle containing the vascular layer termed the uvea (subdivided into the iris, ciliary body, and choroid), and the innermost being the retina i.e. the nervous tissue extension of the brain proper (Purves D et al., 2001). Ocular disorders can originate in any one of these layers. For example, in the case of diabetic retinopathy, the site of damage is in the endothelium of retinal vessels. In the case of retinoblastoma, the cell of origin is believed to be the neuronal cone precursor (Bremner & Sage, 2017), while in many other disorders, including an early-onset retinal dystrophy known as retinitis punctata albescens and several types of inherited RPs, the causing mutations are believed to occur the genes expressed in the retinal pigment epithelium (RPE or just “retinal epithelium).

The RPE is a monolayer of pigmented cells that absorbs the light focused on the retina and directly covers the light-sensitive outer segments of the rods and cones. It forms a part of the BRB and delivers nutrients and ions to the photoreceptors through abundantly expressed transcytotic receptors like Na,K-ATPase, GLUT1, GLUT3, insulin and transferrin (CD71) receptors. It also performs the reisomerisation of cis/trans retinal for the photoreceptors, which is necessary for maintaining their excitability, and secretes immunosuppressive factors in support of the immune privilege of the eye. Mutations in genes that are expressed in the RPE (e.g. MerTK or RPE65) can lead to photoreceptor degeneration and vice versa (e.g. ABCR expressed in the photoreceptors). In fact, the RPE and the photoreceptor function is so tightly linked and interdependent that both of these cell types are often regarded as a single functional unit within the retina (Strauss 2005). The above is perhaps the best reflected by the fact that accumulation of photo-oxidized products in the RPE is believed to be the underlying cause of the AMD (Kevany & Palczewski, 2010), which is the most prevalent retinal disease in the Western world and the most he most common cause of blindness in developed nations (Wong et al., 2014).

Depending on the aetiology, there exist many different treatment options for ocular disorders, and depending on the given therapeutic, also different formulations and administration routes.

Progress in the understanding of molecular basis of the ocular diseases and their underlying genetic factors has resulted in development of many nucleic acid therapeutics for ocular therapy. Their extensive review can be found in A Guzman-Aranguez et al., 2013, British Journal of Pharmacology, 170:730-747. Doi: 10.1111/bph.12330.

For serious ocular diseases that cannot be cured, considerable hope is currently associated with induction of the RNA interference (RNAi) process, in which a short non-coding RNA (siRNA or miRNA) collaborates with cytosolic proteins to interfere with gene expression by causing degradation or translationally repressing target mRNA (Lam J K D et al., Mol Ther Nucleic Acids., 2015). However, the clinical exploitation of RNAi and other nucleic acid-induced therapeutic effects is notoriously hampered by the poor bioavailability of these therapeutics, stemming from their high susceptibility to enzymatic hydrolysis, rapid elimination from the circulatory system, extremely poor cellular uptake, as well as potential immunogenicity and other off-target effects (Cai X et al., 2017).

To overcome these shortcomings, different vector systems for nucleic acid delivery have been developed, which can be divided as viral or non-viral. Compared to the non-viral vectors, the viral vectors have limited payload capacity and higher potential immunogenicity. Consequently, non-viral vectors are not only more socially accepted, but also considered safer and preferred for their lower immunogenicity and associated costs. There are many types of non-viral vectors, such as polymers and peptides, which to date, have proven to be promising tools for gene delivery thanks to their capability of incorporating ligands for targeting specific cell types (Vicentini FTMDC et al. Pharm Res 2013).

Due to stability and clearance considerations, choice of an administration route is an important factor for nucleic acid therapeutics. Delivery methods for ocular drugs are mainly divided into systemic and local i.e. peri- or intraocular, administration. Compared with the local administration, drugs administered systemically are usually prevented from entering into the eye due to the presence of ocular-blood barrier (Patel et al., 2010; Cabrera et al., 2019), and are more likely to cause a systemic immune response (Urtti, 2006). Consequently, local administration is frequently the only choice of treatment for many ocular disorders.

Local ocular administration can be divided as non-invasive and invasive. Invasive routes provide better control over the therapeutic delivery and dose control.

Although non-invasive ways like the instillation of eye drops appears to be an easy approach for treating anterior segment ocular disorders, topical ocular administration of therapeutic nucleic acids faces many challenges due to nuclease degradation and the fact that the anterior segment exhibits a continuous flow of tear fluid causing faster clearance. The above processes further limit bioavailability of the nucleic acid therapeutics, which necessitates more frequent drug administration, potentially resulting in patient compliance and safety issues from the long-term management perspective. Consequently, for achieving a desired therapeutic outcome, current formulation strategies for topical ocular therapeutics aim at increased retention in the tear film (for example, for treatment of dry eyes). Further considerations for choosing topical delivery include if the drug can be absorbed by the cornea or the conjunctiva (e.g., for treating keratitis or conjunctivitis), or if it can penetrate or permeate across the cornea and/or conjunctiva to reach deeper target tissues, such as the trabecular meshwork, iris, or ciliary body. Consequently, where topical administration fails, intracameral (ICM) injections or ICM implant insertions can be considered, which are much more complicated and require skilled personnel (Liebmann et al., 2020).

Delivery and uptake of therapeutic nucleic acids into the posterior segment is even more challenging, primarily because of its deeper positioning into the skull (and hence, the even further limited ability to reach to the diseased cells), together with its anatomical and physiological separation from the anterior chamber, as well as high vascularisation, in particular of the retina. For a topically instilled therapeutic to treat, for example, the retinal epithelium (RPE), it would have to be able to pass through both the aqueous as well as the vitreous body for reaching its target site. As a consequence, for the posterior segment treatment, nucleic acid-based drugs are typically administered intraocularly, via intravitreal or, less frequently, subretinal route, or particularly i.e. in the area surrounding the eyeballs but still within the bony orbits.

Examples of US FDA-approved intravitreally deliverable nucleic acid therapeutics include fomivirsen (Vitravene®) that is 21-base phosphorothioated antisense oligodeoxynucleotide for treatment of cytomegalovirus retinitis, and Pegaptanib (Macugen®) that is an VEGF-targeting aptamer for treatment of neovascular age-related macular degeneration. Several other nucleic acid-based therapeutics at currently different stages of clinical development include: VEGF targeting siRNA bevasiranib developed by Opko Health Inc.; VEGF-receptor 1 (VEGFR1)-targeting siRNA-027 developed by Allergan for treatment of neovascular eye diseases like age-related macular degeneration, and RTP801-targeting siRNA PF-655 developed by Quark Pharmaceuticals for treatment of diabetic retinopathy and corneal neovascularization. In addition to VEGF, and VEGFR1 and 2, other potential targets known for nucleic acid-based intraocular therapy include RhoA, cochlin (COCH), Na-K-ATPase, purine receptor P2Y₂, c-Jun, apoptosis regulators Bax, Apaf-1, caspase-2, TGF-β2, and IκB kinase β (IKK β) for glaucoma; rhodopsin, Retinal Degeneration Slow (RDS) peripherin, guanylate-cyclase 2 and cyclic nucleotide-gated ion channels for retinitis pigmentosa; RTP801 for AMD; and HIF1A, fibronectin, connective tissue growth factor (CTGF) and thioredoxin-interacting protein (TXNIP) for DR (Guzman-Aranguez et al., 2013).

With regard to the invasive routes of local ocular administration, the advantages of the present compositions stem from their potential of lowering the volume or the general size of the therapeutic load that needs to be introduced to or around the eye. This not only is likely to cause less frequent or severe side-effects but also will contribute to lesser discomfort of the patient, and thus can further positively influence compliance with the treatment. Further, as the bioavailability of the therapeutic nucleic acid is increased at lower doses, the treatments are expected to become more efficient in general, thus possibly reducing the frequency for repeated injections or implant insertions.

Advantageous invasive routes for local ocular delivery comprise the intraocular route and the periocular route.

In an embodiment, compatible with preceding embodiments, a saponin component or a pharmaceutical (ophthalmic) composition for the disclosed herein use is provided, wherein the administration is intraocular; preferably selected from intrascleral, suprachoroidal, subretinal, intracameral, intravitreal, and vitreoretinal; more preferably wherein the administration comprises any one selected from intracameral injection, intracameral implant, intravitreal injection, subretinal injection, intravitreal implant, and a scleral plug; even more preferably wherein the administration is intravitreal, even more preferably wherein the administration comprises intravitreal injection or an intravitreal implant, most preferably wherein the administration comprises intravitreal injection.

Alternative potential route for drug delivery involves periocular administration, which includes subconjunctival, subtenon, retrobulbar, peribulbar and posterior juxtascleral delivery routes. The drugs given by these routes can be delivered to the different layers of the eye, in that order, depending on source concentration and the barrier properties of these and other intermediate layers between this site of administration and target side. Periocular routes of delivery have been noticed as potentially safer alternatives for delivering drugs to the retina, avoiding the risks of intraocular damage posed by intravitreal injection. There is an increased risk of systemic drug exposure, but it is still considerably less when compared with systemic or topical drug delivery. Periocular delivery therefore might be capable of delivering the drug to the desired site in a large concentration while avoiding most of the systemic side-effects, with a relatively long duration of action.

Hence, in a possible embodiment, compatible with preceding embodiments, a saponin component or a pharmaceutical (ophthalmic) composition for the disclosed herein use is provided, wherein the local administration is periocular and preferably is subconjunctival, more preferably comprises subconjunctival injection or a subconjunctival implant.

There are still some limitations to the periocular route such as lower bioavailability of the drugs in the retina than by the intravitreal route, however it still has the advantage of avoiding the blood-tissue barrier. Furthermore, it appears that the provision of the saponin component according to the disclosure is able to at least partially remove them, even when administered in a separate formulation.

Similar considerations apply to the non-invasive routes involving topical application.

With the non-invasive administration route, the eye damage is minimal. Consequently, whenever possible, non-invasive treatments are preferred by medical professionals and patients. However, due to their low bioavailability, this route is frequently less suitable for nucleic acid therapeutics, but it still has the advantage as avoiding the blood-tissue barrier and could be explored in combination with the saponin component as disclosed herein. Hence, topical application routes are still of potential value for developing the ophthalmic compositions and saponin components for uses according to the disclosure, primarily because even with limited delivery successes they can be of enormous benefit to the patients. This is because the invasive administration route, are unfortunately usually painful, lead to many side effects and hence are associated with poor compliance (Geroski et al., 2001). Consequently, there also exists the need to improve bioavailability in topical ocular treatments, and the presented herein saponin components and compositions appear suitable for addressing also this objective.

Hence, in a possible embodiment, compatible with preceding embodiments, a saponin component or a pharmaceutical (ophthalmic) composition for the disclosed herein use is provided, wherein the local administration is topical, preferably comprising any one of the following: eye drops, an ointment, and a lens adapted to release the pharmaceutical composition.

Concerning the topical route, the disclosed compositions have the potential of lowering the toxicity associated with high concentrations of various ingredients that are otherwise necessary for higher nucleic acid doses. Furthermore, thanks to generally increasing the bioavailability of the therapeutic upon delivery to its target zone, they can to certain extent correct for human errors in self-medication protocols. Last but not least, because of their potential to be formulated as milder and less aggressive compositions, they are likely to contribute to improved patients' compliance.

The choice of the delivery route for a nucleic acid therapeutic will ultimately depend on the location of the disease-affected zone within the eye. Based on the anatomical location, ocular disorders can be associated with the anterior or the posterior segment of the eye.

Commonly occurring anterior segment diseases include dry eye conditions, blepharitis, conjunctivitis, infections, cataract, or traumas of various types. Some of the most common posterior segment diseases include glaucoma, neovascular eye diseases such as AMD, choroidal neovascularization (CNV) or diabetic retinopathy (DR), and retinal or choroidal diseases including congenital disorders such as retinitis pigmentosa (RP).

In an embodiment, compatible with preceding embodiments, a saponin component or a pharmaceutical (ophthalmic) composition for the disclosed herein use is provided, wherein the ocular disorder is a disorder of the posterior segment of the eye; preferably being the disorder of the retina, of the choroid, of the optic nerve, or the vitreous body; more preferably wherein the disorder is selected from one or more of glaucoma, posterior uveitis, posterior scleritis, retinitis, wet- or dry-age related macular degeneration (AMD) such as neovascular AMD (nvAMD, also known as wet AMD), geographic atrophy, diabetic retinopathy, diabetic- or non-diabetic macular edema, choroidal neovascularization, retinoblastoma, and a congenital disorder of any one of the retina, of the choroid, of the optic nerve, or the vitreous body; even more preferably being a congenital disorder of the retina selected from any one of retinitis pigmentosa (RP), Leber congenital amaurosis (LCA), USH2A retinopathy, and neurofibromatosis type 2 (NFT2).

In a particular embodiment, compatible with preceding embodiments, a saponin component or a pharmaceutical (ophthalmic) composition for the disclosed herein use is provided, wherein the ocular disorder involves abnormalities in the retinal pigment epithelium (RPE). For example, many of afore-mentioned disorders of the posterior segment of the eye already fall under this category as they typically exhibit not only the typical characteristics of neuronal degeneration, but also show abnormal changes and/or depositions in the RPE. For example, AMD pathology is characterised macular atrophy of the RPE (Horani et al., 2019, Horani et al., 2020) and by yellowish extracellular deposits of lipids and proteins between the RPE and Bruch's membrane, which are termed drusen and believed to cause retinal cells abnormalities indicative of photoreceptor degeneration and Müller glial activation. RPE abnormalities are in general also frequently observed in so called “macular dystrophies”, historically combining a group of possibly unrelated but inherited in a Mendelian fashion diseases that, similarly to AMD, begin to show lesions within the zone of the macula when symptoms first occur. Macular dystrophies for example include Stargardt disease (implicated gene: ABCA4), Stargardt-like dominant macular dystrophy (ELOVL4), pattern dystrophy (PRPH2), Best macular dystrophy (BEST1), Sorsby fundus dystrophy (TIMP3), autosomal dominant radial drusen (EFEMP1), North Carolina macular dystrophy, spotted cystic dystrophy, dominant cystoid macular oedema, and fenestrated sheen macular dystrophy (genes uncertain). In line with the above, approaches targeting abnormal processes like gene expression not only in the ocular neurons but also in other cell types like RPE can form a part of particular embodiments according to the present disclosure directed to therapies for different forms of ocular degenerative diseases, in particular those leading to blindness.

For the anterior segment ocular diseases, conventional local ophthalmic delivery systems typically will aim for a non-invasive topical administration. Indeed, direct instillation of siRNA into the ocular surface has typically been attempted for the treatment of ocular surface and anterior segment disorders in vivo (Crooke et al., 2009; Martin-Gil et al., 2012). Exemplary formulations include ointments, gels, or eye drops in different forms like solutions, suspensions, or emulsions etc. The first compound based on RNAi and administered in eye drops in humans was B2-adrenergic receptor (ADRB2)-targeting siRNA SYL040012 developed by Sylentis for treatment of glaucoma. Currently, there are several other nucleic acid based therapeutics at various stages of clinical development utilising this delivery route.

In a further embodiment, compatible with preceding embodiments, a saponin component or a pharmaceutical (ophthalmic) composition for the disclosed herein use is provided, wherein the ocular disorder is a disorder of the anterior segment of the eye; preferably wherein the disorder is selected from one or more of anterior uveitis, iritis, blepharitis, conjunctivitis, blepharoconjuctivitis, keratitis, anterior scleritis, episcleritis, dry eye disease, cataract, corneal abrasion, corneal neovascularization, and a trauma of the anterior segment or a part thereof,

The treatment of the posterior-segment ocular diseases, such as age-related eye diseases (AMD) or diabetic retinopathy (DR), present a particular challenge for ophthalmologists due to the complex anatomy and physiology of the eye. This specialized organ is composed of various static and dynamic barriers that restrict drug delivery into the target site of action. Despite numerous efforts, effective intraocular drug delivery remains unresolved and, therefore, it is highly desirable to improve the current treatments of diseases affecting the posterior cavity.

In a further embodiment, compatible with preceding embodiments, a saponin component or a pharmaceutical (ophthalmic) composition for the disclosed herein use is provided, wherein the ocular disorder is selected from: cytomegalovirus retinitis infection; age-related macular degeneration (AMD); autosomal dominant retinitis pigmentosa; Leber's hereditary optic neuropathy; Stargardt disease; Usher syndrome; ocular disorders related to acromegaly, or ocular disorders related to myotonic dystrophy.

In a further embodiment, compatible with preceding embodiments, a saponin component or a pharmaceutical (ophthalmic) composition for the disclosed herein use is provided, wherein the nucleic acid therapeutic targets a gene selected from VEGF e.g. VEGF-A, VEGFR1, VEGFR2, RHO, NF2, CMV virus IE2, CEP290, USH2A, CASP2, TRPV1, RPGR, ITGA4, PCED, USH2A, GJA1, C5, OPA1, TGFB2, RTP801, TTR, MAPT (tau gene), APP (amyloid precursor protein gene), BACE1, IL-4, IL-6, IL-7, AR, FAS, ADRB2, COCH, VEGF-165, P2RX7, JUN, BAX, APAF1, IKBKB, RDS, GUCY1A1, GUCY1A2, CNG (e.g. CNGA1, CNGA2, CNGA3, CNGB1, CNGB3), DDIT4, HIF1A, FN1, CTGF, TXNIP, CYP4B1, CNR1 and CNR2, STAT3, KRAS, TGFB2, MIR21, BCL2, TP53, FOXP3, GRB2, ADRB2, PTGS2/TGFB1, CEBPA, Malat1, AHA1, and MMP14.

In a further embodiment, compatible with preceding embodiments, a saponin component or a pharmaceutical (ophthalmic) composition for the disclosed herein use is provided, wherein the nucleic acid therapeutic is an oligonucleotide therapeutic selected from the group consisting of:
fomivirsen (ASO against CMV virus IE2), pegatinib (aptamer that binds and blocks VEGF), bevasiranib (siRNA against VEGF-A), siRNA-027 (siRNA against VEGFR-1), aganirsen (ASO against IRS1), sepofarsen (ASO for CEP290 splicing), lufepirsen (CODA-001; ASO against, connexin 43 (GJA1)), IONIS-FB-LRx (ASO against CFB), QR-1123 (ASO against RHO), ultevursen (QR-421a; ASO for USH2A), QPI-1007 (siRNA against in NAION), tivanisiran (siRNA against TRPV1); and bamosiran (siRNA against ADRB2). trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma); lademirsen (anti-miR-21),); inotersen (ASO against TTR in hATTR), eplontersen (ASO against TTR in hATTR), vutrisiran (siRNA against TTR in hATTR), patisiran (siRNA against TTR in hATTR), atesidorsen, cimdelirsen (ASOs against GHR in acromegaly),
or is an oligonucleotide therapeutic is designed to reduce or inhibit the expression of VEGF, preferably VEGF-A, or one of its receptors, preferably selected from VEGFR1 or VEGFR2;
or is an oligonucleotide therapeutic designed to induce exon skipping, preferably of the human RPGR gene or SH2A gene or NF2 gene.

In a next embodiment, compatible with preceding embodiments, a saponin component or a pharmaceutical (ophthalmic) composition for the disclosed herein use is provided, comprising the first ligand and/or the second ligand, wherein the first endocytic receptor and/or the second endocytic is present on the cells and/or tissue within the eye, preferably wherein the cells are cells of the retina or cells of the retinal blood vessels; most preferably wherein the first endocytic receptor and/or the second endocytic receptor is selected from:
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR
vascular endothelial growth factor receptor 1 or 2 (VEGFR1 or VEGFR2)
STRA6 (Retinol-binding protein (RBP) receptor).

In a next embodiment, compatible with preceding embodiments, a saponin component or a pharmaceutical (ophthalmic) composition for the disclosed herein use is provided, comprising the first ligand and/or the second ligand, wherein the first ligand and/or the second ligand is selected from:
antibody or a binding fragment thereof binding to any one of the receptors listed in the preceding embodiment
natural ligand or a fragment thereof recognised by any one of the receptors listed in in the preceding embodiment;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFB or a fragment thereof;
EGF or a fragment thereof;
vascular endothelial growth factor A (VEGF-A) or a fragment thereof;
retinol (vitamin A) or other forms of vitamin A;
retinol-binding protein (RBP) or a fragment thereof;
antibody or a binding fragment thereof binding to an endocytic receptor selected from: CD71, CD63, IGF1R, GLUT4, CI-MPR, LDL receptor, VEGFR1, VEGFR2, and STRA6;
more preferably wherein the first ligand and/or the second ligand is an antibody or a binding fragment thereof binding to CD71, even preferably being a monoclonal or a single domain antibody binding to CD71, most preferably being a monoclonal antibody binding to CD71.

In a particular embodiment, compatible with preceding embodiments, a saponin component or a pharmaceutical (ophthalmic) composition for the disclosed herein use is provided, wherein the effector component comprises an oligonucleotide therapeutic targeting any one of STS3, SOD1, Malat1, AHA1, MMP14, TTR, and HTT, or is an oligonucleotide therapeutic selected from, fomivirsen, pegatinib, inotersen, eplontersen, vutrisiran, patisiran, sepofarsen, QR-421a, ultevursen, tivanisiran, and QPI-1007; and wherein the saponin preferably is SO1861 or SO1861 wherein the aldehyde function at position C-23 is substituted by the acid sensitive covalent bond configured to break under acidic conditions such to create the aldehyde function at position C-23 of the aglycone core, most preferably wherein the administration is intravitreal, and preferably comprises the 2-component free saponin formulation or the 2-component linker-saponin formulation or 1-component formulation.

### EXAMPLES

The following examples serve to illustrate the broad applicability of local co-administration of different saponin components with a variety of oligonucleotides. Together these examples show that co-dosing of saponin components with oligonucleotides markedly improves their efficacy:
(1) in relevant target tissues, such as (but not limited to) the central nervous system and the eye, including examples for treatment of sporadic and inherited (familiar) genetic diseases, as well as non-inherited diseases that profit from gene/RNA modulation and various types of cancers originating or spreading to the CNS or eye;
(2) by targeting disease relevant genes for the preferred tissues, including but not limited to STAT3, SOD1, Malat1, AHA1, MMP14, TTR;
(3) by using a variety of oligonucleotide modalities to target these genes (such as PMO, or phosphorothioated (PS) 2′-MOE ASOs, or PS 2′-locked nucleic acid (LNA) ASOs, or siRNAs with different stabilizations), enabling different mechanisms of action such as
(a) exon skip to induce frame shift to
   (i) lead to a premature termination codon and nonsense mediated mRNA decay (RNA degradation), or
   (ii) lead to an altered viable transcript and subsequently a different/functional protein (isoform), or
(b) splice-site blocking to induce alternative splicing/aberrant transcripts leading to RNA degradation, or by
(c) stimulating RNA cleavage (degradation) through the recruitment of ribonuclease (RNase) H to cleave the RNA strand of a DNA-RNA duplex, or by
(d) post-transcriptionally halting, or silencing, gene expression of a target mRNA by siRNA;
(4) by use of specific, pentacyclic 12,13-dehydrooleanane-type saponin components, but not steroid(-like) saponins/molecules,
(5) by use of specific, pentacyclic 12,13-dehydrooleanane-type saponin components that are either co-administered free/unconjugated (e.g., SO1861, or SO1861-AH-Block, or SO1861-SC-Mal) or as covalently conjugated components (either to the oligonucleotide, e.g. as ASO-SC-SO1861 or Cet-SO1861-STAT3_ST6 PMO or Cet-SO1861-STAT3_ST2 PMO, or GN3-SC-SO1861), i.e. either with or without a cell-receptor targeting ligand,
(6) for different routes of administration including but not limited to in vivo such as intravitreal (IVT), intracerebroventricular (ICV).

The presented herein data shows regardless of whether directly conjugated, ligand-conjugated or unconjugated, the pentacyclic 12,13-dehydrooleanane-type saponin component increases the potency of the oligonucleotide therapeutic provided to a tissue of CNS or ocular origin without inducing/substantially increasing neural toxicity associated with the treatment.

The data also suggests that a direct conjugation of an oligonucleotide therapeutic and the pentacyclic 12,13-dehydrooleanane-type saponin component appears to be beneficial in reaching certain brain regions, in particular including those that are far from the injection site or less exposed to CSF flow.

It further shows that such covalent conjugates ensure the desired synchronization of the cellular delivery of an oligonucleotide therapeutic and the saponin component, which results in improved therapeutic efficacy as compared to ASO alone but also to ASO co-administered with saponin

Last but not least, the data further suggests that ligand-targeted conjugates of an oligonucleotide therapeutic and the pentacyclic 12,13-dehydrooleanane-type saponin (“1-component conjugates”) are particularly advantageous for performing an endosomally enriched and synchronized delivery of the saponin component and the therapeutic payload into the same cellular compartment.

### Example 1: In Vivo Efficacy Enhancement by Local Co-Administration of Saponin Components to ASO Compounds in the Brain/CNS

Malat1 (also referred to as MALAT1) has been involved in Parkinson's Disease pathology and has been shown to enhance alpha-synuclein protein stability, which leads to aggregation and Lewy body formation, resulting in neuronal degradation. Malat1 acts as a decoy to repress miR-124, leading to enhanced apoptotic signaling. This effect causes neuronal degeneration (Liu et al., 2017; Front. Biosci. (Landmark Ed) 2019, 24 (7), 1203-1240). It also plays a pivotal role in proliferative vitreoretinopathy, and affects apoptosis of retinal ganglion cells in glaucoma rats by regulating PI3K/Akt signaling pathway (Li et al., Long Non-Coding RNA-MALAT1 Mediates Retinal Ganglion Cell Apoptosis Through the PI3K/Akt Signaling Pathway in Rats with Glaucoma, Cellular Physiology and Biochemistry (2018) 43 (5): 2117-2132, 2017), and targeting Malat1 alleviates retinal neurodegeneration in diabetic mice (Zhang et al., Targeting long non-coding RNA MALAT1 alleviates retinal neurodegeneration in diabetic mice, Int J Ophthalmol 2020, 13 (2), 213-219).

### In Vivo Study Design

Male C57BI/6 (n=15; 7-8 weeks at arrival) were randomly allocated to 5 treatment groups (n=3). Mice received a unilateral intracerebroventricular (ICV; right lateral ventricle) administration of 10 μL allocated treatment solution, namely vehicle (PBS), SO1861 (2.23 μg), Malat1-ASO (10 μg), Malat1-ASO (3 μg), or co-administration of Malat1-ASO (3 μg)+SO1861, according to Table A1. Mice were terminated at study day 10 post dosing. Brains were dissected immediately and separated into cerebrum (left/right), cerebellum and brainstem tissue samples. Tissue samples were preserved in RNALater for 24 h and subsequently frozen at −80° C. until analysis for the expression of Malat1 RNA levels.

TABLE A1
CNS in vivo dosing schedule

| Group | | Dose volume | Dose | ASO | SO1861 |
|---|---|---|---|---|---|
| No. | Treatment name | [μL] | [μg/μL] | [μg] | [μg] |
| 1 | Vehicle (PBS) | 10 | NA | NA | NA |
| 2 | SO1861 | 10 | 0.223 | NA | 2.23 |
| | (2.23 μg) | | | | |
| 3 | Malat1-ASO | 10 | 1.0 | 10 | NA |
| | (10 μg) | | | | |
| 4 | Malat1-ASO | 10 | 0.3 | 3.0 | NA |
| | (3 μg) | | | | |
| 5 | Malat1-ASO (3 μg) + | 10 | 0.3 + 0.223 | 3.0 | 2.23 |
| | SO1861 (2.23 μg) | | | | |

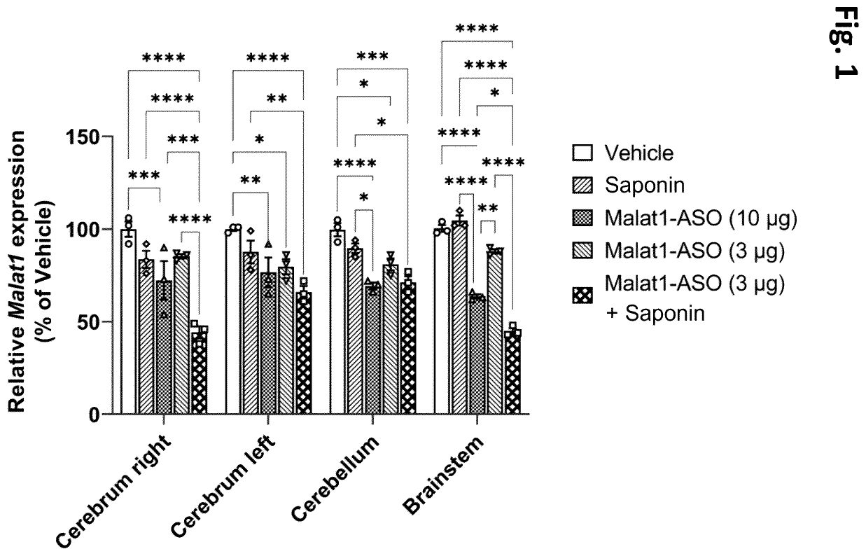

Analysis of Malat1 levels in brain tissues shows that local co-administration of an ASO with a saponin component significantly enhances the ASO potency (FIG. 1): to this end, Malat1 RNA expression after local ICV administration of either 10 μg Malat1 ASO, 3 μg Malat1 ASO, or co-administration of 3 μg Malat1 ASO+saponin component SO1861 into the right lateral ventricle was compared to control conditions (SO1861 only and vehicle groups) in different brain regions close to or peripheral from the injection site. The co-administration of 3 μg Malat1 ASO+SO1861 leads to a pronounced and significant reduction of Malat1 RNA levels, not only in tissue close to injections site (cerebrum right) but, surprisingly, also in the tissue most distal to the injection site (brainstem). Most importantly, when compared to the dose matched condition (i.e., 3 μg Malat1 ASO, without SO1861; ca 83.7% Malat1 RNA), treatment with SO1861 alone also reaches ca 85.3% Malat1 RNA in the right cerebrum. In contrast, the effect of local co-administration of 3 μg Malat1 ASO+SO1861 is not only larger (44.3% remaining Malat1), but it is even larger than the effect of the 3.33-times higher ASO dose (10 μg Malat1 ASO; 72.3% remaining Malat1 RNA) in the right cerebrum. Also, in the left cerebrum (further away from the injection site due to cerebrospinal fluid (CSF) flow), the local co-dose of 3 μg Malat1 ASO+saponin component SO1861 is still the most potent condition in reducing Malat1 RNA levels and the co-dosing also has a significant effect in the cerebellum (least exposed to CSF flow). Notably, also in the brainstem, which is the most distal brain region and furthest away from the injection site but highly exposed to the CSF, the local co-administration of 3 μg Malat1 ASO+SO1861 had an equal effect size in reducing RNA levels (45.0% Malat1 remaining) as in the right cerebrum. In conclusion, the combination of Malat1-ASO (3 μg)+SO1861 effectively reduced Malat1 expression in all brain regions tested compared to vehicle and SO1861. The combination of Malat1-ASO (3 μg)+SO1861 showed improved efficacy compared to Malat1 ASO (10 μg and 3 μg, respectively) in the brainstem and right cerebrum. In the brainstem, and in the right cerebrum, both a dose dependent effect of Malat1 ASO is observed as well as a synergistic effect of the combination dose of Malat1-ASO (3 μg)+SO1861.

### Example 2: Specificity of Enhancement of ASO Efficacy by Co-Administration to Saponin Components According to the Invention, Compared to Steroid(-Like) Saponins/Molecules as Measured by Efficacy of Malat1 mRNA Knockdown in Neuronal Cell Line

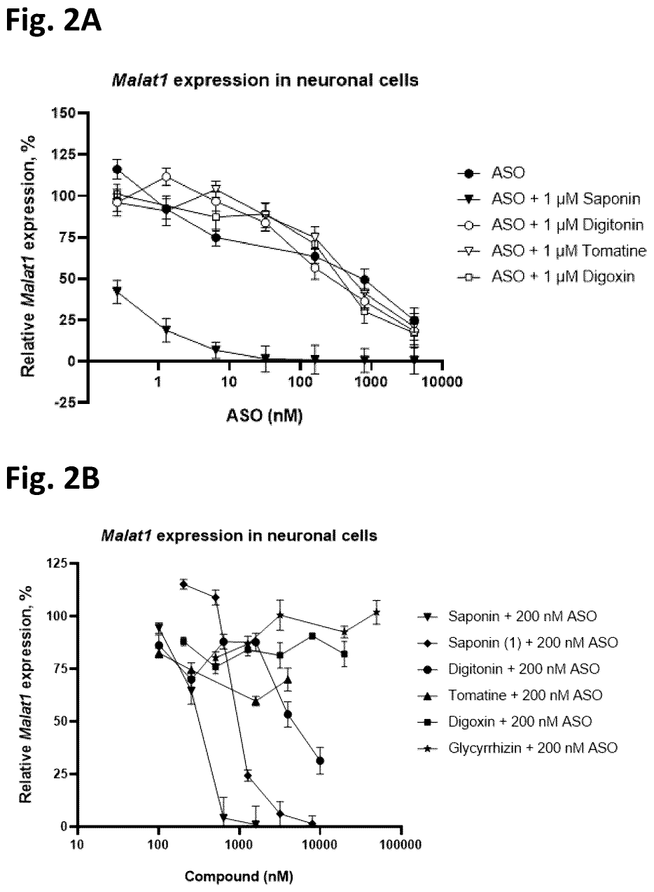

To assess the specificity of the enhancement effect of ASO-induced Malat1 RNA reduction by saponin components in co-administration, a Malat1 ASO was titrated with a fixed amount of either the saponin component SO1861 or different steroid(-like) saponins/molecules (digitonin, digoxin and tomatine) in Neuro-2a cells (FIG. 2A). Surprisingly, in this setting, only Malat1 ASO co-administration with the saponin component SO1861 lead to a marked Malat1 mRNA reduction and an IC50 shift of approximately 4 orders of magnitude, compared to ASO alone or all other co-administrations of ASO with steroid(-like) saponins/molecules digitonin, digoxin and tomatine (FIG. 2A). More remarkably, already around 30 nM ASO in, co-administration with saponin component SO1861 (at 1 μM) lead to complete loss of Malat1 mRNA. Neither digitonin, nor digoxin, nor tomatine (all at 1 μM) enhanced the efficacy of Malat1 ASO in co-administration, when compared to ASO alone. Even at 20.000 nM ASO, Malat1 RNA was still measurable (around 10-15%) for any of the co-administrations of ASO with digitonin, digoxin or tomatine, or for ASO alone. This shows that the co-administration enhancement effect in neuronal cells is specific to the saponin component SO1861 and is not observed with steroid(-like) saponins/molecules digitonin, digoxin and tomatine.

Next, to explore the minimal dose of saponin component needed to achieve maximum enhancement in co-dosing, a titration of saponin components (SO1861 or SO1861-AH-Block) or the steroid(-like) saponins/molecules digitonin, digoxin, glycyrrhizin and tomatine with a fixed amount of 200 nM ASO was performed (FIG. 2B). This analysis showed that co-administration of saponin component SO1861 lead to a marked mRNA reduction and complete loss already at relatively low dose: an exposure concentration of less than 650 nM SO1861 was sufficient to reveal almost full potency of 200 nM ASO (i.e., >95% mRNA reduction). Also, the saponin component SO1861-AH-Block is efficacious when compared with ASO alone or the steroid(-like) saponins/molecules. In contrast, digoxin, glycyrrhizin and tomatine had no enhancing effect in co-administration with 200 nM ASO at all: no mRNA reduction was observed at any of the exposure concentrations tested. As expected, digitonin (known to permeabilize the plasma membrane) shows approximately 70% reduction in Malat1 mRNA only at the relatively high dose of 10.000 nM, and is thus less efficacious to the endosome-selective escape enhancer SO1861-AH-Block, which achieved complete loss of Malat1 mRNA expression already above 3200 nM.

### Example 3 Efficacy Enhancement of Covalently Conjugated ASO-Saponin Component in Neuronal Cells

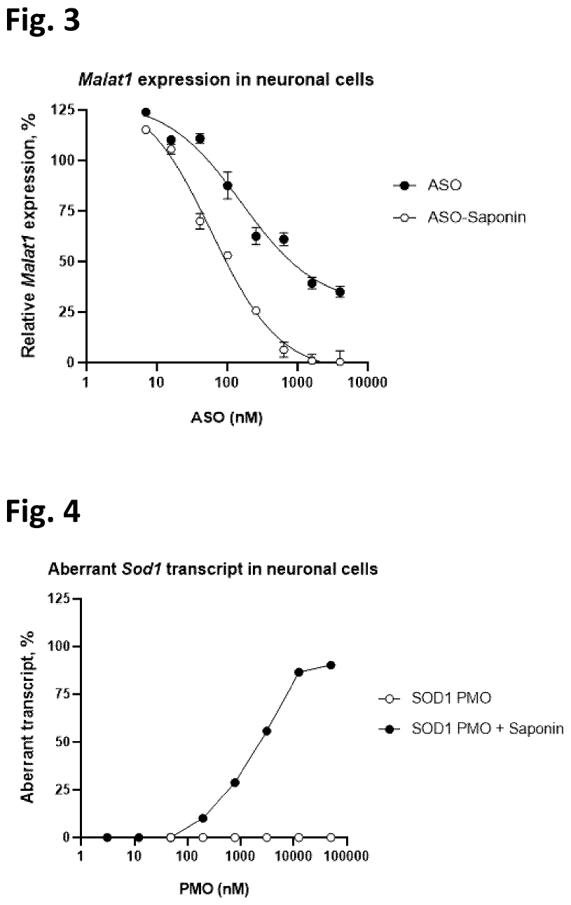

To improve and to synchronize delivery of the ASO and a saponin to the same cells/compartments, the ASO was directly (covalently) conjugated to SO1861 via an SC-containing linker, therewith providing a saponin component comprising the covalently bound ASO. Neuronal cells (Neuro-2a) were treated with ASO-SC-SO1861 conjugate, or ASO alone as comparator, and Malat1 mRNA reduction was measured (FIG. 3). This revealed that covalent conjugation improves the IC50 from 160 nM without saponin component to 60 nM with saponin component. More importantly, 4000 nM ASO alone achieved only 70% reduction in Malat1 mRNA expression, whereas, surprisingly, already 2000 nM ASO-SC-SO1861 conjugate showed complete (100%) reduction in Malat1 mRNA expression (FIG. 3). This data shows that synchronization of the ASO with SO1861 by covalent conjugation results in improved efficacy in neuronal cell line, and that conjugation of saponin molecules to payloads does not create a limiting (effect prohibitive) factor. In contrast, such co-delivery by delivery of the saponin component comprising the bound ASA ensures that ASO and saponin are together delivered to the same cell and to the same cellular compartments to optimize bio-/subcellular distribution and thereby efficacy enhancement.

### Example 4: Efficacy Enhancement of a Sod1-Targeting PMO by Co-Administration of a Saponin Component in Neuronal Cells

Mutations in SOD1 have been linked to familial amyotrophic lateral sclerosis (ALS), and knockdown of mutant SOD1 has been associated with ameliorating disease. Reduction of mutant SOD1 RNA thus arises as a potential treatment; tofersen (Qalsody), an ASO targeting mutant SOD1 RNA was FDA approved in 2023. Here, the knockdown enhancement effect of saponin components on the efficacy of a PMO to reduce Sod1 RNA was therefore assessed in a neuronal cell line. To this end, a splice-site blocking SOD1 PMO (SEQ ID NO: 20) was designed to induce aberrant Sod1 transcripts and to thereby lead to a premature termination codon (i.e. effectively lead to reduction of Sod1 mRNA). The PMO was added to Neuro-2a cells in a dose-range with or without a fixed amount of saponin component (SO1861-SC-Mal) and levels of aberrant Sod1 RNA transcripts was determined by PCR. Notably, the PMO alone (without the saponin component) did not induce any aberrant Sod1 transcripts even at the highest concentration (50 μM PMO; FIG. 4). Remarkably however, co-administration of PMO+3 μM saponin component (SO1861-SC-Mal) lead to a marked increase in aberrant Sod1 transcripts of up to 90% at 50.000 nM (FIG. 4). Similar results in inducing high amounts of aberrant Sod1 transcripts was obtained by co-administration of saponin components with a second PMO (SEQ ID NO: 23) that was designed to bind to a different region on the Sod1 mRNA but inducing same effect (data not shown). This shows that the co-administration of such PMOs with saponin components enhance the delivery of the PMO in neuronal cells and beneficially induce aberrant transcripts of a disease relevant gene, with the aim of thereby reducing the expression of a mutant and pathogenic gene in neuronal cells, in this example resulting in the prophylaxis or treatment of ALS with the nucleic acid therapeutic.

### Example 5: In Vivo Evaluation of Tolerability and Effect in the Eye of Saponin Components with or without an LNA Payload

PS 2′-locked nucleic acids (PS 2′ LNA) ASOs are antisense oligonucleotides (DNA/RNA gapmers) with a phosphorothiated (PS) stabilized backbone and modified RNA nucleotides in the ‘gapmer wings’ in which a methylene bridge has been introduced between the 2′ C- and 4′ C-atoms. PS 2′ LNA ASOs have generally been described as more toxic (in vitro but also in vivo in mouse and NHPs) compared to other 2′ modified PS ASOs (such as 2′-MOE ASOs or 2′-OMe PS ASOs). In the clinic, several different PS 2′ LNA ASOs were shown to be toxic with severe thrombocytopenia, severe liver toxicity, and nephrotoxicity being observed (Crooke et al., Antisense technology: A review, J. Biol. Chem., Volume 296, 2021, 100416, 2021). Corneal pachymetry is a non-invasive method to evaluate corneal thickness. The most common cause of a corneal thickening over time is edema or swelling.

### In Vivo Study Design

Sixteen male Brown Norway (BN) rats were included in the study. The animals were divided into four groups (three or five animals/group, as indicated in Table A2), to receive a single intravitreal (IVT) injection of vehicle or test item. Injections were performed into the right eye (OD); the left eye (OS) was left untreated and served as a control eye.

TABLE A2
IVT in vivo dosing schedule

| Group | Compound | Dose (μg) | Animals | Dose volume (μL) |
|---|---|---|---|---|
| 1 | Vehicle | — | 3 | 3 |
| 2 | SO1861 | 3 μg | 3 | 3 |
| 3 | LNA | 5 μg | 5 | 3 |
| 4 | LNA + | 5 μg LNA + | 5 | 3 |
| | SO1861 | 3 μg SO1861 | | |

On the day of injection, the animals were subjected to health monitoring, using a modified Irwin screen, and clinical examinations including slit-lamp inspection of the anterior chamber, intraocular pressure (IOP) and pachymetric measurements in addition to retinoscopy to verify that the eyes were normal before injections. Thereafter, the animals were anaesthetised to inject the vehicle or test items in a volume of 3 μL into the vitreous humor of the right eye. Directly after each administration, the eye was examined to control the integrity of the eye. Slit-lamp and ophthalmologic examinations were performed pre-dose and at 24 hrs and 72 hrs after injection in both eyes. Body weight measurements and health monitoring were performed at 24 hrs and 72 after injection.

### Effect of an LNA and Saponin Component or Co-Administered LNA+Saponin Component in the Eye

As FIG. 5 shows, IVT injection of vehicle did not affect corneal thickness of the treated right eye over 72 hrs, indicating that the injection procedure itself had no effect. Similarly, IVT injection of either 5 μg LNA, or 3 μg saponin component (SO1861) did not cause an increase of corneal thickness of the treated right eye. In all cases, corneal thickness in the treated right eyes was similar to the left eyes that were not injected (data not shown). Surprisingly, injection of co-dosing LNA+SO1861 (5+3 μg) caused however marked increases in corneal thickness at 24 hrs but more so after 72 h after injection. Notably, the left eyes that were not treated did not show corneal thickening (data not shown). This data shows that the saponin component can be administered to the eye and by itself is not leading to edema or swelling. It also, and more importantly, shows that only in co-administration of saponin compound with LNA ASO corneal thickening processes are activating, indicating that the saponin component effectively releases the LNA.

### Example 6: Efficacy Enhancement by Co-Dosing of Saponin Components with Different Antisense Oligonucleotide Modalities with Different Mechanisms of Action Targeting the Disease Relevant STAT3 Gene

Abnormal activation of the transcription regulator gene STAT3 has been associated with Alzheimer Disease (AD). As such, phosphorylation of STAT3 is dramatically increased in the hippocampus of AD mouse model and in AD post-mortem brain. Moreover, STAT3 may act as a transcriptional regulator of BACE1, the key enzyme in amyloid beta (AB) production. Likewise, STAT proteins are activated by phosphorylation in the spinal cord of patients suffering from amyotrophic lateral sclerosis (Ohgomori et al, Differential activation of neuronal and glial STAT3 in the spinal cord of the SOD1G93A mouse model of amyotrophic lateral sclerosis, EJN, Volume 46, Issue 4, August 2017, Pages 2001-2014). STAT3 is also biologically relevant therapeutic target in H3K27M-mutant diffuse midline glioma (Zhang et al., STAT3 is a biologically relevant therapeutic target in H3K27M-mutant diffuse midline glioma, Neuro Oncol, 2022 Oct. 3; 24 (10): 1700-1711. doi: 10.1093/neuonc/noac093). Antisense oligonucleotides targeting STAT3 are in clinical development (Hong et al., AZD9150, a Next-Generation Antisense Oligonucleotide Inhibitor of STAT3 with Early Evidence of Clinical Activity in Lymphoma and Lung Cancer, Sci Transl Med. 2015 Nov. 18; 7 (314): 314ra185.doi: 10.1126/scitranslmed.aac5272).

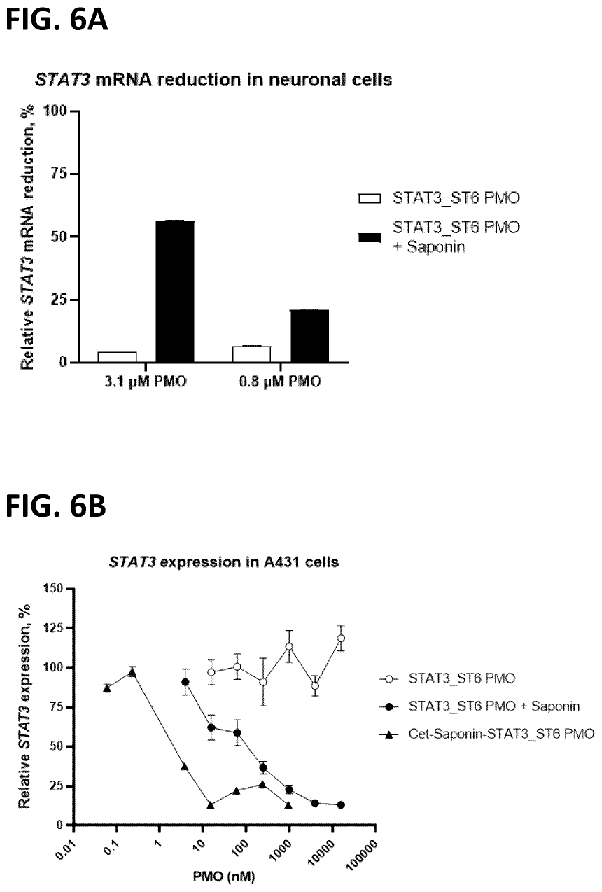

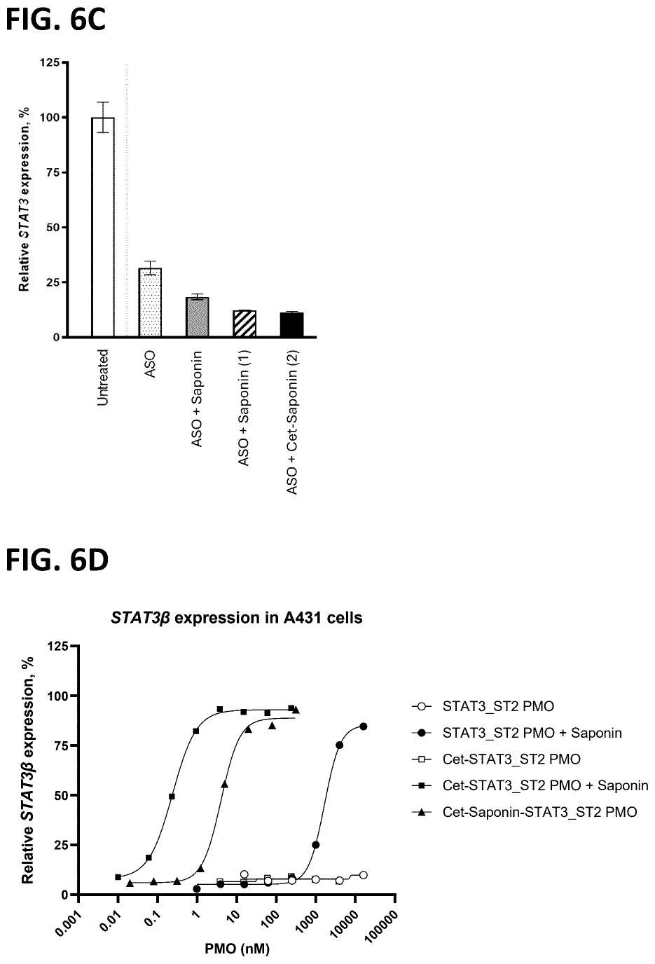

Here, the co-dosing enhancement effect on modulating STAT3 RNA levels by saponin components with different antisense oligonucleotides with different modes of action were assessed. Firstly, mouse neuronal cells were incubated with a STAT3_ST6 PMO ([SEQ ID NO: 36]; Zammarchi et al., Antitumorigenic potential of STAT3 alternative splicing modulation, Proc Natl Acad Sci USA. 2011 Oct. 25; 108 (43): 17779-17784, Published online 2011 Oct. 17. doi: 10.1073/pnas.1108482108 (Zammarchi et al., 2011) with and without saponin component. The resultant effect on Stat3 mRNA expression levels were determined. This STAT3_ST6 PMO has previously been shown to induce nonsense mediated decay of the STAT3 mRNA by inducing skip of STAT3 exon 6 effectively leading to STAT3 mRNA reduction in a variety of human cancer cell lines in vitro and in vivo. As shown here in FIG. 6A, in murine neuronal cells, STAT3_ST6 PMO alone induced only a minimal reduction (4-7%) of the Stat3 mRNA levels at 0.8 UM or 3.1 μM PMO. However, co-administration of STAT3_ST6 PMO+3 μM saponin component (SO1861-SC-Mal) showed markedly improved efficacy in reducing Stat3 mRNA even up to 56% with 3.1 μM STAT3_ST6 PMO (FIG. 6A). Even at 0.8 UM PMO+3 μM saponin component (SO1861-SC-Mal), ca 21% Stat3 mRNA reduction were still observed. This data shows that saponin components can effectively enhance an exon skipping PMO to reduce Stat3 mRNA levels in neuronal cells.

Next, both STAT3_ST6 PMO and saponin component (SO1861-SC-Mal) were covalently conjugated to an EGFR-targeting ligand (monoclonal antibody cetuximab, Cet) to yield Cet-SO1861-STAT3_ST6 PMO and titrated in a dose range on the EGFR-expressing A431 cell line and compared with a dose range of non-conjugated STAT3_ST6 PMO with or without a fixed concentration of saponin component. This confirmed that the STAT3_ST6 PMO alone shows no reduction in STAT3 mRNA levels even at the highest concentrations tested, whereas STAT3_ST6 PMO+3 μM SO1861-SC-Mal showed a dose dependent reduction of STAT3 mRNA levels also on A431 cells (FIG. 6B). Interestingly, synchronization of the cellular delivery of the STAT3_ST6 PMO and saponin component (SO1861-SC) in form of the targeted conjugate Cet-SO1861-STAT3_ST6 PMO showed even higher efficacy resulting in dose dependent reduction of STAT3 mRNA levels at very low concentrations of STAT3_ST6 PMO, with an IC50 of ca. 1 nM (FIG. 6B). This data shows that saponin components work effectively after conjugating and targeting an exon skipping PMO to reduce STAT3 RNA levels.

Next, we assessed enhancement of a STAT3 expression modifying ASO (antisense oligonucleotide with a different mode of action, namely ribonuclease H mediated RNA degradation) by different saponin components (targeted and non-targeted). To this end, human A431 epidermoid carcinoma cells were incubated with the RNA degrading STAT3-ASO (Hong et al., 2015) and various saponin components (SO1861, SO1861-AH-Maleimide-Block, or Cet-AH-SO1861, respectively). Treatment for 48 hrs with ASO alone caused a significant reduction in STAT3 expression in A431 cells to a residual 32%, but co-administration of ASO plus a saponin component (targeted or non-targeted) showed reduction down to 11% to 18% (FIG. 6C), irrespective of which saponin component was used. This data shows that co-dosing of saponin components, whether targeted or not, with an RNA-degradation-inducing ASO also results in superior efficacy, giving credence to the combination and use of different payload types in combination a saponin component to modulate STAT3 mRNA levels.

Additionally, the co-dosing enhancement effect of saponin components was assessed on a splice-switch inducing PMO, STAT3_ST2 PMO ([SEQ ID NO: 37]). This STAT3_ST2 PMO has been shown to modulate the splicing of STAT3 preRNA and thereby promote the expression of the STAT3B isoform over STAT3a isoform, which can have beneficial therapeutics effects (Zammarchi et al., 2011). Notably, nusinersen (Spinraza), is an ASO designed to modulate alternative splicing to allow the SMN2 gene to produce the full-length and functionally normal protein as a treatment for spinal muscular atrophy (SMA), an autosomal recessive disorder caused by loss or mutation of the SMN1 gene and retention of the SMN2 gene. Here, to assess co-dosing enhancement effect of saponin components on STAT3_ST2 PMO, human A431 epidermoid carcinoma cells were titrated in a dose range of (1) STAT3_ST2 PMO with and without saponin component, (2) Cet-STAT3_ST2 PMO (a conjugate in which STAT3_ST2 PMO is covalently conjugated to the EGFR-binding monoclonal antibody cetuximab) with and without saponin component, or (3) Cet-SO1861-STAT3_ST2 PMO (a conjugate in which both STAT3_ST2 PMO and saponin component (SO1861-SC) were covalently conjugated to the EGFR-binding monoclonal antibody cetuximab). Since STAT3_ST2 PMO has previously shown to induce splice switching of the STAT3 mRNA (Zammarchi et al., 2011) from STAT3a to STAT38, the amount of STAT3B mRNA expression levels were determined for the different treatment conditions (FIG. 6D). This revealed that neither STAT3_ST2 PMO nor Cet-STAT3_ST2 PMO alone showed activity over the entire dose range tested. However, co-administration of STAT3_ST2 PMO+saponin component showed dose dependent efficacy with an apparent IC50=1700 nM PMO (FIG. 6D). Remarkably, targeted Cet-STAT3_ST2 PMO+saponin component (SO1861-SC-Mal) showed the strongest increase in efficacy with an IC50=0.2 nM PMO, whereas also the 1-component (Cet-SO1861-STAT3_ST2 PMO; a saponin component comprising both an oligonucleotide and an endocytic cell-surface receptor targeting ligand, here a monoclonal antibody) showed the most strongly improved dose dependent increase in STAT3B mRNA expression with an IC50=4.0 nM PMO (FIG. 6D).

Taken together these data show that enhancing the efficacy of targeting of a disease relevant gene (here, STAT3) by saponin components can be accomplished by different modalities (PMO, ASOs) with different mechanisms of action (exon skip leading to RNA degradation by PMO or RNaseH mediated RNA degradation by ASO, exon skip by PMO leading to a reduction of an isoform/increase of another (beneficial) isoform), or conjugating PMO/ASO and/or conjugating the saponin component, with or without a cell-targeting ligand (endocytic cell-surface receptor targeting ligand).

### Example 7: Efficacy Enhancement by Co-Dosing of Saponin Components with Various Modified AHA1 and MMP14 siRNA In Vitro

The microtubule-associated protein tau (MAPT, tau) forms neurotoxic aggregates that promote cognitive deficits in tauopathies, the most common of which is Alzheimer's disease (AD). AHA1 has been shown to contribute to tau fibril formation and neurotoxicity through Hsp90. This suggests that therapeutics targeting AHA1 may reduce toxic tau oligomers and slow or prevent neurodegenerative disease progression (Shelton et al., Hsp90 activator Aha1 drives production of pathological tau aggregates, Proc Natl Acad Sci USA 2017; 114 (36): 9707-9712). MMP-14 overexpression correlates with the neurodegenerative process in familial amyloidotic polyneuropathy (FAP) (Martins et al., MMP-14 overexpression correlates with the neurodegenerative process in familial amyloidotic polyneuropathy, Dis Model Mech. 2017 Oct. 1; 10 (10): 1253-1260) and its upregulation is associated with glioma expansion. In patients with Alzheimer's disease (AD), MMP-14 was found overexpressed in brain.

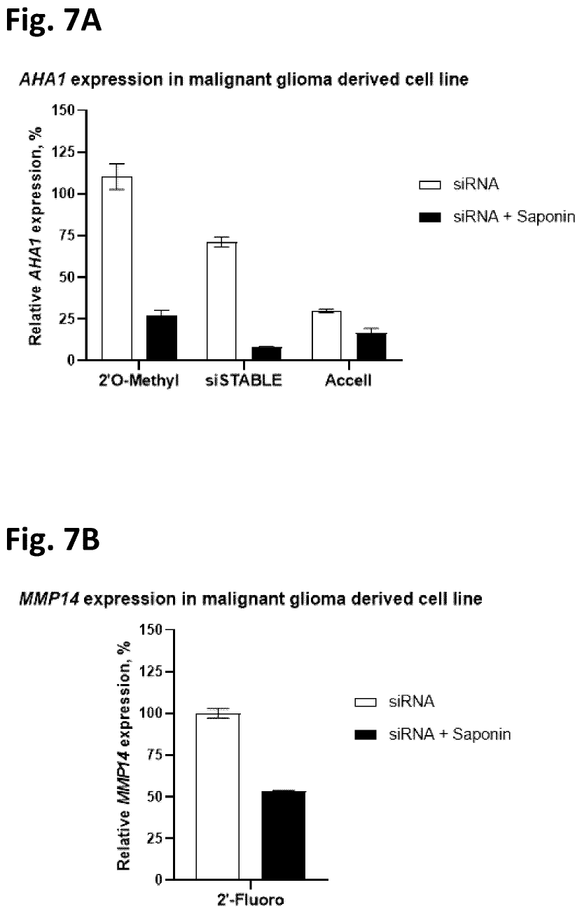

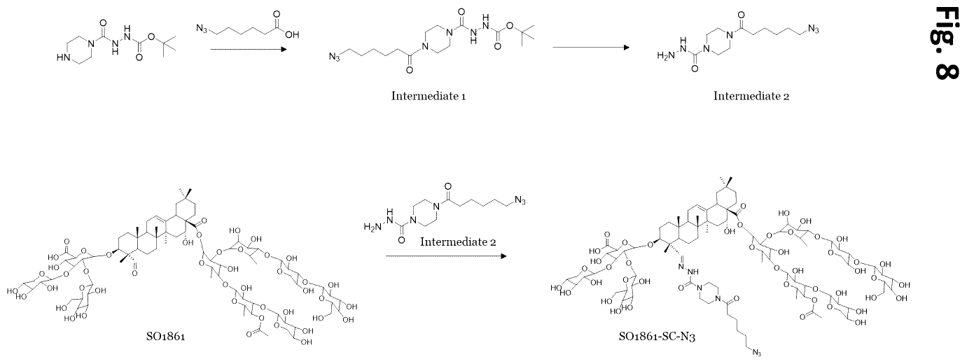

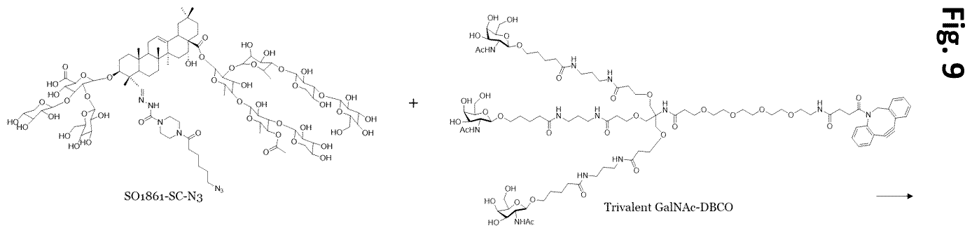

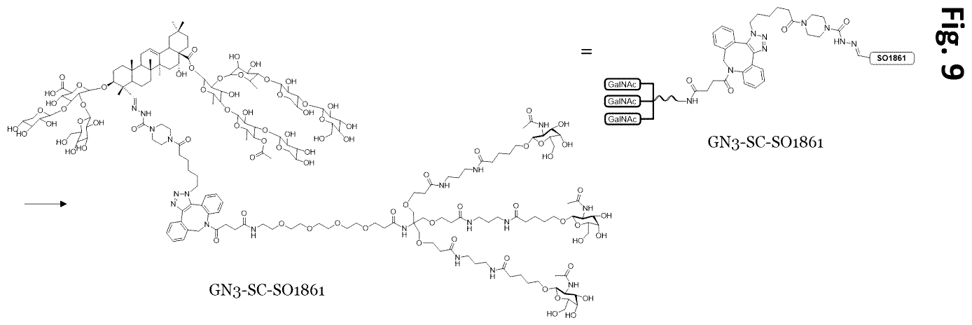

To assess the co-dosing enhancement effect by saponin components of siRNA oligonucleotides in human brain cells, Neuro-2a cells were incubated with 2000 nM AHA1 siRNA with different modifications: (1) modified with 2′O-Methyl, (2) commercial proprietary stabilization chemistry siSTABLE (Thermo Scientific), or (3) commercial proprietary stabilization chemistry Accell (Thermo Scientific), either with or without 1.3 μM saponin component (SO1861). Treatment for 48 hrs on human brain cells revealed that improved stability of the siRNA improves the reduction in AHA1 mRNA expression. Co-administration of SO1861 markedly enhanced this effect (FIG. 7A). Independent of modification, the co-administration with saponin component SO1861 was the most efficacious treatment for all siRNAs.

In another example a stabilized siRNA against MMP14 was tested, (this time with 2′-Fluoro modifications). The siRNA was tested for its efficacy and the co-dosing enhancement effect by saponin component (SO1861) in human brain cells. Treatment with 2000 nM siRNA alone did not reveal any efficacy, while coadministration with saponin component improved the efficacy of the stabilized siRNA. This data shows that also the efficacy of (stabilized) siRNAs can be enhanced by saponin components.

### Example 9: In Vivo Efficacy Enhancement by Local Co-Administration of Saponin Components to (Targeted)-ASO/PMO Compounds in the Brain/CNS

CNS disorders like Parkinson's Disease pathology and familial amyotrophic lateral sclerosis (ALS) have been linked to (mutations in) the MALAT1 and SOD1 genes, which are therefore recognized as potential therapeutic targets for downmodulation. Local co-administration of saponin components significantly enhances the potency of a Malat1-targeting ASO and reduces Malat1 expression levels in several (larger) brain regions in the mouse (FIG. 1). To further show in which particular brain regions marked enhancement is observed, Malat1 gene expression was studied in more detail in (structurally and functionally) defined brain regions (Table A8). Furthermore, and in addition, the effect of saponins was also studied for a Sod1-targeting PMO to analyze the effect of saponin component on a different payload type in the brain/CNS and the efficacy and tolerability was compared between naked (unconjugated) ASO/PMO and ligand-conjugated ASO/PMO, both with or without saponin component co-dosing. Finally, a Malat1 ASO-Saponin conjugate, in which the ASO was directly (covalently) conjugated to a saponin component, that has shown improved activity in vitro in a neuronal cell model (FIG. 3) was included to study the effect of a 1-component approach in vivo.

TABLE A8
CNS in vivo dosing schedule

| Group | | | Volume | Dose | Payload | SO1861 |
|---|---|---|---|---|---|---|
| No. | Treatment name | RoA | [μL] | [μg/μL] | [pmol] | [pmol] |
| A | Vehicle (DPBS) | ICV | 10 | NA | N/A | N/A |
| B | Malat1 ASO (3 μg) + | ICV | 10 | 0.30 + | 401 | 1200 |
| | Saponin (2.23 μg) | | | 0.223 | | |
| C | Saponin (1) (2.62 μg) | ICV | 10 | 0.262 | N/A | 1200 |
| D | Malat1 ASO-Saponin (low) | ICV | 10 | 0.38 | 401 | 401 |
| | (3.8 μg) | | | | | |
| E | Malat1 ASO-Saponin (high) | ICV | 10 | 1.14 | 1200 | 1200 |
| | (11.4 μg) | | | | | |
| F | aCD71-Malat1 ASO | ICV | 10 | 4.09 | 375 | N/A |
| | (40.9 μg) | | | | | |
| G | aCD71-Malat1 ASO | ICV | 10 | 4.09 + | 375 | 1200 |
| | (40.9 μg) + Saponin | | | 0.223 | | |
| | (2.23 μg) | | | | | |
| H | SOD1 PMO (5.9 μg) | ICV | 10 | 0.59 | 741 | N/A |
| I | SOD1 PMO (5.9 μg) + | ICV | 10 | 0.59 + | 741 | 1200 |
| | Saponin (2.23 μg) | | | 0.223 | | |
| J | aCD71-SOD1 PMO | ICV | 10 | 3.93 | 741 | N/A |
| | (Compound 1) (39.3 μg) | | | | | |
| K | aCD71-SOD1 PMO | ICV | 10 | 3.93 + | 741 | 1200 |
| | (Compound 1) (39.3 μg) + | | | 0.223 | | |
| | Saponin (2.23 μg) | | | | | |

SO1861 = saponin component;
RoA = route of administration;
ICV = intraventricular

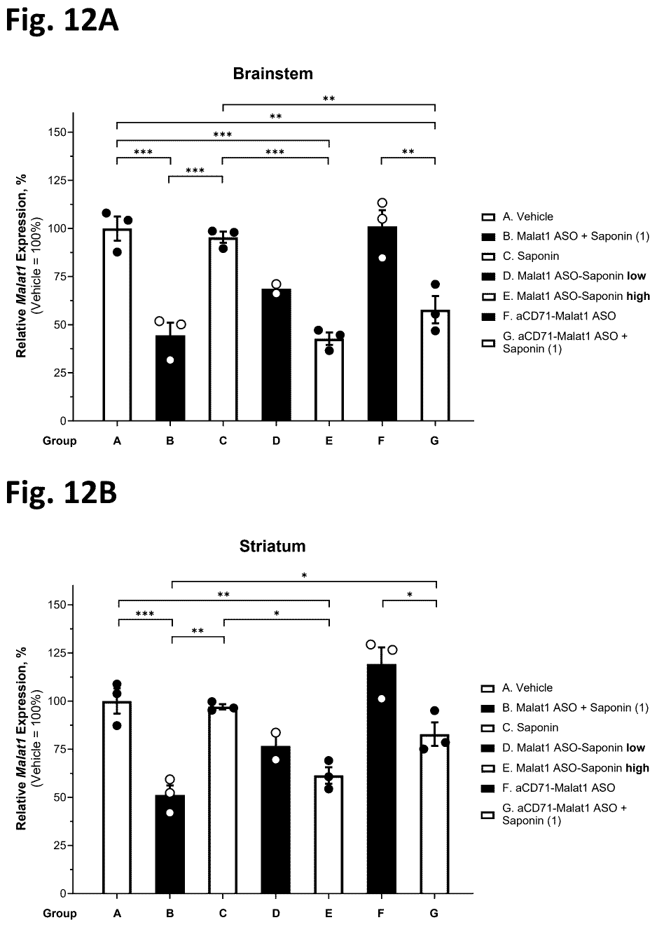

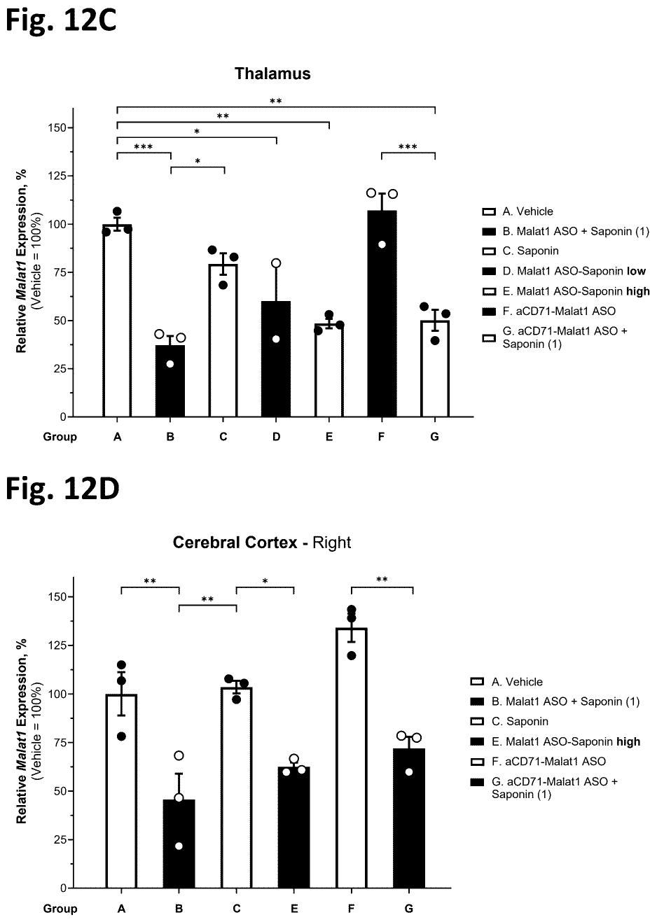

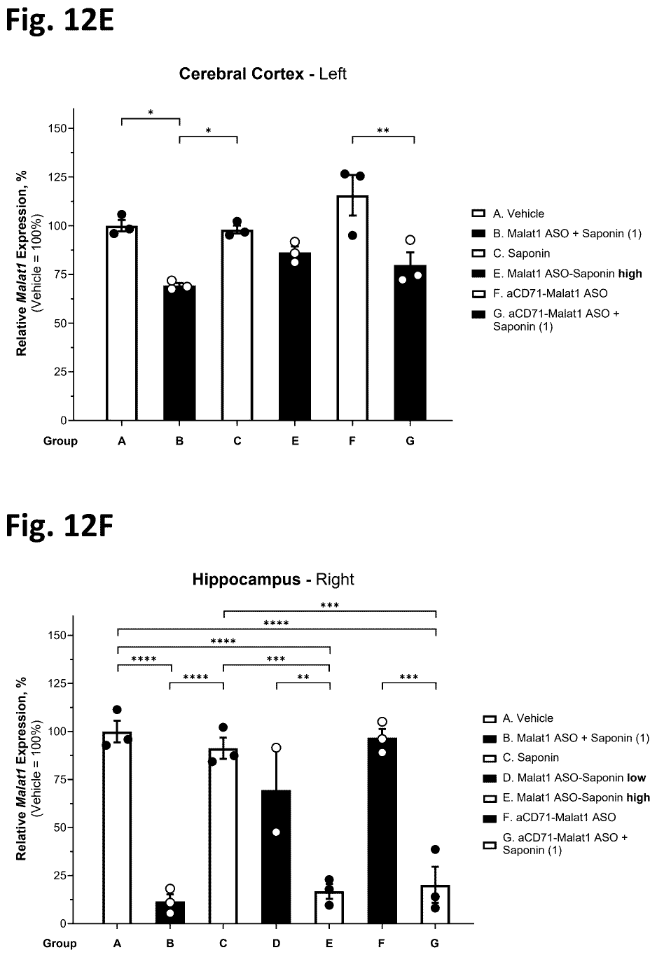

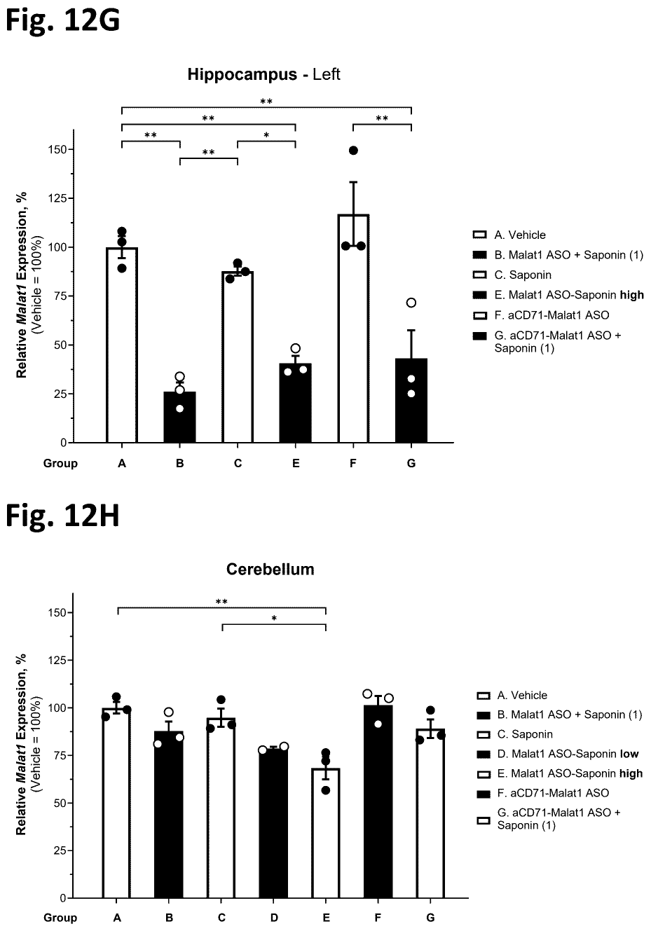

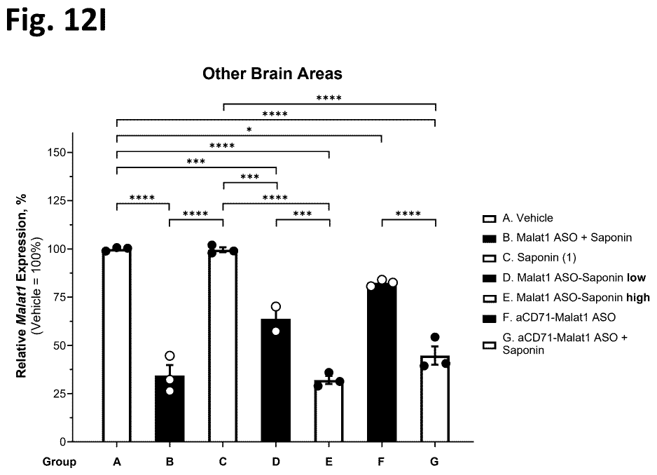

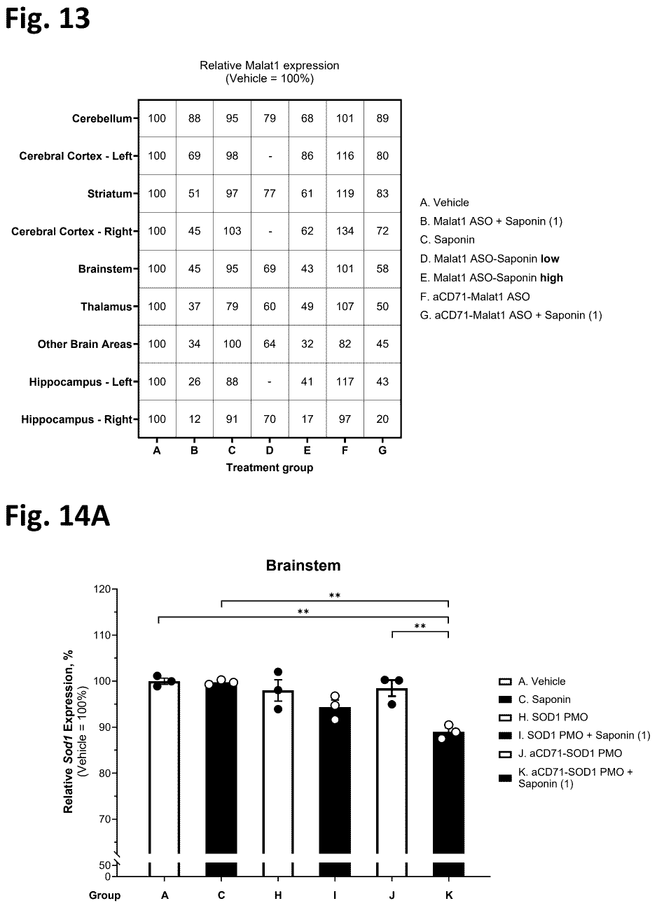

FIG. 12 and FIG. 13 show the relative Malat1 mRNA expression in the different brain regions for each treatment group, with or without saponin component, compared to the vehicle-treated group. A significant reduction of CNS Malat1 mRNA expression was seen in almost all brain regions, 10 days after unilateral ICV administration of the Malat1 ASO in combination with saponin (Group B), which was comparable in effect to study 1 (FIG. 1) and confirmed the initial findings. A significant reduction of CNS Malat1 mRNA expression was also observed for the 1-component Malat1 ASO-Saponin conjugate in almost all brain regions (Group D+Group E), in which the higher dose was, as expected, more potent than a lower dose, also confirming the specificity (dose-dependent) effect. The effectivity of the high dose of directly conjugated Malat1 ASO-Saponin was comparable to the co-dosing treatment of Malat1 ASO+saponin component in most brain regions. The potency of a targeting ligand-conjugated ASO (ie, aCD71-Malat1 ASO conjugate treatment groups, Group F+Group G) was not improved compared to non-conjugated ASO, when either were co-dosed with saponin component. While the addition of the saponin component was required to unlock the high potency of either, aCD71-Malat1 ASO or unconjugated ASO in almost all brain regions, the ligand targeting did not markedly add to potency increase in the case of this ASO with a fully phosphorothioated backbone. It can be concluded that the saponin component enhances the endosomal escape of both unconjugated (naked) and ligand-targeted ASO with a negative backbone charge.

Remarkably, a highly comparable relative efficacy response profile for the various treatments was observed in all brain regions except for in the cerebellum (FIG. 13). Absolute responses (i.e., order of magnitude of the response) exhibited differences, with regions closest to the site of injection (right ventriculum) being the most responsive. As such, the hippocampus (right) shows up to 88% downmodulation of Malat1 for the co-administration of Malat1 ASO+saponin (Group B), while in the cerebellum only 12% downmodulation was observed. Interestingly, the conjugated Malat1 ASO-Saponin outperforms the co-administered Malat1 ASO+saponin or other aCD71-Malat1 ASO+saponin treatments in the cerebellum, meaning a higher potency increase is observed for the conjugated ASO-Saponin than for the co-administration. This shows that direct conjugation of ASO and saponin component is beneficial in reaching certain brain regions, including those that are far from the injection site or less exposed to CSF flow.

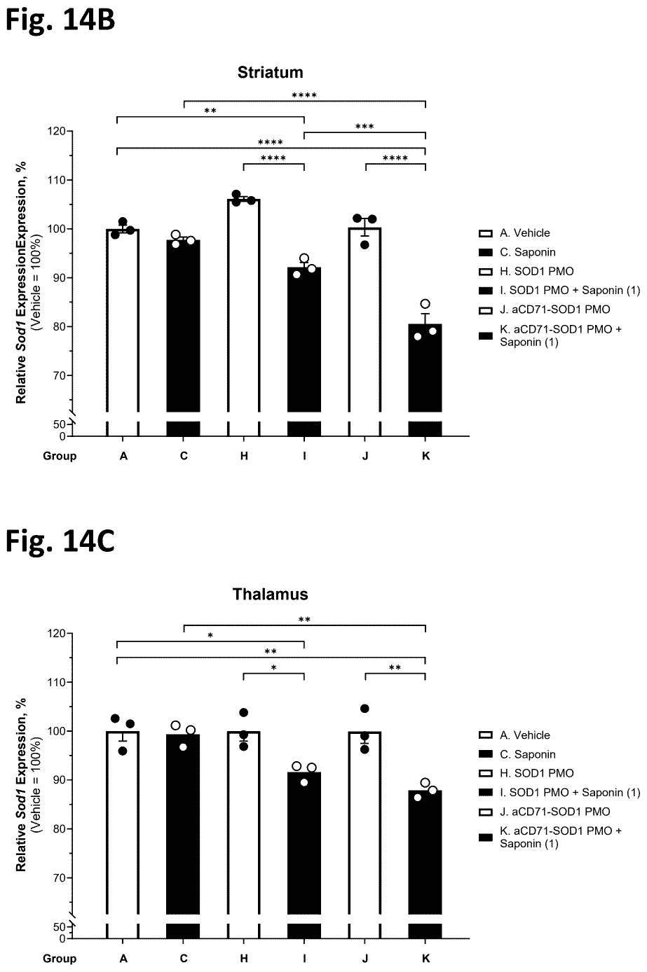

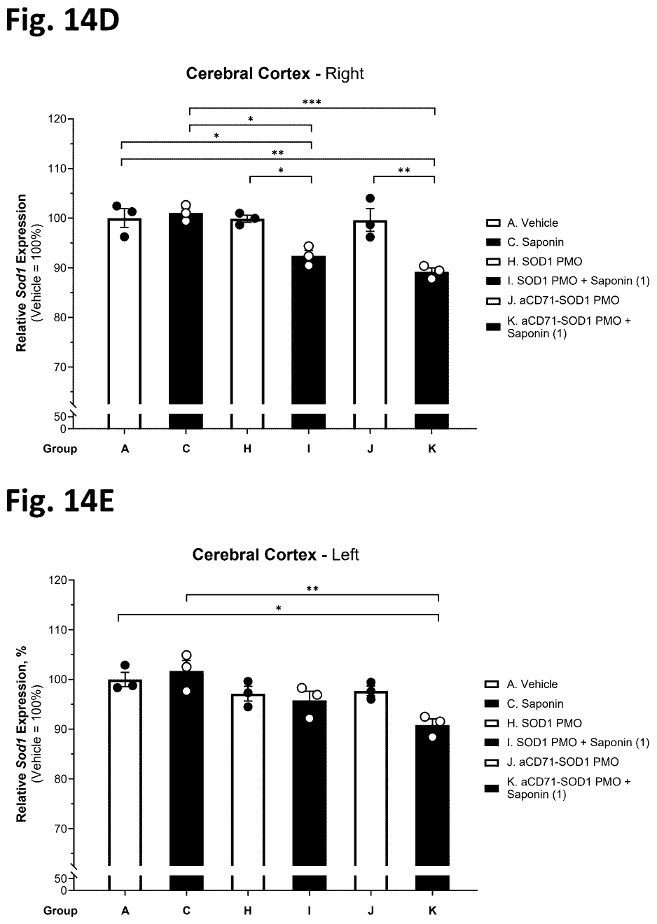

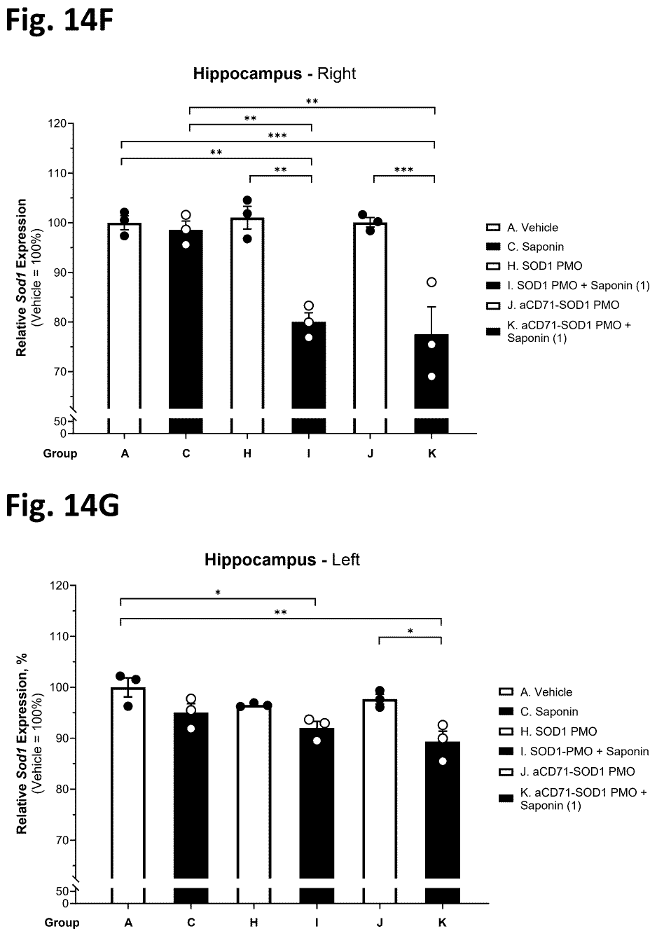

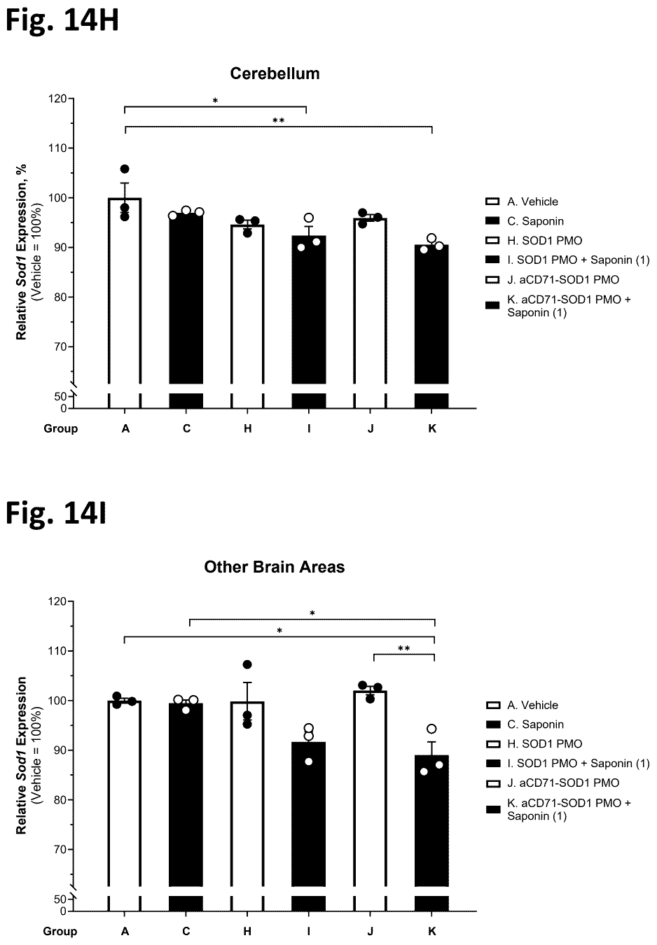

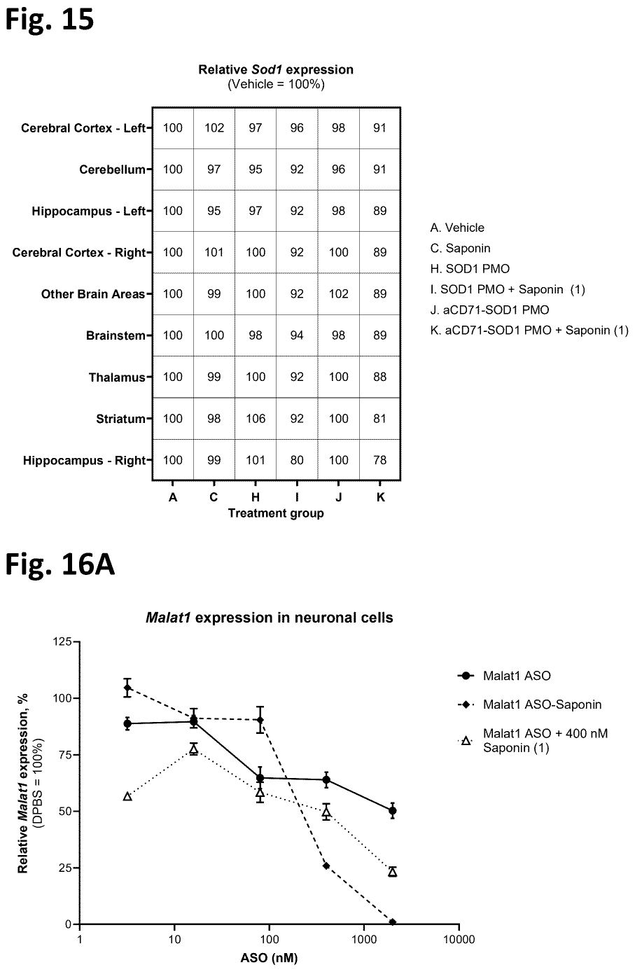

FIG. 14 and FIG. 15 show the relative Sod1 mRNA expression profiles in different brain regions for different treatment groups, with or without saponin component and with or without antibody-conjugation (i.e., aCD71 targeting) of the SOD1 PMO, compared to vehicle treatment. Importantly, no downregulation of Sod1 mRNA was observed in any SOD1 PMO treatment groups without saponin component, i.e., in treatment conditions where a saponin component was absent. In presence of a saponin component (i.e., co-administration) with a SOD1 PMO compound however, a clear and significant reduction of Sod1 mRNA, with a maximal reduction of 22%, compared to vehicle in almost all brain regions was observed. Interestingly, and differently to the aCD71-ASO conjugate, there is a clear potency increase in the group treated with ligand conjugated aCD71-SOD1 PMO+saponin (Group K) compared to treatment of non-conjugated (naked) SOD1 PMO+saponin (Group I). These results indicate that for a (neutrally charged) PMO, ligand conjugation has a beneficial effect in combination with a saponin component in realizing a potency increase, as the ligand is likely increasing the endosomal/cellular uptake of the PMO and the saponin component is mediating the endosomal release. Again, the strongest response is observed close to the site of injection, in the hippocampus (right), and is, as expected, lower in regions further from the injection site and CSF flow (e.g., the cerebellum and cerebral cortex (left) (FIG. 15)).

In conclusion, these analyses show that saponin components, either in co-administration or by (covalent) conjugation, are revealing and strongly potentiating the effect of an oligonucleotide treatment, e.g. an ASO with a negative charge/fully phosphorothioated backbone or a (charge neutral) PMO, in the brain/CNS. Depending on the oligonucleotide chemistry and characteristics (e.g., neutral or negative backbone charge), the conjugation to a targeting ligand further improved and/or revealed the potency (over a non-conjugated oligonucleotide). Whether directly conjugated, ligand-conjugated or unconjugated, the saponin component increased the potency of the oligonucleotide.

### Example 10: Efficacy Enhancement of Covalently Conjugated ASO-Saponin in Neuronal Cells

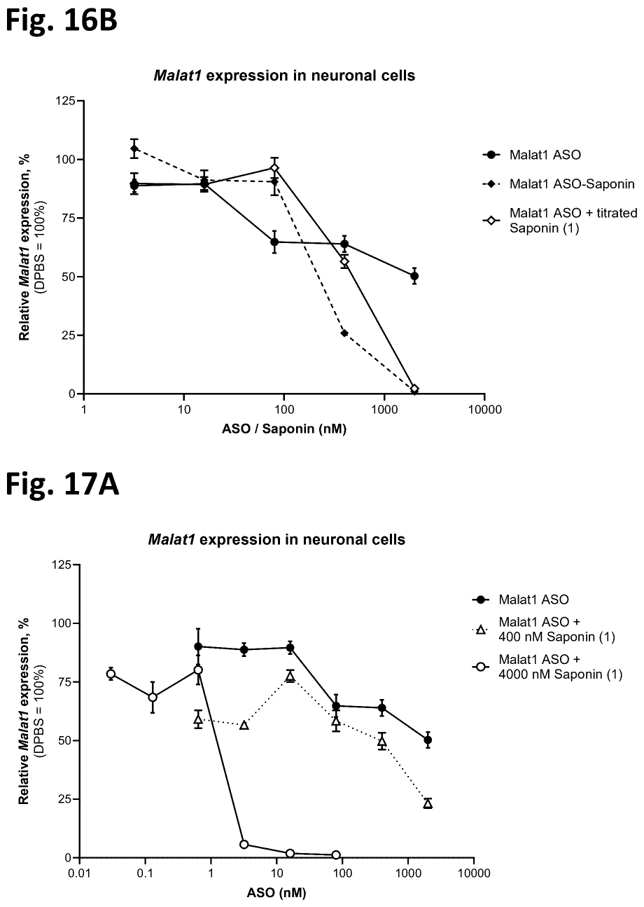

Direct (covalent) conjugation of a saponin component to an ASO (i.e., a ASO-Saponin conjugate) improves the ASO potency in neuronal cells as it synchronizes the delivery of the ASO and the saponin into the same subcellular compartment, where the saponin effects the release of the ASO (FIG. 3). To further assess and strengthen that conjugation of a saponin component to an oligonucleotide, e.g. an ASO, improves ASO efficacy, additional experiments were performed in neuronal cells. To this end, firstly, non-conjugated (naked) ASO was co-dosed with a low (400 nM) dose of saponin (1), which is of the same type and of a similar amount of saponin as in the active range of the ASO-Saponin conjugate. This co-administration was compared to treatment with either ASO-Saponin conjugate or with ASO alone (FIG. 16A). Cell viability was not affected by any of the treatments applied (data not shown). Notably and as previously shown, gene expression analysis confirms that covalent conjugation of the saponin to the ASO (i.e., the ASO-Saponin conjugate) markedly improves the potency compared to ASO alone or co-administered (low dose) saponin to ASO, as target gene expression levels is completely abrogated only for covalent conjugation at 2000 nM. To confirm that conjugation is beneficial, cells were also treated with non-conjugated ASO and the saponin component (ASO+titrated Saponin (1)), both titrated at a compound-ratio equal to the ASO-Saponin conjugate at each data point of the curve (FIG. 16B). This confirmed that conjugated ASO-Saponin is indeed more potent than non-conjugated ASO+titrated Saponin (1), when compared at equal concentrations. Taken together this data proves that synchronization of the cellular delivery of ASO and saponin by covalent conjugation results in improved efficacy in neuronal cell line, compared to ASO alone but also to ASO co-administered with saponin.

### Example 11: Efficacy Enhancement of (Targeted)-ASO by Co-Administration of a Saponin Component in Neuronal Cells

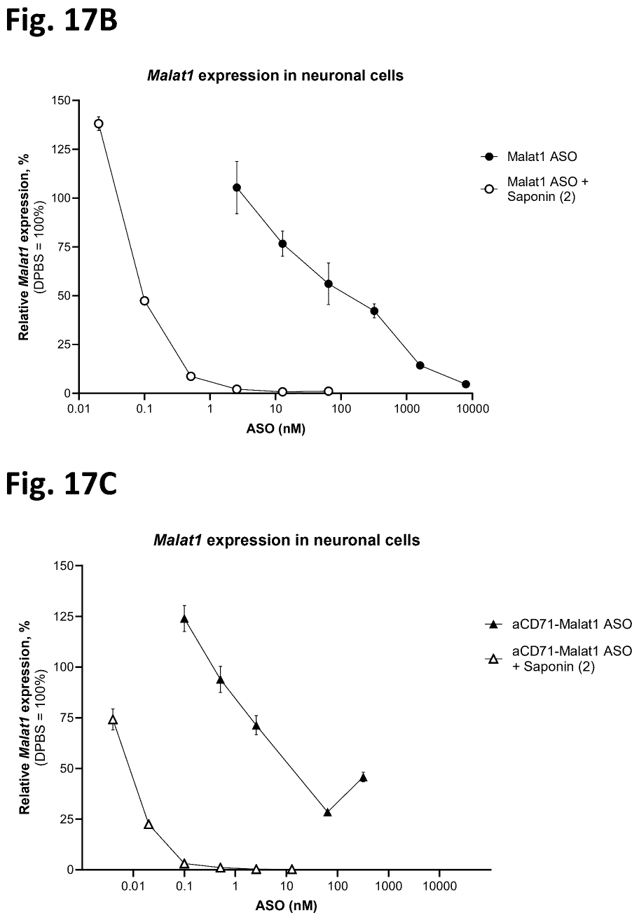

Neuro-2a cells were treated with a Malat1 ASO with and without the saponin component (being 4 μM Saponin (1) or Saponin (2)). Gene expression analysis revealed that a non-conjugated ASO (without saponin component) lead up to 50% reduction of Malat1 transcript at 2000 nM ASO (FIG. 17A). However, when the ASO was co-administered with Saponin (1), the efficacy was remarkably increased by ~2000-fold, resulting in 50% transcript reduction already being reached at around 1 nM ASO (FIG. 17A). In a second example, a non-conjugated ASO (without saponin component) lead up to 50% reduction of Malat1 transcript at around 100 nM ASO (FIG. 17B), and when the ASO was co-administered with Saponin (2), the efficacy was increased by ~1000-fold resulting in 50% transcript reduction at only 0.1 nM ASO (FIG. 17B). Both examples reveal a clear and strong potency increase for co-administration treatments of an ASO with a saponin component.

To evaluate if saponin components enhance the potency of a targeted ASO in a neuronal system, the Malat1 ASO was conjugated to a CD71-targeting mAb, resulting in aCD71-Malat1 ASO. Neuro-2a cells were treated with this targeted ASO in the presence and absence of a saponin component (being 4 μM Saponin (2)). Interestingly, treatment with targeted ASO at only ~10 nM induced a 50% reduction in Malat1 transcript (FIG. 17C), while the targeted ASO (aCD71-Malat1 ASO)+Saponin (2) resulted in even higher potency with only 0.01 nM ASO (absolute concentration in conjugate) being sufficient to result in a 50% reduction in Malat1 transcript. These results suggest that ligand conjugation (i.e, endosomal targeting) of the ASO increases its potency, but only when the ASO (targeted or not) is combined with a saponin component, the efficacy enhancement is at least 1000-fold or more.

### Example 12: Efficacy Enhancement of PMO Conjugates by Saponin Components in Neuronal Cells

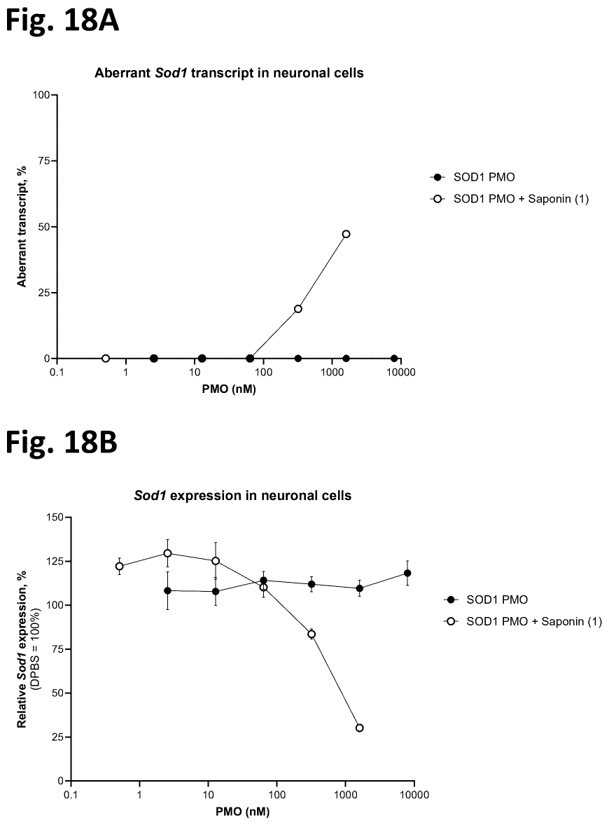

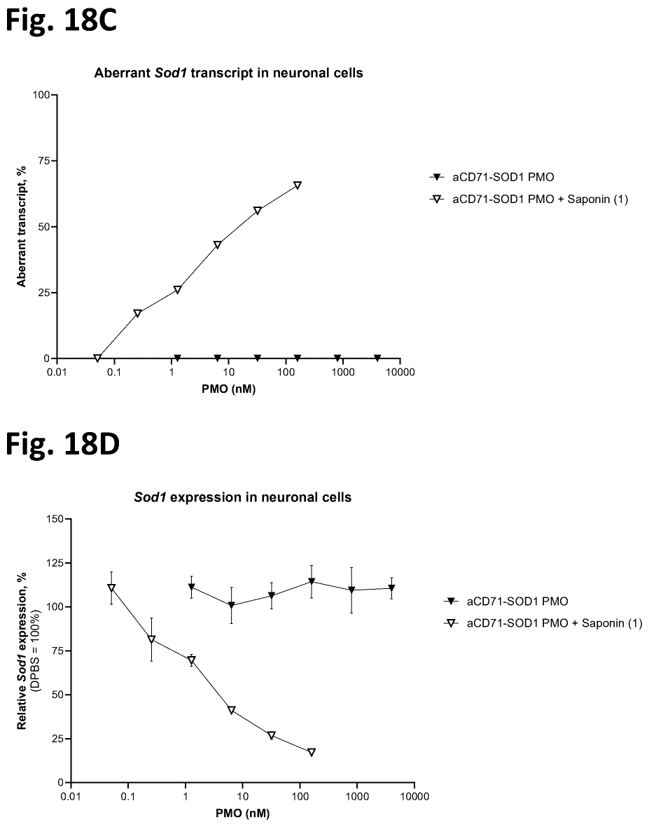

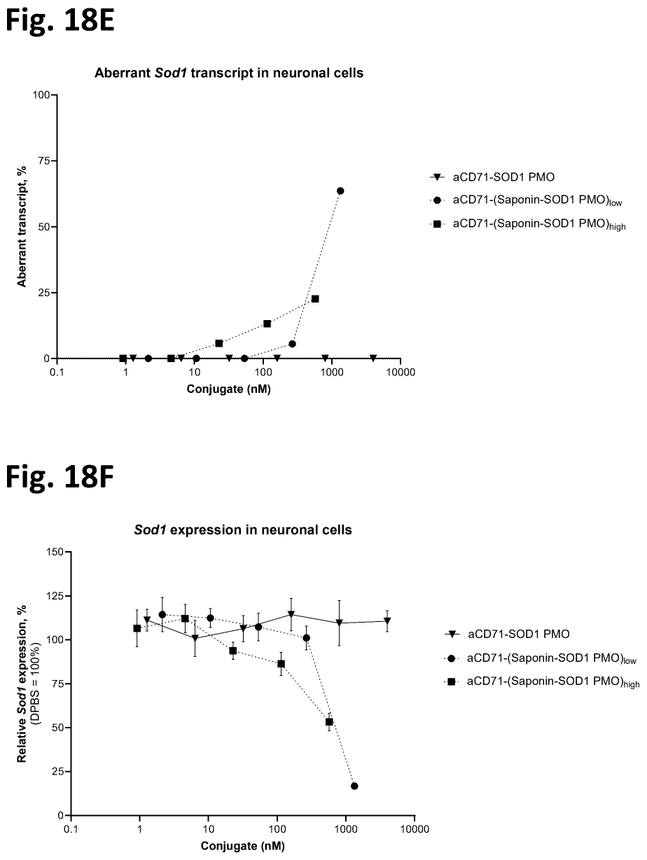

In example 4 we show that treatment of murine neuronal cells with Sod1-targeting PMOs in combination with a saponin component clearly enhances the efficacy of the PMO compared to treatment with PMO alone (cf. EXAMPLE 4). To confirm and expand these findings, aberrant transcript and remaining full length transcript were determined to reveal full potency of treatment, i.e. the effect of the PMO on inducing exon skip or aberrant transcript as well as nonsense mediated mRNA decay was determined. To this end, neuronal cells were treated with the Sod1 PMO in combination with a saponin component. This treatment induced an increased aberrant transcript up to 47% (FIG. 18). When determining the amount full length Sod1 transcript remaining after treatment, it was confirmed that without saponin component, the PMO is not able to reduce transcript. However, in the presence of the saponin component, a 70% reduction in Sod1 transcript was observed at 1600 nM PMO (FIG. 18B). These results indicate that the PMO is highly active and induces nonsense mediated mRNA, but only in presence of saponin this high on-target activity and potency is revealed.

To assess the effect of ligand-mediated uptake (to increase endosomal PMO content), the PMO was conjugated to a CD71 targeting mAb, resulting in aCD71-SOD1 PMO. Neuronal cells were treated with aCD71-SOD1 PMO (Compound 2) in the presence and absence of a saponin component to assess how saponin components enhance the activity of targeted aCD71-SOD1 PMO compared to non-targeted PMO. After treatment, both, non-targeted PMO and targeted aCD71-SOD1 PMO did not show any activity at any of the concentrations tested (i.e. no aberrant transcript induction (FIG. 18C) nor nonsense mediated mRNA decay (FIG. 18D)). However, when the targeted aCD71-SOD1 PMO was co-administered with the saponin component, a surprising effect was observed: in presence of saponin, already at 0.18 nM aCD71-SOD1 (corresponding to 0.26 nM PMO), aberrant transcripts were detected, which increased up to 66% aberrant transcript at 114 nM aCD71-SOD1 (corresponding to 160 nM PMO) (FIG. 18C). When determining the amount full length Sod1 transcript remaining after treatment of cells with the conjugate and co-administration of the saponin compound, as little as 0.18 nM aCD71-SOD1 (corresponding to 0.26 nM PMO) was sufficient to reduce the Sod1 expression, and at the maximum concentration tested (114 nM aCD71-SOD1, corresponding to 160 nM PMO), more than 80% reduction in Sod1 transcript was measured (FIG. 18D).

As a next step, two different 1-component conjugates were produced in which the saponin component was conjugated to the targeted aCD71-SOD1 PMO at either a high or a low conjugation ratio, resulting in aCD71-(Saponin-SOD1 PMO) high and aCD71-(Saponin-SOD1 PMO) low, respectively. These conjugates allow for a targeted (thus endosomally enriched) and synchronized delivery of saponin component and payload into the same cellular compartment. Neuronal cells were treated with these 1-component conjugates as well as with a aCD71-SOD1 PMO (without saponin compound) and aberrant transcripts were quantified (FIG. 18E). The data shows that the aCD71-(Saponin-SOD1 PMO) low and aCD71-(Saponin-SOD1 PMO)_{high} induce aberrant transcript (already starting at exposure concentrations of 267 nM and 23 nM conjugate, respectively). At the highest concentrations tested, aCD71-(Saponin-SOD1 PMO)_{low} achieves 64% aberrant transcript and aCD71-(Saponin-SOD1 PMO)_{high} achieves 23% aberrant transcript, while aCD71-SOD1 PMO (without saponin) has no effect on Sod1 transcripts at any of the concentrations tested (FIG. 18E). When the potency on reducing Sod1 transcript of such conjugates was determined, (ie, measuring the remaining full length Sod1 transcript), both, aCD71-(Saponin-SOD1 PMO)_{low} and aCD71-(Saponin-SOD1 PMO)_{high} were highly efficacious in reducing full length Sod1 RNA, while aCD71-SOD1 PMO (without saponin) again had no effect. A reduction could be measured starting from 23 nM aCD71-(Saponin-SOD1 PMO)_{high} conjugate, which increased up to a 47% reduction in Sod1 transcript at 571 nM conjugate (FIG. 18F). aCD71-(Saponin-SOD1 PMO)_{low} showed up to 83% reduction in Sod1 transcript at 1333 nM conjugate. These data show that conjugation of a saponin component allows to obtain an effect of the PMO on aberrant transcript induction and reduction of full length Sod1 transcript.

### Example 13: Enhancement of Oligonucleotide Efficacy by Saponin Components in Retinal Cells

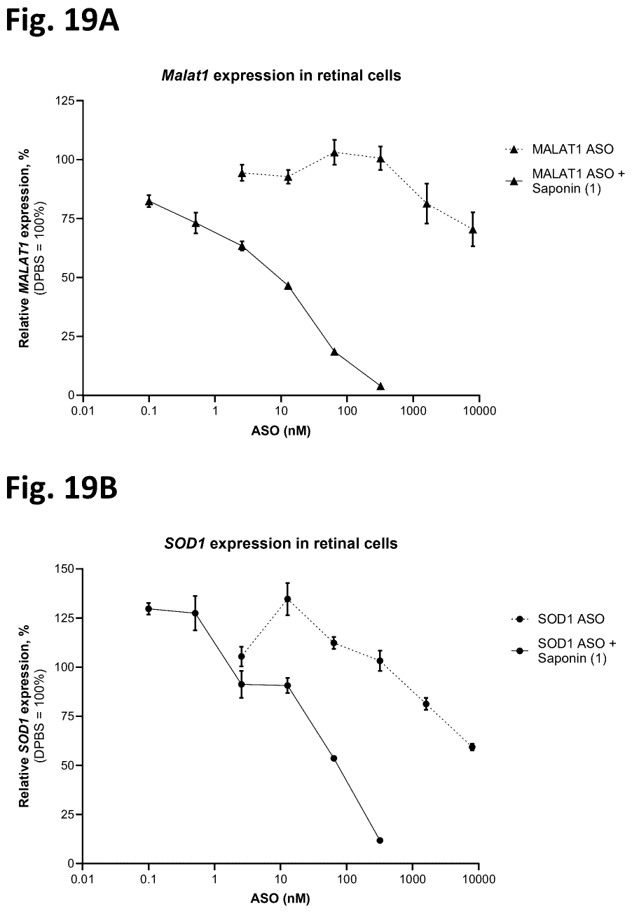

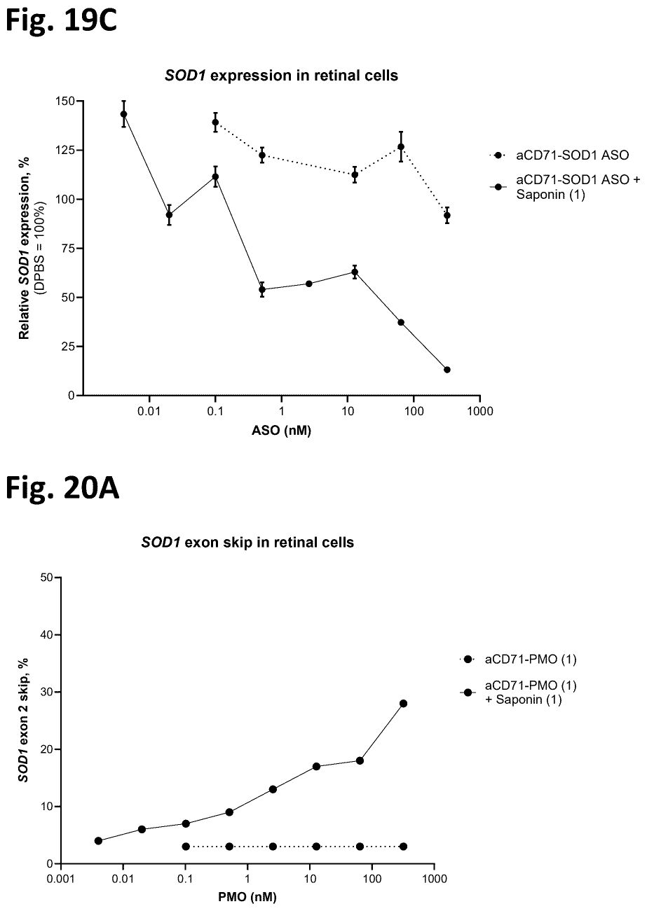

The ARPE-19 cell line is a spontaneously arising retinal pigment epithelia (RPE) cell line, which has functional characteristics akin to native RPE cells, making it a pivotal epithelial cell model in ophthalmic research. Here, the knockdown efficacy enhancement by saponin components of different oligonucleotides targeting CNS/ocular disease relevant RNA targets, such as MALAT1 and SOD1, was assessed on a monolayer of ARPE-19 cells. Cell viability was not affected by any of the applied treatments (data not shown). Cells treated with MALAT1 ASO alone showed little to no reduction in MALAT1 expression at relevant concentrations and only 30% reduction at 8 μM ASO, while co-dosing with a saponin component clearly enhanced the ASO potency (FIG. 19A): when co-dosing, already at 2.5 nM ASO more than 30% reduction of MALAT1 RNA was observed, which increased to almost 100% knockdown at 320 nM ASO in the presence of saponin component. To further substantiate the findings and relevance, an ASO targeting human SOD1 (SOD1 ASO, with the same sequence and modifications as the FDA approved ASO tofersen, sold under the brand name Qalsody) was tested. Tofersen is a mixed backbone structure consisting of 5-10-5 MOE gapmer. It is composed of 19 inter-nucleotide linkages, with 15 of them being 3′-O to 5′-O phosphorothioate diesters, and the remaining four being 3′-O to 5′-O phosphate diesters. Treatment with this ASO alone resulted in only 41% reduction in SOD1 expression at 8 μM ASO (FIG. 19B). When this ASO was co-dosed with saponin component, a clear efficacy enhancement of at least 100× was observed, with 50% reduction at about 64 nM ASO and again almost full knockdown at 320 nM ASO in the presence of saponin component.

The SOD1 ASO was then conjugated to a CD71 targeting mAb, resulting in aCD71-SOD1 ASO. ARPE-19 cells were treated with this targeted ASO, with and without co-dosing of saponin components (FIG. 19C). Cell viability was not affected by any of the applied treatments (data not shown). However, aCD71-SOD1 ASO treatment did also not result in downmodulation of SOD1 at any of the concentrations tested. Importantly, when the targeted ASO (i.e., aCD71-SOD1 ASO) was co-dosed with a saponin component, the potency increased significantly (IC50 of 1-10 nM ASO), realizing an even greater enhancement factor than for the (non-conjugated) ASO+Saponin treatment. Taken together, this data shows that a saponin component can not only enhance the potency of a non-conjugated ASO in retinal cells, but also of a targeted ASO.

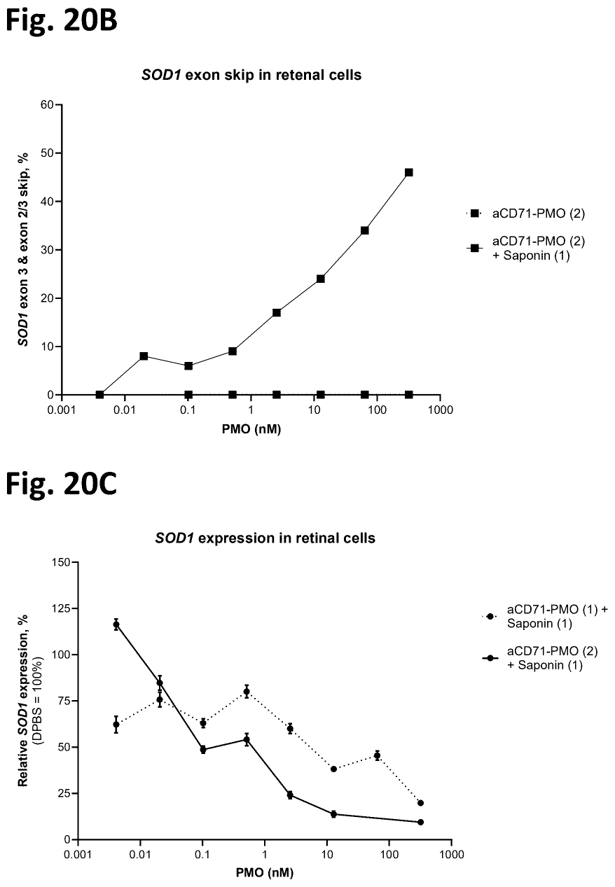

To evaluate if saponin components enhance the potency of neutral-charge oligonucleotide payloads to the same target (SOD1) in retinal cells, PMO (1) and PMO (2) were selected. These PMOs were designed to induce exon skip of exon 2 and (exon 3+exon 2/3), respectively, which causes a pre-mature stop codon in exon 4. Two different targeted-PMO conjugates were generated, comprised of a CD71 targeting mAb and PMO (1) or PMO (2), resulting in aCD71-PMO (1) and aCD71-PMO (2), respectively. The conjugates were evaluated on ARPE-19 retinal cells in the presence and absence of a saponin component. Cell viability was not affected by any of the applied treatments (data not shown). Even without treatment, ARPE-19 retinal cells were shown to exhibit low level basal exon skip activity (~3% SOD1 exon 2 skip). Interestingly, treatment with aCD71-PMO (1) did not enhance this exon skip at any of the tested concentrations (FIG. 20A). However, co-dosing treatment of aCD71-PMO (1)+Saponin component resulted not only in an increase in exon skip at very low concentrations, i.e. already as low as 0.02 nM PMO (equal to 0.01 nM conjugate), but also enhanced the exon skip up to 28% at 320 nM PMO (or 160 nM conjugate). Likewise, treatment with aCD71-PMO (2) alone did not result in exon skip of exon 3 or exon 2/3 at any of the tested concentrations without saponin component (FIG. 20B). Again, in the presence of saponin component, also aCD71-PMO (2) showed clear exon skip at very low concentrations above only 0.02 nM PMO (equal to 0.01 nM conjugate), which was enhanced up to 46% exon skip at 320 nM PMO (or 160 nM conjugate). In addition to exon skip, the amount of residual full length SOD1 gene product was determined by quantitative PCR. These analyses revealed the full potency of the applied treatments, as it measures the combined effect of treatment (i.e., exon skip and non-sense mediated mRNA decay). This analysis confirmed that in the presence of saponin component, the targeted PMO conjugates are active (FIG. 20C), and that in presence of saponin compounds, minimal amounts of the PMO (as part of the aCD71-PMO conjugate) are sufficient to induce a clear reduction in the amount of full length SOD1 transcript. Notably, the maximal combined effect on exon skip and non-sense mediated mRNA decay by aCD71-PMO (1) and aCD71-PMO (2) in the presence of saponin components resulted in a reduction of 80% and 91% full length SOD1 transcript, respectively, at the highest concentration of targeted PMO tested.

### Materials and Methods

### Abbreviations

Ab Antibody
AH Acylhydrazone bond
AEM N-(2-Aminoethyl) maleimide trifluoroacetate salt
AMPD 2-Amino-2-methyl-1,3-propanediol
BOP (Benzotriazol-1-yloxy) tris(dimethylamino)phosphonium hexafluorophosphate
Cet Cetuximab
d2 bivalent dendron (generation 2)
DAR Drug to antibody ratio
DBCO Dibenzocyclooctyne
DCM Dichloromethane
DIPEA N,N-diisopropylethylamine
DMF N,N-dimethylformamide
DMSO Dimethylsulfoxide
DTT Dithiothreitol
EDCI·HCl 3-((Ethylimino) methyleneamino)-N,N-dimethylpropan-1-aminium chloride
EDTA Ethylenediaminetetraacetic acid
EMCH·TFA N-(ε-maleimidocaproic acid) hydrazide, trifluoroacetic acid salt
GalT β-1,4-Galactosyltransferase Y289L
HATU 1-[Bis(dimethylamino)methylene]-1H-1,2,3-triazolo[4,5-b]pyridinium 3-oxid hexafluorophosphate
IPA Isopropyl alcohol
Mal Maleimide
min Minutes
mTz Methyltetrazine
MWCO Molecular weight cut-off
NEM N-Ethylmaleimide
NHS N-Hydroxysuccinimide
NMM 4-Methylmorpholine
PEG Poly(ethylene glycol)
PEG4-SPDP (2-pyridyldithio)-PEG4-NHS ester
PDT Pyridine-3-thiol
RPM revolutions per minute
r.t. Retention time
SC Semicarbazone bond
SH Thiol
SMCC Succinimidyl-4-(N-maleimidomethyl)cyclohexan-1-carboxylat
TBS Tris buffer saline
TCO Trans-cyclooctene
TCEP Tris(2-carboxyethyl) phosphine hydrochloride
Temp Temperature
TFA Trifluoroacetic acid
TFL Trifunctional linker
THF Tetrahydrofuran
THPP Tris(3-hydroxypropyl) phosphine
UDP-GalNAz Uridindiphosphat-N-azidoacetylgalactosamine disodium

### Materials for Examples 1-8

| | | Cat. Code / Product | |
|---|---|---|---|
| Description | Supplier | No. | Lot No. |
| Cetuximab | Merck Europe | Erbitux | |
| Goat anti-Human Kappa - | Southern Biotech | 2060-05 | C4119-VG59B |
| HRP | | | |
| Goat anti-Human IgG - | Southern Biotech | 2040-05 | B3919-XD29C |
| HRP | | | |
| “presentation buffer” | Fleet BioScience | SOP032 Buffer 113 | BF3371 |
| (Dulbecco's PBS pH 7.5) | Ltd | | |
| Dulbecco's PBS pH 7.5, | Fleet BioScience | — | DV254/12-DPBST |
| 0.01% polysorbate 80 | Ltd | | |
| (DPBST pH 7.5) | | | |
| “SEC analysis buffer” | Fleet BioScience | SOP032 Buffer 114 | BF3385 |
| (DPBS:IPA 85:15) | Ltd | | |
| PBS, 0.05% Tween 20 | Fleet BioScience | — | DV238-173 |
| | Ltd | | |
| MOPS running buffer | Life Technologies | NP0001 | 2160459 |
| LDS sample buffer | Life Technologies | NP0007 | 2152677A |
| NuPAGE Transfer Buffer | Thermo | NP0006 | 2148502 |
| (20X) | | | |
| Blocking Buffer | Thermo | 37537 | UK292772 |
| Methanol | VWR | 20847 | 18G164024 |
| DTT | Sigma | 43815 | BCBS8122V |
| Polysorbate (Tween) 80 | Sigma | P1754 | BCBR2510V |
| Glycine | VWR | 104201 | V01604600825 |
| Zeba 10 ml spin desalting | Thermo | 89893 | UF283301 |
| column | | | |
| PD10 G25 | GE | 17085101 | 17046497 |
| Float-a-lyser G2 | Sigma | G235065 | 3317420 |
| Vivaspin T4 | Sartorius | VS04T01 | 193600057 |
| Vivaspin T15 | Sartorius | VS15T02 | 1803004VS |
| 0.2 μm Filter | Sartorius | 17761 | 91500103 |
| BCA Assay kit | Thermo | 23225 | UF281362A |
| BGG standard | Thermo | 23212 | TK273657 |
| Novex protein standards | Life Technologies | LC5800 | 2154538 |
| ladder | | | |
| 4-12% BT SDS-PAGE gel | Life Technologies | NP0323BOX | 19120670 |
| PAGE Blue protein stain | Thermo | 24620 | ON643585 |
| NuPAGE Antioxidant | Thermo | NP0005 | 2171517 |
| Nitrocellulose membrane | Thermo | LC20000 | 1708367 |
| Western Blotting filter | Thermo | 88600 | QA1889445 |
| paper | | | |
| CN/DAB Substrate (10X) | Thermo | 1855900 | UG287470 |
| Stable Peroxide Substrate | Thermo | 1855901 | UF285529 |
| Buffer | | | |
| TLC silica gel 60 | Merck | 1055540001 | HX957486 |
| Biosep s3000 aSEC | Phenomenex | SEC-s3000 | CLM0044 |
| column | | | |
| Digoxin | Merck | PHR1771 | |
| Tomatin | Carl Roth | 5676.1 | |
| Glycyrrhizin | Merck | PHL89217 | |
| Digitonin | Merck | 300410 | |

SO1861 was isolated and purified by either Analyticon Discovery GmbH, Germany or Extrasynthese, France, from raw plant extract obtained from Saponaria officinalis L.

### Analytical Methods

### LC-MS Method 1

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: neg or neg/pos within in a range of 1500-2400 or 2000-3000; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product:

$${}_{}^{A} {t}_{0}^{} = 2 \% A , {t}_{5. min} = 50 \% A , {t}_{6. min} = 98 \% A$$

$${}_{}^{B} {t}_{0}^{} = 2 \% A , {t}_{5. min} = 98 \% A , {t}_{6. min} = 98 \% A$$

Posttime: 1.0 min, Eluent A: acetonitrile, Eluent B: 10 mM ammonium bicarbonate in water (pH=9.5).

### LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C.; column: Waters XSelect™ CSH C18, 50×2.1 mm, 2.5 μm, Temp: 25° C., Flow: 0.5 mL/min, Gradient: t₀ₘᵢₙ=5% A, t_{2.0min}=98% A, t_{2.7min}=98% A, Posttime: 0.3 min, Eluent A: acetonitrile, Eluent B: 10 mM ammonium bicarbonate in water (pH=9.5).

### LC-MS Method 3

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product pos/neg 105-800, 500-1200 or 1500-2500; ELSD: gas pressure 40 psi, drift tube temp: 50° C.; column: Waters XSelect™ CSH C18, 50×2.1 mm, 2.5 μm, Temp: 40° C., Flow: 0.5 mL/min, Gradient: t₀ₘᵢₙ=5% A, t_{2.0 min}=98% A, t_{2.7 min}=98% A, Posttime: 0.3 min, Eluent A: 0.1% formic acid in acetonitrile, Eluent B: 0.1% formic acid in water.

### LC-MS Method 4

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C. column: Waters Acquity Shield RP18, 50×2.1 mm, 1.7 μm, Temp: 25° C., Flow: 0.5 mL/min, Gradient: t₀ₘᵢₙ=5% A, t_{2.0 min}=98% A, t_{2.7 min}=98% A, Posttime: 0.3 min, Eluent A: acetonitrile, Eluent B: 10 mM ammonium bicarbonate in water (pH=9.5).

### LC-MS Method 5

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: neg/pos within in a range of 1500-2700; ELSD: gas pressure 40 psi, drift tube temp: 50° C.; column: Acquity Premier Peptide BEH C18, 50×2.1 mm, 1.7 μm Temp: 25° C., Flow: 0.45 mL/min, Gradient depending on the polarity of the product:

$${}_{}^{A} {t}_{0}^{} = 2 \% B , {t}_{4. min} = 50 \% B , {t}_{5. min} = 98 \% B , {t}_{6. min} = 98 \% B$$

$${}_{}^{B} {t}_{0}^{} = 5 \% B , {t}_{6. min} = 98 \% B , {t}_{6. min} = 98 \% B ,$$

Posttime: 1.0 min, Eluent A: 10 mM ammonium bicarbonate in water (pH=9.5), Eluent B: acetonitrile.

### LC-MS Method 6

Instrument: Agilent 1260 Infinity II, 1260 G7112B Bin. Pump, 1260 G7167A Multisampler, 1260 MCT G7116A Column Comp. 1260 G7115A DAD (210, 220 and 210-320 nm), PDA (210-320 nm), G6130B MSD (ESI pos/neg) mass range 90-1500, Column: XSelect CSH C18 (30×2.1 mm 3.5 μm), Flow: 1 mL/min, Column temp: 25° C., Eluent A: 10 mM ammoniumbicarbonate in water (pH 9.5), Eluent B: acetonitrile, Gradient: 10 min=5% B, t1.6 min=98% B, t3 min=98% B, Postrun: 1.2 min.

### LC-MS Method 7

Apparatus: Waters I-Class UPLC, Binary Solvent Manager (BSM), Sample Manager-FTN (SM-FTN) and Sample Organizer (SO), Column Manager (CM-A), PDA 210-320 nm, SQD2 ESI, mass ranges depending on the molecular weight of the product: pos/neg within in a range of 400-1600 or 1500-2500; ELSD: gaspressure 40 psi, drift tube temp: 50° C.; column: Acquity Premier Peptide BEH C18, 50×2.1 mm, 1.7 μm Temp: 25° C., Flow: 0.45 mL/min, Gradient: to =2% B, t_{4.0 min}=50% B, t_{6.0 min}=98% B, Posttime: 1.0 min, Eluent A: 10 mM ammonium bicarbonate in water (pH=9.5), Eluent B: acetonitrile.

### Preparative Methods

### Preparative MP-LC Method 1

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient:

^{A}t_{0 min} = 5% B, t_{1 min} = 5% B, t_{2 min} = 10% B, t_{17 min} = 50% B, t_{18 min} = 100% B, t_{23 min} = 100% B
^{A}t_{0 min} = 5% B, t_{1 min} = 5% B, t_{2 min} = 20% B, t_{17 min} = 60% B, t_{18 min} = 100% B, t_{23 min} = 100% B;

Detection UV: 210, 235, 254 nm and ELSD.

### Preparative MP-LC Method 2

Instrument type: Reveleris™ prep MPLC; Column: Phenomenex LUNA C18 (3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient:

^{A}t_{0 min} = 5% B, t_{1 min} = 5% B, t_{2 min} = 20% B, t_{17 min} = 60% B, t_{18 min} = 100% B, t_{23 min} = 100% B
^{B}t_{0 min} = 2% B, t_{1 min} = 2% B, t_{2 min} = 2% B, t_{17 min} = 30% B, t_{18 min} = 100% B, t_{23 min} = 100% B
^{C}t_{0 min} = 5% B, t_{1 min} = 5% B, t_{2 min} = 10% B, t_{17 min} = 50% B, t_{18 min} = 100% B, t_{23 min} = 100% B
^{D}t_{0 min} = 5% B, t_{1 min} = 5% B, t_{2 min} = 5% B, t_{17 min} = 40% B, t_{18 min} = 100% B, t_{23 min} = 100% B;

Detection UV: 210, 235, 254 nm and ELSD.

### Preparative LC-MS Method 3

MS instrument type: Agilent Technologies G6130B Quadrupole; HPLC instrument type: Agilent Technologies 1290 preparative LC; Column: Waters XSelect™ CSH (C18, 150×19 mm, 10 μm); Flow: 25 ml/min; Column temp: room temperature; Eluent A: 100% acetonitrile; Eluent B: 10 mM ammonium bicarbonate in water pH=9.0; Gradient:

^{A}t₀ = 20% A, t_{2.5 min} = 20% A, t_{11 min} = 60% A, t_{13 min} = 100% A, t_{17 min} = 100% A
^{B}t₀ = 5% A, t_{2.5 min} = 5% A, t_{11 min} = 40% A, t_{13 min} = 100% A, t_{17 min} = 100% A;

Detection: DAD (210 nm); Detection: MSD (ESI pos/neg) mass range: 100-800; Fraction collection based on DAD.

### Preparative LC-MS Method 4

MS instrument type: Agilent Technologies G6130B Quadrupole; HPLC instrument type: Agilent Technologies 1290 preparative LC; Column: Waters XBridge Protein (C4, 150×19 mm, 10 μm); Flow: 25 ml/min; Column temp: room temperature; Eluent A: 100% acetonitrile; Eluent B: 10 mM ammonium bicarbonate in water pH=9.0; Gradient:

^{A}t₀ = 2% A, t_{2.5 min} = 2% A, t_{11 min} = 30% A, t_{13 min} = 100% A, t_{17 min} = 100% A
^{B}t₀ = 10% A, t_{2.5 min} = 10% A, t_{11 min} = 50% A, t_{13 min} = 100% A, t_{17 min} = 100% A
^{C}t₀ = 5% A, t_{2.5 min} = 5% A, t_{11 min} = 40% A, t_{13 min} = 100% A, t_{17 min} = 100% A;

Detection: DAD (210 nm); Detection: MSD (ESI pos/neg) mass range: 100-800; Fraction collection based on DAD

### Flash Chromatography

Grace Reveleris X2® C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50bar (725 psi); Detection: UV 200-400 nm, combination of up to 4 UV signals and scan of entire UV range, ELSD; Column sizes: 4-330 g on instrument, luer type, 750 g up to 3000 g with optional holder.

### UV-Vis Spectrophotometry

Antibody concentrations, and Sulfo-Cy5 concentrations and incorporations were determined using a Thermo Nanodrop 2000 spectrometer. Antibody concentrations in the conjugates were determined by BCA assay. BCA assays were conducted using a Thermo SkanIT plate reader.

$$Ellmans ( TNB ) ε 412 = 14 , 150 M - 1 cm - 1$$

$$Cetuximab ε 280 = 1.4 ( mg / ml ) - 1 cm - 1$$

Cetuximab-SO1861; mass £280=1.4 (mg/ml)-1 cm-1

STAT3-ST2; molar EC260=201,445 M-1 cm-1; Rz 260:280=1.816

STAT3_ST6; molar EC260=183,491 M-1 cm-1; Rz 260:280=1.653

PDT; molar EC343=8,080 M-1 cm-1.

### SEC

Native antibody and conjugates were analysed by SEC using an Akta purifier 100 system and Biosep SEC-s3000 column eluting with DPBS: IPA (85:15). % purity was determined by integration of the antibody peak with respect to trace aggregate peaks.

### SDS-PAGE and Western Blotting

Native antibody and conjugates were analysed under heat denaturing non-reducing and reducing conditions by SDS-PAGE against a protein ladder using a 4-12% bis-tris gel and MOPS as running buffer (200V, 40 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using ImageJ.

For Western Blotting, washed gel (not Coomassie stained) was transferred to nitrocellulose membrane using the X-Cell blot module with the following setup (BP-BP-FP-Gel-NC-FP-BP-FP-Gel-NC-FP-BP-BP) and conditions (30V, 0.17 Amps, 60 minutes) and freshly prepared transfer buffer. BP—blotting pad; FP—Filter pad; NC—Nitrocellulose membrane. After, the NC were washed thrice with PBS-T (100 ml), non-specific sites blocked with blocking buffer (30 ml) with shaking (10 minutes, 200 rpm) then active sites labelled with a combination of Goat anti-Human Kappa-HRP (1:2000) and Goat anti-Human IgG-HRP (1:2000) (30 ml) diluted in blocking buffer with shaking (60 minutes, 200 rpm). After, the NC were washed with PBS-T (100 ml) and complexed antibody detected with CN/DAB substrate (25 ml) freshly prepared using stable peroxide substrate buffer. Colour development was observed visually and the resulting NC photographed.

### SO1861-AH-Maleimide

SO1861-AH-Maleimide (also referred to as SO1861-AH-Mal or SO1861-EMCH) was produced as previously described in WO 2021/259507A1 (page 72, Example 3, referred to as “SO1861-EMCH synthesis”). To SO1861 (121 mg, 0.065 mmol) and EMCH·TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.1 Fractions corresponding to the product were immediately pooled together, frozen and lyophilized overnight to give the title compound (120 mg, 90%) as a white fluffy solid. Purity based on LC-MS 96%.
LRMS (m/z): 2069 [M−1]1−
LC-MS r.t. (min): 1.084

### SO1861-AH-Maleimide-Block (Saponin Molecule According to Formula (V), Also Referred to as SO1861-AH-Block)

To SO1861-AH-Maleimide (0.1 mg, 48 nmol) 200 μL mercaptoethanol (18 mg, 230 μmol) was added and the solution was shaken for 1 h at 800 rpm and room temperature on a ThermoMixer C (Eppendorf). After shaking for 1 h, the solution was diluted with methanol and dialyzed extensively for 4 h against methanol using regenerated cellulose membrane tubes (Spectra/Por 7) with a MWCO of 1 kDa. After dialysis the SO1861-Ald-EMCH-mercaptoethanol was provided (saponin molecule according to formula (V)), an aliquot was taken out and analyzed via MALDI-TOF-MS.

(RP mode): m/z 2193 Da ([M+K]+, SO1861-AH-Block), m/z 2185 Da ([M+K]+, SO1861-AH-Block), m/z 2170 Da ([M+Na]+, SO1861-AH-Block).

### SO1861-SC-Maleimide Synthesis

SO1861-SC-Maleimide (also referred to as SO1861-SC-Mal) was produced as previously described in WO 2023/038517A1 (page 168, line 1 to line 13, Example 1, referred to as “SO1861-SC-Mal”).

Tert-butyl 2-(4-(6-(2,5-dioxo-2,5-dihydro-1H-pyrrol-1-yl) hexanoyl) piperazine-1-carbonyl) hydrazine-1-carboxylate (25.0 mg, 57.1 μmol) was dissolved in a mixture of dichloromethane (500 μL) and TFA (500 μL) and the reaction mixture was stirred at room temperature. After 30 min the reaction 35 mixture was evaporated in vacuo and co-evaporated with dichloromethane (3×5 mL) and methanol (5 mL). The residue and SO1861 (21.3 mg, 11.4 μmol) were dissolved in methanol (extra dry, 1.00 mL) and the resulting mixture was shaken for 1 min and left standing at room temperature. After 4 hours the reaction mixture was subjected to to preparative MP-LC.2 Fractions corresponding to the product were immediately pooled together, frozen and lyophilized overnight to yield the title compound (13.7 mg, 55%) as a white fluffy solid. Purity based on LC-MS 97%.

LRMS (m/z): 2181 [M−1]1−

LC-MS r.t. (min): 2.133

### Cetuximab-SC-SO1861

An aliquot of cetuximab (305 mg, 5.0 mg/ml, 61 ml) was modified with Tris/Tris·HCl/EDTA concentrate (30 μl/ml, 1830 μl). To cetuximab (305 mg, 2.03×10-3 mmol, 4.871 mg/ml) was added an aliquot of TCEP (2.77 equivalents, 5.63×10-3 mmol, 1.61 mg, 1.61 ml) freshly prepared in TBS pH 7.5 (1 mg/ml) with gentle swirling. The mixture was incubated at 20° C. for 210 minutes with roller mixing. After incubation, an aliquot (0.211 ml) of the reaction mixture was removed and purified by Zeba 7K spin desalting column eluting with TBS pH 7.5. Ab-SH was analyzed by UV-vis spectrophotometry and Ellman's assay (3.321 mg/ml, Thiol to cetuximab ratio=4.2). To the bulk reaction was added an aliquot of SO1861-SC-Mal (8 mole equivalents, 16.2×10-3 mmol, 35.4 mg, 17.70 ml) freshly prepared with TBS pH 7.5 (2 mg/ml) with gentle swirling, the mixture vortexed briefly then incubated for 120 minutes at 20° C. Besides the conjugation reaction, two aliquots of desalted Ab-SH (0.25 mg, 0.075 ml, 1.67×10-6 mmol) were reacted with NEM (8.00 equivalents, 1.34×10-5 mmol, 6.7 μl of a 0.25 mg/ml solution) or TBS pH 7.5 buffer (6.7 μl) for 120 minutes at 20° C., as positive and negative controls, respectively. After incubation, a ca. 1.0 mg aliquot of Ab-SO1861 mixture (0.270 ml) was removed, purified by gel filtration using Zeba 7K spin desalting column into TBS pH 7.5 and characterized by Ellman's assay alongside positive and negative controls to obtain SO1861 incorporation. After reaction, to the bulk Ab-SO1861 mixture was added an aliquot of freshly prepared NEM solution (5 mole equivalents, 10.1×10-3 mmol, 507 μl of a 2.5 mg/ml solution) to quench the reaction. The quenched reaction mixture was stored at 2-8° C. overnight. The conjugate was purified by splitting the bulk into multiple aliquots and carrying out multiple runs (4 in total) using a sanitized 2.6×40 cm Superdex 200 column eluting with DPBS pH 7.5. The aliquots of purified Ab-SO1861 were combined, filtered to 0.2 μm under laminar flow and analyzed by UV-vis spectrophotometry. The aliquot was concentrated to >2.5 mg/ml using a vivacell 100 centrifugal filter, then normalized to 2.5 mg/ml and dispensed into aliquots for product testing, characterization and further conjugation work. The result was cetuximab-SC-SO1861 conjugate. Total yield=289 mg, 95%, Purity: 99% SO1861 to Ab ratio=4.1.

### Cetuximab-S-S-STAT3-ST2_PMO (Also Referred to as “Cet-STAT3_ST2 PMO”)

To an aliquot of cetuximab (127.5 mg, 8.50×10-4 mmol, 2.5 mg/ml) previously buffer exchanged into DPBS pH 7.5, was added an aliquot of freshly prepared PEG4-SPDP solution (10 mg/ml, 10.1 mole equivalents, 8.59×10-3 mmol, 0.480 ml), the mixture vortexed briefly then incubated for 60 minutes at 20° C. with roller-mixing. After incubation, the reaction was quenched by the addition of an aliquot of a freshly prepared glycine solution (50 mg/ml, 50 mole equivalents, 4.29×10-2 mmol, 64 μl), the mixture vortexed briefly then incubated for >15 minutes at 20° C. with roller-mixing. The conjugate was purified using a sanitized 5×50 cm Superdex 200PG column eluting with TBS pH 7.5 and analyzed by UV-vis to give purified Cet-SPDP (133.9 mg, 105%, 1.34 mg/ml, SPDP to Ab ratio=3.6). Ab-SPDP was used immediately.

Separately, the STAT3-ST2_PMO-S-S-amide (104.9 mg, 1.30×10-2 mmol, 10.00 mg/ml), was reconstituted using TBS pH 7.5 and pooled into a single aliquot. To this was added an aliquot of freshly prepared THPP solution (50 mg/ml, 10 mole equivalents, 13.0×10-2 mmol, 292 μl), the mixture vortexed briefly then incubated for 60 minutes at 37° C. with roller-mixing. After incubation, the PMO was purified across multiple PD10 Sephadex G25M columns eluting with TBS pH 7.5, to afford PMO-SH. Total yield=93.0 mg, 89%, Thiol to PMO ratio=0.88.

To an aliquot of Ab-SPDP (120.5 mg, 8.04×10-4 mmol, 1.34 mg/ml) was added an aliquot of PMO-SH (3.63 mg/ml, 7.0 mole equivalents, 5.63×10-3 mmol, 12.51 ml), the mixture vortexed briefly then incubated overnight at 20° C. with roller-mixing. After ca. 16 hours, the conjugate mixture was analyzed by UV-vis to ascertain incorporation by PDT displacement and then purified by 5×50 cm Superdex 200PG column eluting with DPBS pH 7.5 to give purified Cet-S-S-STAT3-ST2_PMO conjugate. The conjugate was analyzed by BCA colorimetric assay. The result was a -etuximab-S-S-STAT3-ST2_PMO conjugate. Total yield=69.6 mg, 55%, Purity: 99%, STAT3-ST2_PMO to Cet ratio=3.1.

### Cetuximab-(SC-SO1861)-(S-S-STAT3-ST2_PMO) and Cetuximab-(SC-SO1861)-(S-S-STAT3-ST6_PMO)

Cetuximab-(SC-SO1861)-(S-S-STAT3-ST2_PMO) is also referred to as “Cet-SO1861-STAT3_ST2 PMO”. Cetuximab-(SC-SO1861)-(S-S-STAT3-ST6_PMO) is also referred to as “Cet-SO1861-STAT3_ST6 PMO”. The following procedure is exemplary described for cetuximab-(SC-SO1861)-(S-S-STAT3-ST2_PMO). Cetuximab-(SC-SO1861)-(S-S-STAT3-ST6_PMO) was synthesis over the same procedure.

To an aliquot of cetuximab-SC-SO1861 (127.5 mg, 8.50×10-4 mmol, 2.5 mg/ml) previously buffer exchanged into DPBS pH 7.5, was added an aliquot of freshly prepared PEG4-SPDP solution (10 mg/ml, 10.1 mole equivalents, 8.59×10-3 mmol, 0.480 ml), the mixture vortexed briefly then incubated for 60 minutes at 20° C. with roller-mixing. After incubation, the reaction was quenched by the addition of an aliquot of a freshly prepared glycine solution (50 mg/ml, 50 mole equivalents, 4.29×10-2 mmol, 64 μl), the mixture vortexed briefly then incubated for >15 minutes at 20° C. with roller-mixing. The conjugate was purified using a sanitized 5×50 cm Superdex 200PG column eluting with TBS pH 7.5 and analyzed by UV-vis to give purified Cet-(SC-SO1861)-(SPDP) (133.9 mg, 105%, 1.34 mg/ml, SPDP to Ab ratio=3.6). Ab-SPDP was used immediately.

Separately, the STAT3-ST2_PMO-S-S-amide (104.9 mg, 1.30×10-2 mmol, 10.00 mg/ml), was reconstituted using TBS pH 7.5 and pooled into a single aliquot. To this was added an aliquot of freshly prepared THPP solution (50 mg/ml, 10 mole equivalents, 13.0×10-2 mmol, 292 μl), the mixture vortexed briefly then incubated for 60 minutes at 37° C. with roller-mixing. After incubation, the PMO was purified across multiple PD10 Sephadex G25M columns eluting with TBS pH 7.5, to afford PMO-SH.

STAT3-ST2_PMO-SH:

93.0 mg, 89%, Thiol to PMO ratio=0.88.

STAT3-ST6_PMO-SH:

47 mg, 56%, Thiol to PMO ratio=0.87

To an aliquot of cetuximab-(SC-SO1861)-SPDP (120.5 mg, 8.04×10-4 mmol, 1.34 mg/ml) was added an aliquot of PMO-SH (3.63 mg/ml, 7.0 mole equivalents, 5.63×10-3 mmol, 12.51 ml), the mixture vortexed briefly then incubated overnight at 20° C. with roller-mixing. After ca. 16 hours, the conjugate mixture was analyzed by UV-vis to ascertain incorporation by PDT displacement and then purified by 5×50 cm Superdex 200PG column eluting with DPBS pH 7.5 to give purified Cet-(SC-SO1861)-(S-S-STAT3-ST2_PMO) conjugate. The conjugate was analyzed by BCA colorimetric assay.

Cetuximab-(SC-SO1861)-(S-S-STAT3-ST2_PMO)

Total yield: 46 mg, 39%, Purity: 97%

SO1861 to Cet ratio=4.1

STAT3-ST2_PMO to Cet ratio=5.1

Cetuximab-(SC-SO1861)-(S-S-STAT3-ST6_PMO)

Total yield: 69 mg, 58%, Purity: 96%

SO1861 to Cet ratio=4.1

STAT3-ST6_PMO to Cet ratio=5.2

Cetuximab-AH-SO1861 (Also Referred to as “Cet-AH-SO1861”) To cetuximab (1087 mg, 4.800 mg/ml, 7.2×10-3 mmol, in TBS, 2.5 mM EDTA, pH 7.5) was added an aliquot of freshly prepared TCEP solution (1 mg/ml, 2.72 mole equivalents, 2.0×10-2 mmol, 5.65 mg), the mixture swirled by hand to mix then incubated for 210 minutes at 20° C. with roller-mixing. After incubation (prior to addition of SO1861-AH-Maleimide), a 2 mg (0.417 ml) aliquot of cetuximab-SH (Ab-SH) was removed and purified by gel filtration using zeba spin desalting column into TBS pH 7.5. This aliquot was characterized by UV-vis analysis and Ellman's assay (3.693 mg/ml, thiol to Ab ratio=4.0). To the bulk Ab-SH was added an aliquot of freshly prepared SO1861-AH-Maleimide solution (2 mg/ml, 5.2 mole equivalents, 3.8×10-2 mmol, 38.9 ml), the mixtures vortexed briefly then incubated for 120 minutes at 20° C. Besides the conjugation reaction, two aliquots of desalted Ab-SH (0.5 mg, 0.135 ml, 3.33×10-6 mmol) were reacted with NEM (8.00 equivalents, 2.66×10-5 mmol, 3.3 μg, 13.3 μl of a 0.25 mg/ml solution) or TBS pH 7.5 buffer (13.3 μl) for 120 minutes at 20° C., as positive and negative controls, respectively. After incubation (prior to addition of NEM), a ca. 2 mg (0.450 ml) aliquot of Ab-SO1861 mixture was removed and purified by gel filtration using zeba spin desalting column into TBS pH 7.5. This aliquot was characterized by UV-vis (3.271 mg/ml) and alongside positive and negative controls were characterized by Ellman's assay to obtain SO1861 incorporation. To the bulk Ab-SO1861 mixture was added an aliquot of freshly prepared NEM solution (2.5 mg/ml, 5 mole equivalents, 3.6×10-2 mmol, 4.54 mg) and the mixture stored at 2-8° C. overnight. The conjugate was purified by 10×40 cm Sephadex G50M column eluting with DPBS pH 7.5 to give purified cetuximab-SO1861 conjugate. The aliquot was filtered to 0.2 μm and dispensed. The result was a cetuximab-SO1861 conjugate. Yield=1056 mg, 97%, SO1861 to Ab ratio=3.9.

### Malat1 ASO

An antisense oligonucleotide targeting murine (Mm) Malat1 mRNA, Malat1 ASO [SEQ ID NO: 16], with the sequence and modifications (5′-C6-disulfide)-[4*33 24G*9*T*G*G*T*T*A*T*G*231*3*2]; with [1=2′-MOE-5Me-rU; 2=2′MOE-rA, 3=2′MOE-5Me-rC; 4=2′MOE-rG; 9=5-Methyl-dC; *=phosphorothioate] was custom-produced by BioSpring Gesellschaft für Biotechnologie mbH, Germany, according to methods known in the art. This ASO was further modified to yield Malat1-S-S-PEG3-OH, as described.

### Malat1-S-S-PEG3-OH

### Intermediate 1:

### 2-(2-(2-(pyridin-2-yldisulfaneyl) ethoxy) ethoxy) ethan-1-ol

Under a N₂ atmosphere, 2,2′-Dithiodipyridine (159 mg, 0.722 mmol) was dissolved in methanol (3.00 mL) and a solution of 2-(2-(2-mercaptoethoxy) ethoxy) ethan-1-ol (100 mg, 0.602 mmol) in methanol (500 μL) was added dropwise. The resulting mixture was stirred at room temperature. After 2 hours the reaction mixture was evaporated in vacuo and co-evaporated with DCM (2×5 mL). The residue was purified by flash chromatography (ethyl acetate-heptane gradient, 0:100 rising to 100:0) to give the title compound (100 mg, 60%) as a colorless oil. Purity based on LC-MS 98%.

LRMS (m/z): 276 [M+1]¹⁺

LC-MS r.t. (min): 1.59⁶

Malat1-S-S-PEG3-OH (also referred to as “Malat1-ASO”)

To Malat1 (10.00 mg, 1.34 μmol) was added a solution of 20 mM ammonium bicarbonate with 2.5 mM TCEP (2.67 mL, 13.4 μmol). The reaction mixture was shaken for 1 min and left standing at room temperature for overnight. The reaction mixture was diluted with water to 10 mL and the resulting mixture was filtered using a centrifugal filter with a molecular weight cut-off of 3000 Da (6000×g for 30 min). The residue solution was diluted with water to 10 mL and the resulting mixture was filtered using the same method described above. The residue solution was diluted with water (1.00 mL) and a solution of 2-(2-(2-(pyridin-2-yldisulfaneyl) ethoxy) ethoxy) ethan-1-ol (1.47 mg, 5.35 μmol) in acetonitrile (500 μL). The resulting solution was shaken for 1 min and left standing at room temperature. After 5 hours the reaction mixture was frozen and lyophilized overnight. The residue was subjected to preparative LC-MS.^{A} Fractions corresponding to the product were immediately pooled together, frozen and lyophilized overnight to give the title compound (7.43 mg, 74%) as a white fluffy solid. Purity based on LC-MS 91%.

LRMS (m/z): 750 [M-10]¹⁰⁻, 834 [M-9]⁹⁻, 938 [M-8]⁸⁻, 1072 [M-7]⁷⁻, 1251 [M-6]⁶⁻, 1501 [M-5]⁵⁻

LC-MS r.t. (min): 1.51⁷

Malat1-SC-SO1861 (also referred to as “Malat1-ASO-SC-SO1861”)

To Malat1 (5.00 mg, 0.688 μmol) was added a solution of 20 mM ammonium bicarbonate with 2.5 mM TCEP (500 μL, 2.50 μmol). The reaction mixture was shaken for 1 min and left standing at room temperature. After 6 hours the reaction mixture was frozen and lyophilized overnight. The residue was dissolved in a solution of 20 mM ammonium bicarbonate with 2.5 mM TCEP (500 μL, 2.50 μmol). The reaction mixture was shaken for 1 min and left standing at room temperature. After 1 hour the reaction mixture was poured in acetonitrile (10 mL). The resulting suspension was shaken and centrifuged (5000 RPM, 15 min). The solution was decanted and the residue was dissolved in a solution of 20 mM ammonium bicarbonate (500 μL). To this solution was added SO1861-SC-Mal in different aliquots until full conversion was observed with LC-MS.² In total 8.60 mg (3.93 μmol) of SO1861-SC-Mal was added. The reaction mixture was frozen and lyophilized overnight. The residue was subjected to preparative LC-MS.^{B} Fractions corresponding to the product were immediately pooled together, frozen and lyophilized overnight to give the title compound (3.50 mg, 55%) as a white fluffy solid. Purity based on LC-MS 96%.

LRMS (m/z): 1587 [M-6]⁶⁻, 1905 [M-5]⁵⁻, 2380 [M-4]⁴⁻

LC-MS r.t. (min): 2.48²

### SOD1 PMO and STAT3 PMOs

Phosphorodiamidate morpholino oligomers targeting murine (Mm) Sod1 (SOD1 PMO [SEQ ID: 20]: GCCAGCCTAGGACCTACCTTGTGTA and SOD1 PMO (2) [SEQ ID: 23]: AGCCTATTTACCAGAAACCAGCAGT), both to induce nonsense-mediated decay (mRNA reduction) via exon skip, were custom-produced by Gene Tools, LLC, according to methods known in the art. A phosphorodiamidate morpholino oligomer targeting both murine and human STAT3 mRNA by inducing exon skip resulting in nonsense-mediated decay (mRNA reduction) via exon skip (STAT3_ST6 PMO [SEQ ID: 36]: CATTTTCTGTTCTAGATCCTGTT) and a phosphorodiamidate morpholino oligomer targeting both murine and human STAT3a mRNA by inducing an isoform splice-switch from STAT3a mRNA to switch to STAT3B, thereby effectively reducing STAT3a mRNA levels (STAT3_ST2 PMO [SEQ ID: 37]: ATTGCTGCAGGTCGTTCTGTAGG) were custom-produced by Gene Tools, LLC, according to methods known in the art.

### STAT3 ASO

A STAT3 antisense oligonucleotide (STAT ASO) with the following sequence and following modification [SEQ ID NO: 8]: 3*1*2*T*T*T*G*G*A*T*G*T*0*2*4*3, with 0=5-Methyl-dC, 1=2′MOE-5Me-rU, 2=2′MOE-rA, 3=2′MOE-5Me-rC, 4=2′MOE-rG, *=phosphorothioate, was produced by BioSpring Gesellschaft für Biotechnologie GmbH, Germany, according to methods known in the art.

### HTRA LNA

An antisense oligonucleotide targeting murine (Mm) Htra mRNA, HTRA LNA [SEQ ID NO: 19] with the sequence and modifications 5′-[TL]*[AL]*[TL]*T*T*A*C*C*T*G*G*T*[TL]*[GL]*[TL]*[TL]; with [TL]=LNA-T; [AL]=LNA-A; [GL]=LNA-G; *=phosphorothioate, was custom-produced by Bio-Synthesis, Inc, according to methods known in the art.

AHA1 siRNAs

Several siRNAs targeting human AHA1 (siAHA1) were custom produced by Thermo Scientific, according to methods known in the art, with the same oligonucleotide sequence and different chemical modifications of backbones and sugars: (1) 2′O-Methyl: modified with 2′O-Methyl on both the sense and antisense strand (sense strand: 5′-GGAmUGAAGmUGGAGAmUmUAGmU-dT*dT-3′ [SEQ ID NO: 38] and antisense strand: 5′-ACmUAAUCUCmCACUUmCAUCCdT*dT-3′ [SEQ ID NO: 40]; with mU=2′-OMe-rU; mC=2′-OMe-rC; *=phosphorothioate) as described in Svenson et al. (2016), Tumor Selective Silencing Using an RNAi-Conjugated Polymeric Nanopharmaceutical, Molecular Pharmaceutics 2016; 13 (3): 737-747, doi: 10.1021/acs.molpharmaceut.5b00608 (Svenson et al., 2016). (2) siSTABLE: proprietary commercial stabilization chemistry siSTABLE (Thermo Scientific), or (3) Accell: proprietary commercial stabilization chemistry Accell (Thermo Scientific).

MMP14 siRNAs

An siRNAs targeting human MMP14 (siMMP14) was custom produced by Eurogentec, according to methods known in the art, modified with 2′-Fluoro on both the sense and antisense strand (sense strand: 5′-AA66AGAAG65GAAGG5AGAA9*9-3′ [SEQ ID NO: 39]; and antisense strand: 5′-5565A66556AG65565GG559*9-3′ [SEQ ID NO: 41]; with 5=2′-Fluoro-rU; 6=2′-Fluoro-rC; 9=dT; *=phosphorothioate).

### RNA Analysis Brain Tissues

Total RNA was isolated from the frozen sections of mouse brain regions using the TissueLyser II (Qiagen) as homogenizer and TRIzol™ Reagent (Thermo Scientific) according to the manufacturer's instruction. Conversion into cDNA was performed using iScript™ CDNA Synthesis Kit (BioRad) using standard protocols. Murine (Mm) Malat1 expression levels and levels of housekeeping genes were determined using quantitative real-time PCR assays (qRT-PCR) using iTaq™ Universal SYBR® Green Supermix (BioRad) and the Light Cycler 480 II (Roche Diagnostics) with specific DNA primers, listed in Table A3. Each analysis reaction was performed in triplicate. Analysis was done by the ΔCt method, to determine Malat1 expression relative to 2 specific housekeeping control mRNAs. Results are expressed as % relative Malat1 expression levels after normalization to the vehicle control.

TABLE A3
Primers used in qRT-PCR analysis

| | | |
|---|---|---|
| Mm Malat1 | Forward | AGGCGGGCAGCTAAGGA |
| | | [SEQ ID NO: 17] |
| | Reverse | CCCCACTGTAGCATCACATCA |
| | | [SEQ ID NO: 18] |
| Mm Ppia | Forward | GCGGCAGGTCCATCTACG |
| | | [SEQ ID NO: 4] |
| | Reverse | GCCATCCAGCCATTCAGTC |
| | | [SEQ ID NO: 5] |
| Mm Sdha | Forward | GAGGAAGCACACCCTCTCAT |
| | | [SEQ ID NO: 6] |
| | Reverse | GGAGCGGATAGCAGGAGGTA |
| | | [SEQ ID NO: 7] |
| Mm STAT3 | Forward | GGGCATTTTTATGGCTTTCAAT |
| | | [SEQ ID NO: 24] |
| | Reverse | GTTAACCCAGGCACACAGACTTC |
| | | [SEQ ID NO: 25] |
| Hs STAT3 | Forward | ACATGCCACTTTGGTGTTTCATAA |
| | | [SEQ ID NO: 9] |
| | Reverse | TCTTCGTAGATTGTGCTGATAGAGAAC |
| | | [SEQ ID NO: 10] |
| Hs HBMS | Forward | CACCCACACACAGCCTACTT |
| | | [SEQ ID NO: 11] |
| | Reverse | GTACCCACGCGAATCACTCT |
| | | [SEQ ID NO: 12] |
| Hs SDHA | Forward | GCATTTGGCCTTTCTGAGGC |
| | | [SEQ ID NO: 13] |
| | Reverse | CTCCATGTTCCCCAGAGCAG |
| | | [SEQ ID NO: 14] |
| Hs BGUS | Forward | GAAAATACGTGGTTGGAGAGCT |
| | | [SEQ ID NO: 28] |
| | Reverse | CCGAGTGAAGATCCCCTTTTTA |
| | | [SEQ ID NO: 29] |
| Hs HPRT | Forward | CCTGGCGTCGTGATTAGTGAT |
| | | [SEQ ID NO: 30] |
| | Reverse | AGACGTTCAGTCCTGTCCATAA |
| | | [SEQ ID NO: 31] |
| Hs AHA1 | Forward | CTTTGCCACCATCACCTTGACC |
| | | [SEQ ID NO: 32] |
| | Reverse | AAAGTAGTACCGCTGCCAGCCC |
| | | [SEQ ID NO: 33] |
| Hs MMP14 | Forward | GAGCATTCCAGTGACCCCTC |
| | | [SEQ ID NO: 34] |
| | Reverse | ACCCTGACTCACCCCCATAA |
| | | [SEQ ID NO: 35] |

### Cell Treatment Malat1 Silencing in Neuro-2a Cells

Neuro-2a (murine neuroblastoma cells) were cultured in DMEM (PAN-Biotech GmbH) supplemented with 10% fetal bovine serum (FBS, PAN-Biotech GmbH) and Pen/Strep (PAN-Biotech GmbH). For experiments, Neuro-2a cells were harvested, resuspended at 60.000 cells/mL and seeded at a density of 36.000 cells/well or 6.000 cells/well for the 24- or 96-well plates (Greiner BioOne). Cells were incubated overnight at 37° C. Before the start of treatment, 210 UL or 35 μL medium was added per well, respectively, followed by the conjugates from a 10× concentrated stock solution in DPBS (PAN-Biotech GmbH). Plates were incubated for 72 hr at 37° C. before the cells of the 24-well plates were harvested for gene expression analysis and the cell viability was assessed on the 96-well plates.

### Cell Treatment Sod1 Exon Skip in Neuro-2a Cells

Neuro-2a (murine neuroblastoma cells) were cultured in DMEM (PAN-Biotech GmbH) supplemented with 10% fetal bovine serum (FBS, PAN-Biotech GmbH) and Pen/Strep (PAN-Biotech GmbH). For experiments Neuro-2a cells were harvested, resuspended at 70.000 cells/mL and seeded at a density of 42.000 cells/well or 7.000 cells/well for the 24 or 96-well plates (Greiner BioOne). Cells were incubated overnight at 37° C. Before the start of treatment 120 μL or 20 μL media was added per well resp., followed by both saponin and/or PMO from a 10× concentrated stock solution in DPBS (PAN-Biotech GmbH). In the control wells, or when only one compound was applied in treatment, additional DPBS was added so that the final volume per well was 900 μL resp. 150 μL for the 24 or 96-well plates. Plates were incubated for 72 hr at 37° C. before the cells of the 24 wp were harvested for gene expression analysis and the cell viability was assessed on the 96 wp.

### Cell Treatment A431 Cells

A431, epidermoid carcinoma, cells were cultured in DMEM (PAN-Biotech GmbH) supplemented with 10% fetal bovine serum (FBS) (PAN-Biotech GmbH) and Pen/Strep (PAN-Biotech GmbH) at 37° C. and 5% CO₂. Cells were seeded at 30.000 cells/well or 6.000 cells/well in 600 μL/well or 100 L/well for the 24-resp. 96-well plates (Greiner BioOne) and incubated overnight at 37° C. The next day, 120 μL or 20 μL media was added per well for the 24-resp. 96-well plate, followed by 10× concentrated compound-mix samples in DPBS (PAN-Biotech GmbH); which contained the compounds, i.e. an antisense oligonucleotide (ASO) or (targeted)-PMO and/or saponin component at 10× final concentration. Cells were treated with compounds for 72 hrs at 37° C., before the cells of the 24 wp were harvested for expression analysis and the cell viability was assessed on the 96 wp.

### Cell Treatment Malignant Glioma Derived, U87, Cells

Malignant glioma derived, U87, cells were cultured in DMEM (PAN-Biotech GmbH) supplemented with 10% fetal bovine serum (FBS) (PAN-Biotech GmbH) at 37° C. and 5% CO₂. Cells were seeded at 100.000 cells/well or 7.000 cells/well in 1 mL/well or 100 μL/well for the 12-resp. 96-well plates (Greiner BioOne) and incubated overnight at 37° C. The next day, media was refreshed by 0.85 mL or 127.5 μL media per well for the 12-resp. 96-well plate. Next, 100 μL or 15 μL media containing SO1861 at 10× final concentration was added per well, followed by 50 μL or 7.5 μL 20× concentrated siRNA compound-mix diluted in DPBS (PAN-Biotech GmbH). Cells were treated with compounds for 48 hrs at 37° C., before the cells of the 12 wp were harvested for expression analysis and the cell viability was assessed on the 96 wp.

### Cell Viability Assay (MTS)

The cells were treated for 72 hr at 37° C. before the cell viability was determined by an MTS-assay, performed according to the manufacturer's instruction (CellTiter 96® AQueous One Solution Cell Proliferation Assay, Promega). Briefly, the MTS solution was diluted 20′ in DMEM without phenol red (PAN-Biotech GmbH) supplemented with 10% FBS. Treatment medium was removed, after which 100 μL diluted MTS solution was added per well. The plate was incubated for approximately 20-30 minutes at 37° C. Subsequently, the OD at 492 nm was measured on a Spectramax iD5 plate reader (Molecular Devices). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

RNA Isolation and Quantitative Gene Expression Analysis from Cells

Total RNA from cells was isolated using TRIzol™ Reagent (Thermo Scientific) according to the manufacturer's instruction. Conversion into cDNA was performed using iScript™ cDNA Synthesis Kit (BioRad) using standard protocols. Gene expression levels of the gene of interest (GOI) and levels of specific housekeeping genes were determined using quantitative real-time PCR assays (qRT-PCR) using iTaq™ Universal SYBR® Green Supermix (BioRad) and the Light Cycler 480 II (Roche Diagnostics) with specific DNA primers, listed in Table A3. Each analysis reaction was performed in triplicate. Analysis was done by the ΔCt method to determine GOI expression relative to 2 specific housekeeping control mRNAs. Results are expressed as % relative GOI expression levels after normalization to DPBS treated control cells.

RNA Isolation and Gel Analysis from Cells

Total RNA from cells was isolated using TRIzol™ Reagent (Thermo Scientific) according to the manufacturer's instruction. Conversion into cDNA was performed using iScript™ cDNA Synthesis Kit (BioRad) using standard protocols. The gene expression was determined on 50 ng cDNA using the SapphireAmp Fast PCR Master Mix (Takara) with specific DNA primers, listed in Table A4. The PCR product was separated on a 2% agarose gel and fragments were analysed on the ChemiDoc XRS+ (BioRad) using Image Lab software (BioRad). For quantification of the % exon skip, the signal intensity of the aberrant transcript(s) (Mm Sod1 448 bp and 378 bp was divided over the total signal intensity of the skipped fragment(s)+full length fragment (Mm Sod1 545 bp)×100.

TABLE A4
Primers used in gel analysis

| | | |
|---|---|---|
| Mm Sod1 | Forward | CGTCTTGCTCTCTCTGGTCC |
| | | [SEQ ID NO: 21] |
| | Reverse | AGACTCAGACCACACAGGGA |
| | | [SEQ ID NO: 22] |
| Hs STAT3 (α/β) | Forward | TGCTACCAATATCCTGGTGTCTCC |
| | | [SEQ ID NO: 26] |
| | Reverse | ACTCCGAGGTCAACTCCATGTCAA |
| | | [SEQ ID NO: 27] |

### Example 8: Efficacy Enhancement by Targeted Saponin Component Co-Administration In Vivo to a Targeted siRNA (GN3-siTTR): Tolerability, Efficacy and Durability of Effect

Transthyretin (TTR) protein is a relevant pathological factor in familial amyloidotic polyneuropathy (FAP), a neurodegenerative disorder characterized by misfolding and deposition of mutant transthyretin (TTR) in the peripheral nervous system (PNS). Hereditary transthyretin amyloidosis (ATTRv amyloidosis; v for variant) is a genetic disease caused by the accumulation of misfolded transthyretin protein in different organs. CNS manifestations seem common in patients especially those with the V30M mutation and longstanding disease. TTR and production by the choroid plexus (in the four ventricles of the brain) escapes the therapeutic effect of liver transplant and other approved disease modifying and TTR targeting therapies, which do not cross the blood-brain barrier. This allows for the continuous accumulation of amyloid in the CNS throughout the disease. Effectively targeting or repairing mutant TTR in the CNS might be therapeutically beneficial.

### In Vivo Study Design

Male C57BL/6 mice were allocated to the dosing groups (vehicle n=3, GN3-siTTR n=6, and variously timed combinations of GN3-siTTR (also referred to as trimeric GalNAc-siRNA targeting murine Ttr, all n=6) and the targeted saponin components (here, GN3-SC-SO1861, also referred to as trimeric GalNAc-SC-SO1861) were dosed with the test compounds (GN3-siTTR always at 0.3 mg/kg and GN3-SC-SO1861 always at 1 mg/kg), see Table A5 for the dosing groups. Blood sampling was generally performed on day-4, 3, 7, 10, 14, 17, 21, 24, 28, 31, 35 and 49, unless indicated otherwise in Table A5. After blood sampling, serum was prepared and aliquoted in 2 tubes. One aliquot was used for serum TTR protein analysis and the other one for serum ALT enzyme analysis. Mice were terminated on day 49.

### Bioanalysis TTR Protein

To assess efficacy of the treatment, serum samples were analyzed for TTR protein content by ELISA using the ALPCO Mouse Prealbumin ELISA® Kit (#41-PALMS-E01, ALPCO) according to the manufacturer's instructions.

### Clinical Chemistry

To assess tolerability, ALT protein as a reporter for liver tolerability was assessed in serum samples with a Roche COBAS 6000 analyzer.

TABLE A5
Dosing groups in vivo tolerability, efficacy and durability of different
dosing regimens of trivalent trimeric-GalNAc-SO1861 (GN3-SC-SO1861)
in combination with trimeric-GalNAc-siRNA (GN3-siTTR)

| Treatment, | Treatment, | Treatment | | |
|---|---|---|---|---|
| Component 1 | Component 2 | day | Mouse | Blood sampling |
| 0.3 mg/kg | 1 mg/kg | Component 2 | ID | Timepoint |
| GN3-siTTR | N/A | N/A | 1-3 | −4d, 3d, d14, d28, d49 |
| | | | 4-6 | −4d, 7d, d21, d35, d49 |
| GN3-siTTR | GN3-AH-SO1861 | 0 (Co-Admin) | 7-9 | −4d, 3d, d14, d28, d49 |
| | | | 10-12 | −4d, 7d, d21, d35, d49 |
| GN3-siTTR | GN3-AH-SO1861 | 7 | 13-15 | −4d, 3d, d10, d21, d35, d49 |
| | | | 16-18 | −4d, 7d, d14, d28, d49 |
| GN3-siTTR | GN3-AH-SO1861 | 14 | 19-21 | −4d, 3d, d14, d21, d35, d49 |
| | | | 22-24 | −4d, 7d, d17, d28, d49 |
| GN3-siTTR | GN3-AH-SO1861 | 21 | 25-27 | −4d, 3d, d14, d24, d35, d49 |
| | | | 28-30 | −4d, 7d, d21, d28, d49 |
| GN3-siTTR | GN3-AH-SO1861 | 28 | 31-33 | −4d, 3d, d14, d28, d35, d49 |
| | | | 34-36 | −4d, 7d, d21, d31, d49 |
| GN3-siTTR | GN3-SC-SO1861 | 0 (Co-Admin) | 37-39 | −4d, 3d, d14, d28, d49 |
| | | | 40-42 | −4d, 7d, d21, d35, d49 |
| GN3-siTTR | GN3-SC-SO1861 | 7 | 43-45 | −4d, 3d, d10, d21, d35, d49 |
| | | | 46-48 | −4d, 7d, d14, d28, d49 |
| GN3-siTTR | GN3-SC-SO1861 | 14 | 49-51 | −4d, 3d, d14, d21, d35, d49 |
| | | | 52-54 | −4d, 7d, d17, d28, d49 |
| GN3-siTTR | GN3-SC-SO1861 | 21 | 55-57 | −4d, 3d, d14, d24, d35, d49 |
| | | | 58-60 | −4d, 7d, d21, d28, d49 |
| GN3-siTTR | GN3-SC-SO1861 | 28 | 61-63 | −4d, 3d, d14, d28, d35, d49 |
| | | | 64-66 | −4d, 7d, d21, d31, d49 |
| Vehicle (PBS) | — | — | 67-69 | −d4, 14d, 28d, 49d |

### Results

Efficacy and In Vivo Durability of a Targeted siRNA (GN3-siTTR) is Markedly Improved by Co-Administration of a Targeted Saponin Component (GN3-SC-SO1861), Especially when Added Delayed and then Independently of Dosing Schedule

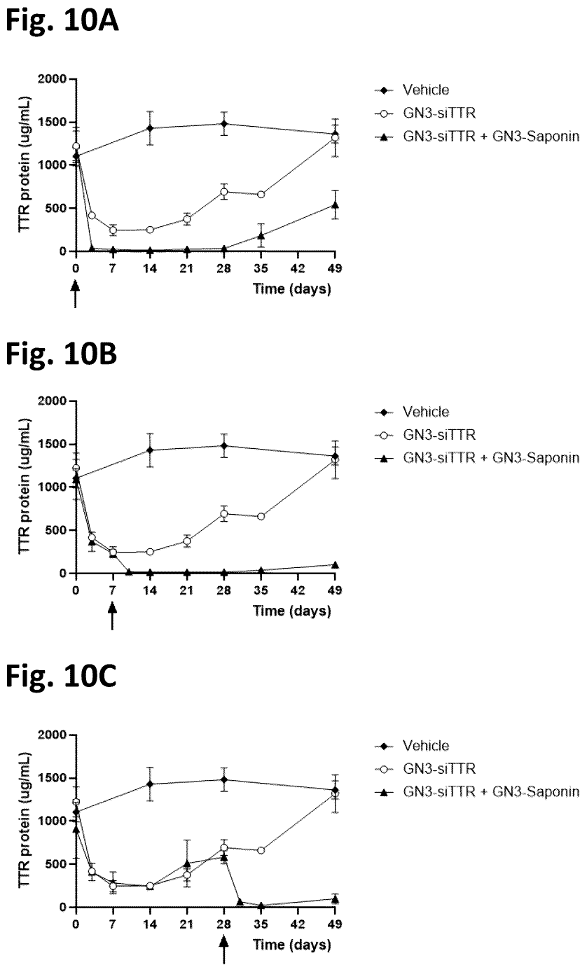

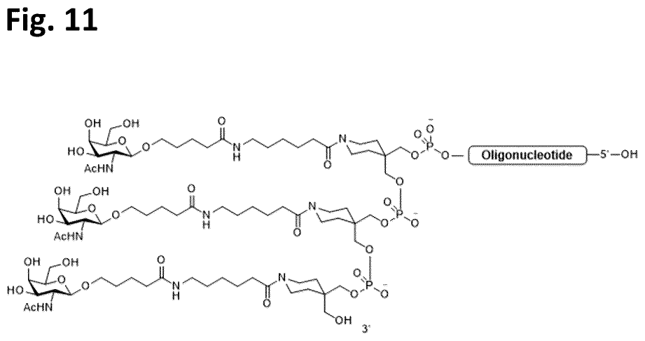

Trivalent-GalNAc is a targeting ligand that recognizes and binds the endocytosing ASGPR1 receptor, and was produced as previously described. SO1861-SC-N₃ was conjugated (in a similar manner as described in FIGS. 8 and 9, to trivalent-GalNAc, with a DAR=1 for the bound SO1861, to yield trivalent-GalNAc-SC-SO1861, also referred to as (GalNAc) 3-SC-SO1861, or GN3-SC-SO1861. Trivalent-GalNAc-siRNA targeting the murine Ttr (also referred to as GN3-siTTR [SEQ ID NO: 1]) was custom-produced (FIG. 11). All mice were intravenously (IV) injected with 0.3 mg/kg targeted siRNA (GN3-siTTR) on day 0, except 3 control mice that were injected with vehicle (PBS) only on day 0. Then, all GN3-siTTR-dosed mice, except one benchmark group of 6 animals, additionally received the targeted saponin component GN3-SC-SO1861 at a dose of 1 mg/kg, either on day 0, or day 7, or day 28. Serum samples were taken at different timepoints before and after dosing (Table A5) to assess the effect of the GN3-siTTR on circulating TTR protein levels. As FIG. 10A, 10B, 10C shows, as expected, mice receiving vehicle (n=3, on day 0) showed constant and high levels of TTR protein in serum throughout the study (i.e. at −4, 14, 28, and 49 days). Mice receiving only GN3-siTTR (n=6, on day 0; 0.3 mg/kg) without a saponin component showed a maximum effect of ~80% TTR knockdown approximately 7-14 days after treatment with said GN3-siTTR, and with a return of TTR protein levels to baseline levels (i.e., the level measured before administration, and also the level comparable to vehicle treated mice) on day 49. In contrast, and surprisingly, especially when compared to vehicle and to GN3-siTTR-only treated mice, mice receiving a combination of GN3-siTTR (on day 0; 0.3 mg/kg) and the saponin component GN3-SC-SO1861 (on day 0; 1 mg/kg) achieved far greater reduction (of >95%) already on day 3, the earliest day of serum TTR protein assessment (FIG. 10A). Surprisingly, mice that had received GN3-siTTR on day 0 and then a dose of the targeted saponin component either 7 days later (FIG. 10B), or 28 days later (FIG. 10D) showed almost complete TTR protein knockdown (>95%) within 3-7 days after treatment with the saponin component. Also, irrespectively of when the targeted saponin component was administered (in relation to the administration GN3-siTTR administration timepoint), there was no loss of durability of effect: saponin component administered at either day 7 or day 28 after initial targeted siRNA (GN3-siTTR) administration, resulted in up to 80% TTR serum level reduction for any of these regimens at the end of study at day 49 (FIG. 10B, 10C). Any of these co-treatment regimens were also equally well tolerated, as in general, no saponin-component-induced increase in the ALT enzyme levels was observed, which absence of an increased level serves as a proxy of liver tolerability was observed (not shown). Taken together, this data shows that co-administration of a targeted saponin component either at the same time, but more so at a delayed time point, to a targeted siRNA (GN3-siTTR) significantly increases efficacy, measured in this example as protein reduction, compared to treatment with the same dose of GN3-siTTR alone. Marked to almost complete reduction (>95%) of TTR protein expression by 0.3 mg/kg GN3-siTTR was reached only following co-dosing with a targeted saponin component GN3-SC-SO1861, but independently of the timepoint of saponin component administration, i.e., either at day 7 or day 28 throughout the study. Taken together these results confirm that saponin components can mediate efficient release of the siRNA from the endosomal (depot) compartment over at least 28 days after initial dosing of the targeted siRNA compound. Additionally, no loss of durability of effect was observed. Remarkably, in all cases where treatment was sequential (i.e., GN3-siTTR on day 0 and saponin component addition after 7 or 28 days), the levels of TTR were reduced by 80% at day 49 after initial GN3-siTTR treatment. Taken together, the saponin components were not only able to enhance the efficacy of a 0.3 mg/kg dose of GN3-siTTR, but a timed, saponin-induced release markedly prolonged the durability of effect.

SO1861-SC-azide synthesis

### Intermediate 1:

### tert-butyl 2-(4-(6-azidohexanoyl) piperazine-1-carbonyl) hydrazine-1-carboxylate

6-azidohexanoic acid (603 mg, 3.84 mmol), tert-butyl 2-(piperazine-1-carbonyl) hydrazine-1-carboxylate (781 mg, 3.20 mmol), EDCI·HCl (735 mg, 3.84 mmol) and Oxyma Pure (591 mg, 4.16 mmol) were dissolved in a mixture of dichloromethane (25 mL) and DIPEA (835 μL, 4.80 mmol) and the reaction mixture was stirred at room temperature. After 2 hours the reaction mixture was evaporated in vacuo and the residue was dissolved in ethyl acetate (50 mL). The resulting solution was washed with 0.5 N potassium bisulphate solution (50 mL), saturated sodium bicarbonate solution (2×50 mL) and brine (50 mL), dried over Na₂SO₄, filtered and evaporated in vacuo. The residue was purified by flash chromatography (DCM-10% methanol in DCM (v/v) gradient 100:0 rising to 40:60) to give the title compound (864 mg, 70%) as a white solid. Purity based on LC-MS 96%.

LRMS (m/z): 284/328/406 [M−99/M−55/M+23]¹⁺

LC-MS r.t. (min): 1.13²

### Intermediate 2:

### 4-(6-azidohexanoyl) piperazine-1-carbohydrazide 2,2,2-trifluoroacetate

tert-butyl 2-(4-(6-azidohexanoyl) piperazine-1-carbonyl) hydrazine-1-carboxylate (50.0 mg, 130 μmol) was dissolved in a mixture of dichloromethane (1.00 mL) and TFA (1.00 mL) and the reaction mixture was stirred at room temperature. After 1 hour the reaction mixture was evaporated in vacuo and co-evaporated with dichloromethane (3×5 mL) to give the crude title product as a white solid.

LRMS (m/z): 284/307 [M+1/M+23]¹⁺

### SO1861-SC-Azide

To SO1861 (60 mg, 0.032 mmol) and 4-(6-azidohexanoyl) piperazine-1-carbohydrazide 2,2,2-trifluoroacetate (51.2 mg, 0.129 mmol) was added methanol (extra dry, 1.5 mL) and the reaction mixture was shaken for 1 min and left standing at room temperature. After 3 hours the reaction mixture was subjected to preparative MP-LC.1A Fractions corresponding to the product were immediately pooled together, frozen and lyophilized overnight to give the title compound (55.6 mg, 81%) as a white solid. Purity based on LC-MS 96%.

LRMS (m/z): 2127 [M−1]¹⁻

LC-MS r.t. (min): 3.39^{5A}

See FIG. 8 for the formula of SO1861-SC-azide (referred to as ‘SO1861-SC-N3’ in FIG. 8).

### Trivalent GalNAc-Azide Synthesis

### Intermediate 1:

### tert-butyl 1-azido-17,17-bis((3-(tert-butoxy)-3-oxopropoxy)methyl)-15-oxo-3,6,9,12,19-pentaoxa-16-azadocosan-22-oate

Intermediate 1 was produced as previously described in WO2022/055351 (page 136, line 3 to page 139, line 1, FIG. 8, Example 1C).

To di-tert-butyl 3,3′-((2-amino-2-((3-(tert-butoxy)-3-oxopropoxy)methyl) propane-1,3-diyl) bis(oxy)) dipropionate (1.27 g, 2.51 mmol) was added a solution of 3-Azido (peg4) propionic acid N-hydroxysuccinimide ester (977 mg, 2.51 mmol) in DMF (10 mL). Next, DIPEA (657 μL, 3.77 mmol) was added and the reaction mixture was stirred overnight at room temperature. The reaction mixture was evaporated in vacuo and the residue was dissolved in ethyl acetate (100 mL). The resulting solution was washed with 0.5 N potassium bisulphate solution (2×100 mL) and brine (100 mL), dried over Na₂SO₄, filtered and evaporated in vacuo. The residue was purified by flash chromatography (DCM-10% methanol in DCM (v/v) gradient 100:0 rising to 0:100) to give the title compound (1.27 g, 65%) as a colorless oil. Purity based on LC-MS 100% (ELSD).

LRMS (m/z): 780 [M+1]¹⁺

LC-MS r.t. (min): 2.10²

### Intermediate 2:

### 1-azido-17,17-bis((2-carboxyethoxy)methyl)-15-oxo-3,6,9,12,19-pentaoxa-16-azadocosan-22-oic acid

Intermediate 2 was produced as previously described in WO2022/055351 (page 136, line 3 to page 139, line 1, FIG. 8, Example 1C).

To a solution of tert-butyl 1-azido-17,17-bis((3-(tert-butoxy)-3-oxopropoxy)methyl)-15-oxo-3,6,9,12,19-pentaoxa-16-azadocosan-22-oate (1.27 g, 1.63 mmol) in DCM (5.0 mL) was added TFA (5.0 mL, 65 mmol). The reaction mixture was stirred at room temperature. After 1.5 hours the reaction mixture was evaporated in vacuo, co-evaporated with toluene (3×10 mL) and DCM (3×10 mL) to give the crude title product as a colorless oil.

LRMS (m/z): 611 [M+1]¹⁺

### Intermediate 3:

### di-tert-butyl (10-(1-azido-3,6,9,12-tetraoxapentadecan-15-amido)-10-(13,13-dimethyl-5,11-dioxo-2,12-dioxa-6,10-diazatetradecyl)-5,15-dioxo-8,12-dioxa-4,16-diazanonadecane-1,19-diyl)dicarbamate

Intermediate 3 was produced as previously described in WO2022/055351 (page 136, line 3 to page 139, line 1, FIG. 8, Example 1C).

1-azido-17,17-bis((2-carboxyethoxy)methyl)-15-oxo-3,6,9,12,19-pentaoxa-16-azadocosan-22-oic acid (997 mg, 1.63 mmol), Oxyma Pure (1.04 g, 7.35 mmol) and EDCI·HCl (1.17 g, 6.12 mmol) were dissolved in DMF (10.0 mL). Next, DIPEA (1.99 mL, 11.4 mmol) was added, followed directly by the addition of a solution of N-BOC-1,3-propanediamine (1.07 g, 6.12 mmol) in DMF (10.0 mL). The reaction mixture was stirred overnight at room temperature. The reaction mixture was evaporated in vacuo and the residue was dissolved in ethyl acetate (100 mL). The resulting solution was washed with 0.5 N potassium bisulphate solution (100 mL), saturated sodium bicarbonate solution (2×100 mL) and brine (100 mL), dried over Na₂SO₄, filtered and evaporated in vacuo. The residue was purified by flash chromatography (DCM-10% methanol in DCM (v/v) gradient 0:100 rising to 100:0, staying at 100:0 until the product eluted) to give the title compound (1.16 g, 66%) as a yellowish viscous oil. LC-MS 99% (ELSD).

LRMS (m/z): 1080 [M+1]¹⁺

LC-MS r.t. (min): 1.51³

### Intermediate 4:

### 3,3′-((2-((3-((3-aminopropyl)amino)-3-oxopropoxy)methyl)-2-(1-azido-3,6,9,12-tetraoxapentadecan-15-amido) propane-1,3-diyl) bis(oxy)) bis(N-(3-aminopropyl) propanamide) tris(2,2,2-trifluoroacetate) synthesis

Intermediate 4 was produced as previously described in WO2022/055351 (page 136, line 3 to page 139, line 1, FIG. 8, Example 1C).

To a solution of di-tert-butyl (10-(1-azido-3,6,9,12-tetraoxapentadecan-15-amido)-10-(13,13-dimethyl-5,11-dioxo-2,12-dioxa-6,10-diazatetradecyl)-5,15-dioxo-8,12-dioxa-4,16-diazanonadecane-1,19-diyl) dicarbamate (1.16 g, 1.08 mmol) in DCM (10 mL) was added TFA (10 mL, 131 mmol). The reaction mixture was stirred at room temperature. After 2 hours the reaction mixture was evaporated in vacuo, co-evaporated with toluene (3×10 mL) and DCM (3×10 mL) to give the crude title product as a yellowish viscous oil.

LRMS (m/z): 260 [M+3]³⁺, 390 [M+2]²⁺, 780 [M+1]¹⁺,

### Intermediate 5:

### (2R,3R,4R,5R,6R)-5-acetamido-2-(acetoxymethyl)-6-((5-((2,5-dioxopyrrolidin-1-yl)oxy)-5-oxopentyl)oxy) tetrahydro-2H-pyran-3,4-diyl diacetate

Intermediate 5 was produced as previously described in WO2022/055351 (page 136, line 3 to page 139, line 1, FIG. 8, Example 1C).

5-(((2R,3R,4R,5R,6R)-3-acetamido-4,5-diacetoxy-6-(acetoxymethyl)tetrahydro-2H-pyran-2-yl)oxy) pentanoic acid (obtain according to Nair et al., Multivalent N-Acetylgalactosamine-Conjugated siRNA Localizes in Hepatocytes and Elicits Robust RNAi-Mediated Gene Silencing, J. Am. Chem Soc., 2014, 136, 16958-16961, 3.00 g, 6.70 mmol) and N-Hydroxysuccinimide (926 mg, 8.05 mmol) were dissolved in DCM (50 mL). Next, EDCI·HCl (1.54 g, 8.05 mmol) and 4-(Dimethylamino)pyridine (82 mg, 0.67 mmol) were added and the reaction mixture was stirred overnight at room temperature. The reaction mixture was diluted with DCM and the resulting solution was washed with 0.5 N potassium bisulphate solution (150 mL), saturated sodium bicarbonate solution (150 mL) and brine (150 mL), dried over Na₂SO₄, filtered and evaporated in vacuo to give the title compound (3.60 g, 99%) as a white foam. Purity based on LC-MS 99% (ELSD).

LRMS (m/z): 545 [M+1]¹⁺

LC-MS r.t. (min): 1.07³

### Intermediate 6:

### [(3R,6R)-3,4-bis(acetyloxy)-6-{4-[(3-{3-[2-(1-azido-3,6,9,12-tetraoxapentadecan-15-amido)-3-(2-{[3-(5-{[(2R,5R)-4,5-bis(acetyloxy)-6-[(acetyloxy)methyl]-3-acetamidooxan-2-yl]oxy}pentanamido) propyl]carbamoyl}ethoxy)-2-[(2-{[3-(5-{[(2R,5R)-4,5-bis(acetyloxy)-6-[(acetyloxy)methyl]-3-acetamidooxan-2-yl]oxy}pentanamido) propyl]carbamoyl}ethoxy)methyl]propoxy]propanamido}propyl) carbamoyl]butoxy}-5-acetamidooxan-2-yl]methyl acetate

Intermediate 6 was produced as previously described in WO2022/055351 (page 136, line 3 to page 139, line 1, FIG. 8, Example 1C).

3,3′-((2-((3-((3-aminopropyl)amino)-3-oxopropoxy)methyl)-2-(1-azido-3,6,9,12-tetraoxapentadecan-15-amido) propane-1,3-diyl) bis(oxy)) bis(N-(3-aminopropyl) propanamide) tris(2,2,2-trifluoroacetate) (1.21 g, 1.08 mmol) was dissolved in a mixture of DMF (10 mL) and DIPEA (1.69 mL, 9.70 mmol). Next, (2R,3R,4R,5R,6R)-5-acetamido-2-(acetoxymethyl)-6-((5-((2,5-dioxopyrrolidin-1-yl)oxy)-5-oxopentyl)oxy) tetrahydro-2H-pyran-3,4-diyl diacetate (2.20 g, 4.04 mmol) was added and the reaction mixture was stirred over the weekend at room temperature. Next, the reaction mixture was evaporated in vacuo and the residue was purified by flash chromatography (DCM-30% methanol in DCM (v/v) gradient 0:100 rising to 100:0) to give the title compound (1.84 g, 83%) as a yellowish foam. LC-MS 95% (ELSD).

LRMS (m/z): 2068 [M+1]¹⁺

LC-MS r.t. (min): 1.18³

### Intermediate 7:

### Trivalent GalNAc-azide

Trivalent GalNAc-azide was produced as previously described in WO2022/055351 (page 136, line 3 to page 139, line 1, FIG. 8, Example 1C).

[(3R,6R)-3,4-bis(acetyloxy)-6-{4-[(3-{3-[2-(1-azido-3,6,9,12-tetraoxapentadecan-15-amido)-3-(2-{[3-(5-{[(2R,5R)-4,5-bis(acetyloxy)-6-[(acetyloxy)methyl]-3-acetamidooxan-2-yl]oxy}pentanamido) propyl]carbamoyl}ethoxy)-2-[(2-{[3-(5-{[(2R,5R)-4,5-bis(acetyloxy)-6-[(acetyloxy)methyl]-3-acetamidooxan-2-yl]oxy}pentanamido) propyl]carbamoyl}ethoxy)methyl]propoxy]propanamido}propyl) carbamoyl]butoxy}-5-acetamidooxan-2-yl]methyl acetate (300 mg, 0.145 mmol) was dissolved in a mixture of triethylamine (2.00 mL, 14.4 mmol), methanol (2.00 mL) and water (2.00 mL) and the reaction mixture was stirred at room temperature. After 2 hours the reaction mixture was evaporated in vacuo. The residue was purified by preparative MP-LC.2B Fractions corresponding to the product were immediately pooled together, frozen and lyophilized overnight to give the title compound (164 mg, 67%) as a white solid. Purity based on LC-MS 97%.

LRMS (m/z): 1688 [M−1]¹⁻

LC-MS r.t. (min): 1.99^{1A}

### Trivalent GalNAc-Amine Formate

Trivalent GalNAc-amine formate was produced as previously described in WO2022/055351 (page 143-144, Example 1D).

Trivalent GalNAc-azide (36.5 mg, 21.6 μmol) was dissolved in a solution of potassium carbonate (5.97 mg, 43.2 μmol) in water (1.00 mL) and acetonitrile (1.00 mL). Next, a 1.0 M trimethylphosphine solution in THF (216 μL, 216 μmol) was added and the resulting mixture was shaken for 1 min and left standing at room temperature. After 45 min the reaction mixture was evaporated in vacuo and the residue was dissolved in water/acetonitrile (9:1, v/v, 1 mL). The resulting solution was directly subjected to preparative MP-LC.^{2B} Fractions corresponding to the product were immediately pooled together, frozen and lyophilized overnight to give the title compound (36.1 mg, 98%) as a white solid. Purity based on LC-MS 100%.

LRMS (m/z): 1662 [M−1]¹⁻

LC-MS r.t. (min): 1.62^{1A}

### Intermediate 8: Trivalent GalNAc-DBCO

Trivalent GalNAc-DBCO was produced as previously described in WO2022/055351 (page 143-144, Example 1D).

Trivalent GalNAc-amine formate (17.4 mg, 10.2 μmol) and DBCO-NHS (6.14 mg, 15.3 μmol) were dissolved in a solution of NMM (2.24 μL, 20.3 μmol) in DMF (0.50 mL). The reaction mixture was shaken for 1 min and left standing at room temperature. After 2 hours the reaction mixture was evaporated in vacuo and the residue was dissolved in water/acetonitrile (8:2, v/v, 1 mL). The resulting solution was directly subjected to preparative MP-LC.20 Fractions corresponding to the product were immediately pooled together, frozen and lyophilized overnight to give the title compound (14.2 mg, 72%) as a white solid. Purity based on LC-MS 96%.

LRMS (m/z): 1950 [M−1]¹

LC-MS r.t. (min): 1.86^{1B}

### GN3-SC-SO1861

To SO1861-SC-N3 (18.0 mg, 8.45 μmol) and trivalent GN3-DBCO (16.5 mg, 8.45 μmol) was added a mixture of acetonitrile (250 μL) and 20 mM ammonium bicarbonate (750 μL). The reaction mixture was shaken for about 1 min and left standing at room temperature. After 1 hour the reaction mixture was subjected to preparative MP-LC.^{1A} Fractions corresponding to the product were immediately pooled together, frozen and lyophilized overnight to give the title compound (29.2 mg, 85%) as a white solid. Purity based on LC-MS 99%.

LRMS (m/z): 2038 [M−2H]²⁻

LC-MS r.t. (min): 2.19^{5B}

See FIG. 9 for the formula of GN3-SC-SO1861.

### GN3-siTTR

Trivalent-GalNAc-siRNA targeting murine transthyretin (also referred to as the nucleic acid component GN3-siTTR [SEQ ID No: 1] for the sense strand, and SEQ ID No. 15 for the antisense strand]) with advanced enhanced stability chemistry backbone was custom-produced by BioSpring Gesellschaft für Biotechnologie mbH, Germany, according to methods known in the art (FIG. 11). GalNAc monomers were conjugated via their phosphate groups in a linear fashion to generate a trimeric GalNAc. The phosphate group of the third GalNAc links to the 3′ end of the oligonucleotide sequence resulting in the following conjugate with sense strand [SEQ ID No: 1]: 5′-6*6*7685451315875756566000; and antisense strand [SEQ ID No: 15]: 5′-5*1*6564643668627275855*5*5; with 0=GalNac, 1=2′-Fluoro-U, 2=2′-Fluoro-A, 3=2′-Fluoro-C, 4=] 2′-Fluoro-G, 5=2′OMe-rU, 6=2′OMe-rA, 7=2′OMe-rC, 8=2′OMe-rG, *=phosphorothioate. Such a linear trimeric GalNAc could also be conjugated to saponin components to generate for example GN3-SC-SO1861 by methods known in the art.

### Materials for Examples 9-13

All chemicals, solvents and buffers were purchased in their highest purity available from either Sigma-Aldrich (Netherlands), Merck KGaA (Netherlands), Thermo-Fisher (Netherlands), VWR (Netherlands) or TCI (Europe) and were used as received unless otherwise stated.

SO1861 was isolated and purified by either Analyticon Discovery GmbH, Germany or Extrasynthese, France, from raw plant extract obtained from Saponaria officinalis L.

### Monoclonal Antibodies

To generate the conjugates
aCD71-SOD1 PMO (both Conjugate 1 and Conjugate 2)
aCD71-(Saponin-SOD1 PMO)_{high}
aCD71-(Saponin-SOD1 PMO)_{low}
aCD71-Malat1 ASO
   the following monoclonal antibody was used: Anti mouse CD71mab rat IgG2a, clone: R17 217.1.3/TIB-219, Lot No 821022M2, catalog No: BE0175, vendor: Bioxcell.

To generate the conjugates
aCD71-PMO (1)
aCD71-PMO (2)
aCD71-SOD1 ASO
   the following monoclonal antibody was used: Anti human CD71mab mouse IgG1, clone: OKT-9, Lot-No 766322F1, catalog-No BE0023, vendor: Bioxcell

### Analytical Methods

### LC-MS Method 1

Apparatus: Agilent 1200 Bin. Pump: G1312A, degasser; autosampler, ColCom, DAD: Agilent G1316A, 210, 220 and 220-320 nm, PDA: 210-320 nm, MSD: Agilent LC/MSD G6130B ESI, pos/neg 100-1000; ELSD Alltech 3300 gas flow 1.5 ml/min, gas temp: 40° C.; column: Waters XSelect™ CSH C18, 30×2.1 mm, 3.5 μm, Temp: 35° C., Flow: 1 mL/min, Gradient:

$${t}_{0} = 5 \% B , {t}_{1.6 min} = 98 \% B , {t}_{3 min} = 98 \% B ,$$

Postrun: 1.3 min, Eluent A: 0.1% formic acid in water, Eluent B: 0.1% formic acid in acetonitrile.

### LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, neg/pos within in a range of 1500-2500 or 2000-3000; ELSD: gaspressure 40 psi, drift tube temp: 50° C.; column: Acquity Premier Peptide BEH, 50×2.1 mm, 1.7 μm Temp: 25° C., Flow: 0.45 mL/min, lin. Gradient depending on the polarity of the product:

$${}_{}^{A} {t}_{0}^{} = 2 \% B , {t}_{4. min} = 50 \% B {t}_{5. min} = 98 \% B , {t}_{6. min} = 98 \% B$$

$${}_{}^{B} {t}_{0}^{} = 5 \% B , {t}_{5. min} = 98 \% B , {t}_{7. min} = 98 \% B , Posttime : 1. min$$

Eluent A: 10 mM ammonium bicarbonate in water (pH=9.5), Eluent B: acetonitrile.

### LC-MS Method 3

Agilent 1260 Infinity II, 1260 G7112B Bin. Pump, 1260 G7167A Multisampler, 1290 MCT G7116B Column Comp. 1260 G7115A DAD (210-320 nm, 210 and 220 nm), PDA (210-320 nm), G6130B MSD (ESI pos/neg) mass range 90-1500, Column: Waters C4 BEH (50×2.1 mm 3.5 μm) Flow: 1 mL/min; Column Temp: 40° C., Eluent A: 0.1% formic acid in water, Eluent B: 0.1% formic acid in acetonitrile, Gradient:

$${}_{}^{A} {t}_{0 min}^{} = 5 \% B , {t}_{2.5 min} = 98 \% B , {t}_{4 min} = 98 \% B$$

$${}_{}^{B} {t}_{0 min}^{} = 5 \% B , {t}_{0.05 min} = 5 \% B , {t}_{5. min} = 98 \% B , \\ {t}_{6 min} = 98 \% B , Postrun : 1.5 min .$$

### LC-MS Method 4

Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5 μm) Flow: 0.8 ml/min Column temp: 40° C., Eluent A: 0.1% Formic acid in Water, Eluent B: 0.1% Formic acid in Acetonitrile, Gradient:

$${t}_{0} = 5 \% B , {t}_{0.5 min} = 5 \% B , {t}_{4.5 min} = 98 \% B , \\ {t}_{5. min} = 98 \% B , Postrun : 0.5 min .$$

### LC-MS Method 5

Apparatus: Waters IClass; Bin. Pump: UPIBSM, SM: UPISMFTN with SO; UPCMA, PDA: UPPDATC, 210-320 nm, SQD: ACQ-SQD2 ESI, mass range 1500-2500 neg/pos; ELSD: gaspressure 40 psi, drift tube temp: 50° C.; column: Acquity Premier Peptide BEH, 50×2.1 mm, 1.7 μm Temp: 25° C., Flow: 0.45 mL/min, lin. Gradient

$${t}_{0} = 2 \% B , {t}_{4. min} = 50 \% B , {t}_{5. min} = 98 \% B , {t}_{7. min} = 98 \% B ,$$

Posttime: 1.0 min, Eluent A: 10 mM ammonium bicarbonate in water (pH=9.5), Eluent B: acetonitrile.

### LC-MS Method 6

Protein RP HPLC ESI MS was performed on a X evo G 2 S TOF mass spectrometer coupled to an Acquity UPLC system using a BioResolve mAb RP Polyphenyl 450 Å column (27 μm, 21×30 mm) H2O with 0.1% formic acid (solvent A) and MeCN with 0.1% formic acid (solvent B) were used as the mobile phase at a flow rate of 0.2 ml/min. The gradient was programmed as follows: 95% A for 0 93 min, then a gradient to 100% B over 4.28 min, then 100% B for 1.04 minutes, then a gradient to 95% A over 1.04 min. The electrospray source was operated with a capillary voltage of 3.0 Kv and a cone voltage of 175 V. Nitrogen was used as the desolvation gas at a total flow rate of 700 L/h. Total mass spectra were reconstructed from the ion series using the Maxent 1 algorithm preinstalled on MassLynx 4.2 software according to the manufacturer's instructions.

### Preparative Methods

### Preparative MP-LC Method 1

Instrument type: Reveleris™ prep MPLC; Column: Dr. Maisch Reprosil (C18, 150×25 mm, 10 μm); Flow: 40 mL/min; Column temp: room temperature; Eluent A: 0.1% (v/v) formic acid in water, Eluent B: 0.1% (v/v) formic acid in acetonitrile; Gradient: t0 min=5% B, t1 min=5% B, t2 min=20% B, t17 min=60% B, t18 min=100% B, t23 min=100% B; Detection UV: 220, 254, 280, 320 nm; Fraction collection based on UV.

### Preparative MP-LC Method 2

Instrument type: Reveleris™ prep MPLC; Column: Phenomenex LUNA (C18, 150×25 mm, 10 μm); Flow: 40 mL/min; Column temp: room temperature; Eluent A: 0.1% (v/v) formic acid in water, Eluent B: 0.1% (v/v) formic acid in acetonitrile; Gradient:

$${t}_{0 min} = 5 \% B , {t}_{1 min} = 5 \% B , {t}_{2 min} = 30 \% B , \\ {t}_{17 min} = 70 \% B , {t}_{18 min} = 100 \% B , {t}_{23 min} = 100 \% B ;$$

Detection UV: 220, 252, 280 nm. Fraction collection based on UV.

### Preparative MP-LC Method 3

Instrument type: Reveleris™ prep MPLC; Column: Phenomenex LUNA (C18, 150×25 mm, 10 μm); Flow: 40 mL/min; Column temp: room temperature; Eluent A: 10 mM ammonium bicarbonate in water pH=9.0, Eluent B: 99% acetonitrile+1% 10 mM ammoniumbicarbonate in water; Gradient:

$${}_{}^{A} {t}_{0 min}^{} = 5 \% B , {t}_{1 min} = 5 \% B , {t}_{2 min} = 10 \% B , \\ {t}_{17 min} = 50 \% B , {t}_{18 min} = 100 \% B , {t}_{23 min} = 100 \% B$$

$${}_{}^{B} {t}_{0 min}^{} = 5 \% B , {t}_{1 min} = 5 \% B , {t}_{2 min} = 20 \% B , \\ {t}_{17 min} = 60 \% B , {t}_{18 min} = 100 \% B , {t}_{23 min} = 100 \% B$$

$${}_{}^{C} {t}_{0 min}^{} = 5 \% B , {t}_{1 min} = 5 \% B , {t}_{2 min} = 30 \% B , \\ {t}_{17 min} = 70 \% B , {t}_{18 min} = 100 \% B , {t}_{23 min} = 100 \% B$$

Detection UV: 210, 235, 254 nm and ELSD.

### Preparative LC-MS Method 4

MS instrument type: Agilent Technologies G6130B Quadrupole; HPLC instrument type: Agilent Technologies 1290 preparative LC; Column: Waters XBridge Protein (C4, 150×19 mm, 10 μm); Flow: 25 ml/min; Column temp: room temperature; Eluent A: 10 mM ammonium bicarbonate in water pH=9.0; Eluent B: 100% acetonitrile; Gradient:

$${}_{}^{A} {t}_{0}^{} = 10 \% B , {t}_{2.5 min} = 10 \% B , {t}_{11 min} = 50 \% B , \\ {t}_{13 min} = 100 \% B , {t}_{17 min} = 100 \% B$$

$${}_{}^{B} {t}_{0}^{} = 30 \% B , {t}_{2.5 min} = 30 \% B , {t}_{11 min} = 70 \% B , \\ {t}_{13 min} = 100 \% B , {t}_{17 min} = 100 \% B$$

$${}_{}^{C} {t}_{0}^{} = 20 \% B , {t}_{2.5 min} = 20 \% B , {t}_{11 min} = 60 \% B , \\ {t}_{13 min} = 100 \% B , {t}_{17 min} = 100 \% B$$

Detection: DAD (210 nm); Detection: MSD (ESI pos/neg) mass range: 100-800; Fraction collection based on DAD

### Preparative LC-MS Method 5

MS instrument type: Agilent Technologies G6120AA Quadrupole; HPLC instrument type: Agilent Technologies 1200 preparative LC; Column: Waters XBridge Protein (C4, 150×19 mm, 10μ); Flow: 25 ml/min; Column temp: room temperature; Eluent A: 0.1% formic acid in water; Eluent B: 100% acetonitrile; Gradient:

$${t}_{0} = 10 \% A , {t}_{2.5 min} = 10 \% A , {t}_{11 min} = 50 \% A , \\ {t}_{13 min} = 100 \% A , {t}_{17 min} = 100 \% A ;$$

Detection: DAD (220-320 nm); Detection: MSD (ESI pos/neg) mass range: 100-1000; Fraction collection based on DAD.

### Preparative LC-MS Method 6

Instrument type: Reveleris™ prep MPLC; Column: Phenomenex LUNA C18 (3) (150×25 mm, 10 μm); Flow: 40 mL/min; Column temp: room temperature; Eluent A: 10 mM ammonium bicarbonate in water pH=9.0, Eluent B: 99% acetonitrile+1% 10 mM ammoniumbicarbonate in water; Gradient:

$${t}_{0 min} = 5 \% B , {t}_{1 min} = 5 \% B , {t}_{2 min} = 10 \% B , \\ {t}_{17 min} = 50 \% B , {t}_{18 min} = 100 \% B , {t}_{23 min} = 100 \% B ;$$

Detection UV: 210, 235, 254 nm and ELSD.

### Flash Chromatography

Grace Reveleris X2° C.-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50bar (725 psi); Detection: UV 200-400 nm, combination of up to 4 UV signals and scan of entire UV range, ELSD; Column sizes: 4-330 g on instrument, luer type, 750 g up to 3000 g with optional holder.

### UV-Vis Spectrophotometry

Antibody, PMO, and ASO concentrations and incorporations were determined using a Thermo Nanodrop 2000 spectrometer. Antibody concentrations in the conjugates were determined by BCA assay. BCA assays were conducted using a Thermo SkanIT plate reader.
SOD1 ASO, EC260=196000 M-1 cm-1.
SOD1 PMO (1), EC265=253730 M-1 cm-1
SOD1 PMO (2), EC265=245060 M-1 cm-1.

### SEC

Native antibody and conjugates were analysed by SEC using an Akta purifier 100 system and either a Biosep SEC-s3000 column eluting with DPBS: IPA (85:15) or a Cytiva Superdex 200PG column eluting with DPBS: IPA (90:10). % purity was determined by integration of the antibody peak with respect to trace aggregate peaks.

### HIC

Native antibody and conjugates were analysed by HIC using an Akta pure M system and a Cytiva HiScreen Phenyl HP column eluting with DPBS. Average DAR was determined by integration of the conjugate peak with respect to trace antibody peaks.

### SDS-PAGE and Western Blotting

Native antibody and conjugates were analysed under heat denaturing non-reducing and reducing conditions by SDS-PAGE against a protein ladder using a 4-12% bis-tris gel and MOPS as running buffer (200V, 40 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using ImageJ.

For Western Blotting, washed gel (not Coomassie stained) was transferred to nitrocellulose membrane using the X-Cell blot module with the following setup (BP-BP-FP-Gel-NC-FP-BP-FP-Gel-NC-FP-BP-BP) and conditions (30V, 0.17 Amps, 60 minutes) and freshly prepared transfer buffer. BP—blotting pad; FP—Filter pad; NC—Nitrocellulose membrane. After, the NC were washed thrice with PBS-T (100 ml), non-specific sites blocked with blocking buffer (30 ml) with shaking (10 minutes, 200 rpm) then active sites labelled with a combination of Goat anti-Human Kappa-HRP (1:2000) and Goat anti-Human IgG-HRP (1:2000) (30 ml) diluted in blocking buffer with shaking (60 minutes, 200 rpm). After, the NC were washed with PBS-T (100 ml) and complexed antibody detected with CN/DAB substrate (25 ml) freshly prepared using stable peroxide substrate buffer. Colour development was observed visually and the resulting NC photographed.

### Synthesis

### SO1861-AH-Maleimide

SO1861-AH-Maleimide (also referred to as SO1861-AH-Mal or SO1861-EMCH) was produced as previously described in WO 2021/259507A1 (page 72, Example 3, referred to as “SO1861-EMCH synthesis”). To SO1861 (121 mg, 0.065 mmol) and EMCH. TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC (method 6). Fractions corresponding to the product were immediately pooled together, frozen and lyophilized overnight to give the title compound (120 mg, 90%) as a white fluffy solid. Purity based on LC-MS (method 5) 96%.

LRMS (m/z): 2069 [M−1]1−

LC-MS r.t. (min): 1.084

### SO1861-AH-Maleimide-Block (Saponin Molecule According to Formula (V), Also Referred to as SO1861-AH-Block or SO1861-AH (Block))

To SO1861-AH-Maleimide (0.1 mg, 48 nmol) 200 μL mercaptoethanol (18 mg, 230 μmol) was added and the solution was shaken for 1 h at 800 rpm and room temperature on a ThermoMixer C (Eppendorf). After shaking for 1 h, the solution was diluted with methanol and dialyzed extensively for 4 h against methanol using regenerated cellulose membrane tubes (Spectra/Por 7) with a MWCO of 1 kDa. After dialysis the SO1861-Ald-EMCH-mercaptoethanol was provided (saponin molecule according to formula (V)), an aliquot was taken out and analyzed via MALDI-TOF-MS.

(RP mode): m/z 2193 Da ([M+K]⁺, SO1861-AH-Block), m/z 2185 Da ([M+K]⁺, SO1861-AH-Block), m/z 2170 Da ([M+Na]⁺, SO1861-AH-Block).

### SO1861-SC-Maleimide Synthesis

SO1861-SC-Maleimide (also referred to as SO1861-SC-Mal) was produced as previously described in WO 2023/038517A1 (page 168, line 1 to line 13, Example 1, referred to as “SO1861-SC-Mal”). Tert-butyl 2-(4-(6-(2,5-dioxo-2,5-dihydro-1H-pyrrol-1-yl) hexanoyl) piperazine-1-carbonyl) hydrazine-1-carboxylate (25.0 mg, 57.1 μmol) was dissolved in a mixture of dichloromethane (500 μL) and TFA (500 μL) and the reaction mixture was stirred at room temperature. After 30 min the reaction 35 mixture was evaporated in vacuo and co-evaporated with dichloromethane (3×5 mL) and methanol (5 mL). The residue and SO1861 (21.3 mg, 11.4 μmol) were dissolved in methanol (extra dry, 1.00 mL) and the resulting mixture was shaken for 1 min and left standing at room temperature. After 4 hours the reaction mixture was subjected to preparative MP-LC (method 6). Fractions corresponding to the product were immediately pooled together, frozen and lyophilized overnight to yield the title compound (13.7 mg, 55%) as a white fluffy solid. Purity based on LC-MS (method 5) 97%.

LRMS (m/z): 2181 [M−1]¹⁻

LC-MS r.t. (min): 2.133

### SO1861-SC

SO1861-SC was produced as previously described in WO2022055352A1 (page 125, line 8 to line 17, Example 1, referred to as “SO1861-SC”).

Morpholine-4-carbohydrazide (3.89 mg, 26.8 μmol) and SO1861 (5.00 mg, 2.68 μmol) were dissolved in methanol (extra dry, 250 μL). Next, TFA (1.03 μL) was added and the resulting mixture was shaken for 1 min and left standing at room temperature. After 1 hour the reaction mixture was subjected to to preparative MP-LC (method 6). Fractions corresponding to the product were immediately pooled together, frozen and lyophilized overnight to yield the title compound (3.29 mg, 62%) as a white fluffy solid. Purity based on LC-MS (method 5) 95%.

LRMS (m/z): 1991 [M−1]¹⁻

LC-MS r.t. (min): 1.99

### TFL-(DIBO)-(d2 [S—S-SOD1 PMO])-(d2 [SC-SO1861]) Synthesis

### Intermediate 1:

### tert-butyl N-[2-(2-{2-[2-(2,2,2-trifluoroacetamido) ethoxy]ethoxy}ethoxy)ethyl]carbamate

t-Boc-N-Amido-PEG3-amine (21.8 g, 74.4 mmol) was dissolved in DCM (200 mL). Next, Ethyl trifluoroacetate (13.3 mL, 112 mmol) and triethylamine (15.5 mL, 112 mmol) were added, and the reaction mixture was stirred at room temperature over the weekend. As follows, the reaction mixture was washed with 10% potassium bisulphate solution (2×200 mL) and saturated sodium bicarbonate solution (2×200 mL), dried over Na₂SO₄, filtered, and evaporated in vacuo. The residue was purified by flash chromatography (ethyl acetate-heptane gradient, 30:70 (v/v) rising to 100% ethyl acetate) to give the title compound (15.1 g, 52%) as a colourless oil. Purity based on LC-MS (method 1) 100%.

LRMS (m/z): 289/333/411 [M-99/M-55/M+23]¹⁺

LC-MS r.t. (min): 1.77

### Intermediate 2:

### tert-butyl N-{2-[2-(2-{2-[N-(2-{2-[2-(2-azidoethoxy) ethoxy]ethoxy}ethyl)-2,2,2-trifluoroacetamido]ethoxy}ethoxy) ethoxy]ethyl}carbamate

Mesyl-PEG4-azide (16.5 g, 55.5 mmol) and tert-butyl N-[2-(2-{2-[2-(2,2,2-trifluoroacetamido) ethoxy]ethoxy}ethoxy)ethyl]carbamate (15.0 g, 38.6 mmol) were dissolved in DMF (200 mL). Next, cesium carbonate (25.2 g, 77.3 mmol) was added, and the reaction mixture was stirred at 80° C. After 7 hours the reaction mixture was evaporated in vacuo. The crude product was suspended in ethyl acetate (300 mL) and the resulting mixture was washed with 10% potassium bisulphate solution (200 mL). The water layer was washed with ethyl acetate (100 mL). The combined organic layers were dried over Na₂SO₄, filtered, and evaporated in vacuo. The residue was purified by flash chromatography (DCM-10% methanol in DCM (v/v) gradient 100:0 rising to 0:100) to give the title compound (14.7 g, 65%) as a slightly yellowish oil. Purity based on LC-MS (method 1) 92%.

LRMS (m/z): 490/613 [M-99/M+23]¹⁺

LC-MS r.t. (min): 2.02

### Intermediate 3:

### tert-butyl N-(23-azido-3,6,9,15,18,21-hexaoxa-12-azatricosan-1-yl) carbamate

A 4 M sodium hydroxide solution (37.1 mL, 148 mmol) was added to methanol (148 mL). Next, this solution was diluted with 1,4-dioxane (557 mL). The resulting mixture was added to tert-butyl N-{2-[2-(2-{2-[N-(2-{2-[2-(2-azidoethoxy) ethoxy]ethoxy}ethyl)-2,2,2-trifluoroacetamido]ethoxy}ethoxy) ethoxy]ethyl}carbamate (12.5 g, 21.2 mmol), and the reaction mixture was stirred at room temperature. After 2 hours the reaction mixture was evaporated in vacuo. The residue was dissolved in DCM (100 mL) and the resulting mixture was washed with saturated sodium bicarbonate solution (100 mL). The water layer was washed with DCM (2×50 mL). The combined organic layers were dried over Na₂SO₄, filtered, and evaporated in vacuo. The crude product (10.65 g, quant.) was used as such in the next step.

LRMS (m/z): 495 [M+1]¹⁺

LC-MS r.t. (min): 1.50 (method 1)

### Intermediate 4:

### (9H-fluoren-9-yl) methyl N-{14-[(2-{2-[2-(2-azidoethoxy) ethoxy]ethoxy}ethyl) (2-{2-[2-(2-{[(tert-butoxy) carbonyl]amino}ethoxy) ethoxy]ethoxy}ethyl) carbamoyl]-3,6,9,12-tetraoxatetradecan-1-yl}carbamate

Fmoc-N-amido-PEG4-acid (10.54 g, 21.62 mmol), tert-butyl N-(23-azido-3,6,9,15,18,21-hexaoxa-12-azatricosan-1-yl) carbamate (10.65 g, 21.58 mmol), EDCI·HCl (4.34 g, 22.65 mmol) and Oxyma Pure (3.37 g, 23.73 mmol) were dissolved in DMF (250 mL) and the reaction mixture was stirred overnight at room temperature. Next, the reaction mixture was evaporated in vacuo. The crude was dissolved in ethyl acetate (200 mL) and the resulting solution was washed with 10% potassium bisulphate solution (200 mL) and saturated sodium bicarbonate solution (2×200 mL). The combined water solution was washed with ethyl acetate (3×200 mL). The combined organic layers were dried over Na₂SO₄, filtered, and evaporated in vacuo. The residue was purified by flash chromatography (DCM-10% methanol in DCM (v/v) gradient 95:5 rising to 20:80) to give the title compound (8.08 g, 39%) as a yellowish oil. Purity based on LC-MS (method 1) 99%.

LRMS (m/z): 864/964/986 [M−99/M+1/M+23]¹⁺

LC-MS r.t. (min): 2.13

### Intermediate 5:

### (9H-fluoren-9-yl) methyl N-{14-[(2-{2-[2-(2-{[(tert-butoxy) carbonyl]amino}ethoxy) ethoxy]ethoxy}ethyl)({2-[2-(2-{2-[{{tricyclo[10.4.0.0^{4,9}]hexadeca-1 (12),4 (9),5,7,13,15-hexaen-10-yn-2-yloxy}carbonyl)amino]ethoxy}ethoxy) ethoxy]ethyl}) carbamoyl]-3,6,9,12-tetraoxatetradecan-1-yl}carbamate

9H-fluoren-9-yl) methyl N-{14-[(2-{2-[2-(2-azidoethoxy) ethoxy]ethoxy}ethyl) (2-{2-[2-(2-{[(tert-butoxy) carbonyl]amino}ethoxy) ethoxy]ethoxy}ethyl) carbamoyl]-3,6,9,12-tetraoxatetradecan-1-yl}carbamate (2.00 g, 2.08 mmol) was dissolved in methanol (20.8 ml) and the flask was flushed with nitrogen. Next, ammonium formate (1.31 g, 20.8 mmol) and zinc dust (1.36 g, 20.8 mmol) were added, and the reaction mixture was stirred at room temperature. After 15 min the solvent was removed under reduced pressure at 30° C. The residue was suspended in a mixture of THF (50 mL) and DCM (50 mL) and stirred at room temperature. After 20 min the suspension was filtered over celite. The filtercake was washed with DCM (20 mL). The combined filtrates were concentrated in vacuo at 30° C. until a volume of approximately 20 mL. To this solution, containing the intermediate amine, was added 11,12-Didehydro-5,6-dihydrodibenzo[a,e]cycloocten-5-yl 4-nitrophenyl carbonate (800 mg, 2.08 mmol), followed by the addition of DIPEA (544 μL, 3.12 mmol). The reaction mixture was stirred at room temperature. After 3.5 hours the reaction mixture was concentrated in vacuo at 30° C. The residue was purified by flash chromatography (DCM-10% methanol in DCM (v/v) gradient 100:0 rising to 50:50) to give the title compound (1.40 g, 57%) as a yellowish oil. Purity based on LC-MS (method 1) 91%.

LRMS (m/z): 882/1084 [M−301/M−99]¹⁺

LC-MS r.t. (min): 2.30

### Intermediate 6:

### (9H-fluoren-9-yl) methyl N-{14-[(2-{2-[2-(2-azaniumylethoxy) ethoxy]ethoxy}ethyl)({2-[2-(2-{2-[{{tricyclo[10.4.0.0^{4,9}]hexadeca-1 (12), 4 (9),5,7,13,15-hexaen-10-yn-2-yloxy}carbonyl)amino]ethoxy}ethoxy) ethoxy]ethyl}) carbamoyl]-3,6,9,12-tetraoxatetradecan-1-yl}carbamate chloride

To (9H-fluoren-9-yl) methyl N-{14-[(2-{2-[2-(2-{[(tert-butoxy) carbonyl]amino}ethoxy) ethoxy]ethoxy}ethyl) ({2-[2-(2-{2-[{{tricyclo[10.4.0.04,9]hexadeca-1 (12), 4 (9),5,7,13,15-hexaen-10-yn-2-yloxy}carbonyl)amino]ethoxy}ethoxy) ethoxy]ethyl}) carbamoyl]-3,6,9,12-tetraoxatetradecan-1-yl}carbamate (1.40 g, 1.18 mmol) was added 4.0 M hydrogen chloride in 1,4-dioxane (22.0 ml, 88.0 mmol), and the resulting reaction mixture was stirred at room temperature. After 1 hour the reaction mixture was evaporated in vacuo and co-evaporated with DCM (3×40 mL) to yield the crude title compound as a yellowish oil, which was used as such in the next step.

LRMS (m/z): 882/1084 [M-201/M+1]¹⁺, M is corresponding to the free-base.

LC-MS r.t. (min): 1.94 (method 1)

### Intermediate 7:

### 4E)-cyclooct-4-en-1-yl N-[2-(2-{2-[2-(1-amino-N-{2-[2-(2-{2-[{{tricyclo[10.4.0.0^{4,9}]hexadeca-1 (12),4 (9),5,7,13,15-hexaen-10-yn-2-yloxy}carbonyl)amino]ethoxy}ethoxy) ethoxy]ethyl}-3,6,9,12-tetraoxapentadecan-15-amido) ethoxy]ethoxy}ethoxy)ethyl]carbamate

To a solution of (9H-fluoren-9-yl) methyl N-{14-[(2-{2-[2-(2-azaniumylethoxy) ethoxy]ethoxy}ethyl) ({2-[2-(2-{2-[{{tricyclo[10.4.0.04,9]hexadeca-1 (12),4 (9),5,7,13,15-hexaen-10-yn-2-yloxy}carbonyl)amino]ethoxy}ethoxy) ethoxy]ethyl}) carbamoyl]-3,6,9,12-tetraoxatetradecan-1-yl}carbamate chloride (1.33 g, 1.18 mmol) and TCO-NHS ester (338 mg, 1.27 mmol) in DMF (7.89 mL) was added DIPEA (249 μL, 1.43 mmol). The resulting reaction mixture was stirred at room temperature. After 1.5 hours diethylamine (3.45 mL, 33.5 mmol) was added, and the resulting mixture was stirred at room temperature. After 1 hour, the reaction mixture was submitted to preparative MP-LC (method 1). Fractions corresponding to the product were immediately pooled together, frozen and lyophilized overnight to give the title compound (746 mg, 62%) as a pale brown oil. Purity based on LC-MS (method 1) 100%.

LRMS (m/z): 1014 [M+1]¹⁺

LC-MS r.t. (min): 1.89

### Intermediate 8:

### TFL-(DIBO)-(TCO)-(Mal)

To a solution of 4E)-cyclooct-4-en-1-yl N-[2-(2-{2-[2-(1-amino-N-{2-[2-(2-{2-[({tricyclo[10.4.0.04,9]hexadeca-1 (12),4 (9),5,7,13,15-hexaen-10-yn-2-yloxy}carbonyl)amino]ethoxy}ethoxy) ethoxy]ethyl}-3,6,9,12-tetraoxapentadecan-15-amido) ethoxy]ethoxy}ethoxy)ethyl]carbamate (55.0 mg, 54.3 μmol) in DMF (2.00 mL) and DIPEA (19.0 μL, 109 μmol) was added N-Succinimidyl 6-maleimidohexanoate (21.8 mg, 70.6 μmol). The resulting mixture was stirred at room temperature. After 1.5 hours the reaction mixture was submitted to preparative MP-LC (method 2). Fractions corresponding to the product were immediately pooled together, frozen and lyophilized overnight to give the title compound (48.6 mg, 74%) as a yellow oil. Purity based on LC-MS (method 1) 93% (major impurity (6%) corresponds to the hydrolyzed maleimide).

LRMS (m/z): 1207/1229 [M+1/M+23]¹⁺

LC-MS r.t. (min): 2.14

### Intermediate 9:

### 2-(dodecyldisulfanyl)pyridine

To a stirring solution of 2,2′-Dipyridyl disulfide (331 mg, 1.50 mmol) in methanol (10.0 mL) under a nitrogen atmosphere at room temperature was added dodecane-1-thiol (300 μl, 1.252 mmol) dropwise, and the mixture was allowed to stir. After 2.5 hours the reaction mixture was evaporated in vacuo. The residue was purified by flash chromatography (ethyl acetate-heptane gradient, 0:100 (v/v) rising to 20% ethyl acetate in heptane) to give the title compound (300 mg, 77%) as a slightly yellowish oil. Purity based on LC-MS (method 1) 100%.

LRMS (m/z): 312 [M+1]¹⁺

LC-MS r.t. (min): 2.69

### Intermediate 10:

### 3-(dodecyldisulfanyl) propanoic acid

To a solution of 2-(dodecyldisulfanyl)pyridine (300 mg, 963 μmol) in ethanol (5.00 mL) was added 3-mercaptopropanoic acid (100 μL, 1.140 mmol) and acetic acid (500 μL). The resulting mixture was stirred at room temperature overnight. Next, the reaction mixture was evaporated in vacuo and co-evaporated with DCM (3×10 mL). The residue was purified by flash chromatography (ethyl acetate-1% (v/v) acetic acid in heptane gradient, 0:100 (v/v) rising to 15:85) to give the title compound (237 mg, 80%) as a white solid. Purity based on LC-MS (method 1) 85% (major impurity (11%) corresponds to the starting disulfide).

LRMS (m/z): 289/329 [M-17/M+23]¹⁺

LC-MS r.t. (min): 2.51

### Intermediate 11:

### 2,5-dioxopyrrolidin-1-yl 3-(dodecyldisulfanyl) propanoate

To a solution of 3-(dodecyldisulfanyl) propanoic acid (209 mg, 682 μmol) and N-Hydroxysuccinimide (190 mg, 1.65 mmol) in DMF (5.00 mL) at 0° C. was added EDCI·HCl (196 mg, 1.02 mmol). The reaction mixture was allowed to reach room temperature and stirred overnight. Next, the reaction mixture was diluted with ethyl acetate (25 mL) and the resulting mixture was washed with water (25 mL), 10% potassium bisulphate solution (35 mL) and brine (25 mL), dried over Na₂SO₄, filtered, and evaporated in vacuo. The residue was purified by flash chromatography (ethyl acetate-heptane gradient, 0:100 (v/v) rising to 50% ethyl acetate in heptane) to give the title compound (239 mg, 87%) as a white solid. Purity based on LC-MS (method 1) 96%.

LRMS (m/z): 426 [M+23]¹⁺

LC-MS r.t. (min): 2.50

### Intermediate 12:

### N,N-bis({2-[3-(acetylsulfanyl) propanamido]ethyl})-6-azidohexanamide

To a solution of N,N-bis(2-aminoethyl)-6-azidohexanamide dihydrochloride (114 mg, 362 μmol) and 4-nitrophenyl 3-(acetylthio) propanoate (303 mg, 1.12 mmol) in DMF (2.00 mL) and methanol (500 μL) at room temperature was added N-Methylmorpholine (642 μL, 5.84 mmol). The resulting mixture was stirred overnight. Next, the reaction mixture was submitted to preparative MP-LC (method 3A). Fractions corresponding to the product were immediately pooled together, frozen and lyophilized overnight to give the title compound (130 mg, 72%) as an off-white solid. Purity based on LC-MS (method 1) 100%.

LRMS (m/z): 503 [M+1]¹⁺

LC-MS r.t. (min): 1.70

### Intermediate 13:

### d2 (SC-SO1861)-NH₂

To a solution of N, N-bis({2-[3-(acetylsulfanyl) propanamido]ethyl})-6-azidohexanamide (9.40 mg, 18.7 μmol) in methanol (300 μL) was added a 1 M solution of sodium hydroxide (41.0 μL, 41.0 μmol). The reaction mixture was shaken for about 1 min and left standing at room temperature. After 40 min a 1 M trimethylphosphine solution in THF (94.0 0 μL, 94.0 μmol) was added. The reaction mixture was shaken for about 1 min and left standing at room temperature. After 30 min the reaction mixture was evaporated in vacuo and the reaction flask was put under an argon atmosphere. Next, SO1861-SC-Mal (90.0 mg, 41.2 μmol) was added, directly followed by a freshly prepared and degassed solution of 20 mM ammonium bicarbonate/acetonitrile (3:1, v/v, 7.00 mL). The mixture was shaken until all solids were dissolved and left standing at room temperature. After 1.5 hours the reaction mixture was lyophilized overnight. As follows, the crude product was dissolved in a freshly prepared solution of 20 mM ammonium bicarbonate/acetonitrile (3:1, v/v, 2.00 mL) and MesNA (10.0 mg, 60.9 μmol) was added. The mixture was shaken for about 1 min and left standing at room temperature. After 30 min the mixture was subjected to preparative LC-MS (method 4A). Fractions corresponding to the product were immediately pooled together, frozen and lyophilized overnight to give the title compound (79.0 mg, 89%) as a white fluffy solid. Purity based on LC-MS (method 2A) 99%.

LRMS (m/z): 2378 [M−2]²⁻

LC-MS r.t. (min): 3.47

### Intermediate 14:

d2 (SC-SO1861)-disulfide

To a solution of d2 (SC-SO1861)-NH₂ (79.0 mg, 16.6 μmol) in DMF (500 μL) and DIPEA (11.6 L, 66.4 μmol) was added 2,5-dioxopyrrolidin-1-yl 3-(dodecyldisulfanyl) propanoate (20.1 mg, 49.8 μmol). The resulting mixture was shaken for about 1 min and left standing at room temperature. After 1 hour the reaction mixture was submitted to preparative LC-MS (method 4B). Fractions corresponding to the product were immediately pooled together, frozen and lyophilized overnight to give the title compound (72.0 mg, 86%) as a white fluffy solid. Purity based on LC-MS (method 2B) 99%.

LRMS (m/z): 2522 [M−2]²⁻

LC-MS r.t. (min): 3.27

### Intermediate 15:

### SOD1-S-S-mTz

To a solution of SOD1 PMO-3′-DSA (86.0 mg, 10.0 μmol) in water (5.00 mL) was added DTT (30.0 mg, 194 μmol). The mixture was shaken until all solids were dissolved and left standing at room temperature. After 2.5 hours the reaction mixture was equally divided and poured in acetonitrile (2×45 mL). The resulting suspensions were shaken and left standing for 30 min. Next, the suspensions were centrifuged (7830 RPM, 5 min). The solutions were decanted, and the residues were treated with acetonitrile (each vial 20 mL) and the mixtures were shaken. The resulting suspensions were centrifuged (7830 RPM, 3 min). The residues were dissolved in water (total 5.00 mL) and the solutions were combined. Next, a solution of 2-[4-(6-methyl-1,2,4,5-tetrazin-3-yl)phenyl]-N-[2-(pyridin-2-yldisulfanyl)ethyl]acetamide (15.9 mg, 40.0 μmol) in acetonitrile/water (14:1, v/v, 2.80 mL) was added. The mixture was shaken for about 1 min and left standing at room temperature. After 30 min the reaction mixture was equally divided and poured in acetonitrile (2×45 mL). The resulting suspensions were shaken and centrifuged (7830 RPM, 3 min). The solutions were decanted, and the residues were treated with acetonitrile (each vial 20 mL). The resulting suspensions were centrifuged (7830 RPM, 3 min). The solutions were decanted, and the residues were dissolved in water/acetonitrile (total 15 mL, 1/1, v/v) and lyophilized overnight to give the title compound (91.9 mg, quant.) as a pink solid. Purity based on LC-MS (method 3A) 96%.

LRMS (m/z): 1469 [M+6]⁶⁺, 1260 [M+7]⁷⁺, 1102 [M+8]⁸⁺, 980 [M+9]⁹⁺, 882 [M+10]¹⁰⁺

LC-MS r.t. (min): 1.77

### Intermediate 16:

### d2 (S—S-SOD1)-DBCO

To a solution of SOD1-S-S-mTz (91.9 mg, 10.4 μmol) in water (2.50 mL) and acetonitrile (1.00 mL) was added a certain of a stock volume solution of (1S,4E)-cyclooct-4-en-1-yl N-[2-(4-{2-azatricyclo[10.4.0.0^{4,9}]hexadeca-1 (12), 4 (9), 5,7,13,15-hexaen-10-yn-2-yl}-N-[2-({[(1S,4E)-cyclooct-4-en-1-yloxy]carbonyl}amino)ethyl]-4-oxobutanamido)ethyl]carbamate (4.35 mg, 6.26 μmol) in acetonitrile (600 μL) and shaken for about 1 min and left standing at room temperature. After every addition, the reaction progress was monitored with LC-MS (method 3). In this way complete conversion to the title product was obtained. The addition of the stock solution was as following; 300 μL, after 15 min-100 μL, after 30 min-25 μL, after 25 min-15 μL, after 25 min-20 μL, after 45 min-10 μL. Next, the reaction mixture was left standing for 2 hours, and afterwards frozen and lyophilized overnight. The crude product was dissolved in acetonitrile/water (1:3, v/v, 3 mL) and the resulting solution was submitted to preparative LC-MS (method 5). Fractions corresponding to the product were immediately pooled together, frozen and lyophilized overnight to give the title compound (68.9 mg, 67%) as a white solid. Purity based on LC-MS (method 3A) 99% (broad peak).

LRMS (m/z): 1143 [M+16]¹⁶⁺, 1075 [M+17]¹⁷⁺, 1016 [M+18]¹⁸⁺

LC-MS r.t. (min): 1.87

### Intermediate 17:

### d2 (S—S-SOD1)-mTz

To a solution of d2 (S—S-SOD1)-DBCO (58.9 mg, 3.23 μmol) in tert-butanol/water (1:3, v/v, 5.00 mL) was added a solution of methyltetrazine-PEG4-azide (6.12 mg, 12.9 μmol) in ethanol (500 μL). The resulting solution was shaken for about 1 min and left standing at room temperature. After 1.5 hours a solution of DBCO-acid (3.94 mg, 12.9 μmol) in DMF/acetonitrile (1:1, v/v, 1 mL). The resulting mixture was shaken for about 1 min and left standing at 4° C. overnight. Next, the mixture was submitted to preparative LC-MS (method 5). Fractions corresponding to the product were immediately pooled together, frozen and lyophilized overnight to give the title compound (43.9 mg, 73%) as a pink solid. Purity based on LC-MS (method 3A) 99% (broad peak).

LRMS (m/z): 1442 [M+13]¹³⁺, 1339 [M+14]¹⁴⁺, 1250 [M+15]¹⁵⁺, 1172 [M+16]¹⁶⁺, 1103 [M+17]¹⁷⁺, 1042 [M+18]¹⁸⁺, 987 [M+19]¹⁹⁺, 938 [M+20]²⁰⁺.

LC-MS r.t. (min): 1.86

### Intermediate 18:

### TFL-(DIBO)-(TCO)-(d2 [SC-SO1861])

To a solution of d2 (SC-SO1861)-disulfide (67.0 mg, 13.3 μmol) in DMF (2.00 mL) was added water (20 μL, 1.11 mmol) and tri-n-butylphosphine (32.8 μL, 133 μmol). The mixture was shaken for about 1 min and left standing at room temperature. After 1 hour the reaction mixture was added to a solution of pentane/diethyl ether (1:1, v/v, 40 mL). The resulting suspension was shaken and centrifuged (7830 RPM, 3 min). The solution was decanted, and the residue was treated with pentane/diethyl ether (1:1, v/v, 40 mL). The resulting mixture was shaken, and the suspension was centrifuged (7830 RPM, 3 min). The solution was decanted, and the residue was dissolved in solution of 20 mM ammonium bicarbonate/acetonitrile (3:1, v/V, 5.00 mL), and directly treated with a solution of TFL (DIBO-TCO-Mal) (17.6 mg, 14.6 μmol) in acetonitrile (133 μL). The resulting mixture was shaken until all solids were dissolved and left standing at room temperature. After 2 hours the reaction mixture was submitted to preparative LC-MS (method 4B). Fractions corresponding to the product were immediately pooled together, frozen and lyophilized overnight to give the title compound (60.9 mg, 73%) as a white fluffy solid. Purity based on LC-MS (method 2A) 96%.

LRMS (m/z): 2016 [M−3]³⁻

LC-MS r.t. (min): 4.48

### TFL-(DIBO)-(d2 [S—S-SOD1 PMO])-(d2 [SC-SO1861])

To a solution of d2 (S—S-SOD1)-mTz (17.0 mg, 0.908 μmol) in 20 mM ammonium bicarbonate/acetonitrile (3:1, v/v, 2.00 mL) was added TFL-(DIBO)-(TCO)-(d2 [SC-SO1861]) (6.59 mg, 1.09 μmol). The resulting mixture was shaken until all solids were dissolved and left standing at room temperature. After 1 hour the reaction mixture was submitted to preparative LC-MS (method 4A). Fractions corresponding to the product were immediately pooled together, frozen and lyophilized overnight to give the title compound (23.0 mg, 65%) as a white fluffy solid. Purity based on LC-MS (method 3B) 90% (broad peak).

LRMS (m/z): 1458 [M+17]¹⁷⁺, 1377 [M+18]¹⁸⁺, 1304 [M+19]⁺¹⁹, 1239 [M+20]²⁰⁺, 1180 [M+21]⁺²¹

LC-MS r.t. (min): 3.28

### TFL-(DIBO)-(d2 [S—S-SOD1 PMO])-(block) Synthesis

### Intermediate 1:

### TFL-(DIBO)-(TCO)-(Block)

To a solution of thiol-PEG3-alcohol (5.00 mg, 30.1 μmol) in DMF (500 μL) was added TFL-(DIBO)-(TCO)-(Mal) (34.0 mg, 28.2 μmol). The reaction mixture was shaken until all solids dissolved and left standing at room temperature. After 1 hour the reaction mixture was submitted to preparative MP-LC (method 3C). Fractions corresponding to the product were immediately pooled together, frozen and lyophilized overnight to give the title compound (48.6 mg, 74%) as a yellow oil. Purity based on LC-MS (method 4) 99%.

LRMS (m/z): 687 [M+2]²⁻, 1373 [M+1]¹⁺

LC-MS r.t. (min): 3.34

### TFL-(DIBO)-(d2 [S—S-SOD1 PMO])-(Block)

To a mixture of TFL-(DIBO)-(TCO)-(block) (2.37 mg, 1.73 μmol) in 20 mM ammonium bicarbonate/acetonitrile (3:1, v/v, 2.00 mL) was added acetonitrile (500 μL) to obtain a solution. Next, d2 (S—S-SOD1)-mTz (26.9 mg, 1.44 μmol) was added and the resulting mixture was shaken until all solids were dissolved, and left standing at room temperature. After 1 hour the reaction mixture was submitted to preparative LC-MS (method 4A). Fractions corresponding to the product were immediately pooled together, frozen and lyophilized overnight to give the title compound (16.3 mg, 57%) as a white fluffy solid. Purity based on LC-MS (method 3B) 99% (broad peak).

LRMS (m/z): 1436 [M+14]¹⁴⁺, 1340 [M+15]¹⁵⁺, 1256 [M+16]¹⁶⁺, 1182 [M+17]¹⁷⁺, 1117 [M+18]¹⁸⁺, 1058 [M+19]¹⁹⁺.

LC-MS r.t. (min): 3.04

### TFL-(DIBO)-(S—S-SOD1 PMO)-(SC-SO1861) Synthesis

### Intermediate 1:

### 9H-fluoren-9-yl) methyl N-(2-{2-[2-(4-{N′-[(tert-butoxy) carbonyl]hydrazinecarbonyl}piperazin-1-yl)-2-oxoethoxy]ethoxy}ethyl) carbamate

To a solution of tert-butyl 2-(piperazine-1-carbonyl) hydrazine-1-carboxylate (336 mg, 1.38 μmol) in DMF (12 mL) was added [2-[2-(Fmoc-amino) ethoxy]ethoxy]acetic acid (529 mg, 1.37 mmol), Oxyma Pure (21.1 mg, 148 μmol) and EDCI·HCl (290 mg, 1.51 mmol). The resulting mixture was stirred at room temperature overnight. Next, the reaction mixture was diluted with ethyl acetate (150 mL) and the resulting mixture was washed with 10% potassium bisulphate solution (150 mL), saturated sodium bicarbonate solution (150 mL) and brine (150 mL), dried over Na₂SO₄, filtered, and evaporated in vacuo. The residue was purified by flash chromatography (DCM-10% methanol in DCM (v/v) gradient 100:0 rising to 0:100) to give the title compound (675 mg, 80%) as an off-white foam. Purity based on LC-MS (method 1) 99%.

LRMS (m/z): 512/612/634 [M−99/M+1/M+23]¹⁺

LC-MS r.t. (min): 1.87

### Intermediate 2:

### (9H-fluoren-9-yl) methyl N-[2-(2-{2-[4-(hydrazinecarbonyl) piperazin-1-yl]-2-oxoethoxy}ethoxy)ethyl]carbamate 2,2,2-trifluoroacetate

To 9H-fluoren-9-yl) methyl N-(2-{2-[2-(4-{N-[(tert-butoxy) carbonyl]hydrazinecarbonyl}piperazin-1-yl)-2-oxoethoxy]ethoxy}ethyl) carbamate (132 mg, 216 μmol) was added TFA (1 mL, 12.98 mmol) and the resulting mixture was stirred at room temperature. After 30 min the reaction mixture was added to a solution of pentane/diethyl ether (1:1, v/v, 20 mL). The resulting suspension was shaken and centrifuged (7830 RPM, 3 min). The solution was decanted, and the residue was treated with pentane/diethyl ether (1:1, v/v, 20 mL). The resulting mixture was shaken, and the suspension was centrifuged (7830 RPM, 3 min). The solution was decanted, and the residue was used as such in the next step. Yield was not determined. Purity based on LC-MS (method 1) 99%.

LRMS (m/z): 512 [M+1]¹⁺, M is corresponding to the free base

LC-MS r.t. (min): 1.59

### Intermediate 3:

### SO1861-SC-NHFmoc

To a solution of (9H-fluoren-9-yl) methyl N-[2-(2-{2-[4-(hydrazinecarbonyl) piperazin-1-yl]-2-oxoethoxy}ethoxy)ethyl]carbamate 2,2,2-trifluoroacetate (135 mg, 216 μmol) in DMF (1.00 mL) was added SO1861 (100 mg, 53.7 μmol) was added. The resulting mixture was shaken until all solids were dissolved and the left standing at room temperature. After 4 hours 4-formylbenzoic acid (100 mg, 667 μmol) was added. The resulting mixture was shaken until all solids were dissolved and the left standing at room temperature. After 2.75 hours the reaction mixture was submitted to preparative MP-LC (method 3A). Fractions corresponding to the product were immediately pooled together, frozen and lyophilized overnight to give the title compound (100 mg, 79%) as white fluffy solid. Purity based on LC-MS (method 2A) 99%.

LRMS (m/z): 2355 [M−1]¹⁻

LC-MS r.t. (min): 3.75

### Intermediate 4:

### SO1861-SC-NH₂

To a solution of SO1861-SC-NHFmoc (100 mg, 42.4 μmol) in DMF (1.60 mL) was added diethylamine (400 μL, 3.83 mmol). The resulting mixture was shaken for about 1 min and left standing at room temperature. After 1 hour the reaction mixture was added to a solution of pentane/diethyl ether (1:1, v/v, 20 mL). The resulting suspension was shaken and centrifuged (7830 RPM, 3 min). The solution was decanted, and the residue was treated with pentane/diethyl ether (1:1, v/v, 20 mL). The resulting mixture was shaken, and the suspension was centrifuged (7830 RPM, 3 min). The solution was decanted, and the residue was used as such in the next step. Yield was not determined.

LRMS (m/z): 2153 [M−1]¹⁻

LC-MS r.t. (min): 3.08 (method 2A)

### Intermediate 5:

### SO1861-SC-disulfide

To a solution of SO1861-SC-NH2 (91.0 mg, 42.4 μmol) in DMF (1.00 mL) was added 2,5-dioxopyrrolidin-1-yl 3-(dodecyldisulfanyl) propanoate (51.6 mg, 128 μmol). The resulting mixture was shaken for about 1 min and left standing at room temperature. After 1 hour the reaction mixture was added to a solution of pentane/diethyl ether (1:1, v/v, 20 mL). The resulting suspension was shaken and centrifuged (7830 RPM, 3 min). The solution was decanted, and the residue was treated with pentane/diethyl ether (1:1, v/v, 20 mL). The resulting mixture was shaken, and the suspension was centrifuged (7830 RPM, 3 min). The solution was decanted, and the residue was dissolved in water/acetonitrile (1:1, v/v, 4 mL) and lyophilized overnight to give the title compound (102 mg, 98%) as white fluffy solid. Purity based on LC-MS (method 2B) 94%.

LRMS (m/z): 2422 [M−1]¹⁻

LC-MS r.t. (min): 4.77

### Intermediate 6:

### TFL-(DIBO)-(TCO)-(SC-SO1861)

To a solution of SO1861-SC-disulfide (50.0 mg, 20.6 μmol) was added water (15 μL, 833 μmol) and tri-n-butylphosphine (25.4 μL, 103 μmol). The mixture was shaken for about 1 min and left standing at room temperature. After 30 min the reaction mixture was added to a solution of pentane/diethyl ether (1:1, v/v, 30 mL). The resulting suspension was shaken and centrifuged (7830 RPM, 3 min). The solution was decanted, and the residue was treated with pentane/diethyl ether (1:1, v/v, 30 mL). The resulting mixture was shaken, and the suspension was centrifuged (7830 RPM, 3 min). The solution was decanted, and a solution of TFL (DIBO-TCO-Mal) (29.9 mg, 24.8 μmol) in DMF (1.50 mL) was added. The mixture was shaken until all solids were dissolved and left standing at room temperature. After 1 hour the reaction mixture was submitted to preparative MP-LC (method 3B). Fractions corresponding to the product were immediately pooled together, frozen and lyophilized overnight to give the title compound (48.5 mg, 69%) as white fluffy solid. Purity based on LC-MS (method 2A) 100%.

LRMS (m/z): 1714 [M−2]²⁻, 2284 [M−3]³⁻

LC-MS r.t. (min): 4.58

### TFL-(DIBO)-(S—S-SOD1 PMO)-(SC-SO1861)

To a solution of SOD1-S-S-mTz (7.21 mg, 0.818 μmol) in 20 mM ammonium bicarbonate/acetonitrile (3:1, v/v, 800 μL) was added TFL-(DIBO)-(TCO)-(SC-SO1861) (3.37 mg, 0.982 μmol). The resulting mixture was shaken until all solids were dissolved and left standing at room temperature. After 30 min the reaction mixture was submitted to preparative LC-MS (method 4C). Fractions corresponding to the product were immediately pooled together, frozen and lyophilized overnight to give the title compound (6.69 mg, 67%) as a white fluffy solid. Purity based on LC-MS (method 2A) 96% (multiple (broad) peaks due to regioisomers).

LRMS (m/z): 2443 [M+5]⁵⁺, 2036 [M+6]⁶⁺, 1745 [M+7]⁷⁺

LC-MS r.t. (min): 3.87

### TFL-(DIBO)-(S—S-SOD1 PMO)-(Block)

To a solution of SOD1-S-S-mTz (6.61 mg, 0.750 μmol) in 20 mM ammonium bicarbonate/acetonitrile (3:1, v/v, 800 L) was added TFL-(DIBO)-(TCO)-(block) (1.03 mg, 0.750 μmol). The resulting mixture was shaken until all solids were dissolved and left standing at room temperature. After 30 min the reaction mixture was submitted to preparative LC-MS (method 4C). Fractions corresponding to the product were immediately pooled together, frozen and lyophilized overnight to give the title compound (4.83 mg, 63%) as a white fluffy solid. Purity based on LC-MS (method 2A) 98% (multiple (broad) peaks due to regioisomers).

LRMS (m/z): 2031 [M+5]⁵⁺, 1693 [M+6]⁶⁺

LC-MS r.t. (min): 3.94

aCD71-SOD1 ASO

To aCD71-IgG (4 mg, 26.7 nmol, 445 μL, 9 mg/mL in DBPS) was added an aliquot of freshly prepared SMCC solution in DMSO (0.08 mg, 0.24 μmol, 9 mole equivalents, 4 μL, 20 mg/ml in DMSO), the mixture vortexed briefly then incubated for 60 minutes at 20° C. with a shaker at 800 rpm. Separately, SOD1 ASO (2 mg, 268 nmol, 10 mole equivalents) was dissolved in 1 mL DBPS pH 7.5. To this solution was added TCEP (1.3 mg, 4.5 μmol, 169 mole equivalents), the mixture vortexed briefly then incubated for 60 minutes at 20° C. with a shaker at 800 rpm. After incubation, the ASO was purified via centrifugal filtration using an Amicon Ultra 0.5 mL filter vial with a MWCO of 3 kDa. The mix was washed in 4 cycles by centrifugal filtration at 13000 rpm, 15° C., 4 min per cycle using DPBS for washing and discarding the filtrate after each cycle, to afford 800 μL of reduced ASO-SH solution.

To the aCD71-IgG-SMCC solution (26.7 nmol, 450 μL) was added the ASO-SH solution (1.26 mg, 169 nmol, 6.3 mole equivalents, 800 μL), the mixture vortexed briefly then incubated overnight at 20° C. with a shaker at 800 rpm. After ca. 16 hours, an 800 μL aliquot of the conjugate reaction mixture was purified over Protein A gravity flow chromatography (1.7 mL Pierce Protein A Agarose resin, 12-19 mg human IgG per mL resin binding capacity, 20 mL column) to give purified aCD71-SOD1-ASO conjugate (1.1 mg, 0.76 mg/mL, 44% yield). DAR was estimated based on SDS-PAGE analysis of purified conjugates and the increment of the 260 nm signal in UV-Vis analysis compared to the signal of the native antibody.

aCD71-PMO (1) and aCD71-PMO (2)

The method is exemplary described for aCD71-PMO (1). The conjugate aCD71-PMO (2) was produced using the very same methodology.

To aCD71-IgG (4 mg, 26.7 nmol, 445 μL, 9 mg/mL in DBPS) was added an aliquot of freshly prepared SMCC solution in DMSO (0.04 mg, 0.12 μmol, 4.5 mole equivalents, 2 μL, 20 mg/ml in DMSO), the mixture vortexed briefly then incubated for 60 minutes at 20° C. with a shaker at 800 rpm.

Separately, PMO (1) (2.75 mg, 323 nmol, 12 mole equivalents) and TCEP (1.3 mg, 4.5 μmol, 169 mole equivalents) were dissolved in 200 μL DBPS pH 7.5. The mixture vortexed briefly then incubated for 60 minutes at 20° C. with a shaker at 800 rpm. After incubation, the PMO was purified via centrifugal filtration using an Amicon Ultra 0.5 mL filter vial with a MWCO of 3 kDa. The mix was washed in 4 cycles by centrifugal filtration at 13000 rpm, 15° C., 4 min per cycle using DPBS for washing and discarding the filtrate after each cycle, to afford 200 μL of reduced PMO (1)-SH solution.

To the aCD71-IgG-SMCC solution (26.7 nmol, 450 μL) was added the PMO (1)-SH solution (323 nmol, 12 mole equivalents, 200 μL), the mixture vortexed briefly then incubated overnight at 20° C. with a shaker at 800 rpm. After ca. 16 hours, a 300 mL aliquot of the conjugate reaction mixture was purified over Protein A agarose resin suspension (300 μL Pierce Protein A Agarose resin suspension, 12-19 mg human IgG per mL resin binding capacity) to give purified aCD71-PMO (1) conjugate. DAR was estimated based on SDS-PAGE analysis of purified conjugates and the increment of the 260 nm signal in UV-Vis analysis compared to the signal of the native antibody.
aCD71-PMO (1): 0.5 mg, 1.2 mg/mL, 26% yield
aCD71-PMO (2): 0.26 mg, 0.64 mg/mL, 13% yield
   aCD71-azide

aCD71-IgG (70 mg, 0.467 μmol) was buffer exchanged into TBS 1× using a vivaspin (6 mL, 50 kDa MWCO), to a final concentration of 10.0 mg/mL. The monoclonal antibody was modified using seven GlyCLICK® 10 mg azide activation kits (Genovis) following instructions reported in the GlyCLICK® 10 mg azide activation kit (Genovis).

The Immobilized GlycINATOR column that trims off the glycans from the Fc domain of the IgG was equilibrated to room temperature before use and was placed in a 15 ml collection tube. The column was centrifuged at 100×g for 1 min to remove the storage solution and flow-through was discarded. After placing the column in the collection tube, 2.5 ml 1×TBS buffer was added on top of the resin and the column was centrifuged at 100×g for 1 minute and the flowthrough was discarded. The bottom cap was put on the column and the sample volume was adjusted to the maximum volume, 1 ml, and immediately the antibody solution was added to the column. The column was sealed then incubated by end-over-end mixing at room temperature for 60 minutes. After incubation, the column was centrifuged at 100×g for 1 minute to elute the antibody sample and 500 μL 1×TBS was added to the column. The column sealed and inverted a couple of times before removing the bottom cap and centrifuging it at 100×g for 1 minute to elute the antibody sample. Eluates were pooled resulting in a total sample volume of approximately 2.5 ml. UDP-GalNAz was reconstituted with 250 μL TBS and transferred to the pooled antibody eluates. To the mixture was added 200 μL of GalT and the mixture was incubated overnight at 30° C. After incubation overnight, the reaction mixture was purified via centrifugal filtration using the column included in the GlyCLICK® 10 mg azide activation kits (Genovis). The reaction mixture was applied in the resin of the column, and the column was centrifuged at 1000×g for 4 minutes to retain the flow-through that contained the azide-modified antibody. The azido modified antibody was buffer exchanged into DPBS 1× using a vivaspin (20 mL, 50 kDa MWCO), to a final concentration of 10.0 mg/mL. Recovery yield: 68 mg (97%)

aCD71-Malat1 ASO

Malat1-ASO-C6-disulfide (6.8 mg, 0.91 μmol) was dissolved in 2 mL 20 mM ammonium carbonate buffer pH 7.8. To this solution was added TCEP (2.6 mg, 9.1 μmol, 10 mole equivalents), the mixture vortexed briefly then incubated for 120 minutes at 22° C. with a shaker at 800 rpm. The mixture was purified via centrifugal filtration using a Vivaspin column with a MWCO of 3 kDa. To the resulting reduced Malat1-ASO-SH solution was added(S)-2-pyridylthio cysteamine HCl (3 mg, 13.62 μmol, 15 mole equivalents) dissolved in 1 mL deionized water, the mixture vortexed briefly then incubated for 180 minutes at 22° C. with a shaker at 800 rpm, modifying the thiol residue of the ASO into an amine residue. To this mixture was then added a freshly prepared solution of DBCO-PEG4-NHS (35 mg, 54.5 μmol, 60 mole equivalents, 1 mL) in 100 mM ammonium carbonate buffer pH 7.8/DMF (1:1), the mixture vortexed briefly then incubated for 120 minutes at 22° C. with a shaker at 800 rpm. Reaction progress was monitored via RP-HPLC: ACE Excel Super C18 column (7.5×2.1 mm) resulting in a retention time shift for the product to 4.66 min compared to the retention time of starting material Malat1-ASO-C6-disulfide at 4.24 min. The reaction mixture was subjected to gel filtration chromatography (P50) into deionized water followed by concentration with a 3 kDa MWCO vivaspin column to yield Malat1-ASO-S-S-PEG4-DBCO (4 mg, 55% yield).

To aCD71-azide (3.5 mg, 23 nmol, 5 mg/mL, 0.7 mL) dissolved in DPBS was added a solution of Malat1-ASO-S-S-PEG4-DBCO (1.8 mg, 230 nmol, 10 mole equivalents, 2.5 mg/mL, 0.7 mL) dissolved in deionized water, the mixture vortexed briefly then incubated for 72 hours at 22° C. with a shaker at 800 rpm. After incubation, the reaction mixture was subjected to preparative SEC (Superdex 200, 10/300 column) into PBS/10% IPA followed by concentration and buffer exchange into DPBS. The resulting aCD71-Malat1 ASO conjugate obtained an average DAR 1.5 (HIC-UV analysis) and a high monomeric purity of 99% (SEC-UV analysis). Yield: 1.68 mg, 48%.

aCD71-SOD1 PMO (Conjugate 1)

To aCD71-azide (6 mg, 40 nmol, 5 mg/mL, 1.2 mL) dissolved in DPBS pH 7.4 was added a solution of TFL-(DIBO)-(d2 [S—S-SOD1 PMO])-(block) (8 mg, 400 nmol, 10 mole equivalents, 67 mg/mL, 120 μL) dissolved in DMSO, the mixture vortexed briefly then incubated for 120 hours at 37° C. with a shaker at 800 rpm. After incubation, the reaction mixture was subjected to preparative SEC (Superdex 200, 16/600 column) into PBS/10% IPA followed by concentration with Vivaspin 30K MWCO filters and buffer exchange into DPBS via Nap 5 gel filtration column. The resulting aCD71-SOD1 PMO conjugate obtained an average degree of labelling 1.7 (HIC-UV analysis), resulting in an average DAR of 3.4 and a high monomeric purity of 99% (SEC-UV analysis). Yield: 0.83 mg, 14%.

aCD71-SOD1 PMO (Conjugate 2)

To aCD71-azide (6 mg, 40 nmol, 5 mg/mL, 1.2 mL) dissolved in DPBS pH 7.4 was added a solution of TFL-(DIBO)-(S—S-SOD1 PMO)-(block) (3.7 mg, 400 nmol, 10 mole equivalents, 40 mg/mL, 92 μL) dissolved in DMSO, the mixture vortexed briefly then incubated for 72 hours at 22° C. with a shaker at 800 rpm. After incubation, the reaction mixture was subjected to preparative SEC (Superdex 200, 16/600 column) into PBS/10% IPA followed by concentration with Vivaspin 30K MWCO filters and buffer exchange into DPBS via Nap 5 gel filtration column. The resulting aCD71-SOD1 PMO conjugate obtained an average DAR 1.4 (HIC-UV analysis) and a high monomeric purity of 99% (SEC-UV analysis). Yield: 4.5 mg, 75%.

aCD71-(Saponin-SOD1 PMO)_{high}

To aCD71-azide (6 mg, 40 nmol, 5 mg/mL, 1.2 mL) dissolved in DPBS pH 7.4 was added a solution of TFL-(DIBO)-(d2 [S—S-SOD1 PMO])-(d2 [SC-SO1861]) (9.9 mg, 400 nmol, 10 mole equivalents, 83 mg/mL, 120 μL) dissolved in DMSO, the mixture vortexed briefly then incubated for 120 hours at 37° C. with a shaker at 800 rpm. After incubation, the reaction mixture was subjected to preparative SEC (Superdex 200, 16/600 column) into PBS/10% IPA followed by concentration with Vivaspin 30K MWCO filters and buffer exchange into DPBS via Nap 5 gel filtration column. The resulting aCD71-(Saponin-SOD1 PMO)_{high} conjugate obtained an average degree of labelling 1.4 (HIC-UV analysis) resulting in an average DAR 2.8 (HIC-UV analysis) and a high monomeric purity of 99% (SEC-UV analysis). Yield: 0.51 mg, 9%.

aCD71-(Saponin-SOD1 PMO)_{low}

To aCD71-azide (6 mg, 40 nmol, 5 mg/mL, 1.2 mL) dissolved in DPBS pH 7.4 was added a solution of TFL-(DIBO)-(S—S-SOD1 PMO)-(SC-SO1861) (4.88 mg, 400 nmol, 10 mole equivalents, 40 mg/ml, 122 μL) dissolved in DMSO, the mixture vortexed briefly then incubated for 72 hours at 22° C. with a shaker at 800 rpm. After incubation, the reaction mixture was subjected to preparative SEC (Superdex 200, 16/600 column) into PBS/10% IPA followed by concentration with Vivaspin 30K MWCO filters and buffer exchange into DPBS via Nap 5 gel filtration column. The resulting aCD71-(Saponin-SOD1 PMO)_{low} conjugate obtained a DAR 1.2 (HIC-UV analysis) and a high monomeric purity of 99% (SEC-UV analysis). Yield: 5.1 mg, 85%.

### SOD1 PMO (1) and PMO (2)

Phosphorodiamidate morpholino oligomers targeting human (Hs) SOD1 (PMO (1) [SEQ ID: 51], with the sequence and modifications 5′-[AACACCCACCTGCTGTATTATCTCC]-(3′-disulfide amide), and PMO (2) [SEQ ID: 52], with the sequence and modifications 5′-[ATCCTTTGGCCCACCGTGTTTTCTG]-(3′-disulfide amide), both designed to induce SOD1 exon skip, were custom-produced by Gene Tools, LLC, according to methods known in the art.

### SOD1 ASO

An antisense oligonucleotide targeting human (Hs) SOD1 mRNA, SOD1 ASO [SEQ ID NO: 50], with the sequence and modifications (5′-C6-disulfide)-[3*24*42*T*A*9*A*T*T*T*9*T*A*32*4 3*1]-3′; with [1=2′-MOE-5Me-rU; 2=2′MOE-rA, 3=2′MOE-5Me-rC; 4=2′MOE-rG; 9=5-Methyl-dC; *=phosphorothioate], was custom-produced by BioSpring Gesellschaft für Biotechnologie mbH, Germany, according to methods known in the art.

TABLE A6
Primers used in qRT-PCR analysis

| | | |
|---|---|---|
| Mm Ppih | Forward | AATCGAGCTCTTTGCAGACG |
| | | [SEQ ID NO: 53] |
| | Reverse | TATCCTATCGGAACGCCATC |
| | | [SEQ ID NO: 54] |
| Mm Ndufa2 | Forward | TTCGCGTTCACTTATGCCAG |
| | | [SEQ ID NO: 55] |
| | Reverse | GCCTTCTTCAGCTCCACGTA |
| | | [SEQ ID NO: 56] |
| Mm Malat1 | Forward | AGGCGGGCAGCTAAGGA |
| | | [SEQ ID NO: 17] |
| | Reverse | CCCCACTGTAGCATCACATCA |
| | | [SEQ ID NO: 18] |
| Mm Sod1 | Forward | GGAACCATCCACTTCGAGCA |
| | | [SEQ ID NO: 57] |
| | Reverse | GGTACAGCCTTGTGTATTGTCC |
| | | [SEQ ID NO: 58] |
| Mm Ppia | Forward | GCGGCAGGTCCATCTACG |
| | | [SEQ ID NO: 4] |
| | Reverse | GCCATCCAGCCATTCAGTC |
| | | [SEQ ID NO: 5] |
| Mm Sdha | Forward | GAGGAAGCACACCCTCTCAT |
| | | [SEQ ID NO: 6] |
| | Reverse | GGAGCGGATAGCAGGAGGTA |
| | | [SEQ ID NO: 7] |
| Hs SDHA | Forward | GCATTTGGCCTTTCTGAGGC |
| | | [SEQ ID NO: 13] |
| | Reverse | CTCCATGTTCCCCAGAGCAG |
| | | [SEQ ID NO: 14] |
| Hs IPO8 | Forward | CAGCAGCCCAGACTCTCTTA |
| | | [SEQ ID NO: 42] |
| | Reverse | CCTAGGGTCAAAGTTCGGGT |
| | | [SEQ ID NO: 43] |
| Hs MALAT1 | Forward | AAAGCAAGGTCTCCCCACAAG |
| | | [SEQ ID NO: 44] |
| | Reverse | GGTCTGTGCTAGATCAAAAGGC |
| | | [SEQ ID NO: 45] |
| Hs SOD1 | Forward | AGCAGATGACTTGGGCAAAG |
| | | [SEQ ID NO: 46] |
| | Reverse | ACCACAAGCCAAACGACTTC |
| | | [SEQ ID NO: 47] |

TABLE A7
Primers used in gel analysis

| | | |
|---|---|---|
| Mm SOD1 | Forward | CGTCTTGCTCTCTCTGGTCC |
| | | [SEQ ID NO: 21] |
| | Reverse | AGACTCAGACCACACAGGGA |
| | | [SEQ ID NO: 22] |
| Hs SOD1 | Forward | CGACGAAGGCCGTGTGCGTG |
| | | [SEQ ID NO: 48] |
| | Reverse | AGTTAAGGGGCCTCAGACTACA |
| | | [SEQ ID NO: 49] |

### Cell Treatment of Neuro-2a Cells for Malat1 or Sod1 Silencing and Sod1 Exon Skip

Neuro-2a cells (murine neuroblastoma cell line) were cultured in DMEM (PAN-Biotech GmbH) supplemented with 10% fetal bovine serum (FBS, PAN-Biotech GmbH) and Pen/Strep (PAN-Biotech GmbH). For experiments, Neuro-2a cells were harvested, resuspended at 60.000 cells/mL and seeded at a density of 36.000 cells/well or 6.000 cells/well for the 24 or 96-well plates (Greiner BioOne). Cells were incubated overnight at 37° C. Before the start of treatment, either 210 μL or 35 μL media was added per well resp., followed by the compounds from a 10× concentrated stock solution in DPBS (PAN-Biotech GmbH). In the control wells, or when only one compound was applied in treatment, additional DPBS was added so that the final volume per well was 900 UL or 150 μL for the 24 or 96-well plates (wp), respectively. Plates were incubated for 72 hr at 37° C. before the cells of the 24 wp were harvested for gene expression analysis or exon skip/aberrant transcript analysis and the cell viability was assessed on the 96 wp.

### Cell Treatment SOD1 Exon Skip or SOD1 Silencing in ARPE-19 Cells

ARPE-19 cells (human retinal pigment epithelia cell line) were cultured in DMEM (PAN-Biotech GmbH) supplemented with 10% fetal bovine serum (FBS, PAN-Biotech GmbH) and Pen/Strep (PAN-Biotech GmbH). For experiments, ARPE-19 cells were harvested, resuspended at 133.333 cells/mL and seeded at a density of 80.000 cells/well or 13.333 cells/well for the 24 or 96-well plates (Greiner BioOne), respectively. Cells were incubated overnight at 37° C. Before the start of treatment, 210 μL or 35 μL media was added per well resp., followed by saponin and/or other compounds from a 10× concentrated stock solution in DPBS (PAN-Biotech GmbH). In the control wells, or when only one compound was applied in treatment, additional DPBS was added so that the final volume per well was 900 μL or 150 μL for the 24 or 96-well plates (wp), respectively. Plates were incubated for 72 hr at 37° C. before the cells of the 24 wp were harvested for gene expression analysis and the cell viability was assessed on the 96 wp.

### Cell Viability Assay (MTS)

The cells were treated for 72 hr at 37° C. before the cell viability was determined by an MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. Treatment medium was removed, after which 100 μL diluted MTS solution was added per well. The plate was incubated for approximately 20-30 minutes at 37° C. Subsequently, the OD at 492 nm was measured on a Spectramax iD5 plate reader (Molecular Devices). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

RNA Isolation and Quantitative Gene Expression Analysis from Cells

Total RNA from cells was isolated using TRIzol™ Reagent (Thermo Scientific) according to the manufacturer's instruction. Conversion into cDNA was performed using iScript™ cDNA Synthesis Kit (BioRad) using standard protocols. Gene expression levels of the gene of interest (GOI) and levels of specific housekeeping genes were determined using quantitative real-time PCR assays (qRT-PCR) using iTaq™ Universal SYBR® Green Supermix (BioRad) and the LightCycler 480 II (Roche Diagnostics) with specific DNA primers, listed in Table A6. Each analysis reaction was performed in triplicate. Analysis was done by the ΔCt method to determine GOI expression relative to 2 specific housekeeping control mRNAs. Results are expressed as % relative GOI expression levels after normalization to DPBS treated control cells.

RNA Isolation and Gel Analysis from Cells

Total RNA from cells was isolated using TRIzol™ Reagent (Thermo Scientific) according to the manufacturer's instruction. Conversion into cDNA was performed using iScript™ cDNA Synthesis Kit (BioRad) using standard protocols. The gene expression was determined on 50 ng cDNA using the SapphireAmp Fast PCR Master Mix (Takara) with specific DNA primers, listed in Table A7. The PCR product was separated on a 2% agarose gel and fragments were analysed on the ChemiDoc XRS+ (BioRad) using Image Lab software (BioRad). For quantification of the exon skip or aberrant transcript, the total signal intensity of the skipped fragment(s) was divided over the total signal intensity of the skipped fragment(s)+full length fragment (496 bp)×100. Hs SOD1: 299 bp (exon 2 skip), 426 bp (exon 3 skip), 329 bp (exon 2+exon 3 skip), and 496 bp (full length fragment) or Mm Sod1: 448 bp (exon 2 skip), 378 bp (exon 2+exon 3 skip), and 545 bp (full length fragment).

### In Vivo Study Design

Male C57BI/6 (N=33; 8-9 weeks at arrival) were randomly allocated to 11 treatment groups (n=3). On study day 0, mice received a unilateral intracerebroventricular (ICV; right lateral ventricle) administration of 10 μL allocated treatment solution, according to Table A8. In group D one mouse received the unilateral intracerebroventricular administration into the right lateral ventricle, and was therefore excluded from analysis. Mice were terminated at study day 10 post dosing. The whole brain was dissected immediately while keeping the tissue at 4° C. First, the cerebellum was dissected. Following that, the brain was hemisected longitudinally and striatum (left and right pooled together in one vial), thalamus (left and right pooled together in one vial), cerebral cortex (left and right separated in two individual vials), hippocampus (left and right separated in two individual vials) and brainstem (pons+medulla; left and right brainstem) were dissected. The remaining brain areas (predominantly hypothalamus and midbrain) were collected as well and pooled in one vial. All samples were preserved in RNAlater for 24 h at 4° C. and subsequently frozen at −80° C. until analysis for the expression of Malat1 or Sod1 RNA levels.

### RNA Analysis Brain Tissues

Total RNA was isolated from the frozen sections of mouse brain regions using the TissueLyser II (Qiagen) as homogenizer and TRIzol™ Reagent (Thermo Scientific) according to the manufacturer's instruction. Conversion into cDNA was performed using iScript™ cDNA Synthesis Kit (BioRad) using standard protocols. Murine (Mm) Malat1 and Sod1 expression levels and levels of housekeeping genes were determined using quantitative real-time PCR assays (qRT-PCR) using iTaq™ Universal SYBR® Green Supermix (BioRad) and the Light Cycler 480 II (Roche Diagnostics) with specific DNA primers, listed in Table A6. Each analysis reaction was performed in triplicate. Analysis was done by the ΔCt method to determine GOI expression relative to 2 specific housekeeping control mRNAs. Results are expressed as % relative GOI expression levels after normalization to the vehicle control.

For statistical analysis between different brain regions a one-way ANOVA (ordinary model) was used, including post-hoc Tukey's multiple comparisons testing between groups per brain region. Data was considered significant when p<0.05.

### REFERENCES

Aguayo Bonniard A, Yeung J Y, Chan C C, et al. Ocular surfacetoxicity from glaucoma topical medications and associated preservatives such as benzalkonium chloride (BAK). ExpertOpin Drug Metab Toxicol. 2016; 12:1279-1289.;
Ashok A., Singh N., Chaudhary S., Bellamkonda V., Kritikos A. E., Wise A. S., Rana N., McDonald D., Ayyagari R. Retinal Degeneration and Alzheimer's Disease: An Evolving Link. Int. J. Mol. Sci. 2020; 21:7290. doi: 10.3390/ijms21197290.
Bremner R and Sage J, The origin of human retinoblastoma, Nature, 2014 Oct. 16; 514 (7522): 312-313. doi: 10.1038/nature13748;
Cabrera F. J. et al., Challenges and opportunities for drug delivery to the posterior of the eye. Drug Discov Today. 2019 August; 24 (8): 1679-1684. doi: 10.1016/j.drudis.2019.05.035
Cai X et al. Highly Efficient and Safe Delivery of VEGF siRNA by Bioreducible Fluorinated Peptide Dendrimers for Cancer Therapy. ACS Appl Mater Interfaces 2017; 9 (11): 9402-9415. doi: 10.1021/acsami.6b16689
Canosa et al. Amyotrophic lateral sclerosis with SOD1 mutations shows distinct brain metabolic changes. European Journal of Nuclear Medicine and Molecular Imaging (2022) 49:2242-2250
Cardamone M, et al., 2008-Cardamone M; Darras B T; Ryan M M, 2008, ‘Inherited myopathies and 15 muscular dystrophies’, Seminars in Neurology, vol. 28, pp. 250-259, http://dx.doi.org/10.1055/s-2008-1062269
Carrella et al., Sophisticated Gene Regulation for a Complex Physiological System: The Role of Non-coding RNAs in Photoreceptor Cells. Front Cell Dev Biol. 2021 Jan. 18:8:629158. doi: 10.3389/fcell.2020.629158. eCollection 2020.
Cassano et al., Biomaterials for Drugs Nose-Brain Transport: A New Therapeutic Approach for Neurological Diseases Materials 2021, 14, 1802. https://doi.org/10.3390/ma14071802
Chan J. W., Chan N. C., Sadun A. A. Glaucoma as Neurodegeneration in the Brain. Eye Brain. 2021; 13:21-28. doi: 10.2147/EB.S293765.
Clochard J et al, A new acetylated triterpene saponin from Agrostemma githago L. modulates gene delivery efficiently and shows a high cellular tolerance, International Journal of Pharmaceutics, Volume 589, 15 Nov. 2020, 119822.
Crooke A. et al., Silencing of P2Y2 receptor delays Ap4A-corneal re-epithelialization process. Molecular Vision 2009; 15:1169-1178
Cynthia Yu-Wai-Man et al., RNA therapeutics in ophthalmology-translation to clinical trials. Experimental Eye Research, 2021, 205:108482. doi: 10.1016/j.exer.2021.108482
De Vries, V. A. et al. The effects of intravitreal injections on intraocular pressure and retinal nerve fiber layer: a systematic review and meta-analysis. Scientific Reports| (2020) 10:13248-13258. Doi: 10.1038/s41598-020-70269-7
Di Virgilio, F. Novartis Found Symp; Purinergic signalling between axons and microglia. 2006; 276:253-8; discussion 259-62, 275-81. doi: 10.1002/9780470032244.ch20.
Dong A, et al. Superoxide dismutase 1 protects retinal cells from oxidative damage. J Cell Physiol 2006 September; 208 (3): 516-26. doi: 10.1002/jcp.20683
Falavarjani K. G. et al., Adverse events and complications associated with intravitreal injection of anti-VEGF agents: a review of literature. Eye 2013, 27:787-794.
Fleck et al. (2019) in Table 1, row no. 28-Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi: 10.3390/molecules24010171
Fuchs H, et al. Glycosylated Triterpenoids as Endosomal Escape Enhancers in Targeted Tumor Therapies. Biomedicines 5 (2017)
Geroski D H, Edelhauser H F. Transscleral drug delivery for posterior segment disease. Adv Drug Deliv Rev 2001; 52 (1): 37-48. doi: 10.1016/S0169-409X (01) 00193-4.
Ghoraba H. H. et al. Ocular Gene Therapy: A Literature Review with Special Focus on Immune and Inflammatory Responses. Clin Ophthalmol. 2022; 16:1753-1771. doi: 10.2147/OPTH.S364200
Guidoboni G, et al., Neurodegenerative Disorders of the Eye and of the Brain: A Perspective on Their Fluid-Dynamical Connections and the Potential of Mechanism-Driven Modeling. Front Neurosci. 2020 Nov. 12:14:566428. doi: 10.3389/fnins.2020.566428
Guzman-Aranguez A et al., 2013, British Journal of Pharmacology, 170:730-747. Doi: 10.1111/bph.12330.
Gilleron J, et al. Image based analysis of lipid nanoparticle-mediated siRNA delivery, intracellular trafficking and endosomal escape. Nat Biotechnol 31:638-646 (2013)
Hong S, Kaer, Van, L; Immune Privilege: Keeping an Eye on Natural Killer T Cells; J Exp Med (1999) 190 (9): 1197-1200. https://doi.org/10.1084/jem.190.9.1197
Horani et al., 2019. Macular Atrophy of the Retinal Pigment Epithelium in Patients with Neovascular Age-Related Macular Degeneration: What is the Link? Part I: A Review of Disease Characterization and Morphological Associations. Ophthalmol Ther (2019) 8:235-249
https://doi.org/10.1007/s40123-019-0177-7
Horani et al., 2020. A Review of Macular Atrophy of the Retinal Pigment Epithelium in Patients with Neovascular Age-Related Macular Degeneration: What is the Link? Part II. Ophthalmol Ther (2020) 9:35-75. https://doi.org/10.1007/s40123-019-00227-8
Imamura Y et. al., 2006 Drusen, choroidal neovascularization, and retinal pigment epithelium dysfunction in SOD1-deficient mice: A model of age-related macular degeneration. Proc Natl Acad Sci USA. 2006 Jul. 25; 103 (30): 11282-7. doi: 10.1073/pnas.0602131103
Jia et al., Major Triterpenoid Saponins from Saponaria officinalis, J. Nat. Prod. 1998, 61, 11, 1368-1373, Publication Date: Sep. 19, 1998, https://doi.org/10.1021/np980167u
Jindal V. Interconnection Between Brain and Retinal Neurodegenerations. Mol. Neurobiol. 2015; 51:885-892. doi: 10.1007/s12035-014-8733-6.
Kabat, E. A., Wu, T. T., Perry, H. M., Gottesman, K. S. and Foeller, C. (1991) Sequences of Proteins of Immunological Interest. NIH publication No. 91-3242, Bethesda.
Kevany B. M., Palczewski K. Phagocytosis of retinal rod and cone photoreceptors. Physiology (Bethesda) 2010; 25:8-15. doi: 10.1152/physiol.00038.2009
Kipriyanov, S. M., et al. (1994) Mol. Immunol. 31:1047-1058
Kipriyanov, S. M., et al. (1995) Human Antibodies and Hybridomas 6:93-101
Kochetkov N. K.; Khorlin A. J.; Ovodov J. S (1963). The structure of gypsoside-triterpenic saponin from gypsophila pacifica kom., 4 (8), 477-482. doi: 10.1016/s0040-4039 (01) 90658-6
Khorkova O., Wahlestedt C. Oligonucleotide therapies for disorders of the nervous system. Nat. Biotechnol. 2017; 35:249-263. doi: 10.1038/nbt.3784
Lam J K D et al., Mol Ther Nucleic Acids., 2015, doi: 10.1038/mtna.2015.23
Lee C S, et al. Adenovirus-Mediated Gene Delivery: Potential Applications for Gene and Cell based Therapies in the New Era of Personalized Medicine. Genes Dis 4:43-63 (2017).
Liebmann J M et al., 2020, Evolving Guidelines for Intracameral Injection; Journal of Glaucoma 29 Suppl 1:1; DOI: 10.1097/IJG.0000000000001451.
Liu K, et al., MiR-124 regulates early neurogenesis in the optic vesicle and forebrain, targeting NeuroD1. Nucleic Acids Res. 2011 April; 39 (7): 2869-79. doi: 10.1093/nar/gkq904. Epub 2010 Dec. 3.
Marchesi N, Fahmideh F, Boschi F, Pascale A, Barbieri. Ocular Neurodegenerative Diseases: Interconnection between Retina and Cortical Areas, Cells, 2021 Sep. 12; 10 (9): 2394. doi: 10.3390/cells10092394.
Mali P, et al. RNA-guided human genome engineering via Cas9. Science 339:823-826 (2013)
Martin-Gil A. et al., Silencing of P2Y2 receptors reduces intraocular pressure in New Zealand rabbits. British Journal of Pharmacology (2012) 165 1163-1172. doi: 10.1111/j.1476-5381.2011.01586.x
Mendell, J. R. et al., Assessment of systemic delivery of rAAVrh74.MHCK7.micro-dystrophin in children with duchenne muscular dystrophy: a nonrandomized controlled trial. JAMA Neurol. Volume 77 (9), pp. 1122-1131 (2020), doi: 10.1001/jamaneurol.2020.1484.]
Mitusova et al., 2022 Overcoming the blood-brain barrier for the therapy of malignant brain tumor: current status and prospects of drug delivery approaches. Journal of Nanobiotechnology volume 20, Article number: 412 (2022).
Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
Nasrolahi et al., 2023 Potential roles of lncRNA MALAT1-miRNA interactions in ocular diseases. J. of Cell Comm, and Signaling (2023) 17:1203-1217. https://doi.org/10.1007/s12079-023-00787-2
Nguyen C, Acosta M, Di Angelantonio S, Salt, T. Seeing Beyond the eye: The Brain Connection; Neurosci., 29 Jun. 2021; Sec. Neurodegeneration; Volume 15-2021|https://doi.org/10.3389/fnins.2021.719717
Purves D, Augustine G J, Fitzpatrick D, et al., editors. Neuroscience. 2nd edition. Sunderland (MA): Sinauer Associates; 2001 Chapter: The retina
Rita Machado A., Carvalho Pereira A., Ferreira F., Ferreira S., Quendera B., Silva E., Castelo-Branco M. Structure-Function Correlations in Retinitis Pigmentosa Patients with Partially Preserved Vision: A Voxel-Based Morphometry Study. Sci. Rep. 2017; 7:11411. doi: 10.1038/s41598-017-11317-7
Roberts T C et al., 2020, Nature Reviews|Drug Discovery, 19:673-694. Doi: 10.1038/s41573-020-0075-7.
Sama S et al., Sapofectosid-Ensuring non-toxic and effective DNA and RNA delivery, International Journal of Pharmaceutics, Volume s 1-2, 20 Dec. 2017, Pages 195-205 (dx.doi.org/10.1016/j.ijpharm.2017.10.016)
Sama S et al., Structure-Activity Relationship of Transfection-Modulating Saponins-A Pursuit for the Optimal Gene Trafficker, Planta Med. Volume 85, pp. 513-518, 2019 (doi: 10.1055/a-0863-4795)
Sama S, et al. Targeted suicide gene transfections reveal promising results in nu/nu mice with aggressive neuroblastoma. J Control Release 275:208-216 (2018)
Singh M. et al., Genes and genetics in eye diseases: a genomic medicine approach for investigating hereditary and inflammatory ocular disorders. Int. J. Ophthalmol 2018, 11 (1): 117-134
Sohn, E. H., Mullins, R. F., & Stone, E. M. (2013). Macular Dystrophies. Retina, 852-890. doi: 10.1016/b978-1-4557-0737-9.00042-4
Soldatov V. O., Kukharsky M. S., Belykh A. E., Sobolev A. M., Deykin A. V. Retinal Damage in Amyotrophic Lateral Sclerosis: Underlying Mechanisms. Eye Brain. 2021; 13:131-146. doi: 10.2147/EB.S299423
Song J, & Kim Y K, Targeting non-coding RNAs for the treatment of retinal diseases. Mol Ther Nucleic Acids 2021 Mar. 1:24:284-293. doi: 10.1016/j.omtn.2021.02.031. eCollection 2021 Jun. 4
Stein J. D., Khawaja A. P., Weizer J. S. Glaucoma in Adults-Screening, Diagnosis, and Management: A Review. JAMA. 2021; 325:164. doi: 10.1001/jama.2020.21899
Strauss O. Physiol Rev. 2005 July; 85 (3): 845-81. doi: 10.1152/physrev.00021.2004
Toonen, L; et al., Intracerebroventricular Administration of a 2′-O-Methyl Phosphorothioate Antisense Oligonucleotide Results in Activation of the Innate Immune System in Mouse Brain; Nucleic Acid Ther. 2018 April; 28 (2): 63-73. doi: 10.1089/nat.2017.0705. Epub 2018 Mar. 22.
Urtti A. Challenges and obstacles of ocular pharmacokinetics and drug delivery. Adv Drug Deliv Rev 2006; 58 (11): 1131-1135. doi: 10.1016/j.addr.2006.07.027
van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9, 660-666 (1995)].
Vicentini FTMDC et al. Delivery systems and local administration routes for therapeutic siRNA. Pharm Res 2013; 30 (4): 915-931. doi: 10.1007/s11095-013-0971-1.
Wong W. L., Su X., Li X., Cheung C. M., Klein R., Cheng C. Y., Wong T. Y. Global prevalence of age-related macular degeneration and disease burden projection for 2020 and 2040: A systematic review and meta-analysis. Lancet Glob. Health. 2014; 2: e106-e116. doi: 10.1016/S2214-109X (13) 70145-1
Wu, G. Y., Wu, C. H., 1987. Receptor-mediated in vitro gene transformation by a soluble DNA carrier system. J. Biol. Chem. 262 (10), 4429-4432.
Yap T E, Balendra S I, Almonte M T, Cordeiro M F. Retinal correlates of neurological disorders. Ther Adv Chronic Dis. 2019; 10:2040622319882205. doi: 10.1177/204062231988220525.
Zhang X, Vadoothker S, Munir W M, et al. Ocular surface disease and glaucoma medications: a clinical approach. Eye Contact Lens. 2019; 45:11-18)
Zhuang J., Madden D. J., Cunha P., Badea A., Davis S. W., Potter G. G., Lad E. M., Cousins S. W., Chen N.-K., Allen K., et al. Cerebral White Matter Connectivity, Cognition, and Age-Related Macular Degeneration. Neuro Image Clin. 2021; 30:102594. doi: 10.1016/j.nicl.2021.102594

1. A saponin component for use in a therapeutic method of treating a subject suffering from a disorder of a neuron-rich organ comprising vasculature with blood-tissue barrier properties, the method comprising administration to the subject of:
the saponin component comprising a penta-cyclic triterpene saponin comprising an aglycone core of 12,13-dehydrooleanane type, and
an effector component comprising a nucleic acid therapeutic intended to be delivered into one or more cells of the organ,
and wherein the administration is performed directly into the organ or into a body cavity or fluid space that is in communication with the cells of the organ, preferably the organ being an organ derived from neural tube.

2. The saponin component for use according to claim 1, wherein the saponin component further comprises a first ligand recognised by a first endocytic receptor, and/or wherein the effector component further comprises a second ligand recognised by a second endocytic receptor,
possibly wherein the second endocytic receptor is the same as the first endocytic receptor, further possibly wherein the second ligand is the same as the first ligand, alternatively wherein the second endocytic receptor differs from the first endocytic receptor with the proviso that the two different endocytic receptors are both present on the same cell;
preferably wherein the first ligand and/or the second ligand is a proteinaceous ligand, for example a naturally existing peptide or protein ligand or a receptor-interacting part thereof, or is an antibody or a binding fragment thereof.

3. The saponin component for use according to any one of the preceding claims, wherein the penta-cyclic triterpene saponin further comprises
an aldehyde function at position C-23 of the aglycone core, or
an acid-sensitive covalent bond configured to break under acidic conditions so as to create the aldehyde function at position C-23 of the aglycone core, preferably wherein the acid-sensitive covalent bond is selected from any one or more of: a semicarbazone bond, a hydrazone bond, an imine bond, an acetal bond including a 1,3-dioxolane bond, a ketal bond, an ester bond, and/or an oxime bond, and preferably is selected from a semicarbazone bond and a hydrazone bond.

4. The saponin component for use according to any one of the preceding claims, wherein the penta-cyclic triterpene saponin is mono-desmosidic or bi-desmosidic, preferably comprising a first saccharide chain bound to a position C-3 of the aglycone core, more preferably wherein the first saccharide chain is selected from Group A listed in Table 1A, even more preferably wherein the first saccharide chain comprises a glucuronic acid group, preferably a terminal glucuronic acid group, most preferably wherein the first saccharide chain comprises: Gal-(1→2)-[Xyl-(1→3)]-GlcA.

5. The saponin component for use according to any one of the preceding claims, wherein the penta-cyclic triterpene saponin comprises the aglycone core selected from quillaic acid, gypsogenin, and an aldehyde-substituted derivative of either one of quillaic acid or gypsogenin defined as a quillaic acid-based or gypsogenin-based aglycone core, respectively, wherein the aldehyde function at position C-23 is substituted by the acid sensitive covalent bond configured to break under acidic conditions so as to create the aldehyde function at position C-23 of the aglycone core, preferably wherein the penta-cyclic triterpene saponin is selected from:
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21 A-apio, QS-21 A-xylo, QS-21 B-apio and QS-21 B-xylo, or the aldehyde-substituted derivative of any one thereof, respectively; or
wherein the penta-cyclic triterpene saponin is selected from:
SA1641, gypsoside A, NP-017772, NP-017774, NP-017777, NP-017778, NP-018109, NP-017888, NP-017889, NP-018108, SO1658 and Phytolaccagenin, or the aldehyde-substituted derivative of any one thereof, respectively.

6. The saponin component for use according to any one of the preceding claims, wherein the penta-cyclic triterpene saponin is isolated from Saponaria officinalis, and is preferably any one or more of Saponarioside B, SO1542, SO1584, SO1658, SO1674, SO1700, SO1730, SO1772, SO1832, SO1861, SO1862 and SO1904, more preferably any one or more of SO1832, SO1861 and SO1862, even more preferably SO1832 or SO1861, most preferably SO1861.

7. The saponin component for use according to any one of the preceding claims, wherein the saponin component comprises an unconjugated saponin molecule.

8. The saponin component for use according to any one of the preceding claims, wherein the saponin component comprises a saponin moiety that is covalently conjugated with at least one non-saponin moiety; preferably via an acid-sensitive covalent bond that breaks under acidic conditions, more preferably being an acid-sensitive covalent bond at the position C-23 of the aglycone core, even more preferably wherein the acid sensitive covalent bond at the position C-23 of the aglycone core is configured to break under acidic conditions so as to create the aldehyde function at position C-23 of the aglycone core thus resulting in a release of the penta-cyclic triterpene saponin comprising the aldehyde function at the position C-23 of the aglycone core from the non-saponin moiety, even more preferably wherein the acid-sensitive covalent bond is selected from any one or more of: a semicarbazone bond, a hydrazone bond, an imine bond, an acetal bond including a 1,3-dioxolane bond, a ketal bond, an ester bond, and/or an oxime bond, most preferably being selected from a semicarbazone bond and a hydrazone bond,
and/or wherein the saponin moiety is covalently conjugated with the at least one non-saponin moiety by an acid-stable bond, preferably via a glucuronic acid group if said group is present.

9. The saponin component for use according to claim 8, wherein the non-saponin moiety comprises any one or more of:
a linker,
the first ligand of claim 2,
the effector component, and/or
a scaffold molecule,
preferably, wherein the saponin moiety is directly covalently conjugated with the linker,
more preferably wherein the linker comprises or is covalently conjugated to the saponin moiety via the acid sensitive covalent bond, more preferably at the position C-23 of the aglycone core, or via the acid-stable bond, preferably at the glucuronic acid group if said group is present;
even more preferably wherein the linker is further covalently conjugated to the first ligand and/or to the effector component, possibly via the scaffold molecule;
for example wherein the scaffold molecule is a multi-functional linker scaffold molecule or a polymeric scaffold molecule possibly comprising a dendron, such as a poly-amidoamine (PAMAM) dendrimer, or a poly-ethylene glycol, such as any of PEG3-PEG30.

10. The saponin component for use according to any one of the claim 8 or 9, wherein the saponin moiety is covalently conjugated with the non-saponin moiety comprising the effector component, which conjugation results in bringing the saponin component and the effector component together in a conjugate further termed a saponin-effector component,
preferably wherein the saponin-effector component further comprises the linker, more preferably wherein the linker is directly covalently conjugated to the saponin moiety
possibly wherein the saponin-effector component further comprises the first ligand.

11. The saponin component for use according to any one of the preceding claims, wherein the administration comprises provision of the effector component and the saponin component formulated as a single pharmaceutical formulation, or formulated as at least two pharmaceutical formulations that can be administered either simultaneously or sequentially, wherein the first pharmaceutical formulation comprises the saponin component and the second pharmaceutical formulation comprises the effector component;
possibly wherein the administration is further followed after an interval of at least 1 day, preferably at least one week, with a boosting application of the saponin component that is further referred to as a boosting saponin component, wherein the boosting saponin component is provided without the effector component and preferably comprises the unconjugated saponin molecule of claim 7 or the saponin moiety of any one of the claim 8 or 9, preferably wherein the saponin moiety is covalently conjugated with the non-saponin moiety being at least the linker or at least the first ligand or at least the linker and the first ligand; and
preferably wherein the boosting application is performed directly into the organ or into a body cavity or fluid space that is in communication with the cells of the organ, most preferably wherein the boosting application is performed at a site of the administration.

12. The saponin component for use according to claim 11, wherein the administration comprises provision of the single pharmaceutical formulation selected from any one or more of the following:
2-component free-saponin formulation defined as comprising the saponin component consisting of the unconjugated saponin molecule of claim 7, wherein the penta-cyclic triterpene saponin is preferably as defined in claim 3, and wherein the 2-component free-saponin formulation further comprises the effector component that possibly comprises a second ligand recognised by a second endocytic receptor;
2-component linker-saponin formulation defined as comprising the saponin component comprising the saponin moiety of any one of the claim 8 or 9, wherein the saponin moiety is covalently conjugated with the linker; wherein the 2-component linker-saponin formulation further comprises the effector component that possibly comprises a second ligand recognised by a second endocytic receptor;
2-component targeted-saponin formulation defined as comprising the saponin component comprising the saponin moiety of any one of the claim 8 or 9, wherein the saponin moiety is covalently conjugated with the first ligand, and preferably wherein the non-saponin moiety comprises the linker; and wherein the 2-component targeted-saponin formulation further comprises the effector component that possibly comprises the second ligand of claim 2;
1-component formulation defined as comprising the saponin-effector component of claim 10, possibly wherein the saponin-effector component further comprises the first ligand.

13. The saponin component for use according to claim 10, wherein the administration comprises provision of the at least two pharmaceutical formulations comprising a combination of the first pharmaceutical formulation with the second pharmaceutical formulation selected from any one or more of the following:
non-targeted combination defined as comprising
the first pharmaceutical formulation, wherein the saponin component does not comprise a ligand and preferably comprises or consists of the unconjugated saponin molecule of claim 7 and/or the saponin moiety of any one of the claim 8 or 9, wherein the saponin moiety is covalently conjugated with the linker, wherein the penta-cyclic triterpene saponin is preferably as defined in claim 3, and the second pharmaceutical formulation, wherein the effector component does not comprise a ligand;
targeted-effector combination defined as comprising
the first pharmaceutical formulation, wherein the saponin component does not comprise a ligand and preferably comprises or consists of the unconjugated saponin molecule of claim 7 and/or the saponin moiety of any one of the claim 8 or 9, wherein the saponin moiety is covalently conjugated with the linker, wherein the penta-cyclic triterpene saponin is preferably as defined in claim 3, and the second pharmaceutical formulation, wherein the effector component comprises the second ligand of claim 2;
targeted-saponin combination defined as comprising
the first pharmaceutical formulation, wherein the saponin component comprises the saponin moiety of any one of the claim 8 or 9, wherein the saponin moiety is covalently conjugated with the first ligand, and preferably wherein the non-saponin moiety comprises the linker, and
the second pharmaceutical formulation, wherein the effector component possibly comprises the second ligand of claim 2.

14. The saponin component for use according to any one of the preceding claims, wherein the nucleic acid therapeutic is selected from:
a gene therapy therapeutic that is capable of treating or ameliorating the disorder by replacing or restoring the function of an abnormal or non-functional gene implicated in the disorder with a functioning variant or by introduction of a reparation within said gene; or
an oligonucleotide therapeutic defined as a nucleic acid therapeutic that is not longer than 200 nt, preferably has a size of 5-150 nt, more preferably 8-100 nt, most preferably 10-50 nt, preferably wherein the oligonucleotide therapeutic is capable of treating or ameliorating the disorder by modulating the expression of a gene implicated in the disorder.

15. The saponin component for use according to any one of the preceding claims, wherein the nucleic acid therapeutic comprises DNA and/or RNA and/or a synthetic nucleic acid defined as modified equivalent of DNA and/or of RNA and comprising one or more nucleotide analogues and/or backbone modifications, preferably wherein the nucleic acid therapeutic is selected from:
DNA therapeutic, preferably selected from plasmid, mini-circle DNA, CRISPR-gene editing related constructs, DNA aptamer, and/or DNA antisense oligonucleotide (ASO, AON), most preferably being a DNA ASO;
RNA therapeutic, preferably selected from RNA ASO, siRNA, miRNA, RNA miRNA inhibitor (anti-microRNA, anti-miRNA, anti-miR) and/or RNA miRNA inhibitor ASO, RNA aptamer, ribozyme, RNA decoy, short hairpin RNA (shRNA), anti-hairpin-shaped microRNA; most preferably selected from RNA ASO, siRNA, miRNA, and/or RNA aptamer;
mixed DNA/RNA and/or synthetic nucleic acid therapeutic, preferably comprising or consisting of any one of the following DNA-based or RNA-based modifications: phosphoramidate morpholino oligomer (PMO, Morpholino), peptide nucleic acid (PNA), phosphorothioate-modified antisense oligonucleotide (PS-ASO), 2′-O-methyl(2′-OMe) phosphorothioate RNA, 2′-O-methoxyethyl(2′-O-MOE) RNA (2′-O-methoxyethyl-RNA (2′-MOE, MOE)), locked nucleic acid (LNA, bridged nucleic acid, BNA; for example 2′-O,4′-aminoethylene bridged nucleic acid (BNA-NC), BNA-based siRNA, BNA-based antisense oligonucleotide (BNA-ASO), BNA-based anti-microRNA etc.), 2′-deoxy-2′-fluoroarabino nucleic acid (FANA), 3′-fluoro hexitol nucleic acid (FHNA), glycol nucleic acid (GNA), threose nucleic acid (TNA), more preferably wherein the mixed DNA/RNA and/or synthetic nucleic acid therapeutic comprises or consists of a gapmer (mixmer), synthetic gapmer, synthetic CpG oligonucleotide, synthetic RNA decoy, synthetic ASO and/or synthetic anti-microRNA,
more preferably wherein the nucleic acid therapeutic is a mixed DNA/RNA and/or synthetic nucleic acid therapeutic selected from: synthetic ASO, substantially DNA-based synthetic ASO, substantially RNA-based synthetic ASO preferably comprising 2′-MOE modification, substantially DNA-based synthetic aptamer, substantially RNA-based synthetic aptamer, synthetic gapmer, synthetic siRNA, synthetic miRNA, synthetic anti-miRNA and/or synthetic anti-miRNA ASO.

16. The saponin component for use according to any one of the preceding claims, wherein the nucleic acid therapeutic is an oligonucleotide therapeutic, preferably an siRNA therapeutic or an antisense oligonucleotide (ASO) therapeutic, preferably comprising one or more nucleotide analogues and/or backbone modifications, more preferably being a mutation specific therapeutic, for example being a mutation specific ASO comprising one or more nucleotide analogues and/or backbone modifications, possibly designed to silence a gene implicated in the disorder and/or to induce exon skipping.

17. The saponin component for use according to any one of the preceding claims, wherein the nucleic acid therapeutic targets a gene selected from: HTT, LRRK2, SNCA, Parkin gene, PINK1, DJ-1, DRP-1,SCN1A, SOD1, TDP-43, FUS, C9orf72, NEK1, UBQLN2, ATXN2, SMN2, SMN1, MAPT (tau gene), APP (amyloid precursor protein gene), BACE1, IL-4, IL-6, IL-7, IL-12RB2, IL-1R1, MBP, MIR29B, AR, FAS, C2orf72 UBE3A, UBE2A, GFAP, DMD, DYN2, DGAT2, MFSD8 (CLN7), TTR, VEGF e.g. VEGF-A, VEGFR1, VEGFR2, RHO, NF2, CMV virus IE2, CEP290, USH2A, CASP2, TRPV1, RPGR, ITGA4, PCED, USH2A, GJA1, C5, OPA1, TGFB2, RTP801, ADRB2, COCH, VEGF-165, P2RX7, JUN, BAX, APAF1, IKBKB, RDS, GUCY1A1, GUCY1A2, CNG (e.g. CNGA1, CNGA2, CNGA3, CNGB1, CNGB3), DDIT4, HIF1A, FN1, CTGF, TXNIP, CYP4B1, CNR1 and CNR2, STAT3, KRAS, TGFB2, MIR21, BCL2, TP53, FOXP3, GRB2, ADRB2, PTGS2/TGFB1, CEBPA, Malat1, AHA1, and MMP14,
preferably wherein the gene is any one of the following genes: HTT, SOD1, MFSD8 (CLN7), SMN1, SMN2, TTR, Malat1, AHA1, or MMP14.

18. The saponin component for use according to any one of the preceding claims, wherein the nucleic acid therapeutic is an oligonucleotide therapeutic, that is preferably capable of silencing a gene or disabling a gene product, more preferably wherein the oligonucleotide therapeutic is selected from the group consisting of: nusinersen (ASO for SMN2 splicing in SMA); inotersen (ASO against TTR in hATTR), eplontersen (ASO against TTR in hATTR), vutrisiran (siRNA against TTR in hATTR), patisiran (siRNA against TTR in hATTR), tofersen (ASO against SOD1 in ALS), QRX-704 (ASO against HTT), jacifusen (ION-363; ASO against FUS); tominersen (IONIS-HTTRx or RG6042; ASO against HTT), WVE-003, (ASO against HTT); zilganersen (ASO against GFAP in Alexander disease); atesidorsen, cimdelirsen (ASOs against GHR in acromegaly), ATL-1102 (ASO against CD49d in relapsing forms of MS); BIIB-080 (ASO against TAU/MAPT in Alzheimer's disease, frontotemporal degeneration, AD dementia); GTX-102 (ASO against UBE2A); ION-464 (ASO against SNCA), ION-541 (ASO against ATXN2); ION-859 (ASO against LRRK2), IONIS-PKKRx (ASO against KLKB1), STK-001 (ASO for splicing SCN1A), WVE-004 (ASO against C9orf72), trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma); lademirsen (anti-miR-21), fomivirsen (ASO against CMV virus IE2), pegatinib (aptamer that binds and blocks VEGF), bevasiranib (siRNA against VEGF-A), siRNA-027 (siRNA against VEGFR-1), aganirsen (ASO against IRS1), sepofarsen (ASO for CEP290 splicing), lufepirsen (CODA-001; ASO against, connexin 43 (GJA1)), IONIS-FB-LRx (ASO against CFB), QR-1123 (ASO against RHO), ultevursen (QR-421a; ASO for USH2A), QPI-1007 (siRNA against in NAION), tivanisiran (siRNA against TRPV1); and bamosiran (siRNA against ADRB2).

19. The saponin component for use according to any one of the claims 2 to 18, wherein the first endocytic receptor and/or the second endocytic receptor is selected from
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR,
Tropomyosin receptor kinase A (TrkA) receptor (NGF receptor)
IL13-R (interleukin-13 receptor)
AMPAR/NMDAR (AMPA- and NMDA-type glutamate receptors)
vascular endothelial growth factor receptor 1 or 2 (VEGFR1 or VEGFR2)
STRA6 (Retinol-binding protein (RBP) receptor).

20. The saponin component for use according to any one of the claims 2 to 19, wherein the first ligand and/or the second ligand is selected from:
antibody or a binding fragment thereof binding to any one of the receptors listed in claim 19;
natural ligand or a fragment thereof recognised by any one of the receptors listed in claim 19;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFβ or a fragment thereof;
EGF or a fragment thereof;
neurotrophin (nerve growth factor, NGF) or fragment thereof;
Interleukin 13 (IL-13) or a fragment thereof;
glutamate or multiple units thereof;
vascular endothelial growth factor A (VEGF-A) or a fragment thereof;
retinol (vitamin A) or other forms of vitamin A;
retinol-binding protein (RBP) or a fragment thereof;
antibody or a binding fragment thereof binding to an endocytic receptor selected from: CD71, CD63, IGF1R, GLUT4, CI-MPR, LDL receptor;
more preferably wherein the first ligand and/or the second ligand is an antibody or a binding fragment thereof binding to CD71, even more preferably being a monoclonal or a single domain antibody binding to CD71, most preferably being a monoclonal antibody binding to CD71.

21. The saponin component for use according to any one of claims 1-20, wherein the organ is part of the central nervous system (CNS), preferably is the brain.

22. The saponin component for use according to claim 21, wherein the administration is selected from epidural, intrathecal, intracerebroventricular, intracisternal, intraparenchymal, intranasal and/or comprises a postoperative injection to the intratumoural cavity formed after surgery within the CNS;
preferably wherein the administration is selected from intrathecal, intracerebroventricular, intracisternal, and/or intranasal;
more preferably wherein the administration is intrathecal.

23. The saponin component for use according to claim 21, wherein the administration is made into the dura mater, or into the arachnoid mater, or into the subarachnoid space, or into the pia mater, and/or into the brain tissue; preferably wherein the administration is made into the arachnoid mater and/or into the subarachnoid space; more preferably wherein the administration is made into the subarachnoid space.

24. The saponin component for use according to any one of claims 21-23, wherein the CNS disorder is selected from:
a neurodegenerative disorder, preferably selected from any one or more of Huntington's disease (HD), Alzheimer's disease (AD), Parkinson's disease (PD), amyotrophic lateral sclerosis (ALS), spinal muscular atrophy (SMA), multiple system atrophy (MSA), multiple sclerosis (MS), and/or dementia with Lewy body (DLB);
neurological disorder, preferably selected from stroke, epilepsy such as Dravet syndrome (DS), and/or a spinal cord disease;
an oncological disorder, preferably selected from any one or more of glioblastoma, meningioma, (oligodendro) glioma, astrocytoma, ependymoma, medulloblastoma, CNS lymphoma, metastasis to the CNS; more preferably selected from glioblastoma, meningioma, (oligodendro) glioma, and/or metastasis to the CNS;
immune disorder, preferably selected from an autoimmune disease of the CNS, an immunity-related disease caused by a gene defect, a disease caused by an infection or inflammation, more preferably selected from meningitis, encephalitis, prion disease, and/or coronavirus disease 2019 (COVID-19);
a psychiatric disorder, preferably selected from any one or more of Tourette syndrome (TS), mood disorder, personality disorder, anxiety disorder, substance use or addictive disorder, obsessive-compulsive disorder, neurodevelopmental disorder, eating disorder; more preferably is selected from an anxiety disorder, obsessive-compulsive disorder, eating disorder, and/or a mood disorder preferably being a treatment-refractory mood disorder.

25. The saponin component for use according to any one of claims 21-24, wherein the CNS disorder is selected from: spinal muscular atrophy, hereditary transthyretin amyloidosis (hATTR), amyotrophic lateral sclerosis (ALS) preferably being SOD1-associated amyotrophic lateral sclerosis, Huntington's disease, Alzheimer's disease, Parkinson's disease, Batten disease, frontotemporal dementia, pinocerebellar ataxia type 3, multiple system atrophy; Rett syndrome, Alexander disease; Angelman syndrome; Lafora disease; GFAP astrocytopathy, a prion disease, and a neurological disorders related to acromegaly.

26. The saponin component for use according to any one of claims 21-25, wherein the nucleic acid therapeutic is an oligonucleotide therapeutic selected from the group consisting of: nusinersen (ASO for SMN2 splicing in SMA); inotersen (ASO against TTR in hATTR), eplontersen (ASO against TTR in hATTR), vutrisiran (siRNA against TTR in hATTR), patisiran (siRNA against TTR in hATTR), tofersen (ASO against SOD1 in ALS), jacifusen (ION-363; ASO against FUS); tominersen (IONIS-HTTRx or RG6042; ASOs against HTT), QRX-704 (ASO against HTT), WVE-003, (ASO against HTT); zilganersen (ASO against GFAP in Alexander disease); atesidorsen, cimdelirsen (ASOs against GHR in acromegaly), ATL-1102 (ASO against CD49d in relapsing forms of MS); BIIB-080 (ASO against TAU/MAPT in Alzheimer's disease, frontotemporal degeneration, AD dementia); GTX-102 (ASO against UBE2A); ION-464 (ASO against SNCA), ION-541 (ASO against ATXN2); ION-859 (ASO against LRRK2), IONIS-PKKRx (ASO against KLKB1), STK-001 (ASO for splicing SCN1A), WVE-004 (ASO against C9orf72), trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma), lademirsen (anti-miR-21).

27. The saponin component for use according to any one of claims 21-26, comprising the first ligand and/or the second ligand of claim 2,
wherein the first endocytic receptor and/or the second endocytic receptor is present on the cells and/or tissue within the CNS, preferably wherein the cells are selected from any one of more of neurons, astrocytes, oligodendrocytes, microglia, endothelial cells, blood cells, and/or tumour cells, more preferably wherein the cells are selected from any one of more of neurons, astrocytes, oligodendrocytes, microglia, endothelial cells, and/or tumour cells;
most preferably wherein the first endocytic receptor and/or the second endocytic receptor is selected from:
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR
Tropomyosin receptor kinase A (TrkA) receptor (NGF receptor)
IL13-R (interleukin-13 receptor)
AMPAR/NMDAR (AMPA- and NMDA-type glutamate receptors).

28. The saponin component for use according to any one of claims 21-27, comprising the first ligand and/or the second ligand of claim 2,
wherein the first ligand and/or the second ligand is selected from:
antibody or a binding fragment thereof binding to any one of the receptors listed in claim 27;
natural ligand or a fragment thereof recognised by any one of the receptors listed in claim 27;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFβ or a fragment thereof;
EGF or a fragment thereof;
neurotrophin (nerve growth factor, NGF) or fragment thereof;
Interleukin 13 (IL-13) or a fragment thereof;
glutamate or multiple units thereof;
antibody or a binding fragment thereof binding to an endocytic receptor selected from: CD71, CD63, IGF1R, GLUT4, CI-MPR, LDL receptor;
more preferably wherein the first ligand and/or the second ligand is an antibody or a binding fragment thereof binding to CD71, even preferably being a monoclonal or a single domain antibody binding to CD71, most preferably being a monoclonal antibody binding to CD71.

29. The saponin component for use according to any one of claims 21-28, wherein the effector component comprises an oligonucleotide therapeutic targeting any one of STAT3, SOD1, Malat1, AHA1, MMP14, TTR, and HTT, or is an oligonucleotide therapeutic selected from nusinersen, tominersen, tofersen, inotersen, eplontersen, vutrisiran, patisiran, and trabedersen; and
wherein the saponin component comprises the penta-cyclic triterpene saponin as defined in claim 3, preferably as defined in claim 5, more preferably being SO1861 or SO1861 wherein the aldehyde function at position C-23 is substituted by the acid sensitive covalent bond configured to break under acidic conditions so as to create the aldehyde function at position C-23 of the aglycone core.

30. The saponin component for use according to claim 29,
wherein the administration is intrathecal, and preferably comprises the 2-component free saponin formulation or the 2-component linker-saponin formulation or 1-component formulation as defined in claim 12.

31. The saponin component for use according to any one of claims 1-20, wherein the organ is the eye and the disorder is further referred to as the ocular disorder.

32. The saponin component for use according to claim 31, wherein the administration is intraocular; preferably selected from intrascleral, suprachoroidal, subretinal, intracameral, intravitreal, and vitreoretinal; more preferably wherein the administration comprises any one selected from intracameral injection, intracameral implant, intravitreal injection, subretinal injection, intravitreal implant, and a scleral plug; even more preferably wherein the administration is intravitreal, even more preferably wherein the administration comprises intravitreal injection or an intravitreal implant, most preferably wherein the administration comprises intravitreal injection; or wherein the local administration is periocular and preferably is subconjunctival, more preferably comprises subconjunctival injection or a subconjunctival implant.

33. The saponin component for use according to any one of the claims 31-32, wherein the ocular disorder is a disorder of the posterior segment of the eye; preferably being the disorder of the retina, of the choroid, of the optic nerve, or the vitreous body; more preferably wherein the disorder is selected from one or more of glaucoma, posterior uveitis, posterior scleritis, retinitis, wet- or dry-age related macular degeneration (AMD), geographic atrophy, diabetic retinopathy, diabetic- or non-diabetic macular edema, choroidal neovascularization, retinoblastoma, and a congenital disorder of any one of the retina, of the choroid, of the optic nerve, or the vitreous body; even more preferably being a congenital disorder of the retina selected from any one of retinitis pigmentosa (RP), Leber congenital amaurosis (LCA), USH2A retinopathy, and neurofibromatosis type 2 (NFT2), or
wherein the ocular disorder is a disorder of the anterior segment of the eye; preferably wherein the disorder is selected from one or more of anterior uveitis, iritis, blepharitis, conjunctivitis, blepharoconjuctivitis, keratitis, anterior scleritis, episcleritis, dry eye disease, cataract, corneal abrasion, corneal neovascularization, and a trauma of the anterior segment or a part thereof,

34. The saponin component for use according to any one of the claims 31-33, wherein the ocular disorder is selected from: cytomegalovirus retinitis infection; age related macular degeneration (AMD); autosomal dominant retinitis pigmentosa; Leber's hereditary optic neuropathy; Stargardt disease; Usher syndrome; ocular disorders related to acromegaly, or ocular disorders related to myotonic dystrophy.

35. The saponin component for use according to any one of the claims 31-34, wherein the nucleic acid therapeutic targets a gene selected from VEGF e.g. VEGF-A, VEGFR1, VEGFR2, RHO, NF2, CMV virus IE2, CEP290, USH2A, CASP2, TRPV1, RPGR, ITGA4, PCED, USH2A, GJA1, C5, OPA1, TGFB2, RTP801, TTR, MAPT (tau gene), APP (amyloid precursor protein gene), BACE1, IL-4, IL-6, IL-7, AR, FAS, ADRB2, COCH, VEGF-165, P2RX7, JUN, BAX, APAF1, IKBKB, RDS, GUCY1A1, GUCY1A2, CNG (e.g. CNGA1, CNGA2, CNGA3, CNGB1, CNGB3), DDIT4, HIF1A, FN1, CTGF, TXNIP, CYP4B1, CNR1 and CNR2, STAT3, KRAS, TGFB2, MIR21, BCL2, TP53, FOXP3, GRB2, ADRB2, PTGS2/TGFB1, CEBPA, Malat1, AHA1, and MMP14.

36. The saponin component for use according to any one of the claims 31-35, wherein the nucleic acid therapeutic is an oligonucleotide therapeutic selected from the group consisting of:
fomivirsen (ASO against CMV virus IE2), pegatinib (aptamer that binds and blocks VEGF), bevasiranib (siRNA against VEGF-A), siRNA-027 (siRNA against VEGFR-1), aganirsen (ASO against IRS1), sepofarsen (ASO for CEP290 splicing), Lufepirsen (CODA-001; ASO against, connexin 43 (GJA1)), IONIS-FB-LRx (ASO against CFB), QR-1123 (ASO against RHO), ultevursen (QR-421a; ASO for USH2A), QPI-1007 (siRNA against in NAION), tivanisiran (siRNA against TRPV1); and bamosiran (siRNA against ADRB2), trabedersen (ASO against TGFB2), ISTH-0036 (ASO against TGFB2), STP-705 (siRNA against PTGS2/TGFB1), danvatirsen (ASO against STAT3), AZD-8701 (ASO against FOXP3); siG-12D-LODER (siRNA against KRAS), IONISAR-2.5Rx (ASO against AR), SR-063 (siRNA against AR), prexigebersen (ASO against GRB2); MTL-CEBPA (saRNA for activation CEBPA), oblimersen (ASO against Bcl-2 in melanoma), Lademirsen (anti-miR-21), inotersen (ASO against TTR in hATTR), eplontersen (ASO against TTR in hATTR), vutrisiran (siRNA against TTR in hATTR), patisiran (siRNA against TTR in hATTR), atesidorsen and cimdelirsen (ASOs against GHR in acromegaly),
or is an oligonucleotide therapeutic designed to reduce or inhibit the expression of VEGF, preferably VEGF-A, or one of its receptors, preferably selected from VEGFR1 or VEGFR2;
or is an oligonucleotide therapeutic designed to induce exon skipping, preferably of the human RPGR gene or SH2A gene or NF2 gene.

37. The saponin component for use according to any one of the claims 31-36, comprising the first ligand and/or the second ligand of claim 2,
wherein the first endocytic receptor and/or the second endocytic receptor is present on the cells and/or tissue within the eye, preferably wherein the cells are cells of the retina or cells of the retinal blood vessels;
most preferably wherein the first endocytic receptor and/or the second endocytic receptor is selected from:
CD71 (transferrin receptor)
CD63 (tetraspanin)
IGF1R (insulin-like growth factor 1 (IGF-I) receptor)
InsR (insulin receptor)
GLUT4 (glucose transporter),
CI-MPR (cation independent mannose 6 phosphate receptor),
LDL receptor
TGFβ receptor;
EGFR
vascular endothelial growth factor receptor 1 or 2 (VEGFR1 or VEGFR2)
STRA6 (Retinol-binding protein (RBP) receptor).

38. The saponin component for use according to any one of claims 31-37, comprising the first ligand and/or the second ligand of claim 2,
wherein the first ligand and/or the second ligand is selected from:
antibody or a binding fragment thereof binding to any one of the receptors listed in claim 35;
natural ligand or a fragment thereof recognised by any one of the receptors listed in claim 35;
preferably wherein the first ligand and/or the second ligand is selected from:
transferrin (Tf) or a fragment thereof recognised by CD71;
insulin or a fragment thereof;
insulin-like growth factor 1 (IGF-I) or a fragment thereof;
insulin-like growth factor 2 (IGF-II) or a fragment thereof;
mannose 6 phosphate, preferably multiple units thereof;
glucose, preferably multiple units thereof, for example zymosan A;
TGFβ or a fragment thereof;
EGF or a fragment thereof;
vascular endothelial growth factor A (VEGF-A) or a fragment thereof;
retinol (vitamin A) or other forms of vitamin A;
retinol-binding protein (RBP) or a fragment thereof;
antibody or a binding fragment thereof binding to an endocytic receptor selected from: CD71, CD63, IGF1R, GLUT4, CI-MPR, LDL receptor, VEGFR1, VEGFR2, and STRA6;
more preferably wherein the first ligand and/or the second ligand is an antibody or a binding fragment thereof binding to CD71, even preferably being a monoclonal or a single domain antibody binding to CD71, most preferably being a monoclonal antibody binding to CD71.

39. The saponin component according to any one of claims 31-38, wherein the effector component comprises an oligonucleotide therapeutic targeting any one of STS3, SOD1, Malat1, AHA1, MMP14, TTR, and HTT, or is an oligonucleotide therapeutic selected from, fomivirsen, pegatinib, inotersen, eplontersen, vutrisiran, patisiran, sepofarsen, QR-421a, ultevursen, tivanisiran, and QPI-1007; and wherein the saponin component comprises the penta-cyclic triterpene saponin as defined in claim 3, preferably as defined in claim 5, more preferably being SO1861 or SO1861 wherein the aldehyde function at position C-23 is substituted by the acid sensitive covalent bond configured to break under acidic conditions so as to create the aldehyde function at position C-23 of the aglycone core.

40. The saponin component for use according to claim 39, wherein the administration is intravitreal, and preferably comprises the 2-component free saponin formulation or the 2-component linker-saponin formulation or 1-component formulation as defined in claim 12.


SEQUENCE LISTING (58 sequences)

SEQ ID NO: 1 - RNA, 21 nt, synthetic construct
  source 1..21: mol_type = other RNA; note = GN3-siTTR (ds, sense strand)
aacagtgttcttgctctataa

SEQ ID NO: 2 - DNA, 20 bp, synthetic construct
  source 1..20: mol_type = other DNA; note = Ttr Forward primer
tttcacagccaacgactctg

SEQ ID NO: 3 - DNA, 20 bp, synthetic construct
  source 1..20: mol_type = other DNA; note = Ttr Reverse primer
agaatgcttcacggcatctt

SEQ ID NO: 4 - DNA, 18 bp, synthetic construct
  source 1..18: mol_type = other DNA; note = Ppia Forward primer
gcggcaggtccatctacg

SEQ ID NO: 5 - DNA, 19 bp, synthetic construct
  source 1..19: mol_type = other DNA; note = Ppia Reverse primer
gccatccagccattcagtc

SEQ ID NO: 6 - DNA, 20 bp, synthetic construct
  source 1..20: mol_type = other DNA; note = Sdha Forward primer
gaggaagcacaccctctcat

SEQ ID NO: 7 - DNA, 20 bp, synthetic construct
  source 1..20: mol_type = other DNA; note = Sdha Reverse primer
ggagcggatagcaggaggta

SEQ ID NO: 8 - DNA, 16 bp, synthetic construct
  source 1..16: mol_type = other DNA; note = STAT3 ASO
  misc_feature 1..3: note = RNA [RNA-mimic 5'flanking region]
  misc_feature 4..12: note = DNA [synthetic-DNA center]
  misc_feature 13..16: note = RNA [RNA-mimic 3'flanking region]
ctatttggatgtcagc

SEQ ID NO: 9 - DNA, 24 bp, synthetic construct
  source 1..24: mol_type = other DNA; note = Hs STAT3 Forward primer (qPCR)
acatgccactttggtgtttcataa

SEQ ID NO: 10 - DNA, 27 bp, synthetic construct
  source 1..27: mol_type = other DNA; note = Hs STAT3 Reverse primer (qPCR)
tcttcgtagattgtgctgatagagaac

SEQ ID NO: 11 - DNA, 20 bp, synthetic construct
  source 1..20: mol_type = other DNA; note = HBMS Forward primer
cacccacacacagcctactt

SEQ ID NO: 12 - DNA, 20 bp, synthetic construct
  source 1..20: mol_type = other DNA; note = HBMS Reverse primer
gtacccacgcgaatcactct

SEQ ID NO: 13 - DNA, 20 bp, synthetic construct
  source 1..20: mol_type = other DNA; note = SDHA Forward primer
gcatttggcctttctgaggc

SEQ ID NO: 14 - DNA, 20 bp, synthetic construct
  source 1..20: mol_type = other DNA; note = SDHA Reverse primer
ctccatgttccccagagcag

SEQ ID NO: 15 - RNA, 23 nt, synthetic construct
  source 1..23: mol_type = other RNA; note = GN3-siTTR (ds, antisense strand)
ttatagagcaagaacactgtttt

SEQ ID NO: 16 - DNA, 20 bp, synthetic construct
  source 1..20: mol_type = other DNA; note = Malat1 ASO
  misc_feature 1..5: note = RNA [RNA-mimic 5'flanking region]
  misc_feature 6..15: note = DNA [synthetic-DNA center]
  misc_feature 16..20: note = RNA [RNA-mimic 3'flanking region]
gccaggctggttatgactca

SEQ ID NO: 17 - DNA, 17 bp, synthetic construct
  source 1..17: mol_type = other DNA; note = Mm Malat1 Forward primer
aggcgggcagctaagga

SEQ ID NO: 18 - DNA, 21 bp, synthetic construct
  source 1..21: mol_type = other DNA; note = Mm Malat1 Reverse primer
ccccactgtagcatcacatca

SEQ ID NO: 19 - RNA, 16 nt, synthetic construct
  source 1..16: mol_type = other RNA; note = HTRA LNA
tatttacctggttgtt

SEQ ID NO: 20 - DNA, 25 bp, synthetic construct
  source 1..25: mol_type = other DNA; note = SOD1 PMO
  modified_base 1..25: mod_base = OTHER; note = phosphorodiamidate morpholino oligomer
gccagcctaggacctaccttgtgta

SEQ ID NO: 21 - DNA, 20 bp, synthetic construct
  source 1..20: mol_type = other DNA; note = Sod1 Forward primer
cgtcttgctctctctggtcc

SEQ ID NO: 22 - DNA, 20 bp, synthetic construct
  source 1..20: mol_type = other DNA; note = Sod1 Reverse primer
agactcagaccacacaggga

SEQ ID NO: 23 - DNA, 25 bp, synthetic construct
  source 1..25: mol_type = other DNA; note = SOD1 PMO (2)
  modified_base 1..25: mod_base = OTHER; note = phosphorodiamidate morpholino oligomer
agcctatttaccagaaaccagcagt

SEQ ID NO: 24 - DNA, 22 bp, synthetic construct
  source 1..22: mol_type = other DNA; note = Mm Stat3 Forward primer (qPCR)
gggcatttttatggctttcaat

SEQ ID NO: 25 - DNA, 23 bp, synthetic construct
  source 1..23: mol_type = other DNA; note = Mm Stat3 Reverse primer (qPCR)
gttaacccaggcacacagacttc

SEQ ID NO: 26 - DNA, 24 bp, synthetic construct
  source 1..24: mol_type = other DNA; note = Hs STAT3 Forward primer (a/b)
tgctaccaatatcctggtgtctcc

SEQ ID NO: 27 - DNA, 24 bp, synthetic construct
  source 1..24: mol_type = other DNA; note = Hs STAT3 Reverse primer (a/b)
actccgaggtcaactccatgtcaa

SEQ ID NO: 28 - DNA, 22 bp, synthetic construct
  source 1..22: mol_type = other DNA; note = BGUS Forward primer
gaaaatacgtggttggagagct

SEQ ID NO: 29 - DNA, 22 bp, synthetic construct
  source 1..22: mol_type = other DNA; note = BGUS Reverse primer
ccgagtgaagatccccttttta

SEQ ID NO: 30 - DNA, 21 bp, synthetic construct
  source 1..21: mol_type = other DNA; note = HPRT Forward primer
cctggcgtcgtgattagtgat

SEQ ID NO: 31 - DNA, 22 bp, synthetic construct
  source 1..22: mol_type = other DNA; note = HPRT Reverse primer
agacgttcagtcctgtccataa

SEQ ID NO: 32 - DNA, 22 bp, synthetic construct
  source 1..22: mol_type = other DNA; note = AHA1 Forward primer
ctttgccaccatcaccttgacc

SEQ ID NO: 33 - DNA, 22 bp, synthetic construct
  source 1..22: mol_type = other DNA; note = AHA1 Reverse primer
aaagtagtaccgctgccagccc

SEQ ID NO: 34 - DNA, 20 bp, synthetic construct
  source 1..20: mol_type = other DNA; note = MMP14 Forward primer
gagcattccagtgacccctc

SEQ ID NO: 35 - DNA, 20 bp, synthetic construct
  source 1..20: mol_type = other DNA; note = MMP14 Reverse primer
accctgactcacccccataa

SEQ ID NO: 36 - DNA, 23 bp, synthetic construct
  source 1..23: mol_type = other DNA; note = STAT3-ST6 PMO
  modified_base 1..23: mod_base = OTHER; note = phosphorodiamidate morpholino oligomer
cattttctgttctagatcctgtt

SEQ ID NO: 37 - DNA, 23 bp, synthetic construct
  source 1..23: mol_type = other DNA; note = STAT3-ST2 PMO
  modified_base 1..23: mod_base = OTHER; note = phosphorodiamidate morpholino oligomer
attgctgcaggtcgttctgtagg

SEQ ID NO: 38 - DNA, 21 bp, synthetic construct
  source 1..21: mol_type = other DNA; note = AHA siRNA (ds, sense strand)
ggatgaagtggagattagttt

SEQ ID NO: 39 - DNA, 23 bp, synthetic construct
  source 1..23: mol_type = other DNA; note = MMP14 siRNA - (ds, sense strand)
aaccagaagctgaaggtagaatt

SEQ ID NO: 40 - RNA, 21 nt, synthetic construct
  source 1..21: mol_type = other RNA; note = AHA siRNA (ds, antisense strand)
actaatctccacttcatcctt

SEQ ID NO: 41 - RNA, 23 nt, synthetic construct
  source 1..23: mol_type = other RNA; note = MMP14 siRNA - (ds, antisense strand)
ttctaccttcagcttctggtttt

SEQ ID NO: 42 - DNA, 20 bp, synthetic construct
  source 1..20: mol_type = other DNA; note = Hs IPO8 Forward primer
cagcagcccagactctctta

SEQ ID NO: 43 - DNA, 20 bp, synthetic construct
  source 1..20: mol_type = other DNA; note = Hs IPO8 Reverse primer
cctagggtcaaagttcgggt

SEQ ID NO: 44 - DNA, 21 bp, synthetic construct
  source 1..21: mol_type = other DNA; note = Hs MALAT1 Forward primer
aaagcaaggtctccccacaag

SEQ ID NO: 45 - DNA, 22 bp, synthetic construct
  source 1..22: mol_type = other DNA; note = Hs MALAT1 Reverse primer
ggtctgtgctagatcaaaaggc

SEQ ID NO: 46 - DNA, 20 bp, synthetic construct
  source 1..20: mol_type = other DNA; note = Hs SOD1 (qPCR) Forward primer
agcagatgacttgggcaaag

SEQ ID NO: 47 - DNA, 20 bp, synthetic construct
  source 1..20: mol_type = other DNA; note = Hs SOD1 (qPCR) Reverse primer
accacaagccaaacgacttc

SEQ ID NO: 48 - DNA, 20 bp, synthetic construct
  source 1..20: mol_type = other DNA; note = Hs SOD1 (skip) Forward primer
cgacgaaggccgtgtgcgtg

SEQ ID NO: 49 - DNA, 22 bp, synthetic construct
  source 1..22: mol_type = other DNA; note = Hs SOD1 (skip) Reverse primer
agttaaggggcctcagactaca

SEQ ID NO: 50 - DNA, 20 bp, synthetic construct
  misc_feature 6..15: note = DNA synthetic-DNA center
  misc_feature 1..5: note = RNA mimic 5'flanking region
  misc_feature 16..20: note = RNA mimic 3'flanking region
  source 1..20: mol_type = other DNA; note = ASO gapmer against Hs SOD1 mRNA
caggatacatttctacagct

SEQ ID NO: 51 - DNA, 25 bp, synthetic construct
  source 1..25: mol_type = other DNA; note = PMO (1) targeting Hs SOD1
  modified_base 1..25: mod_base = OTHER; note = phosphorodiamidate morpholino oligomer
aacacccacctgctgtattatctcc

SEQ ID NO: 52 - DNA, 25 bp, synthetic construct
  source 1..25: mol_type = other DNA; note = PMO (2) targeting Hs SOD1
  modified_base 1..25: mod_base = OTHER; note = phosphorodiamidate morpholino oligomer
atcctttggcccaccgtgttttctg

SEQ ID NO: 53 - DNA, 20 bp, synthetic construct
  source 1..20: mol_type = other DNA; note = Mm Ppih Forward primer
aatcgagctctttgcagacg

SEQ ID NO: 54 - DNA, 20 bp, synthetic construct
  source 1..20: mol_type = other DNA; note = Mm Ppih Reverse primer
tatcctatcggaacgccatc

SEQ ID NO: 55 - DNA, 20 bp, synthetic construct
  source 1..20: mol_type = other DNA; note = Mm Ndufa2 Forward primer
ttcgcgttcacttatgccag

SEQ ID NO: 56 - DNA, 20 bp, synthetic construct
  source 1..20: mol_type = other DNA; note = Mm Ndufa2 Reverse primer
gccttcttcagctccacgta

SEQ ID NO: 57 - DNA, 20 bp, synthetic construct
  source 1..20: mol_type = other DNA; note = Mm Sod1 (qPCR) Forward primer
ggaaccatccacttcgagca

SEQ ID NO: 58 - DNA, 22 bp, synthetic construct
  source 1..22: mol_type = other DNA; note = Mm Sod1 (qPCR) Reverse primer
ggtacagccttgtgtattgtcc